(19) United States
(12) Patent Application Publication
(10) Pub. No.: US 2024/0124875 A1
(43) Pub. Date: Apr. 18, 2024

(54) RNAI CONJUGATES AND USES THEREOF

(71) Applicant: Dicerna Pharmaceuticals, Inc., Lexington, MA (US)

(72) Inventors: Shanthi GANESH, Shrewsbury, MA (US); Marc ABRAMS, Natick, MA (US); Henryk T. DUDEK, Belmont, MA (US); Harini Sivagurunatha KRISHNAN, Lexington, MA (US)

(21) Appl. No.: 18/280,092

(22) PCT Filed: Mar. 4, 2022
(86) PCT No.: PCT/US2022/018911

§ 371 Date: Sep. 1, 2023

Related U.S. Application Data
(60) Provisional application No. 63/214,153, filed on Jun. 23, 2021.
(60) Provisional application No. 63/157,465, filed on Mar. 5, 2021.

(51) Int. Cl. C12N 15/113; A61K 31/713; A61K 47/54; A61P 35/00

(52) U.S. Cl. CPC C12N 15/113; A61K 31/713; A61K 47/542; A61P 35/00

(57) ABSTRACT

The subject matter disclosed herein is directed to modulating gene expression using siRNA compositions and methods directed to affecting key cell populations supporting the growth and metastasis of cancer to affect the beneficial treatment, remission or removal of the underlying tumor in a patient.

### CROSS-RELATED APPLICATIONS

This application claims the benefit of U.S. Provisional Patent Application Ser. No. 63/157,465 filed Mar. 5, 2021, and U.S. Provisional Patent Application Ser. No. 63/214,153, filed Jun. 23, 2021. The entire contents of which is incorporated herein by this reference.

### TECHNICAL FIELD

The disclosure relates to oligonucleotides or oligonucleotides linked to targeting moieties useful in the inhibition, remission, and/or controlling of cancer in patients. In certain embodiments, the disclosure relates to methods of administering to subjects in need thereof a therapeutically effective amount of one or more RNAi oligonucleotides, or one or more RNAi molecules, that inhibit signal transducer and activator of transcription 3 (“STAT3”) expression in a subject.

### BACKGROUND OF THE DISCLOSURE

The growth and progression of cancer is influenced by many factors including the tumor microenvironment (“TME”) which contains components which may control, influence, or enhance tumor development, including blood vessels, immune cells, fibroblasts, bone marrow-derived inflammatory cells, signaling molecules and the extracellular matrix (Yin et al., INT J. CANCER (2019) 144(5):933-46). Despite the existing heterogeneity of various tumors, the development of a tumor is highly dependent upon the physiological state of the TME. Although tumors may come from a variety of anatomical locations and/or cell populations the tumor itself will have many common features that can be used to derive treatment protocols for the tumor. This is particularly true for the TME maturation of epithelial-derived tumors. Genetic alterations in tumor cells result in hyperplasia, uncontrolled growth, resistance to apoptosis, and a metabolic shift towards anaerobic glycolysis (the so-called “Warburg Effect”). These events create hypoxia, oxidative stress, and acidosis within the TME triggering an adjustment of the extracellular matrix (ECM) surrounding the altered or cancerous cells, a response from neighboring stromal cells (e.g., fibroblasts) and immune cells (lymphocytes and macrophages), inducing angiogenesis and, ultimately, resulting in metastasis. The TME profile itself also directly impacts the efficacy of anti-cancer therapies (Giraldo et al., BR. J. CANCER (2019) 120: 45-53).

Currently, chemotherapy is the leading cancer therapy worldwide, often combined with surgery, or surgery and radiotherapy, depending on tumor type and stage (Abbas et al., AN OVERVIEW OF CANCER TREATMENT MODALITIES/INTECHOPEN, 2018). Since the discovery of several important mutations that contribute to carcinogenesis (e.g., epidermal cell alterations (Yamaoka et al., INT. J. MOL. SCI. (2017) 18(11): 2420)) these mutations and the proteins they represent have been extensively used as targets for the development of more selective drugs and drug combinations to treat cancer patients. Despite the effectiveness of these drugs, multidrug resistance (MDR) is often seen in patients, which often results in tumor relapse, limited therapeutic options and low quality of life for patients. In addition, cancer research has often been focused on tumor cells even though the effect of the TME and the ‘normal’ or non-cancerous cells within it that have been shown to play a key role in tumor progression, development and MDR (Klemm et al., TRENDS CELL BIOL (2015) 25(4): 198-213).

At a late stage in development for a solid tumor, the tumor microenvironment is highly complex and heterogeneous (Runa et al., CURR MOL BIOL REP (2017) 3(4): 218-29). The interplay between cancer cells and neighboring cells, including stromal and immune system cells (which frequently appear due to inflammation at the tumor location) results in additional alterations in the TME as well as cellular components, the extracellular matrix, and the formation of vascularization systems, all of which contribute to the metastasis of the tumor (Runa et al., CURR MOL BIOL REP (2017) 3(4): 218-29). During tumor growth, cancer cells and TME constituents are continually adapting to the environment conditions, influencing the overall tumor growth. Accordingly, novel therapies that target different facets of the TME that contribute to tumor growth are needed.

### BRIEF SUMMARY OF THE DISCLOSURE

The TME is a complex system of blood vessels, immune cells, fibroblasts, signaling molecules and the extracellular matrix that interact with tumor tissue. Tumor progression is influenced by interactions of cancer cells with their environment that ultimately determine whether the primary tumor is eradicated, metastasizes or establishes dormant micro metastases. The TME can also impact therapeutic responses and drug or treatment resistance. Cancer cells debilitate antitumor immune responses and create an immunosuppressive environment. Thus, there exists an ongoing need to develop therapeutics capable of overcoming this immunosuppressive environment and/or sensitizing cancer cells to anticancer therapeutics to improve patient outcomes.

The present disclosure provides novel nucleic acids, oligonucleotides or analogues thereof comprising targeting ligands such as hydrophobic ligands, including but not limited to adamantyl and lipid conjugates, which are useful to target immune cells in the TME for therapeutic intervention. The present disclosure relates to nucleic acid-ligand conjugates and oligonucleotide-ligand conjugates, which function to modulate the expression of a target gene in a cell (e.g., an immune cell in a tumor microenvironment), and methods of preparation and uses thereof. Without wishing to be bound by theory, attachment of lipophilic/hydrophobic moieties, such as fatty acids and adamantyl group, to these highly hydrophilic nucleic acids/oligonucleotides substantially enhance plasma protein binding and consequently circulation half-life. As demonstrated herein, incorporation of a hydrophobic moiety such as a lipid facilitates systemic delivery of the novel nucleic acids, oligonucleotides, or analogues thereof into immune cell populations in a tumor microenvironment.

Suitable nucleic acid-ligand conjugates and oligonucleotide-ligand conjugates include nucleic acid inhibitor molecules, such as dsRNA inhibitor molecules, dsRNAi inhibitor molecules, antisense oligonucleotides, miRNA, ribozymes, antagomirs, aptamers, and single-stranded RNAi inhibitor molecules. In some aspects, the present disclosure provides nucleic acid-lipid conjugates, oligonucleotide-lipid conjugates, and analogues thereof, which find utility as modulators of intracellular RNA levels. Nucleic acid inhibitor molecules of the disclosure modulate RNA expression through a diverse set of mechanisms, for example by RNA interference (RNAi). An advantage of the nucleic acid-ligand conjugates, oligonucleotide-ligand conjugates and analogues thereof provided herein is that a broad range of pharmacological activities is possible, consistent with the modulation of intracellular RNA levels. In addition, the disclosure provides methods of using an effective amount of the conjugates described herein for the treatment or amelioration of a disease condition by modulating the intracellular RNA levels.

In some aspects, the present disclosure relates to oligonucleotide-ligand conjugates comprising one or more nucleic acid-ligand conjugate units that modulate target gene expression in an immune cell in the tumor microenvironment via RNA interference (RNAi). In some aspects, the present disclosure relates to oligonucleotide-ligand conjugates comprising one or more hydrophobic moiety ligand(s), including, but not limited to, lipid moieties, that modulate (e.g., reduce or inhibit) target gene expression in an immune cell in the tumor microenvironment, compositions of said oligonucleotide-ligand conjugates, and methods of preparation and uses thereof. In some aspects, the oligonucleotide-ligand conjugates target a gene encoding a regulator of immune suppression, such that reducing or inhibiting expression of the regulator overcomes an immunosuppressive tumor microenvironment. In some embodiments, reducing or inhibiting expression of the regulator induces or enhances an antitumor immune response.

The present disclosure is based, at least in part, on the discovery of oligonucleotide-ligand conjugates that effectively reduce target gene expression in immune cells present within a tumor microenvironment. Without being bound by theory, as described herein, a hydrophobic moiety (e.g., lipid) facilitates delivery and distribution of an RNAi oligonucleotide-lipid conjugate into immune cells, such as those expressing lipid trafficking receptors, of the tumor microenvironment, thereby increasing efficacy and durability of gene knockdown. Accordingly, the disclosure provides methods of treating cancer and/or reducing tumor growth by modulating target gene expression, e.g., of a gene encoding a regulator of immune suppression, in immune cells within a tumor microenvironment by administering the oligonucleotide ligand conjugates of the disclosure, and pharmaceutically acceptable compositions thereof, as described herein. The disclosure further provides methods of using the oligonucleotide ligand conjugates in the manufacture of a medicament for treating cancer and/or reducing tumor growth by modulating target gene expression in immune cells in a tumor microenvironment.

In some aspects, the disclosure provides a method of treating, ameliorating, or preventing cancer, and/or preventing metastasis of cancer in a subject in need thereof. The disclosure further provides RNAi oligonucleotide molecules that can limit, control, or eliminate the expression of key genes associated with cancer and/or an immune suppressive tumor microenvironment. Such RNAi oligonucleotide molecules are a variety of double-stranded RNAi oligonucleotides that target signal transducer and activator of transcription 3 (STAT3). In certain embodiments, the method comprises administering to the subject a therapeutically effective amount of a composition that inhibits STAT3 expression or activity in the subject. Such RNAi oligonucleotide molecules are used to treat a subject having cancer and associated pathologies and may thereby therapeutically benefit a subject suffering from carcinoma, sarcoma, melanoma, lymphoma, and leukemia, prostate cancer, breast cancer, hepatocellular carcinoma (HCC), colorectal cancer, and glioblastoma.

STAT3 is an important transcription factor that is crucial for then maintenance of carcinogenesis and for chemoresistance to anticancer agents. STAT3 is found in the cytoplasm and is activated in response to stimuli from the cytokines. Activated STAT3 regulates the transcription of genes controlling cell survival and proliferation and regulates the expression of antiapoptotic and immune response genes. Constitutive activation of STAT3 is necessary for the proliferation and survival of different cancers (Groner, B. et al, SEMINARS IN CELL & DEVELOPMENTAL BIOLOGY, Vol. 19(4): 341-50 (2008)). Activation of STAT-3 provides an advantage for survival of the cancer cells. Like NF-κB, the inhibition of STAT-3 in different cancer types has been demonstrated to induce apoptosis and chemosensitization of cells (da Hora, C. C. et al. CELL DEATH DISCOV, Vol. 5(72) https://doi.org/10.1038/s41420-019-0155-9 (2019)). The mRNA sequence of human STAT3 (NM_001369512.1) is set forth as SEQ ID NO:85 or SEQ ID NO: 1217 (NM_139276.3).

Accordingly, in one aspect, the disclosure provides an oligonucleotide-ligand conjugate comprising a nucleotide sequence that reduces expression of a target mRNA in an immune cell associated with a tumor microenvironment and one or more targeting ligands, wherein one or more nucleosides of the nucleotide sequence conjugated with one or more targeting ligands is represented by formula I-a:

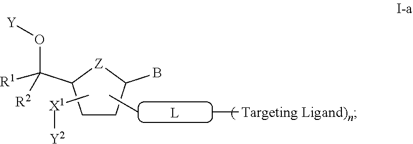

or a pharmaceutically acceptable salt thereof, wherein each variable is as defined and described herein.

In another aspect, the present disclosure provides an oligonucleotide-ligand conjugate comprising a nucleotide sequence that reduces expression of a target mRNA in an immune cell associated with a tumor microenvironment and one or more targeting ligands, wherein one or more nucleosides of the nucleotide sequence conjugated with one or more targeting ligands is represented by formula II-a:

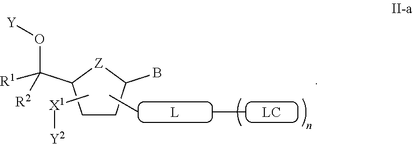

or a pharmaceutically acceptable salt thereof, wherein each variable is as defined and described herein.

In certain embodiments, the oligonucleotide-ligand conjugates are represented by formula II-b, II-c, II-Ib or II-Ic:

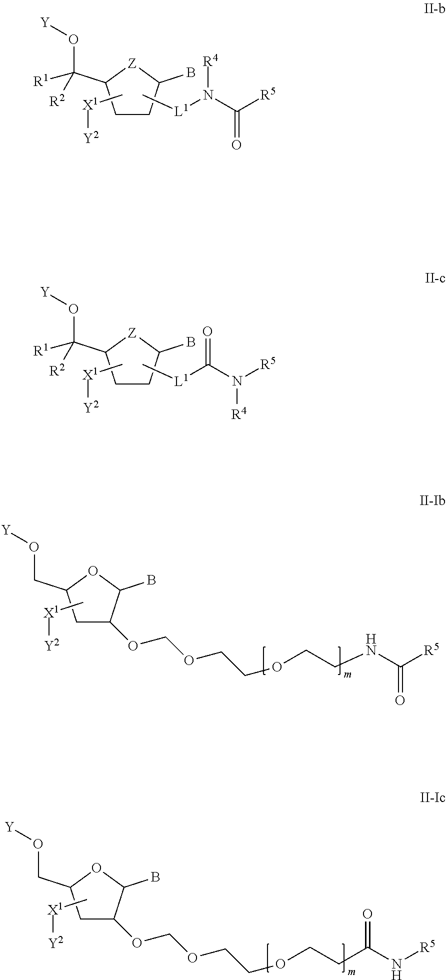

or a pharmaceutically acceptable salt thereof.

In any of the foregoing or related aspects, R⁵ is a saturated or unsaturated, straight or branched C1-C50 hydrocarbon chain. In some aspects, R⁵ is a saturated or unsaturated, straight or branched C8-C30 hydrocarbon chain. In some aspects, R⁵ is a saturated or unsaturated, straight or branched C16 hydrocarbon chain. In some aspects, R⁵ is a saturated or unsaturated, straight or branched C18 hydrocarbon chain.

In any of the foregoing or related aspects, the oligonucleotide-ligand conjugate comprises an antisense strand of 15 to 30 nucleotides and a sense strand of 15 to 40 nucleotide, wherein the sense and antisense strands form a duplex region, wherein the antisense strand comprises a region of complementarity to a target sequence expressed in an immune cell associated with a tumor microenvironment, wherein the sense strand comprises at its 3′ end a stem-loop comprising a tetraloop comprising 4 nucleosides, wherein one or more of the 4 nucleosides conjugated with the targeting ligand is represented by formula II-Ib:

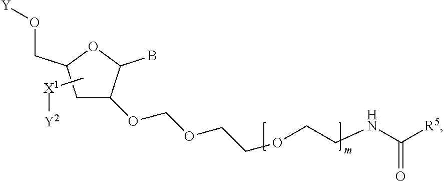

wherein B is selected from an adenine and a guanine nucleobase, and wherein R⁵ is a hydrocarbon chain. In some aspects, wherein the 4 nucleosides of the tetraloop are numbered 1-4 from 5′ to 3′, and wherein position 1 is represented by formula II-Ib. In other aspects, position 2 is represented by formula II-Ib. In yet other aspects, position 3 is represented by formula II-Ib. In further aspects, position 4 is represented by formula II-Ib.

In any of the foregoing or related aspects, the target mRNA encodes a regulator of immune suppression. In some aspects, the regulator of immune suppression is a checkpoint inhibitor polypeptide. In some aspects, the regulator of immune suppression is a transcription factor.

In any of the foregoing or related aspects, the immune cell associated with a tumor microenvironment is a myeloid cell. In some aspects, the immune cell associated with a tumor microenvironment is a T cell. In some aspects, the nucleotide sequence reduces expression of the target mRNA in more than one immune cell associated with the tumor microenvironment. In some aspects, the immune cell is a myeloid cell or a T cell. In some aspects, the myeloid cell is a myeloid derived suppressor cell (MDSC). In some aspects, the MDSC is a granulocytic MDSC (G-MDSC) or monocytic MDSC (M-MDSC). In some aspects, the nucleotide sequence reduces expression of the target mRNA in G-MDSCs and M-MDSCs. In some aspects, the T cell is a CD8+ T cell or Treg cell.

In some aspects, the oligonucleotide-ligand conjugate comprises a single stranded oligonucleotide. In some aspects, the oligonucleotide-ligand conjugate comprises a double stranded oligonucleotide. In some aspects, the double stranded oligonucleotide comprises a sense strand and an antisense strand that form a duplex region, wherein the antisense strand comprises a region of complementarity to the target mRNA in the immune cell associated with a tumor microenvironment.

In another aspect, the present disclosure provides RNAi oligonucleotide molecules capable of inhibiting expression of STAT3. Such molecules can be used alone or in combination with a second therapeutic agent and can vary in dosage. In some embodiments, such RNAi oligonucleotide molecules are comprised of a sense strand and an antisense strand forming a double-stranded region.

In some aspects, an oligonucleotide for reducing STAT3 expression comprises an antisense strand of 15 to 30 nucleotides in length and a sense strand of 15 to 40 nucleotides in length, wherein the sense strand and antisense strand form a duplex region, wherein the antisense strand has a region of complementarity to a target mRNA sequence of STAT3 as set forth in SEQ ID NO: 85 or SEQ ID NO: 1217, and wherein the region of complementarity is at least 15 contiguous nucleotides in length differing by no more than 3 nucleotides from the target sequence. In some aspects, the region of complementarity is fully complementary to the target sequence of STAT3.

In some aspects, an oligonucleotide for reducing STAT3 expression comprises a region of complementarity at least 15 contiguous nucleotides in length to a target sequence selected from SEQ ID NOs: 89-280. In some aspects, the region of complementarity is selected from SEQ ID Nos: 89-280.

In some aspects, an oligonucleotide for reducing STAT3 expression comprises:
(i) an antisense strand of 19-30 nucleotides in length, wherein the antisense strand comprises a nucleotide sequence comprising a region of complementarity to a STAT3 mRNA target sequence, wherein the region of complementarity is selected from SEQ ID NOs: 89-280, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some aspects, the sense strand comprises at least 15 contiguous nucleotides differing by no more than 3 nucleotides from any one of the nucleotide sequences of SEQ ID NOs: 9, 37, 65, or 69, and the antisense strand comprises at least 15 contiguous nucleotides differing by no more than 3 nucleotides from the nucleotide sequences of SEQ ID NOs: 10, 38, 66, or 70. In some aspects, an oligonucleotide for reducing STAT3 expression comprises a sense strand and an antisense strand comprising the nucleotide sequences selected from:
(a) SEQ ID NOS: 9 and 10, respectively;
(b) SEQ ID NOs: 37 and 38, respectively;
(c) SEQ ID NOs: 65 and 66, respectively; and,
(d) SEQ ID NOs: 69 or 70, respectively.

In some aspects, an oligonucleotide for reducing STAT3 expression comprises a sense strand comprising a nucleotide sequence selected from SEQ ID NOs: 857-946.

In some aspects, an oligonucleotide for reducing STAT3 expression comprises an antisense strand comprising a nucleotide sequence selected from SEQ ID NOs: 947-1036.

In some aspects, an oligonucleotide for reducing STAT3 expression comprises a sense strand and an antisense strand comprising the nucleotide sequences selected from:
(a) SEQ ID NOs: 861 and 951, respectively;
(b) SEQ ID NOs: 857 and 947, respectively;
(c) SEQ ID NOs: 858 and 948, respectively;
(d) SEQ ID NOs: 859 and 949, respectively;
(e) SEQ ID NOs: 860 and 950, respectively;
(f) SEQ ID NOs: 862 and 952, respectively;
(g) SEQ ID NOs: 863 and 953, respectively;
(h) SEQ ID NOs: 864 and 954, respectively;
(i) SEQ ID NOs: 865 and 955, respectively;
(j) SEQ ID NOs: 866 and 956, respectively;
(k) SEQ ID NOs: 867 and 957, respectively;
(l) SEQ ID NOs: 868 and 958, respectively;
(m) SEQ ID NOs: 869 and 959, respectively;
(n) SEQ ID NOs: 870 and 960, respectively;
(o) SEQ ID NOs: 871 and 961, respectively;
(p) SEQ ID NOs: 872 and 962, respectively;
(q) SEQ ID NOs: 873 and 963, respectively;
(r) SEQ ID NOs: 874 and 964, respectively;
(s) SEQ ID NOs: 875 and 965, respectively;
(t) SEQ ID NOs: 876 and 966, respectively;
(u) SEQ ID NOs: 877 and 967, respectively;
(v) SEQ ID NOs: 878 and 968, respectively;
(w) SEQ ID NOs: 879 and 969, respectively;
(x) SEQ ID NOs: 880 and 970, respectively;
(y) SEQ ID NOs: 881 and 971, respectively;
(z) SEQ ID NOs: 882 and 972, respectively;
(aa) SEQ ID NOs: 883 and 973, respectively;
(bb) SEQ ID NOs: 884 and 974, respectively;
(cc) SEQ ID NOs: 885 and 975, respectively;
(dd) SEQ ID NOs: 886 and 976, respectively;
(ee) SEQ ID NOs: 887 and 977, respectively;
(ff) SEQ ID NOs: 888 and 978, respectively;
(gg) SEQ ID NOs: 940 and 1030, respectively;
(hh) SEQ ID NOs: 896 and 986, respectively; and
(ii) SEQ ID NOs: 920 and 1010, respectively.

In some aspects, an oligonucleotide for reducing STAT3 expression comprises a sense strand and an antisense strand comprising the nucleotide sequences selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively.

In some aspects, an oligonucleotide for reducing STAT3 expression comprises a sense strand and an antisense strand comprising the nucleotide sequences selected from:
(a) SEQ ID NOs: 940 and 1030, respectively;
(b) SEQ ID NOs: 937 and 1027, respectively; and
(c) SEQ ID NOs: 939 and 1029, respectively.

In some aspects, an oligonucleotide for reducing STAT3 expression comprises a sense strand and an antisense strand comprising the nucleotide sequences selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively.

In some aspects, an oligonucleotide for reducing STAT3 expression comprises a sense strand comprising the nucleotide sequence of SEQ ID NO: 862 and an antisense strand comprising the nucleotide sequence of SEQ ID NO: 952.

In some aspects, an oligonucleotide for reducing STAT3 expression comprises a sense strand comprising the nucleotide sequence of SEQ ID NO: 875 and an antisense strand comprising the nucleotide sequence of SEQ ID NO: 965.

In some aspects, an oligonucleotide for reducing STAT3 expression comprises a sense strand comprising the nucleotide sequence of SEQ ID NO: 876 and an antisense strand comprising the nucleotide sequence of SEQ ID NO: 966.

In some aspects, an oligonucleotide for reducing STAT3 expression comprises a sense strand comprising the nucleotide sequence of SEQ ID NO: 920 and an antisense strand comprising the nucleotide sequence of SEQ ID NO: 966.

In any of the foregoing or related aspects, the antisense strand is 19 to 27 nucleotides in length or 21 to 27 nucleotides in length. In some embodiments, the antisense strand is 22 nucleotides in length.

In any of the foregoing or related aspects, the sense strand is 19 to 40 nucleotides in length. In some embodiments, the sense strand is 36 nucleotides in length.

In any of the foregoing or related aspects, the oligonucleotide has a duplex region of at least 19 nucleotides in length. In any of the foregoing or related aspects, the oligonucleotide has a duplex region of at least 21 nucleotides in length. In some embodiments, the duplex region is 20 nucleotides in length.

In some embodiments, the region of complementarity to STAT3 is at least 19 contiguous nucleotides in length. In some embodiments, the region of complementarity to STAT3 is at least 21 contiguous nucleotides in length.

In any of the foregoing or related aspects, the oligonucleotide comprises on the sense strand at its 3′ end a stem-loop set forth as: S1-Loop-S2, wherein S1 is complementary to S2, and wherein Loop forms a loop between S1 and S2 of 3 to 5 nucleotides in length.

In some embodiments, an oligonucleotide for reducing STAT3 expression for treating or preventing cancer, and/or preventing metastasis of cancer, comprises an antisense strand and a sense strand, wherein the antisense strand is 21 to 27 nucleotides in length and has a region of complementarity to a target mRNA sequence of STAT3 set forth in SEQ ID NO: 85 or SEQ ID NO: 1217, wherein the sense strand comprises at its 3′ end a stem-loop set forth as: S1-Loop-S2, wherein S1 is complementary to S2, and wherein Loop forms a loop between S1 and S2 of 3 to 5 nucleotides in length, and wherein the antisense strand and the sense strand form a duplex structure of at least 19 nucleotides in length.

In some embodiments, Loop is a tetraloop. In some embodiments, Loop is 4 nucleotides in length. In some embodiments, Loop comprises a sequence GAAA.

In some embodiments, the oligonucleotide comprises an antisense strand which is 27 nucleotides in length and a sense strand which is 25 nucleotides in length. In some embodiments, the oligonucleotide comprises an antisense strand which is 22 nucleotides in length and a sense strand which is 36 nucleotides in length.

In any of the foregoing or related aspects, the duplex region of the oligonucleotide of the present disclosure comprises a 3′-overhang sequence on the antisense strand. In some embodiments, the 3′-overhang sequence on the antisense strand is 2 nucleotides in length. In some embodiments, the 3′-overhang sequence is GG.

In some embodiments, the oligonucleotide comprises an antisense strand and a sense strand that are each in a range of 21 to 23 nucleotides in length. In some embodiments, the oligonucleotide comprises a duplex structure in a range of 19 to 21 nucleotides in length. In some such embodiments, the oligonucleotide comprises a 3′-overhang sequence of one or more nucleotides in length, wherein the 3′-overhang sequence is present on the antisense strand, the sense strand, or the antisense strand and sense strand. In some embodiments, the 3′-overhang sequence of 2 nucleotides in length, wherein the 3′-overhang sequence is on the antisense strand, and wherein the sense strand is 21 nucleotides in length and the antisense strand is 23 nucleotides in length, such that the sense strand and antisense strand form a duplex of 21 nucleotides in length.

In some embodiments, the oligonucleotide comprises at least one modified nucleotide. In some embodiments, the modified nucleotide comprises a 2′-modification. In some embodiments, all the nucleotides of the oligonucleotide are modified, for example with a 2′-modification. In some embodiments, about 10-15%, 10%, 11%, 12%, 13%, 14% or 15% of the nucleotides of the sense strand comprise a 2′-fluoro modification. In some embodiments, about 25-35%, 25%, 26%, 27%, 28%, 29%, 30%, 31%, 32%, 33%, 34% or 35% of the nucleotides of the antisense strand comprise a 2′-fluoro modification. In some embodiments, about 25-35%, 25%, 26%, 27%, 28%, 29%, 30%, 31%, 32%, 33%, 34% or 35% of the nucleotides of the oligonucleotide comprise a 2′-fluoro modification. In some embodiments, the sense strand comprises 36 nucleotides with positions 1-36 from 5′ to 3′, wherein positions 8-11 comprise a 2′-fluoro modification. In some embodiments, the antisense strand comprises 22 nucleotides with positions 1-22 from 5′ to 3′, and wherein positions 2, 3, 4, 5, 7, 10 and 14 comprise a 2′-fluoro modification. In some embodiments, the remaining nucleotides comprise a 2′-O-methyl modification.

In some embodiments, the oligonucleotide comprises at least one modified internucleotide linkage, preferably a phosphorothioate linkage.

In some embodiments, the 4′-carbon of the sugar of the 5′-nucleotide of the antisense strand comprises a phosphate analog, for example, an oxymethylphosphonate, vinylphosphonate or malonyl phosphonate.

In some embodiments, at least one nucleotide of the oligonucleotide is conjugated to one or more targeting ligands, such as a carbohydrate, amino sugar, cholesterol, polypeptide, or lipid.

In some embodiments the targeting ligand is a saturated fatty acid moiety. In some embodiments the saturated fatty acid moiety varies in length from C10 to C24. In some embodiments the saturated fatty acid moiety has a length of C16. In some embodiments the saturated fatty acid moiety has a length of C18. In some embodiments the saturated fatty acid moiety has a length of C22.

In some embodiments, the targeting ligand comprises a N-acetyl galactosamine (GalNAc) moiety. In some embodiments, the (GalNAc) moiety comprises a monovalent GalNAc moiety, a bivalent GalNAc moiety, a trivalent GalNAc moiety, or a tetravalent GalNAc moiety.

In some aspects, an oligonucleotide for reducing STAT3 expression comprises a sense strand and an antisense strand comprising the nucleotide sequences selected from:
(a) SEQ ID NOs: 1041 and 1131, respectively;
(b) SEQ ID NOs: 1037 and 1127, respectively;
(c) SEQ ID NOs: 1038 and 1128, respectively;
(d) SEQ ID NOs: 1039 and 1129, respectively;
(e) SEQ ID NOs: 1040 and 1130, respectively;
(f) SEQ ID NOs: 1042 and 1132, respectively;
(g) SEQ ID NOs: 1043 and 1133, respectively;
(h) SEQ ID NOs: 1044 and 1134, respectively;
(i) SEQ ID NOs: 1045 and 1135, respectively;
(j) SEQ ID NOs: 1046 and 1136, respectively;
(k) SEQ ID NOs: 1047 and 1137, respectively;
(l) SEQ ID NOs: 1048 and 1138, respectively;
(m) SEQ ID NOs: 1049 and 1139, respectively;
(n) SEQ ID NOs: 1050 and 1140, respectively;
(o) SEQ ID NOs: 1051 and 1141, respectively;
(p) SEQ ID NOs: 1052 and 1142, respectively;
(q) SEQ ID NOs: 1053 and 1143, respectively;
(r) SEQ ID NOs: 1054 and 1144, respectively;
(s) SEQ ID NOs: 1055 and 1145, respectively;
(t) SEQ ID NOs: 1056 and 1146, respectively;
(u) SEQ ID NOs: 1057 and 1147, respectively;
(v) SEQ ID NOs: 1058 and 1148, respectively;
(w) SEQ ID NOs: 1059 and 1149, respectively;
(x) SEQ ID NOs: 1060 and 1150, respectively;
(y) SEQ ID NOs: 1061 and 1151, respectively;
(z) SEQ ID NOs: 1062 and 1152, respectively;
(aa) SEQ ID NOs: 1063 and 1153, respectively;
(bb) SEQ ID NOs: 1064 and 1154, respectively;
(cc) SEQ ID NOs: 1065 and 1155, respectively;
(dd) SEQ ID NOs: 1066 and 1156, respectively;
(ee) SEQ ID NOs: 1067 and 1157, respectively;
(ff) SEQ ID NOs: 1068 and 1158, respectively;
(gg) SEQ ID NOs: 1120 and 1210, respectively;
(hh) SEQ ID NOs: 1076 and 1166, respectively; and
(ii) SEQ ID NOs: 1100 and 1190, respectively.

In some aspects, an oligonucleotide for reducing STAT3 expression comprises a sense strand and an antisense strand comprising the nucleotide sequences selected from:
(a) SEQ ID NOs: 1081 and 1171, respectively;
(b) SEQ ID NOs: 1090 and 1180, respectively;
(c) SEQ ID NOs: 1079 and 1169, respectively;
(d) SEQ ID NOs: 1076 and 1166, respectively;
(e) SEQ ID NOs: 1072 and 1162, respectively;
(f) SEQ ID NOs: 1070 and 1160, respectively; and
(g) SEQ ID NOs: 1069 and 1159, respectively.

In some aspects, an oligonucleotide for reducing STAT3 expression comprises a sense strand and an antisense strand comprising the nucleotide sequences selected from:
(a) SEQ ID NOs: 1120 and 1210, respectively;
(b) SEQ ID NOs: 1117 and 1207, respectively; and
(c) SEQ ID NOs: 1119 and 1209, respectively.

In some aspects, an oligonucleotide for reducing STAT3 expression comprises a sense strand and an antisense strand comprising the nucleotide sequences selected from:
(a) SEQ ID NOs: 1095 and 1185, respectively;
(b) SEQ ID NOs: 1104 and 1194, respectively;
(c) SEQ ID NOs: 1093 and 1183, respectively; and
(d) SEQ ID NOs: 1100 and 1190, respectively.

In some aspects, an oligonucleotide for reducing STAT3 expression comprises a sense strand comprising the nucleotide sequence of SEQ ID NO: 1042 and an antisense strand comprising the nucleotide sequence of SEQ ID NO: 1132.

In some aspects, an oligonucleotide for reducing STAT3 expression comprises a sense strand comprising the nucleotide sequence of SEQ ID NO: 1055 and an antisense strand comprising the nucleotide sequence of SEQ ID NO: 1145.

In some aspects, an oligonucleotide for reducing STAT3 expression comprises a sense strand comprising the nucleotide sequence of SEQ ID NO: 1056 and an antisense strand comprising the nucleotide sequence of SEQ ID NO: 1146.

In some aspects, an oligonucleotide for reducing STAT3 expression comprises a sense strand comprising the nucleotide sequence of SEQ ID NO: 1100 and an antisense strand comprising the nucleotide sequence of SEQ ID NO: 1190.

In some embodiments, the targeting ligand is conjugated to one or more nucleotides of Loop of the stem loop. In some embodiments, up to 4 nucleotides of Loop of the stem-loop are each conjugated to a monovalent GalNAc moiety.

In some embodiments, the oligonucleotides of the present disclosure are RNAi oligonucleotides.

In some embodiments, the disclosure of the present disclosure is a pharmaceutical composition comprising one or more oligonucleotides and a pharmaceutically acceptable carrier, delivery agent or excipient.

In some aspects the oligonucleotide of the present disclosure is provided in the form of a kit for treating a cancer. In a further aspect, the oligonucleotide of the present disclosure is provided in the form of a kit for treating a disease, disorder or condition associated with STAT3 expression. In some embodiments, the kit comprises an oligonucleotide described herein, and a pharmaceutically acceptable carrier. In some embodiments, the kit further includes a package insert comprising instructions for administration of the oligonucleotide to a subject having a cancer. In some embodiments, the kit further includes a package insert comprising instructions for administration of the oligonucleotide to a subject having a disease, disorder or condition associated with STAT3 expression.

In some embodiments, the present disclosure provides a method of delivering an oligonucleotide to a subject, the method comprising administering a pharmaceutical composition to a subject. In some embodiments, the present disclosure provides a method of delivering an oligonucleotide to an immune cell associated with a tumor microenvironment, comprising administering an oligonucleotide-ligand conjugate described herein.

In some embodiments the oligonucleotide-ligand conjugate is delivered to tumor associated cells. In some embodiments the oligonucleotide-ligand conjugate is delivered to immune cells. In some embodiments the immune cells are myeloid derived suppressor cells (MDSCs). In some embodiments, the immune cells are T cells.

In some embodiments the oligonucleotide described herein targets STAT3. In some embodiments the oligonucleotide targets STAT3 and the siRNA also modulates PD-LI mRNA expression.

In some aspects, the present disclosure provides a method of reducing expression of a target mRNA in a cell, a population of cells associated with a tumor microenvironment in a subject by administering an oligonucleotide of the disclosure. In another aspect, the present disclosure provides a method of reducing STAT3 expression in a cell, a population of cells or a subject by administering an oligonucleotide of the disclosure. In some embodiments, a method of reducing STAT3 expression in a cell, a population of cells or a subject comprises the step of: contacting the cell or the population of cells or administering to the subject an effective amount of an oligonucleotide or oligonucleotides described herein, or a pharmaceutical composition thereof. In some embodiments, the method for reducing STAT3 expression comprises reducing an amount or a level of STAT3 and PD-L1 mRNA, an amount, or a level of STAT3 and PD-L1 protein, or both.

In some embodiments the present disclosure provides a pharmaceutical product for use as a therapeutic agent. In some embodiments a therapeutic agent is administered as a monotherapy and is an inhibitor of STAT3 expression.

In some embodiments, a method of treating human subjects that are resistant to anti-PD1 or anti-PD-L1 therapy is provided comprising administering any one of the STAT3 targeting oligonucleotides described herein. Subjects who are resistant to anti-PD1 or anti-PD-L1 include subject whose benefit from the anti-PD1 or anti-PD-L1 therapy remained diminished by at least one standard deviation as compared to a non-resistant control for greater than three months.

In some embodiments a therapeutic agent is administered as a monotherapy and is an inhibitor of STAT3 and PD-L1 expression. In some embodiments, the present disclosure provides a pharmaceutical product comprising at least a first and second therapeutic agent, wherein the first therapeutic agent is an inhibitor of STAT3. In some embodiments a therapeutic agent is administered prior to, or intermittently with, administration of a second therapeutic agent. In some embodiments, a first therapeutic agent is administered concurrently or simultaneously with a second therapeutic agent. In some embodiments, the present disclosure provides a pharmaceutical product comprising more than two therapeutic agents, wherein the first therapeutic agent is an inhibitor of STAT3.

In some aspects, the disclosure provides a method of treating cancer in a subject, the method comprising administering to the subject an effective amount of an oligonucleotide-ligand conjugate described herein that targets a regulator of immune suppression, provided by the disclosure, in combination with one or more additional therapeutic agents or procedures. In some embodiments, the disclosure provides a method of treating cancer in a subject, the method comprising administering to the subject an effective amount of an oligonucleotide that targets STAT3, provided by the disclosure, in combination with one or more additional therapeutic agents or procedures. In some aspects, the second therapeutic agent or procedure is selected from the group consisting of: a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, gene therapy or a combination thereof.

In some embodiments, the disclosure provides a method of treating a subject having a disease, disorder or condition associated with STAT3 expression, the method comprising administering to the subject a therapeutically effective amount of an oligonucleotide or oligonucleotide-ligand conjugate described herein. In some embodiments, the oligonucleotide or oligonucleotide-ligand conjugate is administered in combination with a second composition or therapeutic agent. In some embodiments, the second composition or therapeutic agent targets TGFB, CXCR2, CCR2, ARG1, PTGS2, SOCS1 or PD-L1.

In some embodiments, the one or more additional therapeutic agents is a PD-1 antagonist, a CTLA-4 inhibitor, a TGFB inhibitor, a CXCR2 inhibitor, a CCR2 antagonist, an ARG1 inhibitor, a PTGS2 inhibitor, a SOCS1 modulator or a combination thereof.

In some embodiments, the one or more additional therapeutic agents is a PD-1 antagonist.

In some embodiments, the PD-1 antagonist is selected from the group consisting of: PDR001, nivolumab, pembrolizumab, pidilizumab, MEDI0680, REGN2810, TSR-042, PF-06801591, and AMP-224. In some embodiments, the PD-1 antagonist is selected from the group consisting of: FAZ053, Atezolizumab, Avelumab, Durvalumab, and BMS-936559.

In some embodiments, the one or more additional therapeutic agents is a CTLA-4 inhibitor. In some embodiments, the CTLA-4 inhibitor is Ipilimumab or Tremelimumab.

In some embodiments, the one or more additional therapeutic agents is a TGFB inhibitor. In some embodiments, the TGFB inhibitor is Frisolimumab, LY3022859 or PF-03446962.

In some embodiments, the one or more additional therapeutic agents is an ARG1 inhibitor. In some embodiments, the ARG1 inhibitor is CB-1158.

### BRIEF DESCRIPTION OF THE DRAWINGS

FIG. 1A provides structures of RNAi oligonucleotide molecules having chemical modifications with GalNAc (top) or lipid (bottom) conjugated to the base molecule to generate oligonucleotide-ligand conjugates.

FIG. 1B provides structures of lipid tails suitable for conjugation to RNAi oligonucleotide molecules.

FIG. 2A is a graph representing remaining human ALDH2 mRNA levels in human LS411N tumor xenograft epithelium from mice three days following treatment with 10 mg/kg ALDH2 RNAi-GalXC lipid conjugates with varying acyl chain lengths and unsaturation.

FIG. 2B is a graph representing remaining mouse Aldh2 mRNA levels in tumor microenvironment (TME) isolated from human LS411N tumor xenografts. TME was isolated from mice three days following treatment with 10 mg/kg ALDH2-GalXC lipid conjugates with varying acyl chain lengths and unsaturation

FIG. 3A is a graph demonstrating remaining human ALDH2 mRNA following treatment with various doses of GalXC-ALDH2-C22 conjugate in human LS411N tumor xenograft epithelium. Samples were collected from mice on Days 3, 7, and 14 post-treatment.

FIG. 3B is a graph demonstrating remaining mouse Aldh2 mRNA following treatment with various doses of GalXC-ALDH2-C22 conjugate in host mouse tissue in the tumor microenvironment collected from human LS411N tumors. Samples were collected on Days 3, 7, and 14 post-treatment.

FIGS. 4A and 4B are graphs demonstrating remaining mouse Aldh2 mRNA following treatment with 25 mg/kg of GalXC-ALDH2-C22 conjugate in the tumor draining lymph nodes of human LS411N tumor xenograft bearing mice (FIG. 4A) and in lymph nodes of mice with no tumors (FIG. 4B).

FIG. 5A is a graph showing remaining mouse Aldh2 mRNA levels following treatment with GalXC-ALDH2-C22 conjugate or PBS in murine tumor draining lymph nodes (TdLN) compared to non-TdLN over time in human LS411N tumor xenografts. Normalized mRNA is relative to a PBS treated mouse.

FIG. 5B provides graphs showing the Pdl1 mRNA levels in murine tumor draining lymph nodes (TdLN) compared to Non-TdLN from LS411N tumor xenograft mice treated with GalXC-ALDH2-C22.

FIG. 6 is a graph demonstrating the expression of Arg1 in isolated tumor associated CD11b⁺ myeloid derived suppressor cells (MDSCs) and normal spleen myeloid cells from human LS411N tumor xenografts treated with 25 mg/kg GalXC-ALDH2-C22. Three days after treatment, MDSCs and tumor cells were isolated from mice and measured using CD11b mRNA. BLOQ=below limit of quantification.

FIGS. 7A and 7B are graphs showing the level of remaining mouse Aldh2 mRNA in isolated CD11b+ MDSCs (FIG. 7A) and tumor cells (FIG. 7B) from mice with human LS411N tumor xenografts treated with GalXC-ALDH2-C22 conjugate.

FIGS. 8A and 8B are graphs demonstrating remaining mouse Aldh2 mRNA from bulk tumor (FIG. 8A), and liver (FIG. 8B) of Pan02 xenografts. Mice were treated with 25 mg/kg of the specified GalXC-ALDH2-lipid conjugate and mRNA was measured on day 3.

FIGS. 8C and 8D are graphs demonstrating remaining mouse Aldh2 mRNA from bulk tumor (FIG. 8C) and tumor draining lymph node (TdLN) from mice with Pan02 xenografts on day 7 and day 14 after treatment with 25 mg/kg of the specified GalXC-ALDH2-lipid conjugate.

FIG. 9 provides graphs showing expression of differentiating mRNA markers (Ly6G, Cxcr2, Slc27a2, and Ptgs2) in G-MDSC isolated from TME of untreated (control) PANO2 tumors.

FIG. 10 provides graphs showing the expression of differentiating mRNA markers (Ly6G, Cxcr2, Slc27a2, and Ptgs2) in M-MDSC isolated from TME.

FIGS. 11 and 12 provide graphs showing the differential expression of lipid trafficking receptors in G-MDSC and M-MDSC in untreated (control) tissue.

FIGS. 13A and 13B provide graphs showing remaining mouse Aldh2 mRNA levels after treatment with 25 mg/kg of GalXC-ALDH2-C18 conjugate in isolated G-MDSCs and M-MDSCs from Pan02 (FIG. 13A) and B16F10 (FIG. 13B) TME. Mice were randomized into groups once tumors reached 300-500 mm then treated on day 1 and tissue was collected for analysis on day 3.

FIGS. 13C and 13D provide graphs showing remaining mouse Aldh2 mRNA levels after treatment with 50 mg/kg GalXC-ALDH2-C18 conjugate in G-MDSCs and M-MDSCs from Pan02 TME of mice on days 3 (FIG. 13C) and 7 (FIG. 13D).

FIGS. 14A-14C are graphs showing the relative expression of Stat3 in G-MDSC (FIG. 14A), M-MDSC (FIG. 14B) and TdLN (FIG. 14C) from Pan02 xenografts implanted in mice.

FIGS. 15A and 15B are graphs showing remaining mouse Stat3 mRNA levels in the livers of mice treated with GalXC-STAT3-conjugates (GalNAc conjugates) targeting different regions of Stat3 mRNA. Mice were administered a single dose (3 mg/kg) (FIG. 15A) and multi dose to determine dose responsiveness (FIG. 15B). Arrows indicate constructs selected for further study.

FIGS. 16A and 16B are graphs showing mouse Stat3 mRNA expression after treatment with GalXC-STAT3-C18 conjugates in G-MDSCs and M-MDSCs derived from Pan02 xenografts implanted in mice. Tumors were dosed at 25 mg/kg (FIG. 16A) and 50 mg/kg (FIG. 16B).

FIGS. 17A and 17B are graphs showing mouse Stat3 mRNA expression after treatment of Pan02 xenograft mice with GalXC-STAT3-C18 conjugates in bulk tumor (TME) (FIG. 17A) and TdLNs (FIG. 17B) at doses of 25 and 50 mg/kg.

FIG. 18A provides graphs showing the effect of GalXC-STAT3-C18-4123 on Stat3 and Pdl1 mRNA levels in G/M-MDSCs in TME and TdLNs of Pan02 xenograft mice on day 3 after a dose of 25 or 50 mg/kg of conjugate.

FIG. 18B provides graphs showing the effect of GalXC-STAT3-C18-4123 on Stat3 and Pdl1 mRNA levels in TdLN of Pan02 xenograft mice on day 7 after a 25 mg/kg dose of conjugate.

FIGS. 19A and 19B are graphs showing the in vivo effect of subcutaneous treatment with a total dose of 50 mg/kg GalXC-STAT3-C18-4123 on tumor volume in immunocompetent mice bearing Pan02 murine pancreatic tumors. Mice were treated with either four 12.5 mg/kg (FIG. 19A) or two 25 mg/kg (FIG. 19B) doses of conjugate.

FIG. 20 provides a graph depicting the percent (%) of human STAT3 mRNA remaining in Huh7 cells endogenously expressing human STAT3, after 24-hour treatment with 1 nM of DsiRNA targeting various regions of the STAT3 gene. 192 DsiRNAs were designed and screened. Two primer pairs were used. Expression was normalized between samples using the HPRT and SFRS9 housekeeping genes (Forward 1—SEQ ID NO: 1219, Reverse 1—SEQ ID NO: 1220; Probe 1—SEQ ID NO: 1221; Forward 2—SEQ ID NO: 1222, Reverse 2—SEQ ID NO: 1223; Probe 2—SEQ ID NO: 1224).

FIGS. 21A and 21B provide graphs depicting the percent (%) of human STAT3 mRNA remaining in Huh7 cells endogenously expressing human STAT3, after 24-hour treatment with 0.05 nM, 0.3 nM, or 1 nM of DsiRNA targeting various regions of the STAT3 gene. 48 GalNAc-conjugated STAT3 oligonucleotides s were assayed in FIG. 21A and 34 of those oligonucleotides were selected for further testing in vivo (FIG. 21B).

FIGS. 22A and 22B provide graphs depicting the percent (%) of human STAT3 mRNA remaining in liver of mice exogenously expressing human STAT3 (hydrodynamic injection model) after treatment with GalNAc-conjugated STAT3 oligonucleotides. Mice were dosed subcutaneously with 1 mg/kg of the indicated GalNAc-STAT3 oligonucleotides formulated in PBS. Three days post-dose mice were hydrodynamically injected (HDI) with a DNA plasmid encoding human STAT3. The level of human STAT3 mRNA was determined from livers collected 18 hours after injection. Arrows indicate oligonucleotides selected for dose response analysis. Hs/Mf=human/monkey common sequence; Hs/Mm=human/mouse common sequence; Hs/Mf/Mm=human/monkey/mouse triple common sequence.

FIG. 23 provides a graph depicting the dose response of GalNAc-conjugated STAT3 oligonucleotides. The percent (%) of human STAT3 mRNA remaining in liver of mice exogenously expressing STAT3 (HDI model) after treatment with human GalNAc-conjugated STAT3 oligonucleotides at three doses (0.3 mg/kg, 1 mg/kg) was measured. The level of human STAT3 mRNA was determined from livers collected 18 hours after injection with plasmid encoding human STAT3. Arrows indicate oligonucleotides selected for dose response analysis. Hs/Mf=human/monkey common sequence; Hs/Mm=human/mouse common sequence.

FIG. 24 provides a graph depicting the normalized (to Ppib) relative mouse STAT3 mRNA remaining in liver of mice endogenously expressing mouse STAT3 after treatment with GalNAc-conjugated STAT3 oligonucleotides. Mice were dosed subcutaneously with 3 mg/kg of the indicated GalNAc-STAT3 oligonucleotides formulated in PBS. Five days post-dose liver was collected and the level of mouse STAT3 mRNA was determined. Arrows indicate top oligonucleotides and those selected for dose response study.

FIG. 25 provides a graph depicting the normalized (to Ppib) relative mouse STAT3 mRNA remaining in liver of mice endogenously expressing mouse STAT3 after treatment with GalNAc-conjugated STAT3 oligonucleotides. Mice were dosed subcutaneously with 3 mg/kg of the indicated GalNAc-STAT3 oligonucleotides formulated in PBS. Five days post-dose liver was collected and the level of mouse STAT3 mRNA was determined. Arrows indicate oligonucleotides selected for dose response study.

FIGS. 26A and 26B provide graphs depicting the dose response of GalNAc-conjugated STAT3 oligonucleotides. The percent (%) of mouse STAT3 mRNA remaining in liver of mice endogenously expressing STAT3 after treatment with human GalNAc-conjugated STAT3 oligonucleotides at three doses (0.3 mg/kg, 1 mg/kg, and 3 mg/kg) was measured. The level of mouse STAT3 mRNA was determined from livers collected 5 days later. TC=triple common (mouse/human/monkey); Hs_Mm=human/mouse.

FIG. 27 provides a graph depicting the percent (%) of human STAT3 mRNA remaining in liver of mice exogenously expressing human STAT3 (hydrodynamic injection model) after treatment with GalNAc-conjugated STAT3 oligonucleotides. Mice were dosed subcutaneously with 1 mg/kg of the indicated GalNAc-STAT3 oligonucleotides formulated in PBS. Three days post-dose mice were hydrodynamically injected (HDI) with a DNA plasmid encoding human STAT3. The level of human STAT3 mRNA was determined from livers collected 18 hours after injection. Arrows indicate oligonucleotides selected for dose response study.

FIG. 28 provides a graph depicting the dose response of GalNAc-conjugated STAT3 oligonucleotides. The percent (%) of human STAT3 mRNA remaining in liver of mice exogenously expressing human STAT3 (hydrodynamic injection model) after treatment with GalNAc-conjugated STAT3 oligonucleotides. Mice were dosed subcutaneously with three doses (0.3 mg/kg, 1 mg/kg, and 3 mg/kg) of the indicated GalNAc-STAT3 oligonucleotides formulated in PBS. Three days post-dose mice were hydrodynamically injected (HDI) with a DNA plasmid encoding human STAT3. The level of human STAT3 mRNA was determined from livers collected 18 hours after injection. TC=triple common (mouse/human/monkey); Hs_Mm=human/mouse; Hs=human.

FIG. 29 provides a graph depicting the dose response of GalNAc-conjugated STAT3 oligonucleotides. The percent (%) of human STAT3 mRNA remaining in liver of mice exogenously expressing human STAT3 (hydrodynamic injection model) after treatment with GalNAc-conjugated STAT3 oligonucleotides. Mice were dosed subcutaneously with two doses (0.3 mg/kg and 1 mg/kg) of the indicated GalNAc-STAT3 oligonucleotides formulated in PBS. Three days post-dose mice were hydrodynamically injected (HDI) with a DNA plasmid encoding human STAT3. The level of human STAT3 mRNA was determined from livers collected 18 hours after injection.

FIG. 30 provides a graph depicting the percent (%) remaining human STAT1 mRNA in Huh7 cells endogenously expressing STAT3 and STAT1 treated with GalNAc-conjugated STAT3 oligonucleotides. Cells were treated for 24 hours with three doses (0.05 nM, 0.3 nM, and 1 nM) of oligonucleotide.

### DETAILED DESCRIPTION

The present disclosure now will be described more fully hereinafter with reference to the accompanying drawings, in which illustrative embodiments of the disclosure are shown. The disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

In some aspects, the disclosure provides oligonucleotide-ligand conjugates (e.g., RNAi oligonucleotide-lipid conjugates) that reduce expression of a target gene (e.g., encoding a regulator of immune suppression) in immune cells within a tumor microenvironment. In other aspects, the disclosure provides methods of treating a disease or disorder (e.g., cancer) using the oligonucleotide-ligand conjugates, or pharmaceutically acceptable compositions thereof, described herein. In other aspects, the disclosure provides methods of using the oligonucleotide-ligand conjugates described herein in the manufacture of a medicament for treating cancer. In other aspects, the oligonucleotide-ligand conjugates provided herein are used to treat cancer by modulating (e.g., inhibiting or reducing) expression of a target gene encoding a regulator of immune suppression in an immune cell in the tumor microenvironment. In some aspects, the disclosure provides methods of treating cancer by reducing expression of a target encoding a regulator of immune suppression in an immune cell in the tumor microenvironment.

### Definitions

The publications discussed throughout the text are provided solely for their disclosure prior to the filing date of the present application. Nothing herein is to be construed as an admission that the inventors are not entitled to antedate such disclosure by virtue of prior disclosure.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, GUIDE TO MOLECULAR CLONING TECHNIQUES, METHODS IN ENZYMOLOGY, volume 152, (Academic Press, Inc., San Diego, Calif.) (“Berger”); Sambrook et al., MOLECULAR CLONING—A LABORATORY MANUAL, 2d ed., Vol. 1-3, Cold Spring Harbor Laboratory, Cold Spring Harbor, 1989 (“Sambrook”) and CURRENT PROTOCOLS IN MOLECULAR BIOLOGY, F. M. Ausubel et al., eds., CURRENT PROTOCOLS, A JOINT VENTURE BETWEEN GREENE PUBLISHING ASSOCIATES, INC. AND JOHN WILEY AND SONS, INC., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al., (1987) U.S. Pat. No. 4,683,202; Innis et al., eds. (1990); PCR PROTOCOLS: A GUIDE TO METHODS AND APPLICATIONS (Academic Press Inc. San Diego, Calif.) (“Innis”); Arnheim and Levinson (Oct. 1, 1990) Cand EN 36-47; J. NIH RES. (1991) 3:81-94; Kwoh et al., (1989) PROC. NATL. ACAD. SCI. USA 86: 1173; Guatelliet et al., (1990) PROC. NAT'L. ACAD. SCI. USA 87: 1874; Lomell et al., (1989) J. CLIN. CHEM 35: 1826; Landegren et al., (1988) SCIENCE 241: 1077-80; Van Brunt (1990) BIOTECHNOLOGY 8: 291-94; Wu and Wallace (1989) GENE 4:560; Barringer et al., (1990) GENE 89:117; and, Sooknanan and Malek (1995) BIOTECHNOLOGY 13: 563-564. Improved methods for cloning in vitro amplified nucleic acids are described in Wallace et al., U.S. Pat. No. 5,426,039. Improved methods for amplifying large nucleic acids by PCR are summarized in Cheng et al., (1994) NATURE 369: 684-85 and the references cited therein, in which PCR amplicons of up to 40 kb are generated.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one value, and/or to “about” another value. When such a range is expressed, another embodiment includes from the one value and/or to the other value. Similarly, when values are expressed as approximations, by use of the antecedent “about,” it will be understood that the value forms another embodiment. It will be further understood that the endpoints of each of the ranges are significant both in relation to the other endpoint, and independently of the other endpoint. It is also understood that there are several values disclosed herein, and that each value is also herein disclosed as “about” that value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in several different formats, and that this data, represents endpoints and starting points, and ranges for any combination of the data points. For example, if a particular datapoint “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims, which follow, reference will be made to several terms which shall be defined to have the following meanings:

The term “cancer” or “tumor” includes, but is not limited to, solid tumors and blood borne tumors. These terms include diseases of the skin, tissues, organs, bone, cartilage, blood, and vessels. These terms further encompass primary and metastatic cancers.

The term “PD-1” refers to a protein found on T cells that helps keep the immune responses in check. When PD-1 is bound to another protein called PD-L1, it helps keep T cells from killing other cells, including cancer cells. Some anticancer drugs, called immune checkpoint inhibitors, are used to block PD-1. When this protein is prevented from acting on T cells, they can act to kill cancer cells.

The term “STAT3” refers to Signal transducer and activator of transcription 3 (STAT3) which is a transcription factor which in humans is encoded by the STAT3 gene (STAT3 Human (Hs) NM_001369512.1 Genbank RefSeq #, or NM_139276.3). STAT3 mediates the expression of a variety of genes in response to cell stimuli, and thus plays a key role in many cellular processes such as cell growth and apoptosis, as well as the growth and progression of cancer.

The term “TGF-β” refers to Transforming growth factor beta (TGF-β) which is a cytokine involved in immune and stem cell regulation and differentiation. TGF-β is an important cytokine with identified roles in many pathologies including cancer, infectious disease, and autoimmunity. Its immunosuppressive functions in the tumor microenvironment contribute to oncogenesis (Massague et al., CELL., 103 (2): 295-309 (2000)).

The term “CXCR2” refers to C—X—C motif chemokine receptor 2 (CXCR2) which is a receptor for interleukin 8 (IL-8) and a member of the G-protein-coupled receptor family. CXCR2 can mediate neutrophil migration to areas of inflammation.

The term “CCR2” refers to C—C chemokine receptor type 2 (CCR2) which is a receptor for monocyte chemoattractant protein 1. The inflammatory response in some cancers can be partially mediated by the activities of monocyte chemoattractant protein 1.

The term “ARG1” refers to Arginase-1 (ARG1) which is an enzyme that converts L-arginine to urea and L-ornithine. L-arginine and its downstream metabolites contribute to a suppressive tumor microenvironment through modulation of T-cell activity (Kim et al., FRONTIERS IN ONCOLOGY, 8:67 (2018)).

The term “PTGS2” refers to Prostaglandin-endoperoxide synthase 2 (PTGS2) which is also known as cyclooxygenase-2 or COX-2. PTGS2 is a key enzyme in prostaglandin synthesis. Prostaglandins can inhibit anti-tumor activities of some immune cells, contributing to a suppressive tumor microenvironment.

The term “CTLA-4” refers to Cytotoxic T-lymphocyte-associated protein 4 (CTLA-4) or cluster of differentiation 152 (CD152) which is a protein found on T cells that helps keep the immune responses in check. CTLA-4 was the first immune checkpoint target and CTLA-4 inhibitors have been developed as breakthrough anti-cancer treatments.

The term “SOCS1” refers to Suppressor of cytokine signaling 1 (SOCS1) which is a member of the STAT-induced STAT inhibitor (SSI) family. SOCS1 is a cytokine-inducible negative regulator of cytokine signaling.

As used herein, the term “cold tumor” or “non-inflamed tumor” refers to a tumor or tumor microenvironment wherein there is minimal to no presence of anti-tumor immune cells, such as tumor infiltrating lymphocytes (TILs), and/or contain cell subsets associated with immune suppression including regulatory T cells (Treg), myeloid-derived suppressor cells (MDSCs) and M2 macrophages. Specifically, in some embodiments, a cold tumor is characterized by a low number or even absence of infiltration of anti-tumor immune cells that such cells may be present but remain stuck in the surrounding stroma, thus unable to colonize the tumor microenvironment to provide their antitumor functions.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “species cross-reactive oligonucleotide” refers to an oligonucleotide capable of inhibiting expression of a target mRNA in more than one species. For example, in some embodiments a species cross-reactive oligonucleotide is capable of inhibiting expression of a target mRNA in human and non-human primates. Example species include but is not limited to human, non-human primates, mouse, and rat. In some embodiments, species cross-reactive oligonucleotides are capable of targeting and inhibiting mRNA in at least two, at least three, or at least four species.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA” or “dsRNA” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, the term “hot tumor” or “inflamed tumor” refers to a tumor or tumor microenvironment wherein there is a considerable presence of anti-tumor immune cells especially TILs and thus are typically immuno-stimulatory.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”). The loop may refer to a loop comprising four nucleotides as a tetraloop (tetraL). The loop may refer to a loop comprising three nucleotides as a triloop (triL).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single stranded (ss) or double-stranded (ds). An oligonucleotide may or may not have duplex regions. An oligonucleotide may comprise deoxyribonucleotides, ribonucleosides, or a combination of both. In some embodiments, a double-stranded oligonucleotide comprising ribonucleotides is referred to as “dsRNA”. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded RNA (dsRNA) is an RNAi oligonucleotide.

The terms “RNAi oligonucleotide conjugate” and “oligonucleotide-ligand conjugate” are used interchangeably and refer to an oligonucleotide comprising one or more nucleotides conjugated with one or more targeting ligands.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In certain embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al., (2015) NUCLEIC ACIDS RES. 43:2993-3011).

As used herein, “reduced expression” of a gene (e.g., STAT3) refers to a decrease in the amount or level of RNA transcript (e.g., STAT3 mRNA) or protein encoded by the gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide herein (e.g., an oligonucleotide comprising an antisense strand having a nucleotide sequence that is complementary to a nucleotide sequence comprising STAT3 mRNA) may result in a decrease in the amount or level of STAT3 mRNA, protein and/or activity (e.g., via degradation of STAT3 mRNA by the RNAi pathway) when compared to a cell that is not treated with the dsRNA. Similarly, and as used herein, “reducing expression” refers to an act that results in reduced expression of a gene (e.g., STAT3). As used herein, “reduction of STAT3 expression” refers to a decrease in the amount or level of STAT3 mRNA, STAT3 protein and/or STAT3 activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA.

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, non-human primates (NHP), and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule or “moiety” (e.g., a carbohydrate, amino sugar, cholesterol, polypeptide, or lipid) that selectively binds to a cognate molecule (e.g., a receptor) of a tissue or cell of interest and/or that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “loop”, “triloop”, or “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a loop (e.g., a tetraloop or triloop) can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a loop (e.g., a tetraloop) may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al., (1990) NATURE 346:680-82; Heus and Pardi (1991) SCIENCE 253:191-94). In some embodiments, a loop comprises or consists of 3 to 6 nucleotides and is typically 4 to 5 nucleotides. In certain embodiments, a loop comprises or consists of 3, 4, 5 or 6 nucleotides, which may or may not be modified (e.g., which may or may not be conjugated to a targeting moiety). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a loop consisting of 4 nucleotides is a tetraloop. Any nucleotide may be used in the loop (e.g., a tetraloop) and standard IUPAC-IUB symbols for such nucleotides may be used as described in Cornish-Bowden ((1985) NUCLEIC ACIDS RES. 13:3021-3030). For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., (1990) PROC. NATL. ACAD. SCI. USA 87:8467-71; Antao et al., (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (See, e.g., Nakano et al., (2002) BIOCHEM. 41:4281-92; Shinji et al., (2000) NIPPON KAGAKKAI KOEN YOKOSHU 78:731). In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

As used herein, the term “tumor microenvironment” relates to the cellular environment in which any given tumor exists, including the tumor stroma, surrounding blood vessels, immune cells, fibroblasts, other cells, signaling molecules, and the ECM. It is understood that the tumor microenvironment harbors and/or surrounds the tumor cells with which it interacts.

### Oligonucleotide Conjugates for Delivery to Immune Cells in the Tumor Microenvironment

The tumor microenvironment (TME) plays a key role in sustaining tumor growth, invasion, and ultimately metastasis. The complex TME is comprised in part by immune cells, fibroblasts, and blood vessels. The immune cell composition in the TME is typically categorized as a “cold” or “hot” tumor. Cold tumors have a dampened immune response due at least in part to the presence of myeloid-derived suppressor cells (MDSC) and T regulatory cells (Tregs). Both MDSCs and Tregs dampen the ability of T-cells to infiltrate the tumor and induce an anti-tumor response. Hot tumors show infiltration of cancer-fighting T cells demonstrating a combative anti-tumor response. Cold tumors are generally less responsive to immunotherapy treatments compared to hot tumors. Therapies to convert the tumor immune environment from a cold to hot environment are needed.

mRNA Target Sequences

In some embodiments, the oligonucleotide-ligand conjugate is targeted to an mRNA target sequence in an immune cell associated with a tumor microenvironment via the targeting ligand. In some embodiments, the oligonucleotide-ligand conjugate, or a portion, fragment, or strand thereof (e.g., an antisense strand or a guide strand of a double-stranded oligonucleotide) binds or anneals to a target mRNA sequence, thereby reducing expression of the target mRNA. In some embodiments, the oligonucleotide-ligand conjugate is targeted to an mRNA target sequence in an immune cell associated with a tumor microenvironment via the targeting ligand for the purpose of reducing expression of the target mRNA in vivo. In some embodiments, the amount or extent of reduction of expression of the target mRNA by an oligonucleotide-ligand conjugate correlates with the potency of the oligonucleotide-ligand conjugate. In some embodiments, the amount or extent of reduction of expression of the target mRNA by an oligonucleotide-ligand conjugate correlates with the amount or extent of therapeutic benefit in a subject or patient having cancer treated with the oligonucleotide-ligand conjugate.

Through examination of the nucleotide sequence of target mRNAs, including mRNAs of multiple different species (e.g., human, cynomolgus monkey, mouse, and rat) and as a result of in vitro and in vivo testing, it has been discovered that certain target mRNA sequences are more amenable than others to oligonucleotide-mediated reduction and are thus useful as target sequences for the oligonucleotide-ligand conjugate herein. In some embodiments, a sense strand of an oligonucleotide-ligand conjugate (e.g., RNAi oligonucleotide-lipid conjugate), or a portion or fragment thereof, described herein, comprises a nucleotide sequence that is similar (e.g., having no more than 4 mismatches) or is identical to a target mRNA sequence. In some embodiments, a portion or region of the sense strand of a double-stranded oligonucleotide described herein comprises a target mRNA sequence.

In some embodiments, the oligonucleotide-ligand conjugate targets an mRNA encoding a regulator of immune suppression expressed by an immune cell in a TME. In some embodiments, the regulator of immune suppression directly or indirectly impacts immune regulation. For example, in some embodiments, the regulator of immune suppression is a regulatory protein, an enzymatic protein, or a signaling protein. In some embodiments, the regulator of immune suppression is a polypeptide that controls immune signaling. In some embodiments, the regulator of immune suppression is an enzyme involved in processing a polypeptide involved in immune regulation. In some embodiments, the regulator of immune suppression is a checkpoint inhibitor polypeptide. In some embodiments, the regulator of immune suppression is a transcription factor. In some embodiments, the regulator of immune suppression is a cytokine. In some embodiments, the regulator of immune suppression is a chemokine receptor.

Both wild-type and mutated genes encoding immune regulators are capable of modifying the immune response in the TME or tumor draining lymph node (TdLN). In some embodiments, the oligonucleotide-ligand conjugate targets a wild-type mRNA encoding a regulator of immune suppression expressed by an immune cell in a TME. In some embodiments, the oligonucleotide-ligand conjugate targets a wild-type mRNA encoding a regulator of immune suppression expressed by an immune cell in a TdLN. In some embodiments, the oligonucleotide-ligand conjugate targets a mutated mRNA encoding a regulator of immune suppression expressed by an immune cell in a TME. In some embodiments, the oligonucleotide-ligand conjugate targets a mutated mRNA encoding a regulator of immune suppression expressed by an immune cell in a TdLN. Mutated mRNA molecules produce misfolded proteins or hyperactive proteins.

In some embodiments, the oligonucleotide-ligand conjugate directly or indirectly reduces expression of proteins that contribute to the suppressive function of M-MDSC's. In some embodiments, the oligonucleotide-ligand conjugate directly or indirectly reduces expression of proteins that contribute to the suppressive function of G-MDSC's.

In some embodiments, the oligonucleotide-ligand conjugate reduces target mRNA expression by at least 10%, at least 20%, at least 30%, at least 40%, at least 50%, at least 60%, at least 70%, at least 80%, at least 90%, or at least 95% in an immune cell of the TME. In some embodiments, the oligonucleotide-ligand conjugate reduces expression of the regulator of immune suppression by about 10%, about 20%, about 30%, about 40%, about 50%, about 60%, about 70%, about 80%, or about 90% in an immune cell of the TME.

In some embodiments, the oligonucleotide-ligand conjugate reduces target mRNA expression by at least 10%, at least 20%, at least 30%, at least 40%, at least 50%, at least 60%, at least 70%, at least 80%, at least 90%, or at least 95% in an immune cell of the TdLN. In some embodiments, the oligonucleotide-ligand conjugate reduces expression of the regulator of immune suppression by about 10%, about 20%, about 30%, about 40%, about 50%, about 60%, about 70%, about 80%, or about 90% in an immune cell of the TdLN.

### Immune Cells in a Tumor Microenvironment

In some aspects, the disclosure provides oligonucleotide-ligand conjugates that reduce expression of a target mRNA expressed in an immune cell present in a tumor and/or tumor microenvironment. In some embodiments, the oligonucleotide-ligand conjugate targets a suppressive immune cell in the tumor microenvironment. In some embodiments, the targeting ligand of the conjugate delivers the oligonucleotide to an immune cell present in a tumor.

In healthy individuals, immature myeloid cells produced from bone marrow differentiate into mature granulocytes, macrophages or dendritic cells and go on to become part of the innate immune system (Weiskopf et al., MICROBIOL SPECTR. October; 4(5) (2016)). In pathological conditions such as cancer, a partial block in the differentiation of immature myeloid cells into mature myeloid cells can result in an expansion of the population of immature myeloid cells (Gabrilovitch et al., NAT REV IMMUNOL. March; 9(3): 162-74 (2009)) incapable of assisting in cancer monitoring or removal. Under the influence of GM-CSF secreted by cancer cells, these excess myeloid cells are recruited from bone marrow to the tumor site (Schmid and Varner. JOURNAL OF ONCOLOGY (2010)). Once within the TME, the myeloid cell population expands, and the cells exert immune suppressive functions that enables them to suppress T cells and NK cells through different mechanisms (Yang et al., FRONT. IN IMMUNOL. 11:1371 (2020)) directly inhibiting a response to the cancer tumor.

Myeloid derived suppressor cells (MDSCs) contribute to immunotherapeutic resistance by actively inhibiting anti-tumor T-cell proliferation and cytotoxic activity, as well as by promoting expansion of immunosuppressive T regulatory cells (Gabrilovich et al., NAT REV IMMUNOL (2009) 9(3): 162-74, Law et al., CELLS (2020) 9: 561). In this way MDSCs can inhibit or attenuate the host immune response against a tumor. In addition, these MDSCs can also assist in cell dissemination through the promotion of angiogenesis, EMT and MET transition as well as in the secretion of tumorigenic factors. (Law et al., CELLS (2020) 9: 561). Given their importance in the development, maintenance, and assistance in the expansion of tumors with which they are associated MDSCs are potential therapeutic targets for many tumor types if they can be attacked specifically. MDSCs can also be found in tumor draining lymph nodes (TdLN) where they can have a suppressive effect on naïve T cells also found in tumor draining lymph nodes (Swatz et al., NAT REV CANCER (2012) 12: 210-19). Suppression of naïve T cells can then set the stage for tumors to metastasize into the lymph nodes and beyond (Swatz et al., NAT REV CANCER (2012) 12: 210-19). Collectively, MDSCs are characterized by the co-expression of cell surface or mRNA markers CD11b (a marker for the myeloid cells of the macrophage lineage) and Gr-1 (a marker for the myeloid lineage differentiation antigen) and denoted as CD11b⁺Gr-1⁺ cells. Gr-1 is further comprised of 2 components Ly6G and Ly6C. MDSCs consist of two subsets: Granulocytic MDSC (G-MDSC), further characterized as CD11b⁺Ly6G⁺Ly6C^{lo}, and monocytic MDSC (M-MDSC) characterized as CD11b⁺Ly6G⁻Ly6C^{hi}. mRNA markers Ly6G, CxCr2, Slc27a2 and Ptgs2 are preferentially expressed by G-MDSCs and not by M-MDSCs. Expression of specific markers such as CxCr2, Scl27a2 and Ptgs2 suggest the recruitment and suppression activity of G-MDSCs in the TME. Likewise, mRNA markers Ly6C, Scarb1, Ldlr and Arg1 are highly expressed by M-MDSCs compared to G-MDSCs. Higher expression of lipid trafficking receptors such as Scarb1 and Ldlr in M-MDSCs may play key role in lipid uptake.

In some embodiments, the oligonucleotide-ligand conjugate targets a tumor resident immune cell. In some embodiments, the oligonucleotide-ligand conjugate targets an immune cell in the tumor draining lymph node (TdLN). In some embodiments, the oligonucleotide-ligand conjugate targets an mRNA in a tumor resident immune cell. In some embodiments, the oligonucleotide-ligand conjugate targets an mRNA in an immune cell in the tumor draining lymph node (TdLN).

In some embodiments, the immune cell is a suppressive myeloid cell. In some embodiments, the immune cell is a myeloid derived suppressor cell (MDSC). In some embodiments, the MDSC is a granulocytic MDSC (G-MDSC). In some embodiments, the MDSC is a monocytic MDSC (M-MDSC).

In some embodiments, the immune cell is a T-cell. In some embodiments, the T cell is a CD8+ T cell. In some embodiments, the T-cell is a Treg cell.

In some embodiments, the oligonucleotide-ligand conjugate reduces a target mRNA in a tumor resident and/or tumor draining lymph node MDSC. In some embodiments, the oligonucleotide conjugate reduces a target mRNA in a tumor resident and/or tumor draining lymph node G-MDSC. In some embodiments, the oligonucleotide-ligand conjugate reduces a target mRNA in a tumor resident and/or tumor draining lymph node M-MDSC. In some embodiments, the oligonucleotide-ligand conjugate reduces a target mRNA in a tumor resident and/or tumor draining lymph node Treg cell. In some embodiments, the oligonucleotide-ligand conjugate reduces a target mRNA in more than one type tumor resident and/or tumor draining lymph node immune cell. For example, in some embodiments, the oligonucleotide-ligand conjugate reduces a target mRNA in a MDSC (e.g., M-MDSC and/or G-MDSC) and a T cell (e.g., CD8+ T cell and/or Treg cell).

In some embodiments, the immunosuppressive activity of the immune cell (e.g. MDSC or Treg cell) is reduced after contact with the oligonucleotide-ligand conjugate. Immunosuppressive activity is measured using known methods in the art. In one such method, Arginase I levels are measured in isolated tumor immune cells compared to control immune cells. High Arginase I levels in tumor resident immune cells (e.g. myeloid cells) is indicative of an immunosuppressive environment. Additionally, in some embodiments the number of immune suppressive tumor resident cells indicates the level of suppressive activity. In some embodiments, T-cell suppression assays and/or cytokine release assays are used to measure the suppressive activity of an immune cell.

### Cancers

In some embodiments, the oligonucleotide-ligand conjugate described herein targets immune cells in a tumor. In some embodiments, the tumor is a primary tumor. In some embodiments, the tumor is a metastatic tumor. In some embodiments, the tumor is a refractory tumor. In some embodiments, the tumor is a Stage I, Stage II, Stage III, or Stage IV tumor. In some embodiments, the tumor is a solid-tumor. Solid-tumors refer to conditions where the cancer forms a mass

In some embodiments, the cancer is a thyroid cancer, papillary thyroid carcinoma, head and neck cancer, liver cancer, colorectal cancer, pancreatic cancer, breast cancer, ovarian cancer, lung cancer, carcinoma, blastoma, medulloblastoma, retinoblastoma, sarcoma, liposarcoma, synovial cell sarcoma, neuroendocrine tumors, carcinoid tumors, gastrinoma, islet cell cancer, mesothelioma, schwannoma, acoustic neuroma, meningioma, adenocarcinoma, lymphoid malignancies, squamous cell cancer, epithelial squamous cell cancer, small-cell lung cancer (SCLC), non-small cell lung cancer (NSCLC), adenocarcinoma of the lung, squamous carcinoma of the lung, cancer of the peritoneum, hepatocellular cancer, gastric or stomach cancer, gastrointestinal cancer, glioblastoma, cervical cancer, bladder cancer, hepatoma, metastatic breast cancer, colon cancer, rectal cancer, endometrial or uterine carcinoma, salivary gland carcinoma, kidney or renal cancer, prostate cancer, vulval cancer, hepatic carcinoma, anal carcinoma, penile carcinoma, Merkel cell cancer, testicular cancer, esophageal cancer, or tumors of the biliary tract. In some embodiments, the cancer is refractory to anti-PD1, anti-PDL1 and/or anti-CTLA4 therapy. In some embodiments, the cancer is a pancreatic cancer or lung cancer. In some embodiments, the cancer comprises tumors with immunosuppressive tumor microenvironments.

In some embodiments, the oligonucleotide-ligand conjugate is delivered to the tumor and reduces a target mRNA's expression in a tumor resident immune cell.

In some embodiments, the oligonucleotide-ligand conjugate reduces tumor volume. Tumor volume is measured using methods know to one of skill in the art. For example, extracted tumors are measured manually using calipers. Other methods include imagine methods such as ultrasound and MRI. In some embodiments, the oligonucleotide conjugate reduces tumor volume by at least 5%, at least 10%, at least 20%, at least 30%, at least 40%, at least 50%, at least 60%, at least 70%, at least 80%, or at least 90% compared to an untreated tumor.

Tumor draining lymph nodes (TdLN) are the generally the first site of metastasis for cancer. In some embodiments, the oligonucleotide conjugate targets immune cells in the tumor draining lymph node. In some embodiments, the tumor draining lymph node is the subsegmental, segmental, lobar, interlobar, hilar, mediastinal, supratrochlear, deltoideopectoral, lateral, pectoral, subscapular, intermediate, subclavicular, superficial inguinal, deep inguinal, popliteal, facial buccinators, facial nasolabial, prostate, mandibular, submental, occipital, mastoid/retroauricular, parotid, deep preauricular, deep infra-auricular, deep intraglandular, deep cervical, deep anterior cervical, pretracheal, paratracheal, prelaryngeal, thyroid, deep lateral cervical, superior deep cervical, inferior deep cervical, retropharyngeal, jugulodigastric, anterior cervical, lateral cervical, supraclavicular, retroaortic, lateral aortic, celiac, gastric, hepatic, splenic, superior mesenteric, mesenteric, ileocolic, mesocolic, inferior mesenteric, or pararectal lymph node. In some embodiments, the tumor draining lymph node is a primary tumor draining lymph node. In some embodiments, the tumor draining lymph node is a lymph node that drains a tumor metastasis.

In some embodiments, the oligonucleotide-ligand conjugate does not target immune cells in the non-TdLN. In some embodiments, the oligonucleotide-ligand conjugate does not target cancer cells.

In some embodiments, the oligonucleotide-ligand conjugate targets immune cells in both the tumor and tumor draining lymph nodes. In some embodiments, the oligonucleotide-ligand conjugate reduces target mRNA in immune cells in a TdLN by at least 10%, at least 20%, at least 30%, at least 40%, at least 50%, at least 60%, at least 70%, at least 80%, or at least 90%.

### Structure of Oligonucleotide-Targeting Ligand Conjugates

In some embodiments, an oligonucleotide-ligand conjugate described herein comprises a nucleotide sequence and one or more targeting ligands, wherein the nucleotide sequence comprises one or more nucleosides (nucleic acids) conjugated with one or more targeting ligands represented by formula I-a:

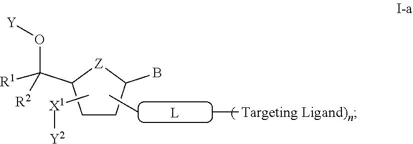

or a pharmaceutically acceptable salt thereof,
wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each targeting ligand is selected from lipid conjugate moiety (LC), carbohydrate, amino sugar or

GalNAc; and wherein each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

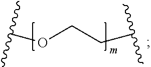

m is 1-50;
X¹, V¹ and W¹ are independently —C(R)₂—, —OR, —O—, —S—, —Se—, or —NR—;
Y is hydrogen, a suitable hydroxyl protecting group,

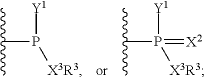

R³ is hydrogen, a suitable protecting group, a suitable prodrug, or an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

In some embodiments, the oligonucleotide-ligand conjugate comprises one or more nucleic acids conjugated with targeting ligands represented by formula II-a:

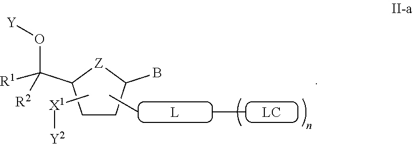

or a pharmaceutically acceptable salt thereof.

In some embodiments, the oligonucleotide-ligand conjugate comprises one or more nucleic acids conjugated with targeting ligands represented by formula II-b or II-c:

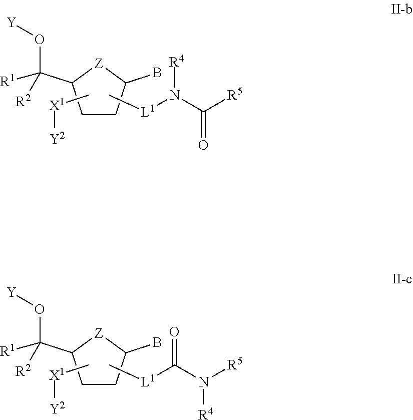

or a pharmaceutically acceptable salt thereof, wherein:
L¹ is a covalent bond, a monovalent or a bivalent saturated or unsaturated, straight or branched
C¹⁻⁵⁰ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

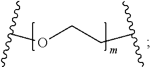

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR. In some embodiments, R⁵ is selected from

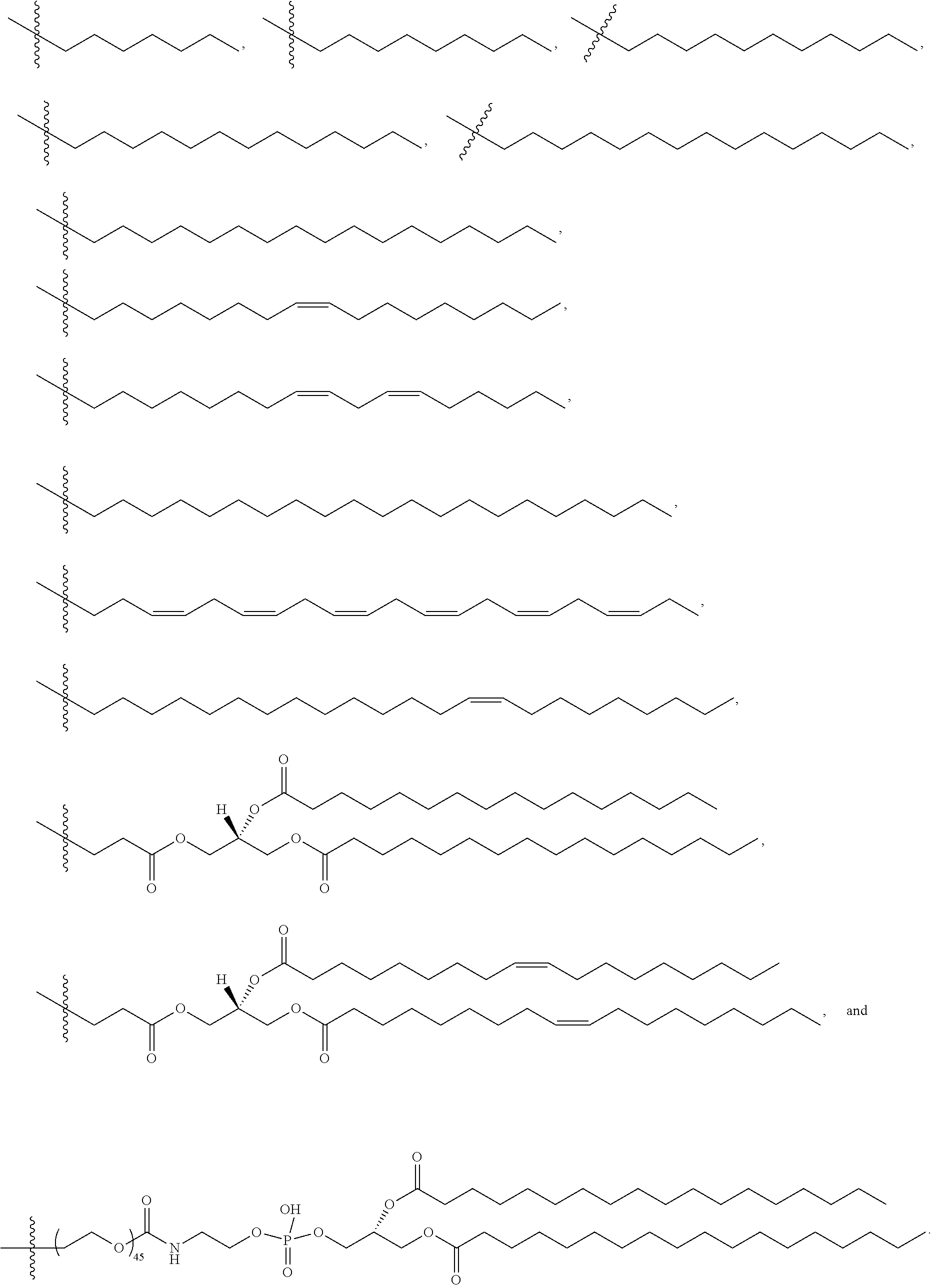

In some embodiments, R⁵ is selected from:

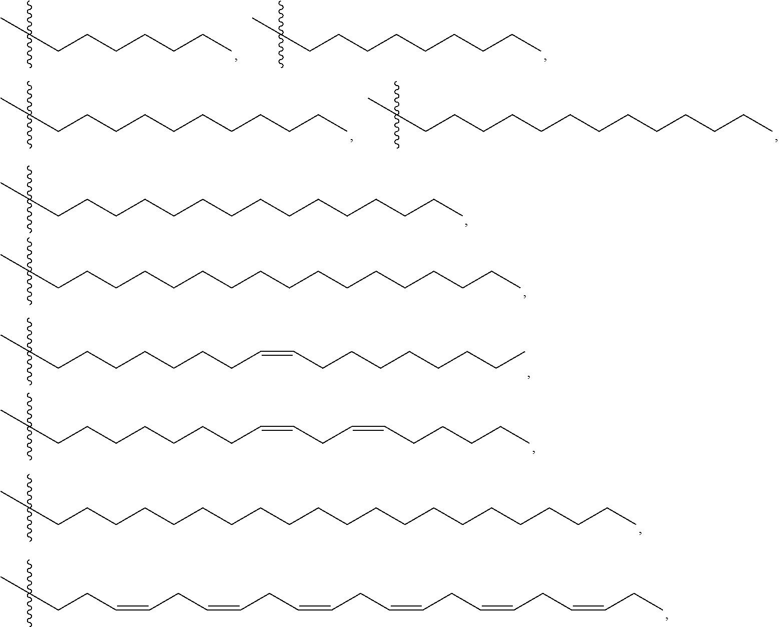

In some embodiments, R⁵ is

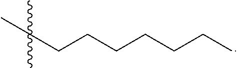 [SMILES: *CCCCCCC]

In some embodiments, R⁵ is

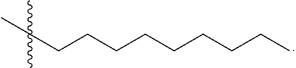 [SMILES: *CCCCCCCCC]

In some embodiments, R⁵ is

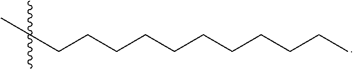 [SMILES: *CCCCCCCCCCC]

In some embodiments, R⁵ is

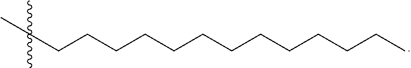 [SMILES: *CCCCCCCCCCCCC]

In some embodiments, R⁵ is

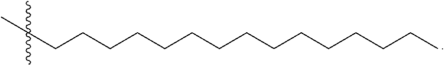 [SMILES: *CCCCCCCCCCCCCCC]

In some embodiments, R⁵ is

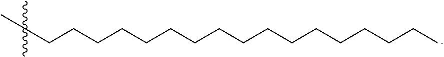 [SMILES: *CCCCCCCCCCCCCCCCC]

In some embodiments, R⁵ is

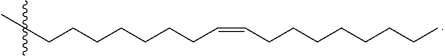 [SMILES: *CCCCCCC/C=C\CCCCCCCC]

In some embodiments, R⁵ is

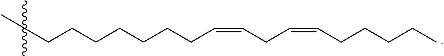 [SMILES: *CCCCCCC/C=C\C/C=C\CCCCC]

In some embodiments, R⁵ is

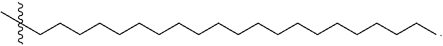 [SMILES: *CCCCCCCCCCCCCCCCCCCCC]

In some embodiments, R⁵ is

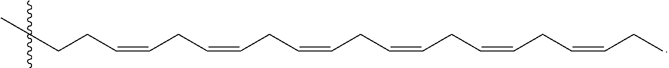 [SMILES: *CC/C=C\C/C=C\C/C=C\C/C=C\C/C=C\C/C=C\CC]

In some embodiments, R⁵ is

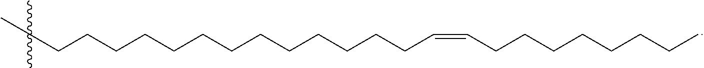 [SMILES: *CCCCCCCCCCCCC/C=C\CCCCCCCC]

In some embodiments, R⁵ is

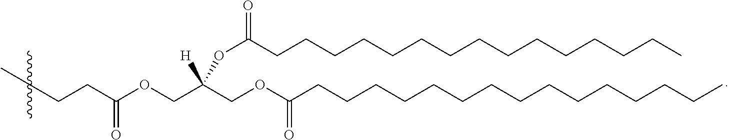 [SMILES: *CCC(=O)OC[C@@H](COC(=O)CCCCCCCCCCCCCCC)OC(=O)CCCCCCCCCCCCCCC]

In some embodiments, R⁵ is

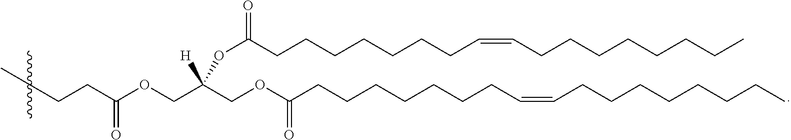 [SMILES: *CCC(=O)OC[C@@H](COC(=O)CCCCCCC/C=C\CCCCCCCC)OC(=O)CCCCCCC/C=C\CCCCCCCC]

In some embodiments, R⁵ is

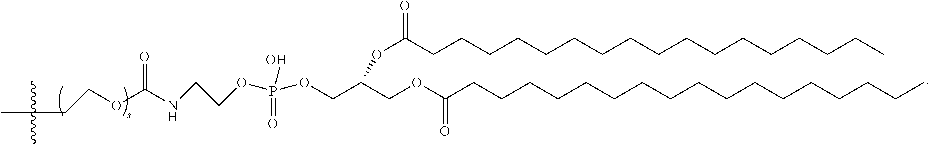

In some embodiments, the oligonucleotide-ligand conjugate comprises one or more nucleic acids conjugated with targeting ligands represented by formula II-Ib or II-Ic:

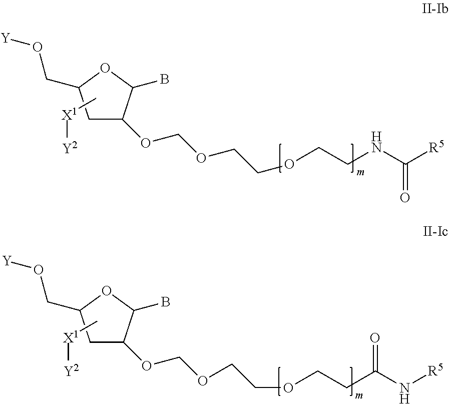

or a pharmaceutically acceptable salt thereof; wherein
B is a nucleobase or hydrogen;
m is 1-50;
X¹ is —O—, or —S—;
Y is hydrogen,

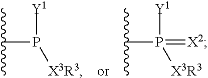

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(O)OR—; and
R is hydrogen, a suitable protecting group, or an optionally substituted group selected from C¹⁻⁶ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁵ is selected from

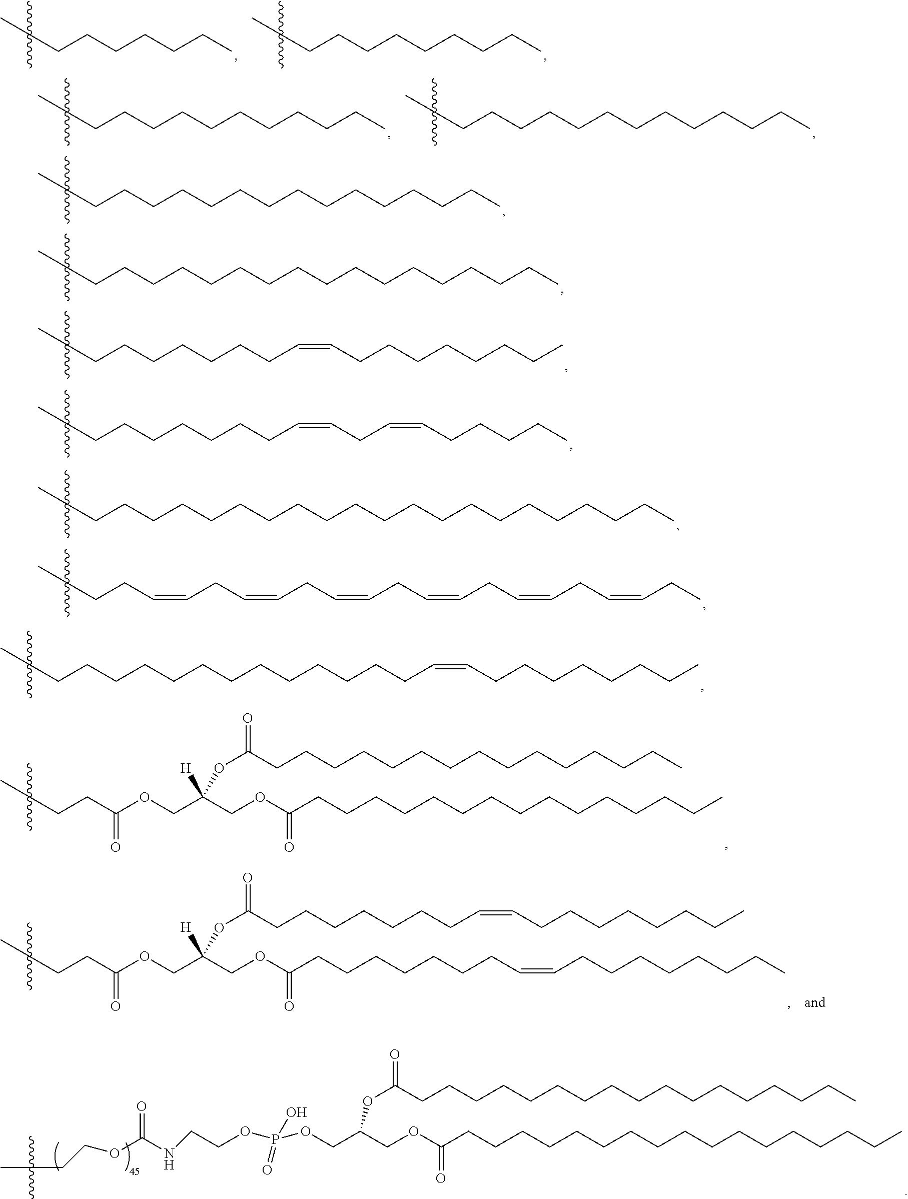

In some embodiments, R⁵ is

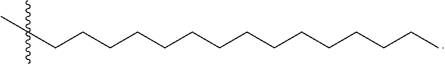 [SMILES: *CCCCCCCCCCCCCCC]

In some embodiments, R⁵ is

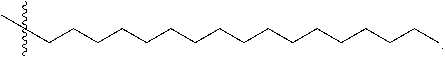 [SMILES: *CCCCCCCCCCCCCCCCC]

In some embodiments, the nucleotide sequence of the oligonucleotide comprises 1-10 targeting ligands. In some embodiments, the nucleotide sequence comprises 1, 2 or 3 targeting ligands.

In some embodiments, the oligonucleotide of the oligonucleotide-ligand conjugate is a double-stranded molecule. In some embodiments, the oligonucleotide is an RNAi molecule. In some embodiments, the double stranded oligonucleotide comprises a stem loop. In some embodiments, the ligand is conjugated to any of the nucleotides in the stem loop. In some embodiments, the ligand is conjugated to the first nucleotide from 5′ to 3′, in the stem loop. In some embodiments, the ligand is conjugated to the second nucleotide from 5′ to 3′ in the stem loop. In some embodiments, the ligand is conjugated to the third nucleotide from 5′ to 3′ in the stem loop. In some embodiments, the ligand is conjugated to the fourth nucleotide from 5′ to 3′ in the stem loop. In some embodiments, the ligand is conjugated to one, two, three, or four of the nucleotides in the stem loop. In some embodiments, the ligand is conjugated to three of the nucleotides in the stem loop.

In some embodiments, the oligonucleotide-ligand conjugate comprises a sense strand of 36 nucleotides with positions numbered 1-36 from 5′ to 3′. In some embodiments, the oligonucleotide-ligand conjugate comprises a lipid conjugated to position 27 of a 36-nucleotide sense strand. In some embodiments, the oligonucleotide-ligand conjugate comprises a lipid conjugated to position 28 of a 36-nucleotide sense strand. In some embodiments, the oligonucleotide conjugate comprises a lipid conjugated to position 29 of a 36-nucleotide sense strand. In some embodiments, the oligonucleotide conjugate comprises a lipid conjugated to position 30 of a 36-nucleotide sense strand.

In some embodiments, an oligonucleotide-ligand conjugate comprises an antisense strand of 15 to 30 nucleotides and a sense strand of 15 to 40 nucleotide, wherein the sense and antisense strands form a duplex region, wherein the antisense strand comprises a region of complementarity to a target sequence expressed in an immune cell associated with a tumor microenvironment, wherein the sense strand comprises at its 3′ end a stem-loop comprising a tetraloop comprising 4 nucleosides, wherein one or more of the 4 nucleosides is represented by formula II-Ib:

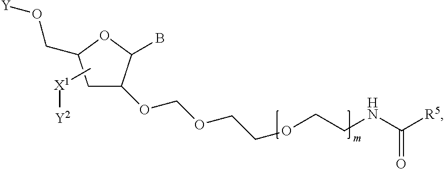

wherein B is selected from an adenine and a guanine nucleobase, and wherein R⁵ is a hydrocarbon chain. In some embodiments, m is 1, X1 is O, Y2 is an internucleotide linking group attaching to the 5′ terminal of a nucleoside,

Y is represented by

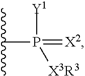

Y1 is a linking group attaching to the 2′ or 3′ terminal of a nucleotide, X2 is O, X3 is O, and R3 is H. In some embodiments, the hydrocarbon chain is a C8-C30 hydrocarbon chain. In some embodiments, the hydrocarbon chain is a C16 hydrocarbon chain. In some embodiments, the C16 hydrocarbon chain is represented by

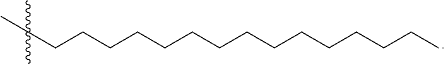 [SMILES: *CCCCCCCCCCCCCCC]

In some embodiments, the 4 nucleosides of the tetraloop are numbered 1-4 from 5′ to 3′ and position 1 is represented by formula II-Ib. In some embodiments, position 2 is represented by formula II-Ib. In some embodiments, position 3 is represented by formula II-Ib. In some embodiments, position 4 is represented by formula II-Ib. In some embodiments, the sense strand is 36 nucleotides with positions numbered 1-36 from 5′ to 3′, wherein the stem-loop comprises nucleotides at positions 21-36, and wherein one or more nucleosides at positions 27-30 are represented by formula II-Ib. In some embodiments, the antisense strand is 22 nucleotides.

In some aspects, the disclosure provides oligonucleotide-ligand conjugates for targeting a target mRNA (e.g., a target mRNA regulating immune suppression) and inhibiting or reducing target gene expression (e.g., via the RNAi pathway), wherein the oligonucleotide-ligand conjugate is a double-stranded (ds) nucleic acid molecule comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds or anneals to one another in a complementary manner (e.g., by Watson-Crick base pairing).

In some embodiments, the sense strand has a first region (R1) and a second region (R2), wherein R2 comprises a first subregion (S1), a loop (L), such as a tetraloop (tetraL) or triloop (triL), and a second subregion (S2), wherein L or triL is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 19 nucleotides in length. In some embodiments, D1 is 20 nucleotides in length. In some embodiments, D1 comprising the sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

It should be appreciated that, in some embodiments, sequences presented in the Sequence Listing may be referred to in describing the structure of an oligonucleotide (e.g., a oligonucleotide-ligand conjugate) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, an oligonucleotide-ligand conjugate herein comprises a 25-nucleotide sense strand and a 27-nucleotide antisense strand that when acted upon by a Dicer enzyme results in an antisense strand that is incorporated into the mature RISC. In some embodiments, the sense strand of the oligonucleotide-ligand conjugate is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide-ligand conjugate is longer than 25 nucleotides (e.g., 26, 27, 28, 29 or 30 nucleotides).

In some embodiments, the oligonucleotide-ligand conjugates herein have one 5′ end that is thermodynamically less stable when compared to the other 5′ end. In some embodiments, an asymmetric oligonucleotide-ligand conjugate is provided that comprises a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, an oligonucleotide-ligand conjugate has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., a target mRNA regulating immune suppression). In some embodiments, the two terminal nucleotides on the 3′ end of the antisense strand are not complementary with the target mRNA. In some embodiments, the two terminal nucleotides on the 3′ end of the antisense strand of an oligonucleotide-ligand conjugate herein are unpaired. In some embodiments, the two terminal nucleotides on the 3′ end of the antisense strand of an oligonucleotide-ligand conjugate herein comprise an unpaired GG. In some embodiments, the two terminal nucleotides on the 3′ end of the antisense strand of an oligonucleotide-ligand conjugate herein are not complementary to the target mRNA. In some embodiments, two terminal nucleotides on each 3′ end of an oligonucleotide-ligand conjugate are GG. Typically, one or both of the two terminal GG nucleotides on each 3′ end of a double-stranded oligonucleotide (e.g., an RNAi oligonucleotide conjugate) is not complementary with the target mRNA.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of an oligonucleotide-ligand conjugate herein improves or increases the potency and/or efficacy of the oligonucleotide-ligand conjugate.

In some embodiments, the targeting ligand is a GalNAc as described herein. In some embodiments, the targeting ligand is a carbohydrate. In some embodiments, the targeting ligand is an amino sugar.

In some embodiments, the oligonucleotide-ligand conjugate comprises two or more targeting ligands, wherein the targeting ligands are different. In some embodiments, the oligonucleotide-ligand conjugate comprises two or more targeting ligands, wherein the targeting ligands are the same.

### Exemplary Oligonucleotides

In some embodiments, the oligonucleotide-ligand conjugate comprises an oligonucleotide conjugated with a fatty acid. In some embodiments, the fatty acid is a saturated fatty acid. In some embodiments, the fatty acid is an unsaturated fatty acid. In some embodiments, the oligonucleotide is conjugated with a lipid. In some embodiments, the lipid is a carbon chain. In some embodiments, the carbon chain is saturated. In some embodiments, the carbon chain is unsaturated. In some embodiments, the oligonucleotide is conjugated with a 16-carbon (C16) lipid. In some embodiments, the C16 lipid comprises at least one double bond. In some embodiments, the oligonucleotide is conjugated with an 18-carbon (C18) lipid. In some embodiments, the C18 lipid comprises at least one double bond. In some embodiments, the oligonucleotide is conjugated with a 22-carbon (C22) lipid. In some embodiments, the C22 lipid comprises at least one double bond. In some embodiments, the oligonucleotide is conjugated with a 24-carbon (C24) lipid. In some embodiments, the C24 lipid comprises at least one double bond.

In some embodiments, the oligonucleotide of the oligonucleotide-ligand conjugate comprises a loop wherein at least one nucleotide of the loop is conjugated with a C16 lipid. In some embodiments, the second nucleotide of the loop is conjugated with a C16 lipid. In some embodiments, the oligonucleotide of the oligonucleotide-ligand conjugate comprises a loop wherein at least one nucleotide of the loop is conjugated with a C18 lipid. In some embodiments, the second nucleotide of the loop is conjugated with a C18 lipid. In some embodiments, the oligonucleotide of the oligonucleotide-ligand conjugate comprises a loop wherein at least one nucleotide of the loop is conjugated with a C22 lipid. In some embodiments, the second nucleotide of the loop is conjugated with a C22 lipid. In some embodiments, the oligonucleotide of the oligonucleotide-ligand conjugate comprises a loop wherein at least one nucleotide of the loop is conjugated with a C24 lipid. In some embodiments, the second nucleotide of the loop is conjugated with a C24 lipid.

In some embodiments, the oligonucleotide of the oligonucleotide-ligand conjugate comprises a tetraloop wherein at least one nucleotide of the tetraloop is conjugated with a C16 lipid. In some embodiments, the second nucleotide of the tetraloop is conjugated with a C16 lipid. In some embodiments, the oligonucleotide of the oligonucleotide-ligand conjugate comprises a tetraloop wherein at least one nucleotide of the tetraloop is conjugated with a C18 lipid. In some embodiments, the second nucleotide of the tetraloop is conjugated with a C18 lipid. In some embodiments, the oligonucleotide of the oligonucleotide-ligand conjugate comprises a tetraloop wherein at least one nucleotide of the tetraloop is conjugated with a C22 lipid. In some embodiments, the second nucleotide of the tetraloop is conjugated with a C22 lipid. In some embodiments, the oligonucleotide of the oligonucleotide-ligand conjugate comprises a tetraloop wherein at least one nucleotide of the tetraloop is conjugated with a C24 lipid. In some embodiments, the second nucleotide of the tetraloop is conjugated with a C24 lipid.

In some embodiments, an oligonucleotide-ligand conjugate comprises a nucleotide sequence having at least one modified nucleoside. In some embodiments, an oligonucleotide-ligand conjugate comprises an antisense strand and a sense strand, wherein each strand comprises at least one modified nucleoside.

In some embodiments, the oligonucleotide-ligand conjugate is represented by the following formula:

### Sense Strand:

[mXs][mX][mX][mX][mX][mX][mX][fX][fX][fX][fX][mX][mX][mX][mX][mX][mX][mX][mX][mX][mX][mX][mX][mX][mX][mX][mX][ademX-TL][mX][mX][mX][mX][mX][mX][mX][mX]
Hybridized to
Antisense Strand: [MePhosphonate-4O-mXs][fXs][fX][fX][fX][mX][fX][mX][mX][fX][mX][mX][mX][fX][mX][mX][mX][mX][mX][mXs][mXs][mX]
Or
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-C #][mX][mX][mX][mX][mX][mX][mX][mX]
Hybridized to
Antisense Strand: [MePhosphonate-4O-mXs][fXs][fX][fX][fX][mX][fX][mX][mX][fX][mX][mX][mX][fX][mX][mX][mX][mX][mX][mXs][mXs][mX]

In some embodiments, the oligonucleotide-ligand conjugate is represented by the following formula:
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-TL][mX][mX][mX][mX][mX][mX][mX][mX]
Hybridized to
Antisense Strand: [MePhosphonate-4O-mXs][fXs][fXs][fX][fX][mX][fX][mX][mX][fX][mX][mX][mX][fX][mX][mX][mX][mX][mX][mXs][mXs][mX]
Or
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-C#][mX][mX][mX][mX][mX][mX][mX][mX]
Hybridized to
Antisense Strand: [MePhosphonate-4O-mXs][fXs][fXs][fX][fX][mX][fX][mX][mX][fX][mX][mX][mX][fX][mX][mX][mX][mX][mX][mXs][mXs][mX]

TABLE 1
Modification Key

| | |
|---|---|
| [MePhosphonate-4O-mX] | 4′-O-monomethylphosphonate-2′-O-methyl |
| | modified nucleotide |
| ademX-TL | 2′-aminodiethoxymethanol-nucleotide-targeting |
| | ligand |
| | (i.e., a targeting ligand attached to a nucleotide) |
| ademX-C# | 2′-aminodiethoxymethanol-nucleotide- |
| | hydrocarbon chain (e.g., a C16 or C18 lipid |
| | conjugate attached to a nucleotide) |
| [mXs] | 2′-O-methyl modified nucleotide with a |
| | phosphorothioate linkage to the neighboring |
| | nucleotide |
| [fXs] | 2'-fluoro modified nucleotide with a |
| | phosphorothioate linkage to the neighboring |
| | nucleotide |
| [mX] | 2′-O-methyl modified nucleotide with |
| | phosphodiester linkages to neighboring |
| | nucleotides |
| [fX] | 2′-fluoro modified nucleotide with |
| | phosphodiester linkages to neighboring |
| | nucleotides |

In some embodiments, the oligonucleotide of the oligonucleotide-ligand conjugate is conjugated to a C16 lipid as shown in:

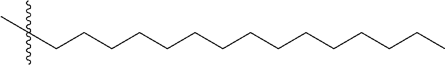 [SMILES: *CCCCCCCCCCCCCCC]

In some embodiments, the oligonucleotide of the oligonucleotide-ligand conjugate is conjugated to a C18 lipid as shown in:

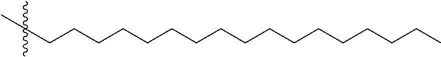 [SMILES: *CCCCCCCCCCCCCCCCC]

In some embodiments, the oligonucleotide-ligand conjugate reduces target mRNA in immune cells of the TME or TdLN but does not reduce mRNA in tumor epithelial cells.

### Methods of Use

i. Reducing Target Gene Expression

In some embodiments, the disclosure provides methods for contacting or delivering to an immune cell or population of immune cells of a tumor microenvironment (e.g., tumor resident immune cells) an effective amount of any of the oligonucleotide-ligand conjugates herein to reduce target gene expression (e.g., reduce expression of a target gene encoding a regulator of immune suppression). In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art.

Methods provided herein are useful in any appropriate tumor resident immune cell type. In some embodiments, a cell is any cell that expresses the target mRNA. In some embodiments, the cell is a primary cell obtained from a subject. In some embodiments, the primary cell has undergone a limited number of passages such that the cell substantially maintains is natural phenotypic properties. In some embodiments, a cell to which the oligonucleotide-ligand conjugate is delivered is ex vivo or in vitro (i.e., can be delivered to a cell in culture or to an organism in which the cell resides).

In some embodiments, the oligonucleotide-ligand conjugates disclosed herein are delivered to an immune cell or population of immune cells of a tumor microenvironment using a nucleic acid delivery method known in the art including, but not limited to, injection of a solution or pharmaceutical composition containing the oligonucleotide-ligand conjugate, bombardment by particles covered by the oligonucleotide-ligand conjugate, exposing the cell or population of cells to a solution containing the oligonucleotide-ligand conjugate, or electroporation of cell membranes in the presence of the oligonucleotide-ligand conjugate. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which an oligonucleotide-ligand conjugate provided herein reduces target gene expression (e.g., reduces expression of a target gene encoding a regulator of immune suppression) is evaluated by comparing target gene expression in a cell or population of cells contacted with the oligonucleotide-ligand conjugate to a control cell or population of cells (e.g., a cell or population of cells not contacted with the oligonucleotide-ligand conjugate or contacted with a control oligonucleotide-ligand conjugate). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

Measuring mRNA in the immune cells can be done using techniques known to those of skill in the art. For example, after a tumor is extracted, the tissue is manually or chemically dissociated into single cells. MACS sorting is then used to isolate the cells of interest (e.g. MDSCs) which are collected and prepared for RNA analysis. In some embodiments, the oligonucleotide conjugate reduces target mRNA expression in immune cells of the TME or TdLN for one day to at least 4 weeks. In some embodiments, the oligonucleotide-ligand conjugate reduces target mRNA expression in immune cells of the TME or TdLN for one day, three days, 7 days, 14 days, 21 days, 28 days, or 34 days. In some embodiments, the oligonucleotide-ligand conjugate reduces target mRNA expression in immune cells of the TME or TdLN for at least 1-4 weeks. In some embodiments, the oligonucleotide-ligand conjugate reduces target mRNA expression in immune cells of the TME or TdLN for up to 2 weeks. In some embodiments, the oligonucleotide-ligand conjugate reduces target mRNA expression in immune cells of the TME or TdLN for up to 4 weeks.

In some embodiments, the oligonucleotide-ligand conjugate reduces target mRNA expression in M-MDSCs for one day to at least 4 weeks. In some embodiments, the oligonucleotide-ligand conjugate reduces target mRNA expression in M-MDSCs for one day, three days, 7 days, 14 days, 21 days, 28 days, or 34 days. In some embodiments, the oligonucleotide-ligand conjugate reduces target mRNA expression in in M-MDSCs for at least 1-4 weeks. In some embodiments, the oligonucleotide-ligand conjugate reduces target mRNA expression in in M-MDSCs for up to 2 weeks. In some embodiments, the oligonucleotide-ligand conjugate reduces target mRNA expression in immune cells of the in M-MDSCs for up to 4 weeks.

In some embodiments, the oligonucleotide-ligand conjugate reduces target mRNA expression in G-MDSCs for one day to at least 4 weeks. In some embodiments, the oligonucleotide-ligand conjugate reduces target mRNA expression in G-MDSCs for one day, three days, 7 days, 14 days, 21 days, 28 days, or 34 days. In some embodiments, the oligonucleotide-ligand conjugate reduces target mRNA expression in in G-MDSCs for at least 1-4 weeks. In some embodiments, the oligonucleotide-ligand conjugate reduces target mRNA expression in in G-MDSCs for up to 2 weeks. In some embodiments, the oligonucleotide-ligand conjugate reduces target mRNA expression in immune cells of the in G-MDSCs for up to 4 weeks.

In some embodiments, the oligonucleotide-ligand conjugate reduces target mRNA expression in Tregs for one day to at least 4 weeks. In some embodiments, the oligonucleotide-ligand conjugate reduces target mRNA expression in Tregs for one day, three days, 7 days, 14 days, 21 days, 28 days, or 34 days. In some embodiments, the oligonucleotide-ligand conjugate reduces target mRNA expression in in M-MDSCs for at least 1-4 weeks. In some embodiments, the oligonucleotide-ligand conjugate reduces target mRNA expression in in Tregs for up to 2 weeks. In some embodiments, the oligonucleotide-ligand conjugate reduces target mRNA expression in immune cells of the in Tregs for up to 4 weeks.

In some embodiments, contacting or delivering an oligonucleotide-ligand conjugate described herein to an immune cell or a population of immune cells of a tumor microenvironment (e.g., a tumor resident immune cell) results in a reduction in target gene expression. In some embodiments, the reduction in target gene expression is relative to a control amount or level of target gene expression in a cell or population of cells not contacted with the oligonucleotide-ligand conjugate or contacted with a control oligonucleotide-ligand conjugate. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the reduction in target gene expression in an immune cell in the TME is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the reduction in target gene expression in an immune cell in the TdLN is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the reduction in target gene expression in an M-MDSC is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the reduction in target gene expression in an G-MDSC is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the reduction in target gene expression in an Treg is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with an oligonucleotide-ligand conjugate herein. In some embodiments, the effect of delivery of an oligonucleotide-ligand conjugate to an immune cell or a population of immune cells of a tumor microenvironment (e.g., a tumor resident immune cell) according to a method herein is assessed after any finite period or amount of time (e.g., minutes, hours, days, weeks, months). For example, in some embodiments, target gene expression is determined in an immune cell or a population of immune cells of a tumor microenvironment (e.g., a tumor resident immune cell) at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide-ligand conjugate to the cell or population of cells. In some embodiments, target gene expression is determined in an immune cell or a population of immune cells of a tumor microenvironment (e.g., a tumor resident immune cell) at least about 1 month, about 2 months, about 3 months, about 4 months, about 5 months, or about 6 months or more after contacting or delivering the oligonucleotide-ligand conjugate to the cell or population of cells.

Reducing the activity of immunosuppressive cells in a tumor, such as Tregs or MDSCs is a potential strategy to convert cold tumors into hot tumors. In some embodiments, the oligonucleotide-ligand conjugate converts a cold tumor into a hot tumor. In some embodiments, the oligonucleotide-ligand conjugate enhances anti-tumorigenic immune activity by reducing immunosuppressive activity. In some embodiments, the oligonucleotide-ligand conjugate enhances anti-tumorigenic T-cell activity by reducing the activity of immunosuppressive cells (e.g. MDSCs).

In some embodiments, the oligonucleotide-ligand conjugate enhances anti-tumorigenic activity by reducing the immunosuppressive activity of MDSCs. In some embodiments, the oligonucleotide-ligand conjugate enhances anti-tumorigenic activity by reducing the immunosuppressive activity of M-MDSCs. In some embodiments, the oligonucleotide-ligand conjugate enhances anti-tumorigenic activity by reducing the immunosuppressive activity of G-MDSCs. In some embodiments, the oligonucleotide-ligand conjugate enhances anti-tumorigenic activity by reducing the immunosuppressive activity of Tregs. In some embodiments, methods for measuring anti-tumorigenic activity include, but are not limited to, measuring the number of tumor infiltrating lymphocytes in the tumor.

In some embodiments, the oligonucleotide-ligand conjugate reduces the immunosuppressive activity of M-MDSCs to a sufficient amount to convert a cold tumor into a hot tumor. In some embodiments, the oligonucleotide-ligand conjugate reduces the immunosuppressive activity of G-MDSCs to a sufficient amount to convert a cold tumor into a hot tumor. In some embodiments, the oligonucleotide-ligand conjugate reduces the immunosuppressive activity of Tregs to a sufficient amount to convert a cold tumor into a hot tumor. Methods for determine whether a cold tumor has been converted to a hot tumor include, but are not limited to, measuring the response of the tumor to an immunotherapy (e.g., checkpoint inhibitor polypeptide).

ii. Treatment Methods and Medical Use

In some aspects, the disclosure provides oligonucleotide-ligand conjugates for use, or adaptable for use, to treat a subject (e.g., a human) with cancer that would benefit from reducing a target gene (e.g., a target gene encoding a regulator of immune suppression). In some respects, the disclosure provides oligonucleotide-ligand conjugates for use, or adapted for use, to treat a subject having cancer. In some respects, the disclosure provides oligonucleotide-ligand conjugates for use, or adapted for use, to treat a subject having cancer associated with an immunosuppressive TME. The disclosure also provides oligonucleotide-ligand conjugates for use, or adaptable for use, in the manufacture of a medicament or pharmaceutical composition for treating cancer. In some embodiments, the oligonucleotide-ligand conjugates for use, or adaptable for use, target a regulator of immune suppression (e.g., a transcription factor or checkpoint inhibitor polypeptide). In some embodiments, the oligonucleotide-ligand conjugates for use, or adaptable for use, target a regulator of immune suppression and reduce the amount or level of the regulator's mRNA, or the regulator's protein and/or activity.

As detailed below, the methods also may include steps such as measuring or obtaining a baseline value for a marker of a regulator of immune suppression, and then comparing such obtained value to one or more other baseline values or values obtained after being administered the oligonucleotide to assess the effectiveness of treatment.

In some embodiments, the disclosure provides oligonucleotide-ligand conjugates for reducing immune suppression in a tumor microenvironment. In some embodiments, reduction of immune suppression is determined by an appropriate assay or technique to evaluate one or more properties or characteristics of immune suppression in a tumor (e.g. the presence of suppressive cells such as MDSCs) or by an assay or technique that evaluates molecules that are directly indicative of immune suppression (e.g., high Arg1 expression). In some embodiments, the extent to which an oligonucleotide-ligand conjugate herein reduces immune suppression is evaluated by comparing immune suppression in the TME contacted with the oligonucleotide-ligand conjugate to an appropriate control (e.g., an appropriate tumor not contacted with the oligonucleotide or contacted with a control oligonucleotide). In some embodiments, an appropriate control level of mRNA expression into protein may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide-ligand conjugate herein results in a reduction in target mRNA in a tumor resident immune cell. In some embodiments, the reduction in target mRNA is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower when compared with an appropriate control level of mRNA. The appropriate control level may be a level of mRNA expression and/or protein translation in a cell or population of cells that has not been contacted with an oligonucleotide-ligand conjugate herein. In some embodiments, the effect of delivery of an oligonucleotide-ligand conjugate to a cell according to a method herein is assessed after a finite period. For example, levels of mRNA may be analyzed in a cell at least about 8 hours, about 12 hours, about 18 hours, about 24 hours; or at least about 1, 2, 3, 4, 5, 6, 7 or even up to 14 days after introduction of the oligonucleotide-ligand conjugate into the tumor.

In some embodiments, an oligonucleotide-ligand conjugate is delivered in the form of a transgene that is engineered to express in a cell the oligonucleotide-ligand conjugate or strands comprising the oligonucleotide-ligand conjugate (e.g., its sense and antisense strands). In some embodiments, an o oligonucleotide-ligand conjugate is delivered using a transgene engineered to express any oligonucleotide-ligand conjugate disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

In some aspects, the disclosure provides methods of treating a subject having, suspected of having, or at risk of developing a cancer. In some embodiments, the disclosure provides methods of treating or attenuating the onset or progression of cancer using the oligonucleotide-ligand conjugates described herein. In some embodiments of the methods herein, a subject is treated by administering a therapeutically effective amount of any one or more of the oligonucleotide-ligand conjugates herein. In some embodiments, the subject is a mammal. In some embodiments, the subject is a human.

In some embodiments of the methods herein, one or more oligonucleotide-ligand conjugates herein, or a pharmaceutical composition comprising one or more oligonucleotide-ligand conjugates, is administered to a subject having cancer. In some embodiments, the oligonucleotide-ligand conjugate reduces a target mRNA in a tumor (e.g., in an immune cell in a tumor microenvironment). In some embodiments, the amount of target mRNA and/or protein is reduced in the subject.

In some embodiments of the methods herein, an oligonucleotide-ligand conjugate herein, or a pharmaceutical composition comprising the oligonucleotide-ligand conjugate, is administered to a subject having cancer and expression of a target gene (e.g., regulator of immune suppression) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to expression of the target prior to administration of one or more oligonucleotide-ligand conjugates or pharmaceutical composition. In some embodiments, the target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the target mRNA expression in a subject (e.g., a reference or control subject) not receiving the oligonucleotide-ligand conjugate or pharmaceutical composition or receiving a control oligonucleotide-ligand conjugate or pharmaceutical composition or treatment.

In some embodiments of the methods herein, an oligonucleotide-ligand conjugate or oligonucleotide-ligand conjugates herein, or a pharmaceutical composition comprising the oligonucleotide-ligand conjugate (s), is administered to a subject having cancer such that an amount or level of target mRNA (e.g., gene encoding a regulator of immune suppression) is reduced in tumor resident immune cells of the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide-ligand conjugate or pharmaceutical composition. In some embodiments of the methods herein, an oligonucleotide-ligand conjugate or oligonucleotide-ligand conjugates herein, or a pharmaceutical composition comprising the oligonucleotide-ligand conjugate (s), is administered to a subject having cancer such that an amount or level of target mRNA (e.g., gene encoding a regulator of immune suppression) is reduced in TdLN immune cells of the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide-ligand conjugate or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide-ligand conjugate or oligonucleotide-ligand conjugates or pharmaceutical composition or receiving a control oligonucleotide-ligand conjugate or oligonucleotide-ligand conjugates, pharmaceutical composition or treatment.

In some embodiments of the methods herein, an oligonucleotide-ligand conjugate or oligonucleotide-ligand conjugates herein, or a pharmaceutical composition comprising the oligonucleotide-ligand conjugate(s), is administered to a subject having cancer with an immune suppressive environment such that an amount or level of a target protein regulating immune suppression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein regulating immune suppression prior to administration of the oligonucleotide-ligand conjugate or pharmaceutical composition. In some embodiments, an amount or level of protein regulating immune suppression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein regulating immune suppression in a subject (e.g., a reference or control subject) not receiving the oligonucleotide-ligand conjugate(s) or pharmaceutical composition or receiving a control oligonucleotide-ligand conjugate(s), or pharmaceutical composition or treatment.

In some embodiments of the methods herein, an oligonucleotide-ligand conjugate or oligonucleotide-ligand conjugates herein, or a pharmaceutical composition comprising the oligonucleotide-ligand conjugate or oligonucleotide-ligand conjugates, is administered to a subject having cancer with an immunosuppressive TME such that an amount or level of an mRNA or protein regulating immune suppression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of the mRNA or protein regulating immune suppression prior to administration of the oligonucleotide-ligand conjugate or pharmaceutical composition. In some embodiments, an amount or level of target mRNA regulating immune suppression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide-ligand conjugate or pharmaceutical composition or receiving a control oligonucleotide-ligand conjugate, pharmaceutical composition or treatment.

Because of their high specificity, the oligonucleotide-ligand conjugates herein specifically target mRNAs of target genes of diseased cells and tissues. In some embodiments, the oligonucleotide-ligand conjugate delivers the oligonucleotide to a target cell. In some embodiments, the target cell is an immune cell found in a tumor microenvironment. In some embodiments, the target cell is an immune cell found in an immune suppressive tumor microenvironment. In some embodiments, the oligonucleotide-ligand conjugate delivers the oligonucleotide to one or more MDSC cell populations. In some embodiments, the oligonucleotide-ligand conjugate delivers the oligonucleotide to a G-MDSC. In some embodiments, the oligonucleotide-ligand conjugate delivers the oligonucleotide to a M-MDSC. In some embodiments, the oligonucleotide-ligand conjugate delivers the oligonucleotide to a G-MDSC and a M-MDSC. In some embodiments, the oligonucleotide-ligand conjugate delivers the oligonucleotide to a T cell in a tumor microenvironment. In some embodiments, the oligonucleotide-ligand conjugate delivers the oligonucleotide nucleotide to a Treg cell.

As described herein, the oligonucleotide-ligand conjugate for targeting an mRNA encoding a regulator of immune suppression is capable of converting a cold tumor to a hot tumor. Hot tumors enable other therapeutic approaches to be more effective at treating disease. Therefore, in some embodiments, an oligonucleotide-ligand conjugate described herein is administered in combination with a second therapeutic agent. In some embodiments, the second therapeutic agent is selected from, but not limited to a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, or a combination thereof.

Methods described herein typically involve administering to a subject in an effective amount of an oligonucleotide-ligand conjugate or oligonucleotide-ligand conjugates, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). In some embodiments, an oligonucleotide-ligand conjugate or pharmaceutical composition thereof is administered intravenously or subcutaneously.

As a non-limiting set of examples, in some embodiments, the oligonucleotide-ligand conjugates herein are administered quarterly (once every three months), bi-monthly (once every two months), monthly or weekly. For example, the oligonucleotide-ligand conjugates may be administered every week or at intervals of two, or three weeks. Alternatively, the oligonucleotide-ligand conjugates may be administered daily. In some embodiments, a subject is administered one or more loading doses of the oligonucleotide-ligand conjugate followed by one or more maintenance doses of the oligonucleotide-ligand conjugate.

In some embodiments the oligonucleotide-ligand conjugate herein are administered alone or in combination. In some embodiments the oligonucleotides herein are administered in combination concurrently, sequentially (in any order), or intermittently. For example, two oligonucleotide-ligand conjugates may be co-administered concurrently. Alternatively, one oligonucleotide-ligand conjugate may be administered and followed any amount of time later (e.g., one hour, one day, one week or one month) by the administration of a second oligonucleotide-ligand conjugate.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Types of Oligonucleotides

A variety of oligonucleotide types and/or structures are useful for targeting a target sequence in the methods herein including, but not limited to, RNAi oligonucleotides, antisense oligonucleotides, miRNAs, etc. Any of the oligonucleotide types described herein or elsewhere are contemplated for use as a framework to incorporate a targeting sequence herein.

In some embodiments, the oligonucleotides herein inhibit expression of a target sequence by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include ss extensions (on one or both sides of the molecule) as well as ds extensions.

In some embodiments, the oligonucleotides herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotide sin length capable of reducing target mRNA expression are produced. In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 26 (e.g., 17 to 26, 20 to 25 or 21-23) nucleotides in length. In some embodiments, the oligonucleotides herein comprise sense and antisense strands that are both in the range of about 17 to 36 (e.g., 17 to 36, 20 to 25 or 21-23) nucleotides in length. In some embodiments, the oligonucleotides described herein comprise an antisense strand of 19-30 nucleotides in length and a sense strand of 19-50 nucleotides in length, wherein the antisense and sense strands are separate strands which form an asymmetric duplex region having an overhand of 1-4 nucleotides at the 3′ terminus of the antisense strand. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY. Blackburn (ed.), Royal Society of Chemistry, 2006), shRNAs (e.g., having 19 bp or shorter stems; (see, e.g., Moore et al., (2010) METHODS MOL. BIOL. 629:141-58), blunt siRNAs (e.g., of 19 bps in length; see, e.g., Kraynack and Baker (2006) RNA 12:163-76), asymmetrical siRNAs (aiRNA; see, e.g., Sun et al., (2008) NAT. BIOTECHNOL. 26:1379-82), asymmetric shorter-duplex siRNA (see, e.g., Chang et al., (2009) MOL. THER. 17:725-32), fork siRNAs (see, e.g., Hohj oh (2004) FEBS LETT. 557:193-98), ss siRNAs (Elsner (2012) NAT. BIOTECHNOL. 30:1063), dumbbell-shaped circular siRNAs (see, e.g., Abe et al., (2007) J. AM. CHEM. SOC. 129:15108-09), and small internally segmented interfering RNA (siRNA; see, e.g., Bramsen et al., (2007) NUCLEIC ACIDS RES. 35:5886-97). Further non-limiting examples of an oligonucleotide structures that may be used in some embodiments to reduce or inhibit the expression of STAT3 are microRNA (miRNA), short hairpin RNA (shRNA) and short siRNA (see, e.g., Hamilton et al., (2002) EMBO J. 21:4671-79; see also, US Patent Application Publication No. 2009/0099115).

Still, in some embodiments, an oligonucleotide for reducing or inhibiting expression of a target sequence herein is ss. Such structures may include but are not limited to ss RNAi molecules. Recent efforts have demonstrated the activity of ss RNAi molecules (see, e.g., Matsui et al., (2016) MOL. THER. 24:946-55). However, in some embodiments, oligonucleotides herein are antisense oligonucleotides (ASOs). An antisense oligonucleotide is a ss oligonucleotide that has a nucleobase sequence which, when written in the 5′ to 3′ direction, comprises the reverse complement of a targeted segment of a particular nucleic acid and is suitably modified (e.g., as a gapmer) to induce RNaseH-mediated cleavage of its target RNA in cells or (e.g., as a mixmer) to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al., (2017) ANNU. REV. PHARMACOL. 57:81-105).

In some embodiments, the antisense oligonucleotide shares a region of complementarity with a target mRNA. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### Double-Stranded Oligonucleotides

In some embodiments, the disclosure provides double-stranded dsRNAs for targeting and inhibiting expression of a target sequence (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a loop (L), such as a tetraloop (tetraL) or triloop (triL), and a second subregion (S2), wherein L, tetraL, or triL is located between S1 and S2, and wherein S1 and S2 form a second duplex (D2). D2 may have various length. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

It should be appreciated that, in some embodiments, sequences presented in the Sequence Listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a double-stranded RNA (dsRNA) herein comprises a 25-nucleotide sense strand and a 27-nucleotide antisense strand that when acted upon by a Dicer enzyme results in an antisense strand that is incorporated into the mature RISC. In some embodiments, the sense strand of the dsRNA is longer than 27 nucleotides (e.g., 28, 29, 30, 31, 32, 33, 34, 35, 36, 37, 38, 39 or 40 nucleotides). In some embodiments, the sense strand of the dsRNA is longer than 27 nucleotides (e.g., 28, 29, 30, 31, 32, 33, 34, 35, 36, 37, 38, 39, 40, 41, 42, 43, 44, 45, 46, 47, 48, 49, or 50 nucleotides). In some embodiments, the sense strand of the dsRNA is longer than 25 nucleotides (e.g., 26, 27, 28, 29 or 30 nucleotides).

In some embodiments, oligonucleotides herein have one 5′ end that is thermodynamically less stable when compared to the other 5′ end. In some embodiments, an asymmetry oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, an oligonucleotide for RNAi has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA. In some embodiments, the two terminal nucleotides on the 3′ end of the antisense strand are not complementary with the target mRNA. In some embodiments, the two terminal nucleotides on the 3′ end of the antisense strand of an oligonucleotide herein comprise an unpaired GG. In some embodiments, the two (2) terminal nucleotides on the 3′ end of an antisense strand of an oligonucleotide herein are not complementary to the target mRNA. In some embodiments, two terminal nucleotides on each 3′ end of an oligonucleotide in the nicked tetraloop structure are GG. In some embodiments, one or both of the two (2) terminal GG nucleotides on each 3′ end of an oligonucleotide herein is not complementary with the target mRNA. Typically, one or both two terminal GG nucleotides on each 3′ end of an oligonucleotide is not complementary with the target.

In some embodiments, there is one or more (e.g., 1, 2, 3, 4 or 5) mismatch between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of the oligonucleotide improved the potency of synthetic duplexes in RNAi, possibly through facilitating processing by Dicer.

a. Antisense Strands

In some embodiments, a dsRNA comprises an antisense strand of up to about 40 nucleotides in length (e.g., up to 40, up to 35, up to 30, up to 27, up to 25, up to 21, up to 19, up to 17 or up to 12 nucleotides in length). In some embodiments, an oligonucleotide herein (e.g., an RNAi oligonucleotide) comprises an antisense strand of up to about 50 nucleotides in length (e.g., up to 50, up to 40, up to 35, up to 30, up to 27, up to 25, up to 21, up to 19, up to 17 or up to 12 nucleotides in length). In some embodiments, an oligonucleotide may have an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide may have an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide comprises antisense strand of 15 to 30 nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, an antisense strand of an oligonucleotide may be referred to as a “guide strand.” For example, if an antisense strand can engage with RNA-induced silencing complex (RISC) and bind to an Argonaute protein such as Ago2, or engage with or bind to one or more similar factors, and direct silencing of a target gene, it may be referred to as a guide strand. In some embodiments, a sense strand complementary to a guide strand may be referred to as a “passenger strand.”

b. Sense Strands

In some embodiments, an oligonucleotide comprises a sense strand (or passenger strand) of up to about 40 nucleotides in length (e.g., up to 40, up to 36, up to 30, up to 27, up to 25, up to 21, up to 19, up to 17 or up to 12 nucleotides in length). In some embodiments, an oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide herein comprises a sense strand of 15 to 50 nucleotides in length. In some embodiments, an oligonucleotide herein comprises a sense strand of 18 to 36 nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an oligonucleotide herein comprises a sense strand of 36 nucleotides in length.

In some embodiments, an oligonucleotide provided herein (e.g., an RNAi oligonucleotide) comprises a sense strand comprising a stem-loop structure at the 3′ end of the sense strand. In some embodiments, the stem-loop is formed by intrastrand base pairing. In some embodiments, a sense strand comprises a stem-loop structure at its 5′ end. In some embodiments, the stem of the stem-loop comprises a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the oligonucleotide protection against degradation (e.g., enzymatic degradation), facilitates or improves targeting and/or delivery to a target cell, tissue, or organ (e.g., the liver), or both. For example, in some embodiments, the loop of a stem-loop is comprised of nucleotides comprising one or more modifications that facilitate, improve, or increase targeting to a target, inhibition of target gene expression, and/or delivery, uptake, and/or penetrance into a target cell, tissue, or organ (e.g., the liver), or a combination thereof. In some embodiments, the stem-loop itself or modification(s) to the stem-loop do not affect or do not substantially affect the inherent gene expression inhibition activity of the oligonucleotide, but facilitates, improves, or increases stability (e.g., provides protection against degradation) and/or delivery, uptake, and/or penetrance of the oligonucleotide to a target cell, tissue, or organ. In certain embodiments, an oligonucleotide herein comprises a sense strand comprising (e.g., at its 3′ end) a stem-loop set forth as: S1-L-S2, in which S1 is complementary to S2, and in which L forms a single-stranded loop of linked nucleotides between S1 and S2 of up to about 10 nucleotides in length (e.g., 3, 4, 5, 6, 7, 8, 9 or 10 nucleotides in length). In some embodiments, the loop (L) is 3 nucleotides in length (referred to herein as “triloop”. In some embodiments, the loop (L) is 4 nucleotides in length (referred to herein as “tetraloop”). In some embodiments, the loop (L) is 5 nucleotides in length. In some embodiments, the loop (L) is 6 nucleotides in length. In some embodiments, the loop (L) is 7 nucleotides in length. In some embodiments, the loop (L) is 8 nucleotides in length. In some embodiments, the loop (L) is 9 nucleotides in length. In some embodiments, the loop (L) is 10 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 86).

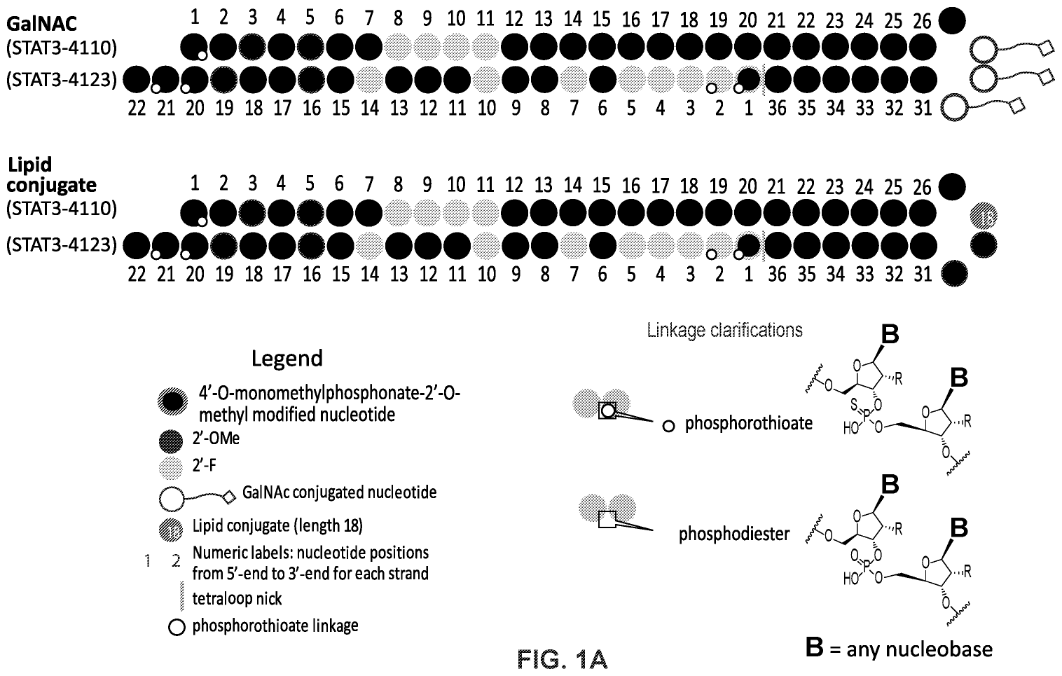

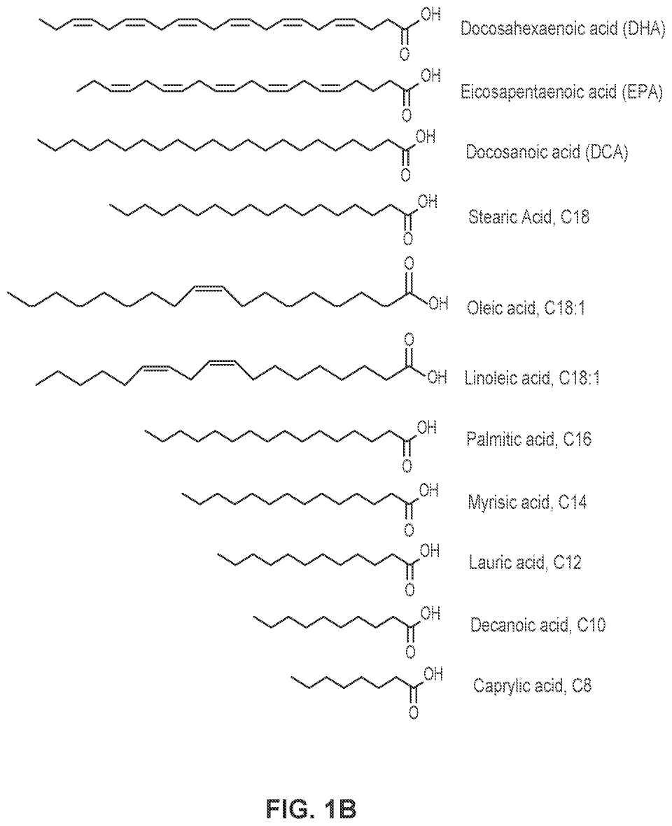

In some embodiments, a sense strand comprises a stem-loop structure at its 3′ end. In some embodiments, a sense strand comprises a stem-loop structure at its 5′ end. In some embodiments, a stem is a duplex of 2, 3, 4, 5, 6, 7, 8, 9, 10, 11, 12, 13 or 14 bp in length. In some embodiments, a stem-loop provides the molecule protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is herein in which the sense strand comprises (e.g., at its 3′ end) a stem-loop set forth as: S1-L-S2, in which S1 is complementary to S2, and in which L forms a loop between S1 and S2 of up to about 10 nucleotides in length (e.g., 3, 4, 5, 6, 7, 8, 9 or 10 nucleotides in length). FIG. 1 depicts non-limiting examples of such an oligonucleotide.

In some embodiments, a loop (L) of a stem-loop having the structure S1-L-S2 as described herein is a triloop. In some embodiments, the triloop comprises ribonucleotides, deoxyribonucleotides, modified nucleotides, ligands (e.g., delivery ligands), and combinations thereof.

In some embodiments, a loop of a stem-loop is a tetraloop (e.g., within a nicked tetraloop structure). A tetraloop may contain ribonucleotides, deoxyribonucleotides, modified nucleotides and combinations thereof. Typically, a tetraloop has 4 to 5 nucleotides.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 12 nucleotides in length. In some embodiments, a duplex formed between a sense and antisense strand is 13 nucleotides in length. In some embodiments, a duplex formed between a sense and antisense strand is 14 nucleotides in length. In some embodiments, a duplex formed between a sense and antisense strand is 15 nucleotides in length. In some embodiments, a duplex formed between a sense and antisense strand is 16 nucleotides in length. In some embodiments, a duplex formed between a sense and antisense strand is 17 nucleotides in length. In some embodiments, a duplex formed between a sense and antisense strand is 18 nucleotides in length. In some embodiments, a duplex formed between a sense and antisense strand is 19 nucleotides in length. In some embodiments, a duplex formed between a sense and antisense strand is 20 nucleotides in length. In some embodiments, a duplex formed between a sense and antisense strand is 21 nucleotides in length. In some embodiments, a duplex formed between a sense and antisense strand is 22 nucleotides in length. In some embodiments, a duplex formed between a sense and antisense strand is 23 nucleotides in length. In some embodiments, a duplex formed between a sense and antisense strand is 24 nucleotides in length. In some embodiments, a duplex formed between a sense and antisense strand is 25 nucleotides in length. In some embodiments, a duplex formed between a sense and antisense strand is 26 nucleotides in length. In some embodiments, a duplex formed between a sense and antisense strand is 27 nucleotides in length. In some embodiments, a duplex formed between a sense and antisense strand is 28 nucleotides in length. In some embodiments, a duplex formed between a sense and antisense strand is 29 nucleotides in length. In some embodiments, a duplex formed between a sense and antisense strand is 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Termini

In some embodiments, an oligonucleotide disclosed herein (e.g., an RNAi oligonucleotide) comprises a sense strand and an antisense strand, wherein the termini of either or both strands comprise a blunt end. In some embodiments, an oligonucleotide herein comprises sense and antisense strands that are separate strands which form an asymmetric duplex region having an overhang at the 3′ terminus of the antisense strand. In some embodiments, an oligonucleotide herein comprises a sense strand and an antisense strand, wherein the termini of either or both strands comprise an overhang comprising one or more nucleotides. In some embodiments, the one or more nucleotides comprising the overhang are unpaired nucleotides. In some embodiments, an oligonucleotide herein comprises a sense strand and an antisense strand, wherein the 3′ termini of the sense strand and the 5′ termini of the antisense strand comprise a blunt end. In some embodiments, an oligonucleotide herein comprises a sense strand and an antisense strand, wherein the 5′ termini of the sense strand and the 3′ termini of the antisense strand comprise a blunt end.

In some embodiments, an oligonucleotide herein comprises a sense strand and an antisense strand, wherein the 3′ terminus of either or both strands comprise a 3′-overhang comprising one or more nucleotides. In some embodiments, an oligonucleotide herein comprises a sense strand and an antisense strand, wherein the sense strand comprises a 3′-overhang comprising one or more nucleotides. In some embodiments, an oligonucleotide herein comprises a sense strand and an antisense strand, wherein the antisense strand comprises a 3′-overhang comprising one or more nucleotides. In some embodiments, an oligonucleotide herein comprises a sense strand and an antisense strand, wherein both the sense strand and the antisense strand comprises a 3′-overhang comprising one or more nucleotides.

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, one or more (e.g., 2, 3, 4, 5, or more) nucleotides comprising the 3′ terminus or 5′ terminus of a sense and/or antisense strand are modified. For example, in some embodiments, one or two terminal nucleotides of the 3′ terminus of the antisense strand are modified. In some embodiments, the last nucleotide at the 3′ terminus of an antisense strand is modified, such that it comprises 2′ modification, or it comprises, a 2′-O-methoxyethyl. In some embodiments, the last one or two terminal nucleotides at the 3′ terminus of an antisense strand are complementary with the target. In some embodiments, the last one or two nucleotides at the 3′ terminus of the antisense strand are not complementary with the target.

In some embodiments, an oligonucleotide disclosed herein (e.g., an RNAi oligonucleotide) comprises a sense strand and an antisense strand, wherein the 3′ terminus of the sense strand comprises a step-loop described herein and the 3′ terminus of the antisense strand comprises a 3′-overhang described herein. In some embodiments, an oligonucleotide herein (e.g., an RNAi oligonucleotide) comprises a sense strand and an antisense strand that form a nicked tetraloop structure described herein, wherein the 3′ terminus of the sense strand comprises a stem-loop, wherein the loop is a tetraloop described herein, and wherein the 3′ terminus of the antisense strand comprises a 3′-overhang described herein. In some embodiments, the 3′-overhang is two (2) nucleotides in length. In some embodiments, the two (2) nucleotides comprising the 3′-overhang both comprise guanine (G) nucleobases. Typically, one or both of the nucleotides comprising the 3′-overhang of the antisense strand are not complementary with the target mRNA.

### Oligonucleotide Modifications

a. Sugar Modifications

In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al., (1998) TETRAHEDON 54:3607-3630), unlocked nucleic acids (“UNA”; see, e.g., Snead et al., (2013) MOL. THER-NUCL. ACIDS 2:e103) and bridged nucleic acids (“BNA”; see, e.g., Imanishi and Obika (2002) CHEM COMMUN. (CAMB) 21:1653-1659).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-(3-d-arabinonucleic acid). In some embodiments, the modified nucleotide comprises a 2′-modification (e.g., a 2′-F or 2′-OMe).

In some embodiments, the disclosure provides oligonucleotides having different modification patterns. In some embodiments, an oligonucleotide herein comprises a sense strand having a modification pattern as set forth in the Examples and Sequence Listing and an antisense strand having a modification pattern as set forth in the Examples and Sequence Listing.

In some embodiments, an oligonucleotide disclosed herein (e.g., an RNAi oligonucleotide) comprises an antisense strand having nucleotides that are modified with 2′-F. In some embodiments, an oligonucleotide herein comprises an antisense strand comprising nucleotides that are modified with 2′-F and 2′-OMe. In some embodiments, an oligonucleotide disclosed herein comprises a sense strand having nucleotides that are modified with 2′-F. In some embodiments, an oligonucleotide disclosed herein comprises a sense strand comprises nucleotides that are modified with 2′-F and 2′-OMe.

In some embodiments, an oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the dsRNAi oligonucleotide comprise a 2′-fluoro modification.

In some embodiments, the modified oligonucleotides comprise a sense strand sequence having a modification pattern as set forth in FIG. 1 or Example 12 and an antisense strand having a modification pattern as set forth in FIG. 1 or Example 12. In some embodiments, for these oligonucleotides, one or more of positions 8, 9, 10 or 11 of the sense strand is modified with a 2′—F group. In other embodiments, for these oligonucleotides, the sugar moiety at each of nucleotides at positions 1-7 and 12-20 in the sense strand is modified with a 2′-OMe.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In some embodiments, the sugar moiety at positions 2, 5 and 14 and optionally up to 3 of the nucleotides at positions 3, 4, 7 and 10 of the antisense strand are modified with a 2′-F. In other embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In other embodiments, the sugar moiety at each of the positions at positions 2, 4, 5 and 14 of the antisense strand is modified with the 2′-F. In still other embodiments, the sugar moiety at each of the positions at positions 1, 2, 3, 5, 7 and 14 of the antisense strand is modified with the 2′-F. In other embodiments, the sugar moiety at each of the positions at positions 2, 3, 4, 5, 7 and 14 of the antisense strand is modified with the 2′-F. In yet another embodiment, the sugar moiety at each of the positions at positions 1, 2, 3, 5, 10 and 14 of the antisense strand is modified with the 2′-F. In other embodiments, the sugar moiety at each of the positions at positions 2, 3, 4, 5, 10 and 14 of the antisense strand is modified with the 2′-F. In another embodiment, the sugar moiety at each of the positions at positions 2, 3, 5, 7, 10 and 14 of the antisense strand is modified with the 2′-F. In yet another embodiment, the sugar moiety at each of the positions at positions 2, 3, 4, 5, 7, 10 and 14 of the antisense strand is modified with the 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-M0E), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 3, 8, 9, 10, 12, 13 and 17 modified with 2′-F. In some embodiments, an oligonucleotide provided herein comprises a sense strand having the sugar moiety at positions 1-7 and 12-17 or 12-20 modified with 2′OMe. In some embodiments, an oligonucleotide provided herein comprises a sense strand having the sugar moiety at positions 1-7, 12-27 and 31-36 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-f3-d-arabinonucleic acid (2′-FANA). In some embodiments, an oligonucleotide provided herein comprises a sense strand having the sugar moiety at positions 1-2, 4-7, 11, 14-16 and 18-20 modified with 2′OMe. In some embodiments, an oligonucleotide provided herein comprises a sense strand having the sugar moiety of each of the nucleotides at positions 1-2, 4-7, 11, 14-16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-WA), and 2′-deoxy-2′-fluoro-β-d-arabinonucleic acid (2′-FANA).

### b. 5′ Terminal Phosphates

In some embodiments, 5′-terminal phosphate groups of oligonucleotides enhance

the interaction with Ago2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate or malonyl phosphonate. In certain embodiments, the 1′ end of an oligonucleotide strand is attached to chemical moiety that mimics the electrostatic and steric properties of a natural 5′-phosphate group (“phosphate mimic”).

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethyl phosphonate or an amino methyl phosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethyl phosphonate. In some embodiments, an oxymethyl phosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si (CH₃)₃ or a protecting group. In certain embodiments, the alkyl group is CH₂CH₃. More typically, R is independently selected from H, CH₃ or CH₂CH₃.

In some embodiments, an oligonucleotide provided herein comprises an antisense strand comprising a 4′-phosphate analog at the 5′-terminal nucleotide, wherein 5′-terminal nucleotide comprises the following structure:

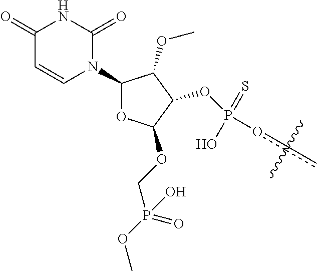

### 4′-O-monomethylphosphonate-2′-O-methyluridine phosphorothioate [MePhosphonate-4O-mUs]

Chem 1

c. Modified Internucleotide Linkages

In some embodiments, an oligonucleotide may comprise a modified internucleoside linkage. In some embodiments, phosphate modifications or substitutions may result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, 4′-O-methylene phosphonate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage. In some embodiments, at least one modified internucleotide linkage of any one of the oligonucleotides as disclosed herein is a 4′-O-methylene phosphonate linkage.

In some embodiments, the oligonucleotide described herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

d. Base Modifications

In some embodiments, oligonucleotides herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al., (1995) NUCLEIC ACIDS RES. 23:4363-4370; Loakes et al., (1995) NUCLEIC ACIDS RES. 23:2361-66; and Loakes and Brown (1994) NUCLEIC ACIDS RES. 22:4039-43).

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See US Patent Application Publication No. 2011/0294869, Intl. Patent Application Publication Nos. WO 2014/088920 and WO 2015/188197, and Meade et al., (2014) NAT. BIOTECHNOL. 32:1256-63. This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (see, Dellinger et al., (2003) J. AM. CHEM. Soc. 125:940-50).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione-sensitive moieties, it is possible to introduce sterically larger chemical groups into the oligonucleotide of interest when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., U.S. Provisional Patent Application No. 62/378,635, entitled Compositions Comprising Reversibly Modified Oligonucleotides and Uses Thereof which was filed on Aug. 23, 2016.

### Oligonucleotide Inhibitors of STAT3

In some aspects, the disclosure provides, inter alia, oligonucleotides that reduce or inhibit STAT3 expression. In some embodiments, an oligonucleotide that inhibits STAT3 expression herein is targeted to a STAT3 mRNA. The sequence of human STAT3 mRNA (NM_001369512.1) is set forth as SEQ ID NO: 85 or NM_139276.3 (SEQ ID NO: 1217). STAT3 is a known target for conventional cancer therapies.

The tolerogenic activities of MDSCs are controlled by an oncogenic transcription factor, signal transducer and activator of transcription 3 (STAT3) (Su et al., INT J. MOL SCI (2018) 19(6): 1803). STAT3 is also known to be highly expressed across a range of cancer types and in in vitro and in vivo preclinical models (Huynh et al., NAT. REV. CANCER (2019) 19: 82-96). The inhibition of STAT3 leads to the selective apoptosis of tumor cells and tumor growth inhibition through modulation of downstream target genes (Wang et al., INTERNATIONAL JOURNAL OF BIOLOGICAL SCIENCES, 15(3): 668-79 (2019)). STAT3 is of particular interest in immuno-oncology due to its well documented contributions to an immunosuppressive tumor microenvironment. STAT3 contributes to an immunosuppressive tumor microenvironment by upregulating the inhibitory receptor expressed by T-cells, and via expression of its ligand (PD-1/PD-L1), through increased secretion of IFNγ ((Bu et al., JOURNAL OF DENTAL RESEARCH, 96(9): 1027-34 (2017)). It has long been known that inhibition of STAT3 signaling in antigen presenting cells (APCs) results in priming of antigen-specific CD4+ T cells in response to otherwise tolerogenic stimuli (Cheng et al., IMMUNITY, 19: 425-36 (2003)). In addition, phosphorylated STAT3 on MDSCs directly contributes to the modulation of the suppressive tumor microenvironment by regulating suppressive components such as the amino acid arginine, through transcriptional control (Vasques-Dunndel et al., J. CLIN. INVEST., 15(3): 668-79 (2013)). Over the years several methodologies have been explored to therapeutically target STAT3. While direct targeting of the protein is attractive, the true target is a protein-protein interaction that has been held up as an example of an ‘undruggable’ target due historical data showing that multiple classes of compounds have failed to effectively inhibit its activity (Lau et al., CANCERS (2019) 11(11): 1681, Zou et al., MOL CANCER (2020) 19: 145). In addition, ubiquitous expression of STAT3 across several tissues have led to concerns about severe on-target toxicities (Wong et al., EXPERT OPINION ON INVESTIGATIONAL DRUGS, 26 (8):883-87 (2017), (Kortylewski et al., CANCER IMMUNOL IMMUNOTHER (2017) 66(8): 979-88).

### STAT3 Target Sequences

In some embodiments, the oligonucleotide is targeted to a target sequence comprising a STAT3 mRNA. In some embodiments, the oligonucleotide, or a portion, fragment, or strand thereof (e.g., an antisense strand or a guide strand of a dsRNA) binds or anneals to a target sequence comprising a STAT3 mRNA, thereby inhibiting STAT3 expression. In some embodiments, the oligonucleotide is targeted to a STAT3 target sequence for the purpose of inhibiting STAT3 expression in vivo. In some embodiments, the amount or extent of inhibition of STAT3 expression by an oligonucleotide targeted to a STAT3 target sequence correlates with the potency of the oligonucleotide. In some embodiments, the amount or extent of inhibition of STAT3 expression by an oligonucleotide targeted to a STAT3 target sequence correlates with the amount or extent of therapeutic benefit in a subject or patient having a disease, disorder or condition associated with the expression of STAT3 treated with the oligonucleotide.

Through examination of the nucleotide sequence of mRNAs encoding STAT3, including mRNAs of multiple different species (e.g., human, cynomolgus monkey, mouse, and rat; see, e.g., Example 11) and as a result of in vitro and in vivo testing (see, e.g., Example 12 and Example 13), it has been discovered that certain nucleotide sequences of STAT3 mRNA are more amenable than others to oligonucleotide-based inhibition and are thus useful as target sequences for the oligonucleotides herein. In some embodiments, a sense strand of an oligonucleotide (e.g., a dsRNA) described herein comprises a STAT3 target sequence. In some embodiments, a portion or region of the sense strand of a dsRNA described herein comprises a STAT3 target sequence. In some embodiments, a STAT3 mRNA target sequence comprises, or consists of, a sequence of SEQ ID NO 85. In some embodiments, a STAT3 mRNA target sequence comprises, or consists of, a sequence of SEQ ID NO: 1217. In some embodiments, a STAT3 mRNA target sequence comprises, or consists of, a sequence of any one of SEQ ID NOs: 89-280. In some embodiments, a STAT3 mRNA target sequence comprises, or consists of, the sequence set forth in SEQ ID NO: 108. In some embodiments, a STAT3 mRNA target sequence comprises, or consists of, the sequence set forth in SEQ ID NO: 140. In some embodiments, a STAT3 mRNA target sequence comprises, or consists of, the sequence set forth in SEQ ID NO: 141. In some embodiments, a STAT3 mRNA target sequence comprises, or consists of, the sequence set forth in SEQ ID NO: 147.

### STAT3 Targeting Sequences

In some embodiments, the oligonucleotides herein have regions of complementarity to STAT3 mRNA (e.g., within a target sequence of STAT3 mRNA) for purposes of targeting the mRNA in cells and reducing or inhibiting its expression. In some embodiments, the oligonucleotides herein comprise a STAT3 targeting sequence (e.g., an antisense strand or a guide strand of a dsRNA) having a region of complementarity that binds or anneals to a STAT3 target sequence by complementary (Watson-Crick) base pairing. The targeting sequence or region of complementarity is generally of a suitable length and base content to enable binding or annealing of the oligonucleotide (or a strand thereof) to a STAT3 mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a target sequence or region of complementarity complementary to a sequence of any one of SEQ ID NOs: 89-280, and the targeting sequence or region of complementarity is 18 nucleotides in length. In some embodiments, an oligonucleotide comprises a target sequence or region of complementarity complementary to a sequence of any one of SEQ ID NOs: 89-280, and the targeting sequence or region of complementarity is 19 nucleotides in length. In some embodiments, an oligonucleotide comprises a target sequence or region of complementarity complementary to a sequence of any one of SEQ ID NOs: 473-664, and the targeting sequence or region of complementarity is 20 nucleotides in length. In some embodiments, an oligonucleotide comprises a targeting sequence or region of complementarity complementary to a sequence of any one of SEQ ID NOs: 473-664, and the targeting sequence or region of complementarity is 21 nucleotides in length. In some embodiments, an oligonucleotide comprises a targeting sequence or region of complementarity complementary to a sequence of any one of SEQ ID NOs: 473-664, and the targeting sequence or region of complementarity is 22 nucleotides in length. In some embodiments, an oligonucleotide comprises a targeting sequence or region of complementarity complementary to a sequence of any one of SEQ ID NOs: 473-664, and the targeting sequence or region of complementarity is 23 nucleotides in length. In some embodiments, an oligonucleotide comprises a targeting sequence or region of complementarity complementary to a sequence of any one of SEQ ID NOs: 473-664 and the targeting sequence or region of complementarity is 24 nucleotides in length.

In some embodiments, an oligonucleotide herein comprises a targeting sequence or a region of complementarity (e.g., an antisense strand or a guide strand of a double-stranded oligonucleotide) that is fully complementary to a STAT3 target sequence. In some embodiments, the targeting sequence or region of complementarity is partially complementary to a STAT3 target sequence. In some embodiments, the oligonucleotide comprises a targeting sequence or region of complementarity that is fully complementary to a sequence of STAT3 or STAT3. In some embodiments, the oligonucleotide comprises a targeting sequence or region of complementarity that is partially complementary to a sequence of STAT3 or STAT3.

In some embodiments, the oligonucleotide comprises a targeting sequence or region of complementarity that is fully complementary to a sequence of any one of SEQ ID NOs: 89-280. In some embodiments, the oligonucleotide comprises a targeting sequence or region of complementarity that is fully complementary to the sequence set forth in SEQ ID NOs: 108, 140, 141, and 147. In some embodiments, the oligonucleotide comprises a targeting sequence or region of complementarity that is partially complementary to a sequence of any one of SEQ ID NOs: 89-280. In some embodiments, the oligonucleotide comprises a targeting sequence or region of complementarity that is partially complementary to the sequence set forth in SEQ ID NOs: 108, 140, 141, and 147.

In some embodiments, the oligonucleotide herein comprises a targeting sequence or region of complementarity that is complementary to a contiguous sequence of nucleotides comprising a STAT3 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising a STAT3 mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising a STAT3 mRNA, wherein the contiguous sequence of nucleotides is 19 nucleotides in length.

In some embodiments, an oligonucleotide herein (e.g., an RNAi oligonucleotide) comprises a targeting sequence or a region of complementary that is complementary to a contiguous sequence of nucleotides of any one of SEQ ID NOs: 89-280, optionally wherein the contiguous sequence of nucleotides is 19 nucleotides in length. In some embodiments, the oligonucleotide comprises a targeting sequence or a region of complementary that is complementary to a contiguous sequence of nucleotides of any one of SEQ ID NOs: 108, 140, 141, and 147, wherein the contiguous sequence of nucleotides is 19 nucleotides in length. In some embodiments, the oligonucleotide comprises a targeting sequence or a region of complementary that is complementary to a contiguous sequence of nucleotides of any one of SEQ ID NOs: 473-664, wherein the contiguous sequence of nucleotides is 20 nucleotides in length. In some embodiments, the oligonucleotide comprises a targeting sequence or a region of complementary that is complementary to a contiguous sequence of nucleotides of any one of SEQ ID NOs: 492, 524, 525, and 531, wherein the contiguous sequence of nucleotides is 20 nucleotides in length.

In some embodiments, a targeting sequence or region of complementarity of an oligonucleotide that is complementary to contiguous nucleotides of STAT3 or STAT3 target sequence spans the entire length of an antisense strand. In some embodiments, a region of complementarity of an oligonucleotide that is complementary to contiguous nucleotides of STAT3 or STAT3 target sequence spans a portion of the entire length of an antisense strand. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-20 of a target sequence of STAT3 or STAT3.

In some embodiments, a targeting sequence or region of complementarity of an oligonucleotide herein (e.g., an RNAi oligonucleotide) is complementary to a contiguous sequence of nucleotides of any one of SEQ ID NOs: 89-280 and spans the entire length of an antisense strand. In some embodiments, a targeting sequence or region of complementarity of the oligonucleotide is complementary to a contiguous sequence of nucleotides of SEQ ID NOs: 89-280 and spans a portion of the entire length of an antisense strand. In some embodiments, an oligonucleotide herein (e.g., an RNAi oligonucleotide) comprises a region of complementarity (e.g., on an antisense strand of a dsRNA) that is at least partially (e.g., fully) complementary to a contiguous stretch of nucleotides spanning nucleotides 1-19 or 1-20 of a sequence as set forth in any one of SEQ ID NOs: 473-664.

In some embodiments, an oligonucleotide herein comprises a targeting sequence or region of complementarity having one or more bp mismatches with the corresponding STAT3 target sequence. In some embodiments, the targeting sequence or region of complementarity may have up to about 1, up to about 2, up to about 3, up to about 4, up to about 5, etc. mismatches with the corresponding STAT3 target sequence provided that the ability of the targeting sequence or region of complementarity to bind or anneal to the STAT3 mRNA under appropriate hybridization conditions and/or the ability of the oligonucleotide to inhibit STAT3 expression is maintained. Alternatively, the targeting sequence or region of complementarity may have no more than 1, no more than 2, no more than 3, no more than 4, or no more than 5 mismatches with the corresponding STAT3 target sequence provided that the ability of the targeting sequence or region of complementarity to bind or anneal to the STAT3 mRNA under appropriate hybridization conditions and/or the ability of the oligonucleotide to inhibit STAT3 expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or where in the mismatches are interspersed throughout the targeting sequence or region of complementarity. In some embodiments, the oligonucleotide comprises a targeting sequence or a region of complementary that is complementary to a contiguous sequence of nucleotides of any one of SEQ ID NOs: 89-280, wherein the targeting sequence or region of complementarity may have up to about 1, up to about 2, up to about 3, up to about 4, up to about 5, etc. mismatches with the corresponding STAT3 target sequence. In some embodiments, the oligonucleotide comprises a targeting sequence or a region of complementary that is complementary to a contiguous sequence of nucleotides of any one of SEQ ID NOs: 89-280, wherein the targeting sequence or region of complementarity may have no more than 1, no more than 2, no more than 3, no more than 4, or no more than 5 mismatches with the corresponding STAT3 target sequence. In some embodiments, the oligonucleotide comprises a targeting sequence or a region of complementary that is complementary to a contiguous sequence of nucleotides of any one of SEQ ID NOs: 108, 140, 141, and 147, wherein the targeting sequence or region of complementarity may have up to about 1, up to about 2, up to about 3, up to about 4, up to about 5, etc. mismatches with the corresponding STAT3 target sequence. In some embodiments, the oligonucleotide comprises a targeting sequence or a region of complementary that is complementary to a contiguous sequence of nucleotides of any one of SEQ ID NOs: 108, 140, 141, and 147, wherein the targeting sequence or region of complementarity may have no more than 1, no more than 2, no more than 3, no more than 4, or no more than 5 mismatches with the corresponding STAT3 target sequence.

### Targeting Ligands

In some embodiments, it is desirable to target the STAT3 targeting oligonucleotides of the disclosure to one or more cells or one or more organs. Such a strategy can help to avoid undesirable effects in other organs or avoid undue loss of the oligonucleotide to cells, tissue or organs that would not benefit from the oligonucleotide. Targeting of oligonucleotides to one or more cells or one or more organs can be achieved through a variety of approaches. Conjugation of oligonucleotides to tissue or cell specific antibodies, small molecules or targeting ligands can facilitate delivery to and modify accumulation of the oligonucleotide in one or more target cells or tissues (Chernolovskaya et al., (2019) FRONT PHARMACOL. 10:444). For example, conjugation of an oligonucleotide to a saturated fatty acid (e.g., C22) may facilitate delivery to cells or tissues like adipose tissue or immune cells which uptake such ligands more readily than conventional oligonucleotide ligands. Accordingly, in some embodiments, oligonucleotides disclosed herein are modified to facilitate targeting and/or delivery of a tissue, cell, or organ (e.g., to facilitate delivery of the oligonucleotide to the liver). In certain embodiments, oligonucleotides disclosed herein are modified to facilitate delivery of the oligonucleotide to cells of the immune system. In certain embodiments, oligonucleotides disclosed herein are modified to facilitate delivery of the oligonucleotide to myeloid derived suppressor cells. In some embodiments, an oligonucleotide comprises at least one nucleotide (e.g., 1, 2, 3, 4, 5, 6 or more nucleotides) conjugated to one or more targeting ligand(s).

In some embodiments, the targeting ligand comprises a carbohydrate, amino sugar, cholesterol, peptide, polypeptide, protein, or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a dsRNA) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a tetraloop, and wherein 3 nucleotides of the tetraloop are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, 4 GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (tetraL) has a monovalent GalNAc moiety attached to any one or more guanine nucleotides of the tetraloop via any linker described herein, as depicted below in Chem 2 (X=heteroatom):

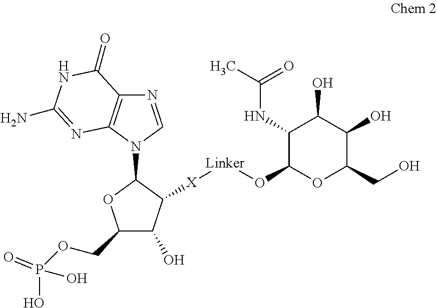

In some embodiments, the tetraloop (tetraL) has a monovalent GalNAc attached to any one or more adenine nucleotides of the tetraloop via any linker described herein, as depicted below in Chem 3 (X=heteroatom):

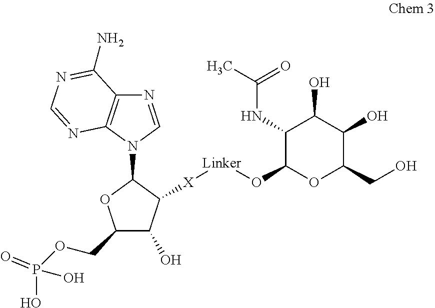

In some embodiments, an oligonucleotide herein comprises a monovalent GalNAc attached to a guanine nucleotide referred to as [ademG-GalNAc] or 2′-aminodiethoxymethanol-Guanine-GalNAc, as depicted below in Chem 4:

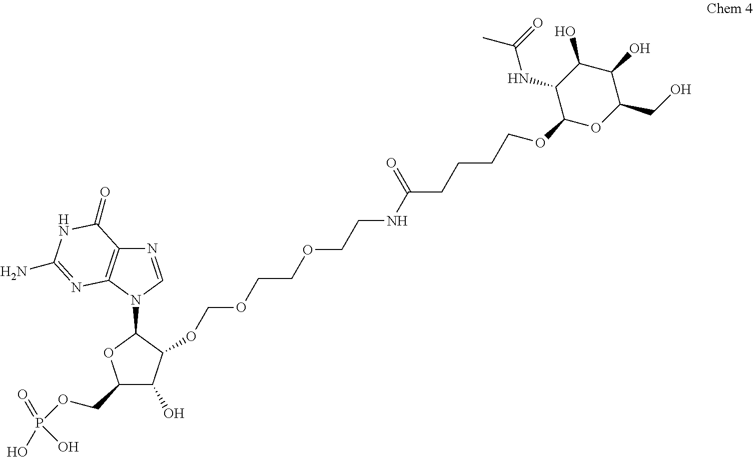 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below in Chem 5:

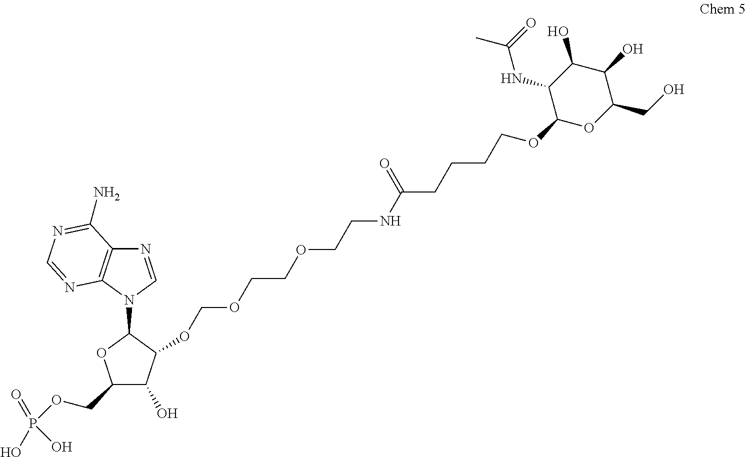

An example of such conjugation is shown below (Chem 6) for a loop comprising from 5′ to 3′ the nucleotide sequence GAAA (L=linker, X=heteroatom) stem attachment points are shown. Such a loop may be present, for example, at positions 27-30 of the sense strand as shown in FIG. 1. In the chemical formula,

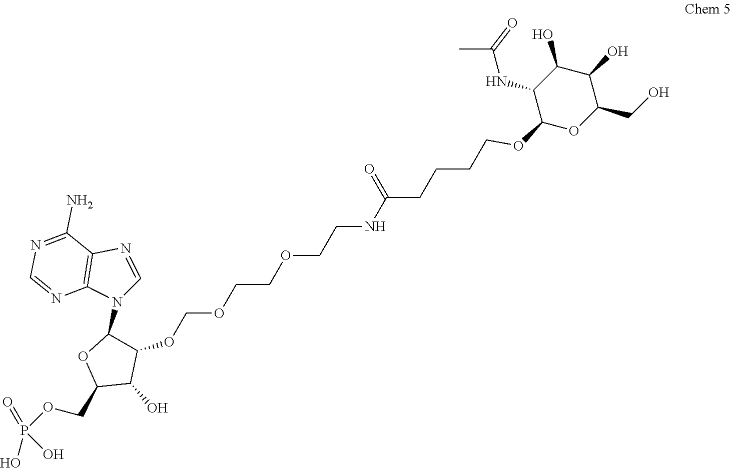

is used to describe an attachment point to the oligonucleotide strand (Chem 6).

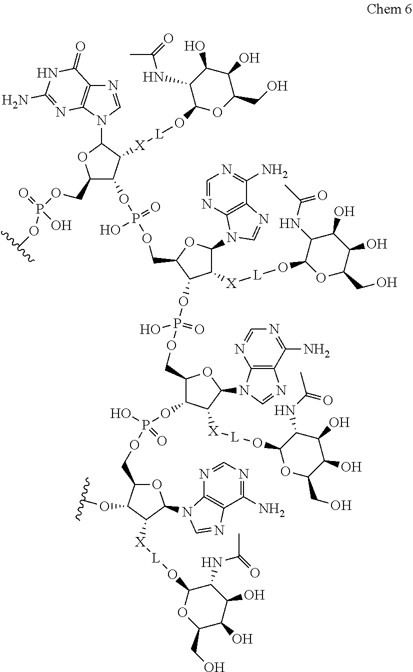

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to nucleotides of the loop using an acetal linker (Chem 7 and Chem 8). Such a loop may be present, for example, at positions 27-30 of the any one of the sense strand as shown in FIG. 1. In the chemical formula,

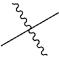

is an attachment point to the oligonucleotide strand (Chem 7 and Chem 8).

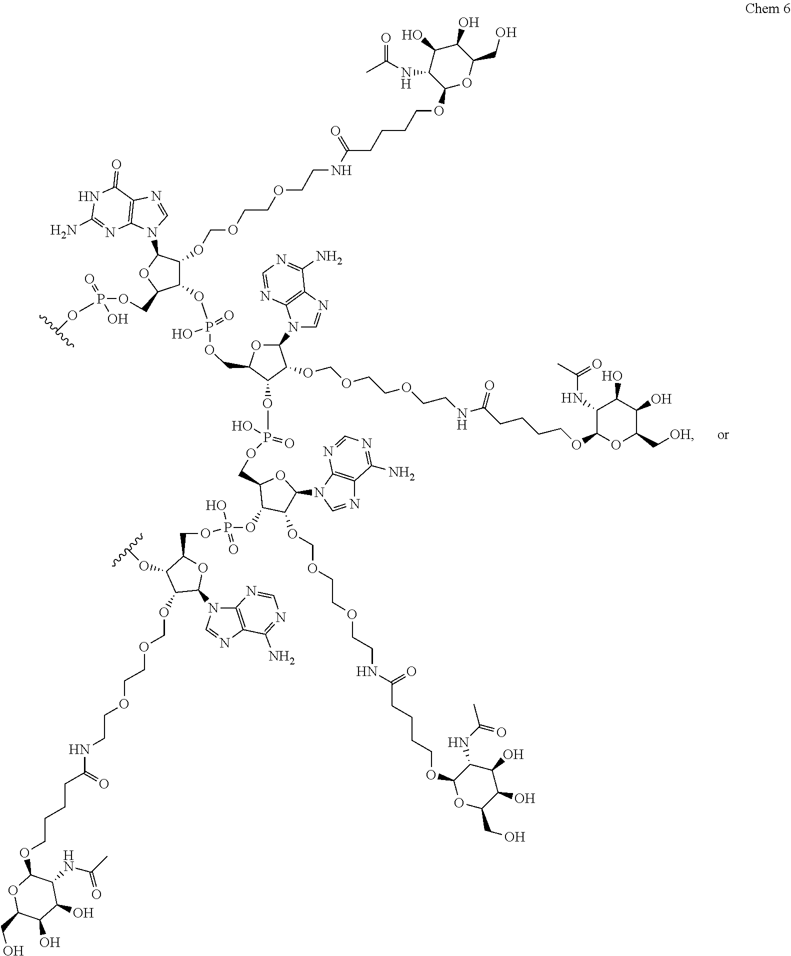 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

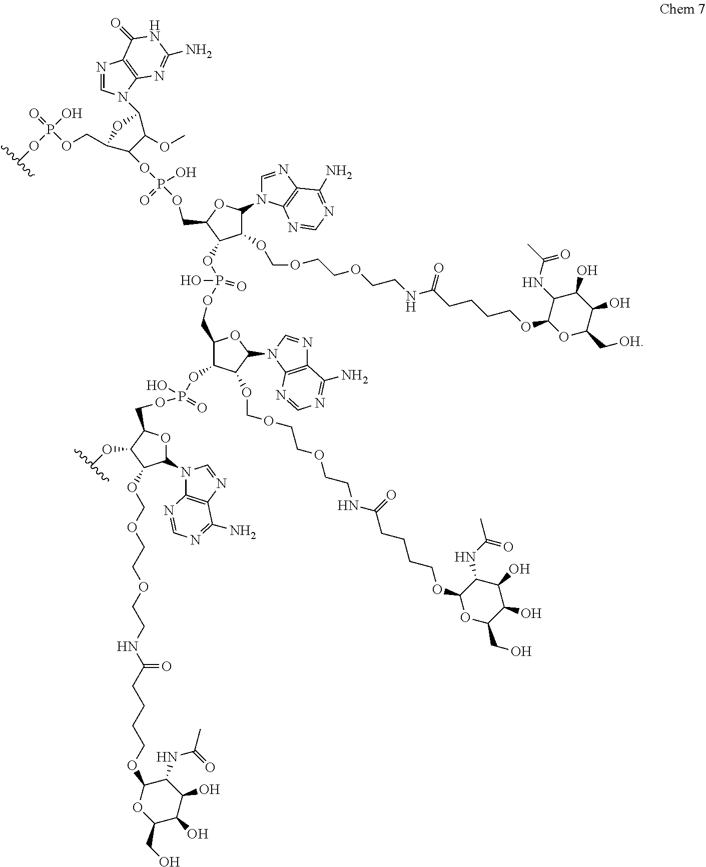

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a dsRNA. In some embodiments, the oligonucleotides herein do not have a GalNAc conjugated thereto.

### Structure of Conjugated STAT3 Targeting Oligonucleotides

In some embodiments, a STAT3 targeting oligonucleotide described herein comprises a nucleotide sequence having a region of complementarity to a STAT3 mRNA target sequence and one or more targeting ligands, wherein the nucleotide sequence comprises one or more nucleosides (nucleic acids) conjugated with one or more targeting ligands represented by formula I-a:

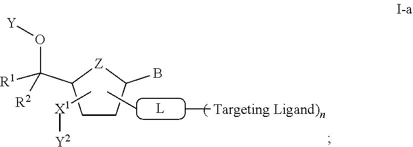

or a pharmaceutically acceptable salt thereof,
wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each targeting ligand is selected from lipid conjugate moiety (LC), carbohydrate, amino sugar or GalNAc; and wherein each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

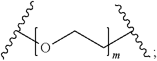

m is 1-50;
X¹, V¹ and W¹ are independently —C(R)₂—, —OR, —O—, —S—, —Se—, or —NR—;
Y is hydrogen, a suitable hydroxyl protecting group,

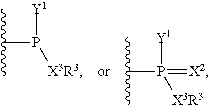

R³ is hydrogen, a suitable protecting group, a suitable prodrug, or an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

In some embodiments, the STAT3 targeting oligonucleotide comprises one or more nucleic acids conjugated with targeting ligands represented by formula II-a:

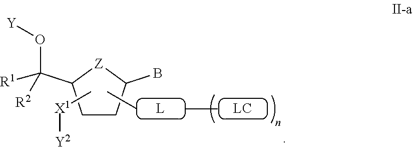

or a pharmaceutically acceptable salt thereof.

In some embodiments, the STAT3 targeting oligonucleotide comprises one or more nucleic acids conjugated with targeting ligands represented by formula II-b or II-c:

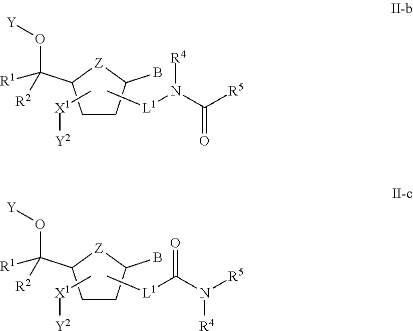

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

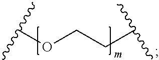

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments, R⁵ is selected from

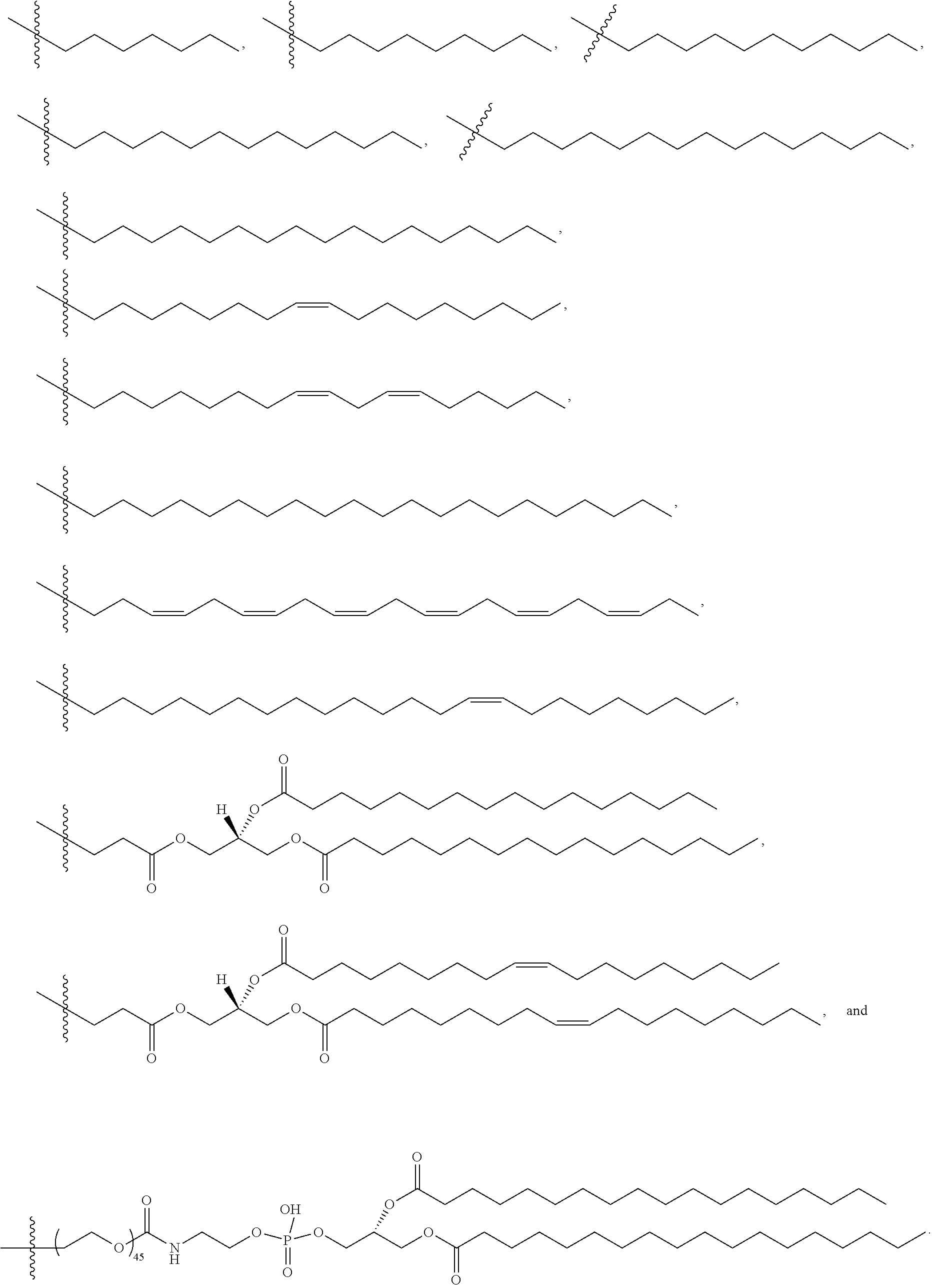

In some embodiments, R⁵ is selected from:

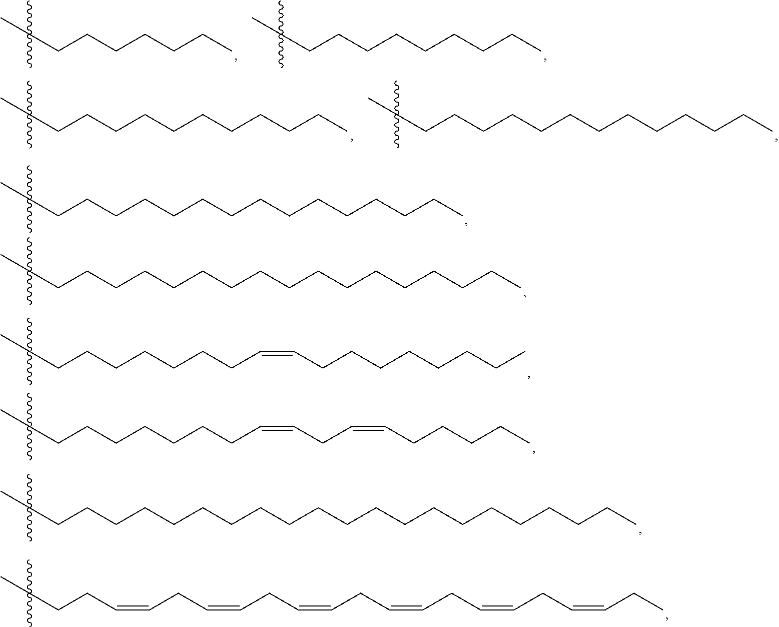

In some embodiments, R⁵ is

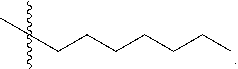 [SMILES: *CCCCCCC]

In some embodiments, R⁵ is

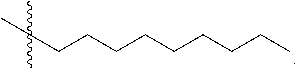 [SMILES: *CCCCCCCCC]

In some embodiments, R⁵ is

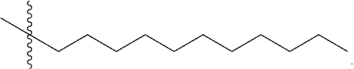 [SMILES: *CCCCCCCCCCC]

In some embodiments, R⁵ is

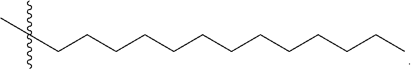 [SMILES: *CCCCCCCCCCCCC]

In some embodiments, R⁵ is

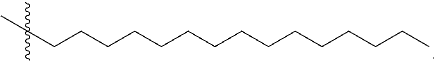 [SMILES: *CCCCCCCCCCCCCCC]

In some embodiments, R⁵ is

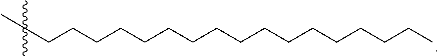 [SMILES: *CCCCCCCCCCCCCCCCC]

In some embodiments, R⁵ is

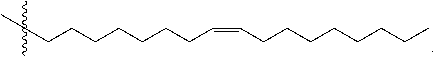 [SMILES: *CCCCCCC/C=C\CCCCCCCC]

In some embodiments, R⁵ is

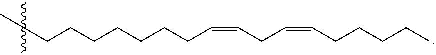 [SMILES: *CCCCCCC/C=C\C/C=C\CCCCC]

In some embodiments, R⁵ is

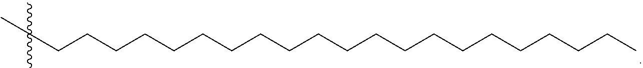 [SMILES: *CCCCCCCCCCCCCCCCCCCCC]

In some embodiments, R⁵ is

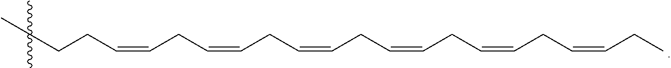 [SMILES: *CC/C=C\C/C=C\C/C=C\C/C=C\C/C=C\C/C=C\CC]

In some embodiments, R⁵ is

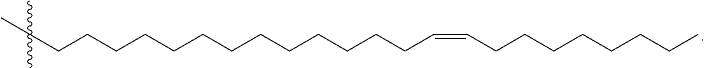 [SMILES: *CCCCCCCCCCCCC/C=C\CCCCCCCC]

In some embodiments, R⁵ is

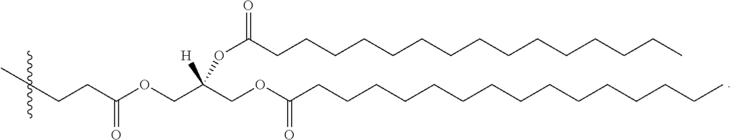 [SMILES: *CCC(=O)OC[C@@H](COC(=O)CCCCCCCCCCCCCCC)OC(=O)CCCCCCCCCCCCCCC]

In some embodiments, R⁵ is

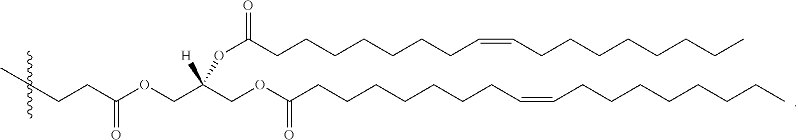 [SMILES: *CCC(=O)OC[C@@H](COC(=O)CCCCCCC/C=C\CCCCCCCC)OC(=O)CCCCCCC/C=C\CCCCCCCC]

In some embodiments, R⁵ is

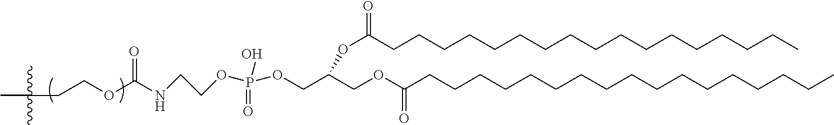

In some embodiments, the STAT3 targeting oligonucleotide comprises one or more nucleic acids conjugated with targeting ligands represented by formula II-Ib or II-Ic:

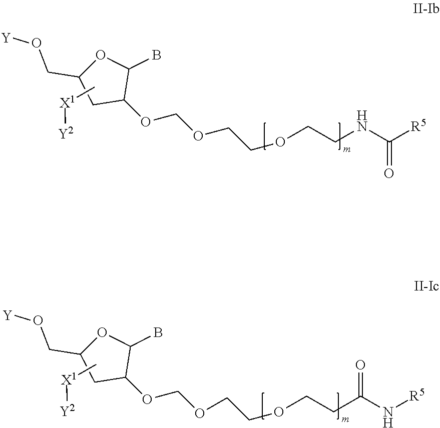

or a pharmaceutically acceptable salt thereof; wherein
B is a nucleobase or hydrogen;
m is 1-50;
X¹ is —O—, or —S—;
Y is hydrogen,

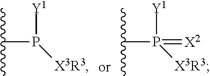

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(O)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁵ is selected from

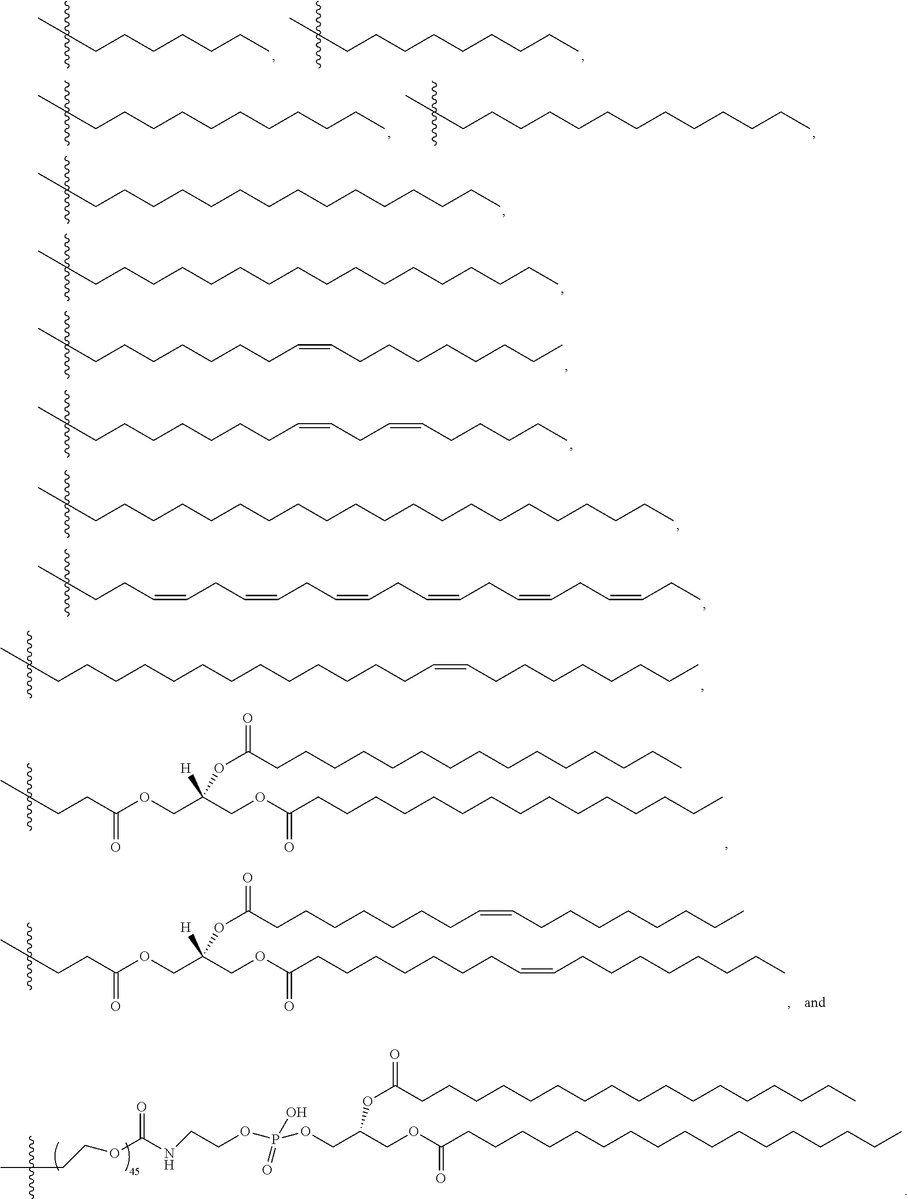

In some embodiments, R⁵ is

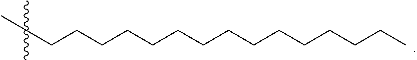 [SMILES: *CCCCCCCCCCCCCCC]

In some embodiments, R⁵ is

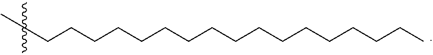 [SMILES: *CCCCCCCCCCCCCCCCC]

In some embodiments, the nucleotide sequence of the STAT3 targeting oligonucleotide comprises 1-10 targeting ligands. In some embodiments, the nucleotide sequence comprises 1, 2 or 3 targeting ligands.

In some embodiments, the STAT3 targeting oligonucleotide is a double-stranded molecule. In some embodiments, the STAT3 targeting oligonucleotide is an RNAi molecule. In some embodiments, the STAT3 targeting double stranded oligonucleotide comprises a stem loop. In some embodiments, the ligand is conjugated to any of the nucleotides in the stem loop. In some embodiments, the ligand is conjugated to the first nucleotide from 5′ to 3′, in the stem loop. In some embodiments, the ligand is conjugated to the second nucleotide from 5′ to 3′ in the stem loop. In some embodiments, the ligand is conjugated to the third nucleotide from 5′ to 3′ in the stem loop. In some embodiments, the ligand is conjugated to the fourth nucleotide from 5′ to 3′ in the stem loop. In some embodiments, the ligand is conjugated to one, two, three, or four of the nucleotides in the stem loop. In some embodiments, the ligand is conjugated to three of the nucleotides in the stem loop.

In some embodiments, the STAT3 targeting double stranded oligonucleotide comprises a stem loop, wherein one or more lipids are conjugated to one or more nucleotides of the stem loop. In some embodiments, the STAT3 targeting double stranded oligonucleotide comprises a stem loop, wherein one or more C16 lipids are conjugated to one or more nucleotides of the stem loop. In some embodiments, the STAT3 targeting double stranded oligonucleotide comprises a stem loop, wherein one or more C18 lipids are conjugated to one or more nucleotides of the stem loop.

In some embodiments, the STAT3 targeting oligonucleotide comprises a sense strand of 36 nucleotides with positions numbered 1-36 from 5′ to 3′. In some embodiments, the STAT3 targeting oligonucleotide comprises a lipid conjugated to position 27 of a 36-nucleotide sense strand. In some embodiments, STAT3 targeting oligonucleotide comprises a lipid conjugated to position 28 of a 36-nucleotide sense strand. In some embodiments, the STAT3 targeting oligonucleotide comprises a lipid conjugated to position 29 of a 36-nucleotide sense strand. In some embodiments, the STAT3 targeting oligonucleotide comprises a lipid conjugated to position 30 of a 36-nucleotide sense strand. In some embodiments, a 36-nucleotide sense strand forms a stem loop having a loop with positions 27-30. In some embodiments, a lipid is conjugated to more than one position of the loop (e.g., positions 27 and 28 of a 36-nucleotide sense strand).

In some embodiments, the STAT3 targeting oligonucleotide comprises a C16 lipid conjugated to position 27 of a 36-nucleotide sense strand. In some embodiments, STAT3 targeting oligonucleotide comprises a C16 lipid conjugated to position 28 of a 36-nucleotide sense strand. In some embodiments, the STAT3 targeting oligonucleotide comprises a C16 lipid conjugated to position 29 of a 36-nucleotide sense strand. In some embodiments, the STAT3 targeting oligonucleotide comprises a C16 lipid conjugated to position 30 of a 36-nucleotide sense strand. In some embodiments, a 36-nucleotide sense strand forms a stem loop having a loop with positions 27-30. In some embodiments, a C16 lipid is conjugated to more than one position of the loop (e.g., positions 27 and 28 of a 36-nucleotide sense strand).

In some embodiments, the STAT3 targeting oligonucleotide comprises a C18 lipid conjugated to position 27 of a 36-nucleotide sense strand. In some embodiments, STAT3 targeting oligonucleotide comprises a C18 lipid conjugated to position 28 of a 36-nucleotide sense strand. In some embodiments, the STAT3 targeting oligonucleotide comprises a C18 lipid conjugated to position 29 of a 36-nucleotide sense strand. In some embodiments, the STAT3 targeting oligonucleotide comprises a C18 lipid conjugated to position 30 of a 36-nucleotide sense strand. In some embodiments, a 36-nucleotide sense strand forms a stem loop having a loop with positions 27-30. In some embodiments, a C18 lipid is conjugated to more than one position of the loop (e.g., positions 27 and 28 of a 36-nucleotide sense strand).

In some embodiments, a STAT3 targeting oligonucleotide comprises an antisense strand of 15 to 30 nucleotides and a sense strand of 15 to 40 nucleotide, wherein the sense and antisense strands form a duplex region, wherein the antisense strand comprises a region of complementarity to a STAT3 mRNA target sequence expressed in an immune cell associated with a tumor microenvironment, wherein the sense strand comprises at its 3′ end a stem-loop comprising a tetraloop comprising 4 nucleosides, wherein one or more of the 4 nucleosides is represented by formula II-Ib:

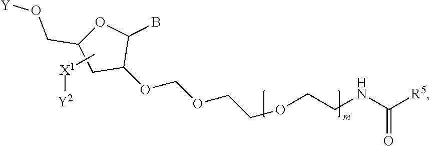

wherein B is selected from an adenine and a guanine nucleobase, and wherein R⁵ is a hydrocarbon chain. In some embodiments, m is 1, X1 is O, Y2 is an internucleotide linking group attaching to the 5′ terminal of a nucleoside,

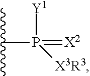

Y is represented by Y1 is a linking group attaching to the 2′ or 3′ terminal of a nucleotide, X2 is O, X3 is O, and R3 is H.

In some embodiments, the hydrocarbon chain is a C8-C30 hydrocarbon chain. In some embodiments, the hydrocarbon chain is a C16 hydrocarbon chain. In some embodiments, the C16 hydrocarbon chain is represented by

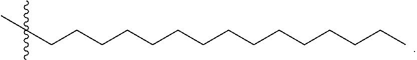 [SMILES: *CCCCCCCCCCCCCCC]

In some embodiments, the hydrocarbon chain is a C18 hydrocarbon chain. In some embodiments, the C18 hydrocarbon chain is represented by

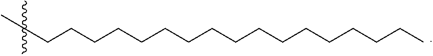 [SMILES: *CCCCCCCCCCCCCCCCC]

In some embodiments, the oligonucleotide comprises a sense strand comprising a sequence selected from SEQ ID NOs: 89-280, wherein the sense strand comprises a C18 lipid. In some embodiments, the 4 nucleosides of the tetraloop are numbered 1-4 from 5′ to 3′ and position 1 is represented by formula II-Ib. In some embodiments, position 2 is represented by formula II-Ib. In some embodiments, position 3 is represented by formula II-Ib. In some embodiments, position 4 is represented by formula II-Ib. In some embodiments, the sense strand is 36 nucleotides with positions numbered 1-36 from 5′ to 3′, wherein the stem-loop comprises nucleotides at positions 21-36, and wherein one or more nucleosides at positions 27-30 are represented by formula II-Ib. In some embodiments, the antisense strand is 22 nucleotides.

### Exemplary STAT3 Targeting Oligonucleotides

In some embodiments, an oligonucleotide targeting STAT3 comprises a sense strand and an antisense strand as set forth in Tables 3, 4, 5, 10, 11, 12, 13, and 14, wherein the oligonucleotide comprises a stem loop structure having a double-stranded stem of about 2-6 base pairs and a loop of 3-4 nucleotides, and wherein the sense and antisense strands comprise the modification pattern set forth in FIG. 1 or Example 12. In some embodiments, an oligonucleotide targeting STAT3 comprises a sense strand and an antisense strand as set forth in Tables 3, 4, 5, 10, 11, 12, 13, and 14, wherein the oligonucleotide comprises a stem loop structure having a double-stranded stem of about 2-6 base pairs and a loop of 3-4 nucleotides, wherein the sense and antisense strands comprise the modification pattern set forth in FIG. 1, and wherein antisense strand is modified with an oxymethylphosphonate at the 4′ carbon of the 5′ terminal nucleotide. In some embodiments, the oligonucleotide comprises a stem loop comprising the nucleotide sequence of SEQ ID NO: 86. In some embodiments, the oligonucleotide comprises a double-stranded stem of 6 base pairs and a stem loop of 4 nucleotides comprising one, two, three or four GalNAc conjugated nucleotides. In some embodiments, the GalNAc conjugated nucleotide is a monovalent GalNAc conjugated to an adenine nucleotide, referred to as [ademA-GalNAc] or 2′-aminodiethoxymethanol-Adenine-GalNAc, as depicted below:

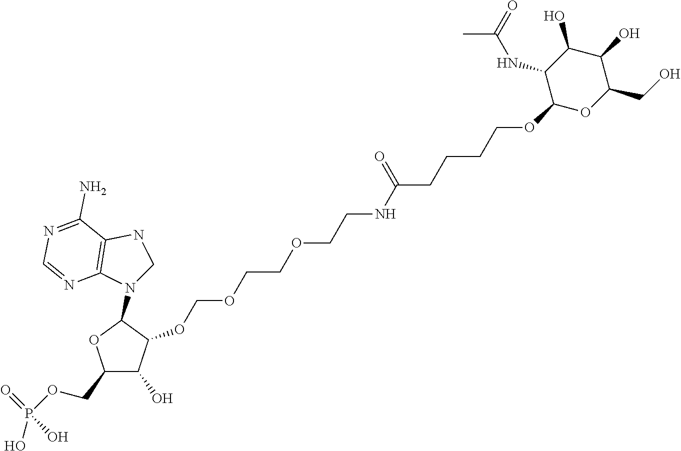

In some embodiments, the stem loop comprises a double-stranded stem of 6 base pairs and a loop comprising the nucleotide sequence GAAA, wherein each adenine nucleotide is ademA-GalNAc.

In some embodiments, an oligonucleotide for reducing expression of STAT3 mRNA comprises a sense and antisense strand comprising nucleotide sequences selected from:
(a) SEQ ID NOs: 9 and 10, respectively;
(b) SEQ ID NOs: 37 and 38, respectively;
(c) SEQ ID NOs: 65 and 66, respectively; and
(d) SEQ ID NOs: 69 and 70, respectively.

In some embodiments, an oligonucleotide for reducing expression of STAT3 mRNA comprises a sense strand and an antisense strand comprising nucleotide sequences selected from:
(a) SEQ ID NOs: 9 and 10, respectively;
(b) SEQ ID NOs: 37 and 38, respectively;
(c) SEQ ID NOs: 65 and 66, respectively; and
(d) SEQ ID NOs: 69 and 70, respectively,
   wherein the sense and antisense strands are modified based on the pattern below
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-C#][mX][mX][mX][mX][mX][mX][mX][mX]
Hybridized to
Antisense Strand: [MePhosphonate-4O-mXs][fXs][fX][fX][fX][mX][fX][mX][mX][fX][mX][mX][mX][fX][mX][mX][mX][mX][mX][mXs][mXs][mX]
   (key provided in Table 1). In some embodiments, C # is C16 or C18.

In some embodiments, an oligonucleotide for reducing expression of STAT3 mRNA comprises a sense strand and an antisense strand comprising nucleotide sequences selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively,
   wherein the sense and antisense strands are modified based on the pattern below
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-C #][mX][mX][mX][mX][mX][mX][mX][mX]
Hybridized to
Antisense Strand: [MePhosphonate-4O-mXs][fXs][fX][fX][fX][mX][fX][mX][mX][fX][mX][mX][mX][fX][mX][mX][mX][mX][mX][mXs][mXs][mX]
   (key provided in Table 1). In some embodiments, C # is C16 or C18.

In some embodiments, an oligonucleotide for reducing expression of STAT3 mRNA comprises a sense strand and an antisense strand comprising nucleotide sequences selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively,
   wherein the sense and antisense strands are modified based on the pattern below
Sense Strand:
[mXs][mX][mX][mX][mX][mX][mX][fX][fX][fX][fX][mX][mX][mX][mX][mX][mX][mX][mX][mX][mX][mX][mX][mX][mX][mX][mX][ademX-C#][mX][mX][mX][mX][mX][mX][mX][mX]
Hybridized to
Antisense Strand: [MePhosphonate-4O-mXs][fXs][fXs][fX][fX][mX][fX][mX][mX][fX][mX][mX][mX][fX][mX][mX][mX][mX][mX][mXs][mXs][mX]
   (key provided in Table 1). In some embodiments, C # is C16 or C18.

In some embodiments, an oligonucleotide for reducing expression of STAT3 mRNA comprises a sense and antisense strand comprising nucleotide sequences selected from:
(a) SEQ ID NOs: 11 and 12, respectively;
(b) SEQ ID NOs: 39 and 40, respectively;
(c) SEQ ID NOs: 67 and 68, respectively; and
(d) SEQ ID NOs: 71 and 72, respectively.

In some embodiments, an oligonucleotide for reducing expression of STAT3 mRNA comprises the sequence set forth in SEQ ID NO: 81. In some embodiments, an oligonucleotide for reducing expression of STAT3 mRNA comprises the sequence set forth in SEQ ID NO: 82. In some embodiments, an oligonucleotide for reducing expression of STAT3 mRNA comprises the sequence set forth in SEQ ID NO: 83. In some embodiments, an oligonucleotide for reducing expression of STAT3 mRNA comprises the sequence set forth in SEQ ID NO: 84.

In some embodiments, an oligonucleotide for reducing expression of STAT3 mRNA comprises a sense and antisense strand having nucleotide sequences set forth in SEQ ID NOs: 87 and 68, respectively. In some embodiments, an oligonucleotide for reducing expression of STAT3 mRNA comprises a sense and antisense strand having nucleotide sequences set forth in SEQ ID NOs: 88 and 71, respectively.

In some embodiments, an oligonucleotide for reducing expression of STAT3 mRNA comprises a sense strand sequence selected from SEQ ID NOs: 89-280. In some embodiments, an oligonucleotide for reducing expression of STAT3 mRNA comprises a sense strand sequence selected from SEQ ID NOs: 857-946. In some embodiments, an oligonucleotide for reducing expression of STAT3 mRNA comprises a sense strand sequence selected from SEQ ID NOs: 857-888. In some embodiments, an oligonucleotide for reducing expression of STAT3 mRNA comprises a sense strand sequence selected from SEQ ID NOs: 889-912. In some embodiments, an oligonucleotide for reducing expression of STAT3 mRNA comprises a sense strand sequence selected from SEQ ID NOs: 913-934. In some embodiments, an oligonucleotide for reducing expression of STAT3 mRNA comprises a sense strand sequence selected from SEQ ID NOs: 935-946.

In some embodiments, an oligonucleotide for reducing expression of STAT3 mRNA comprises an antisense strand sequence selected from SEQ ID NOs: 947-1036. In some embodiments, an oligonucleotide for reducing expression of STAT3 mRNA comprises an antisense strand sequence selected from SEQ ID NOs: 947-978. In some embodiments, an oligonucleotide for reducing expression of STAT3 mRNA comprises an antisense strand sequence selected from SEQ ID NOs: 979-1002. In some embodiments, an oligonucleotide for reducing expression of STAT3 mRNA comprises an antisense strand sequence selected from SEQ ID NOs: 1003-1024. In some embodiments, an oligonucleotide for reducing expression of STAT3 mRNA comprises an antisense strand sequence selected from SEQ ID NOs: 1025-1036.

In some embodiments, an oligonucleotide for reducing expression of STAT3 mRNA comprises a sense strand sequence selected from SEQ ID NOs: 857-946 and an antisense strand selected from SEQ ID NOs: 947-1036. In some embodiments, an oligonucleotide for reducing expression of STAT3 mRNA comprises a sense strand sequence selected from SEQ ID NOs: 857-888 and an antisense strand selected from SEQ ID NOs: 947-978. In some embodiments, an oligonucleotide for reducing expression of STAT3 mRNA comprises a sense strand sequence selected from SEQ ID NOs: 889-912 and an antisense strand selected from SEQ ID NOs: 979-1002. In some embodiments, an oligonucleotide for reducing expression of STAT3 mRNA comprises a sense strand sequence selected from SEQ ID NOs: 913-934 and an antisense strand selected from SEQ ID NOs:1003-1024. In some embodiments, an oligonucleotide for reducing expression of STAT3 mRNA comprises a sense strand sequence selected from SEQ ID NOs: 935-946 and an antisense strand selected from SEQ ID NOs:1025-1036.

In some embodiments, an oligonucleotide for reducing expression of STAT3 mRNA comprises a sense strand sequence selected from SEQ ID NOs: 1037-1126. In some embodiments, an oligonucleotide for reducing expression of STAT3 mRNA comprises a sense strand sequence selected from SEQ ID NOs: 1037-1068. In some embodiments, an oligonucleotide for reducing expression of STAT3 mRNA comprises a sense strand sequence selected from SEQ ID NOs:1069-1092. In some embodiments, an oligonucleotide for reducing expression of STAT3 mRNA comprises a sense strand sequence selected from SEQ ID NOs: 1093-1114. In some embodiments, an oligonucleotide for reducing expression of STAT3 mRNA comprises a sense strand sequence selected from SEQ ID NOs:1115-1126.

In some embodiments, an oligonucleotide for reducing expression of STAT3 mRNA comprises an antisense strand sequence selected from SEQ ID NOs: 1127-1216. In some embodiments, an oligonucleotide for reducing expression of STAT3 mRNA comprises an antisense strand sequence selected from SEQ ID NOs: 1127-1158. In some embodiments, an oligonucleotide for reducing expression of STAT3 mRNA comprises an antisense strand sequence selected from SEQ ID NOs: 1159-1182. In some embodiments, an oligonucleotide for reducing expression of STAT3 mRNA comprises an antisense strand sequence selected from SEQ ID NOs:1183-1204. In some embodiments, an oligonucleotide for reducing expression of STAT3 mRNA comprises an antisense strand sequence selected from SEQ ID NOs:1205-1216.

In some embodiments, an oligonucleotide for reducing expression of STAT3 mRNA comprises a sense strand sequence selected from SEQ ID NOs: 1037-1126 and an antisense strand selected from SEQ ID NOs: 1127-1216. In some embodiments, an oligonucleotide for reducing expression of STAT3 mRNA comprises a sense strand sequence selected from SEQ ID NOs: 1037-1068 and an antisense strand selected from SEQ ID NOs: 1127-1182. In some embodiments, an oligonucleotide for reducing expression of STAT3 mRNA comprises a sense strand sequence selected from SEQ ID NOs: 1069-1092 and an antisense strand selected from SEQ ID NOs: 1159-1182. In some embodiments, an oligonucleotide for reducing expression of STAT3 mRNA comprises a sense strand sequence selected from SEQ ID NOs: 1093-1114 and an antisense strand selected from SEQ ID NOs:1183-1204. In some embodiments, an oligonucleotide for reducing expression of STAT3 mRNA comprises a sense strand sequence selected from SEQ ID NOs: 1115-1126 and an antisense strand selected from SEQ ID NOs:1205-1216.

In some embodiments, an oligonucleotide for reducing expression of STAT3 mRNA comprises a sense and antisense strand comprising nucleotide sequences selected from:
(a) SEQ ID NOs: 857 and 947, respectively;
(b) SEQ ID NOs: 858 and 948, respectively;
(c) SEQ ID NOs: 859 and 949, respectively;
(d) SEQ ID NOs: 860 and 950, respectively;
(e) SEQ ID NOs: 862 and 952, respectively;
(f) SEQ ID NOs: 867 and 957, respectively;
(g) SEQ ID NOs: 875 and 965, respectively; and
(h) SEQ ID NOs: 876 and 966, respectively.

In some embodiments, an oligonucleotide for reducing expression of STAT3 mRNA comprises a sense and antisense strand comprising nucleotide sequences selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively.

In some embodiments, an oligonucleotide for reducing expression of STAT3 mRNA comprises a sense and antisense strand comprising nucleotide sequences selected from:
(a) SEQ ID NOs: 940 and 1030, respectively;
(b) SEQ ID NOs: 937 and 1027, respectively; and
(c) SEQ ID NOs: 939 and 1029, respectively.

In some embodiments, an oligonucleotide for reducing expression of STAT3 mRNA comprises a sense and antisense strand comprising nucleotide sequences selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively.

In some embodiments, an oligonucleotide for reducing expression of STAT3 mRNA comprises a sense and antisense strand comprising nucleotide sequences selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively.

In some embodiments, the sense strand comprises the sequence of SEQ ID NO: 862 and the antisense strand comprises the sequence of SEQ ID NO: 952.

In some embodiments, the sense strand comprises the sequence of SEQ ID NO: 875 and the antisense strand comprises the sequence of SEQ ID NO: 965.

In some embodiments, the sense strand comprises the sequence of SEQ ID NO: 876 and the antisense strand comprises the sequence of SEQ ID NO: 966.

In some embodiments, the sense strand comprises the sequence of SEQ ID NO: 920 and the antisense strand comprises the sequence of SEQ ID NO: 1010.

In some embodiments, an oligonucleotide for reducing expression of STAT3 mRNA comprises a sense and antisense strand comprising nucleotide sequences selected from:
(a) SEQ ID NOs: 1037 and 1127, respectively;
(b) SEQ ID NOs: 1038 and 1128, respectively;
(c) SEQ ID NOs: 1039 and 1129, respectively;
(d) SEQ ID NOs: 1040 and 1130, respectively;
(e) SEQ ID NOs: 1042 and 1132, respectively;
(f) SEQ ID NOs: 1047 and 1137, respectively;
(g) SEQ ID NOs: 1055 and 1145, respectively; and
(h) SEQ ID NOs: 1056 and 1146, respectively.

In some embodiments, an oligonucleotide for reducing expression of STAT3 mRNA comprises a sense and antisense strand comprising nucleotide sequences selected from:
(a) SEQ ID NOs: 1081 and 1171, respectively;
(b) SEQ ID NOs: 1090 and 1180, respectively;
(c) SEQ ID NOs: 1079 and 1169, respectively;
(d) SEQ ID NOs: 1076 and 1166, respectively;
(e) SEQ ID NOs: 1072 and 1162, respectively;
(f) SEQ ID NOs: 1070 and 1160, respectively; and
(g) SEQ ID NOs: 1069 and 1159, respectively.

In some embodiments, an oligonucleotide for reducing expression of STAT3 mRNA comprises a sense and antisense strand comprising nucleotide sequences selected from:
(a) SEQ ID NOs: 1120 and 1210, respectively;
(c) SEQ ID NOs: 1119 and 1209, respectively.

In some embodiments, an oligonucleotide for reducing expression of STAT3 mRNA comprises a sense and antisense strand comprising nucleotide sequences selected from:
(a) SEQ ID NOs: 1095 and 1185, respectively;
(b) SEQ ID NOs: 1104 and 1194, respectively;
(c) SEQ ID NOs: 1093 and 1183, respectively; and
(d) SEQ ID NOs: 1100 and 1190, respectively.

In some embodiments, an oligonucleotide for reducing expression of STAT3 mRNA comprises a sense and antisense strand comprising nucleotide sequences selected from:
(a) SEQ ID NOs: 1042 and 1132, respectively;
(b) SEQ ID NOs: 1055 and 1145, respectively;
(c) SEQ ID NOs: 1056 and 1146, respectively; and
(d) SEQ ID NOs: 1100 and 1190, respectively.

In some embodiments, the sense strand comprises the sequence of SEQ ID NO: 1042 and the antisense strand comprises the sequence of SEQ ID NO: 1132.

In some embodiments, the sense strand comprises the sequence of SEQ ID NO: 1055 and the antisense strand comprises the sequence of SEQ ID NO: 1145.

In some embodiments, the sense strand comprises the sequence of SEQ ID NO: 1056 and the antisense strand comprises the sequence of SEQ ID NO: 1146.

In some embodiments, the sense strand comprises the sequence of SEQ ID NO: 1100 and the antisense strand comprises the sequence of SEQ ID NO: 1190.

In some embodiments, an oligonucleotide for reducing expression of STAT3 mRNA comprises a sense and antisense strand comprising nucleotide sequences selected from:
(a) SEQ ID NOs: 1042 and 1225, respectively;
(b) SEQ ID NOs: 1055 and 1226, respectively;
(c) SEQ ID NOs: 1056 and 1227, respectively; and
(d) SEQ ID NOs: 1100 and 1228, respectively.

In some embodiments, an oligonucleotide for reducing expression of STAT3 mRNA described herein comprises minimal off-target effects. For example, in some embodiments, an oligonucleotide described herein reduces STAT3 expression and does not reduce STAT1 expression or reduces STAT1 expression less than STAT3 expression. In some embodiments, the oligonucleotide comprises a sense strand comprising the nucleotide sequence set forth in SEQ ID NO: 862 and an antisense strand comprising the nucleotide sequence set forth in SEQ ID NO: 952, wherein the oligonucleotide reduces STAT3 expression and does not reduce STAT1 expression or reduces STAT1 expression less than STAT3 expression. In some embodiments, the oligonucleotide comprises a sense strand comprising the nucleotide sequence set forth in SEQ ID NO: 1042 and an antisense strand comprising the nucleotide sequence set forth in SEQ ID NO: 1132, wherein the oligonucleotide reduces STAT3 expression and does not reduce STAT1 expression or reduces STAT1 expression less than STAT3 expression. In some embodiments, the oligonucleotide comprises a sense strand comprising the nucleotide sequence set forth in SEQ ID NO: 875 and an antisense strand comprising the nucleotide sequence set forth in SEQ ID NO: 965, wherein the oligonucleotide reduces STAT3 expression and does not reduce STAT1 expression or reduces STAT1 expression less than STAT3 expression. In some embodiments, the oligonucleotide comprises a sense strand comprising the nucleotide sequence set forth in SEQ ID NO: 1055 and an antisense strand comprising the nucleotide sequence set forth in SEQ ID NO: 1145, wherein the oligonucleotide reduces STAT3 expression and does not reduce STAT1 expression or reduces STAT1 expression less than STAT3 expression.

In some embodiments, an oligonucleotide for reducing expression of STAT3 mRNA described herein is a species cross-reactive oligonucleotide. In some embodiments, an oligonucleotide described herein is capable of reducing expression of STAT3 mRNA of at least two different species. In some embodiments, an oligonucleotide described herein is capable of reducing expression of STAT3 mRNA of at least two different species but does not cross-react with non-STAT3 mRNA (e.g., STAT1). In some embodiments, an oligonucleotide for reducing expression of STAT3 mRNA is cross-reactive between at least two species. In some embodiments, an oligonucleotide for reducing expression of STAT3 cross-reacts with human, non-human primate, and mouse STAT3 mRNA. In some embodiments, an oligonucleotide for reducing expression of STAT3 mRNA cross-reacts with human and mouse STAT3 mRNA. In some embodiments, an oligonucleotide for reducing expression of STAT3 mRNA cross-reacts with human and non-human primate STAT3 mRNA.

In some embodiments, an oligonucleotide for reducing expression of STAT3 mRNA reduces STAT3 mRNA by at least 50%, at least 55%, at least 60%, at least 65%, at least 70%, at least 75%, at least 80%, at least 85%, at least 90%, or at least 95%.

In some embodiments, an oligonucleotide for reducing expression of STAT3 mRNA reduces STAT3 mRNA by at least 50% to at least 75% in human, non-human primate, and mouse (i.e. the oligonucleotide is a species cross-reactive oligonucleotide). In some embodiments, an oligonucleotide for reducing expression of STAT3 mRNA reduces STAT3 mRNA by at least 50%, at least 55%, at least 60%, at least 65%, at least 70%, or at least 75% in human, non-human primate, and mouse (i.e. the oligonucleotide is a species cross-reactive oligonucleotide). In some embodiments, an oligonucleotide for reducing expression of STAT3 mRNA reduces STAT3 mRNA by at least 80%, at least 85%, at least 90%, or at least 95% in human, non-human primate, and mouse (i.e. the oligonucleotide is a species cross-reactive oligonucleotide).

In some embodiments, an oligonucleotide for reducing expression of STAT3 mRNA reduces STAT3 mRNA by at least 50% to at least 75% in human and non-human primate (i.e. the oligonucleotide is a species cross-reactive oligonucleotide). In some embodiments, an oligonucleotide for reducing expression of STAT3 mRNA reduces STAT3 mRNA by at least 50%, at least 55%, at least 60%, at least 65%, at least 70%, or at least 75% in human and non-human primate (i.e. the oligonucleotide is a species cross-reactive oligonucleotide). In some embodiments, an oligonucleotide for reducing expression of STAT3 mRNA reduces STAT3 mRNA by at least 80%, at least 85%, at least 90%, or at least 95% in human and non-human primate (i.e. the oligonucleotide is a species cross-reactive oligonucleotide).

In some embodiments, an oligonucleotide for reducing expression of STAT3 mRNA reduces STAT3 mRNA by at least 50% to at least 75% in human and mouse (i.e. the oligonucleotide is a species cross-reactive oligonucleotide). In some embodiments, an oligonucleotide for reducing expression of STAT3 mRNA reduces STAT3 mRNA by at least 50%, at least 55%, at least 60%, at least 65%, at least 70%, or at least 75% in human and mouse (i.e. the oligonucleotide is a species cross-reactive oligonucleotide). In some embodiments, an oligonucleotide for reducing expression of STAT3 mRNA reduces STAT3 mRNA by at least 80%, at least 85%, at least 90%, or at least 95% in human and mouse (i.e. the oligonucleotide is a species cross-reactive oligonucleotide). In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide reduces STAT3 mRNA in humans, non-human primates, and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide reduces STAT3 mRNA in humans, non-human primates, and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide reduces STAT3 mRNA in humans, non-human primates, and mice (i.e. the oligonucleotide is a species cross-reactive oligonucleotide).

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide reduces STAT3 mRNA in humans and non-human primates (i.e. the oligonucleotide is a species cross-reactive oligonucleotide).

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide reduces STAT3 mRNA in humans.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide reduces STAT3 mRNA in humans.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide reduces STAT3 mRNA in humans.

In some embodiments, an oligonucleotide for reducing expression of STAT3 mRNA comprises a sense and antisense strand selected from:
(a) SEQ ID NOs: 857 and 947, respectively;
(b) SEQ ID NOs: 858 and 948, respectively;
(c) SEQ ID NOs: 859 and 949, respectively;
(d) SEQ ID NOs: 860 and 950, respectively;
(e) SEQ ID NOs: 862 and 952, respectively;
(f) SEQ ID NOs: 867 and 957, respectively;
(g) SEQ ID NOs: 875 and 965, respectively; and
(h) SEQ ID NOs: 876 and 966, respectively,
   wherein the oligonucleotide reduces STAT3 mRNA by at least 75%.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide reduces STAT3 mRNA by at least 75%.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide reduces STAT3 mRNA by at least 75%.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide reduces STAT3 mRNA by at least 75%.

In some embodiments, an oligonucleotide for reducing expression of STAT3 mRNA comprises a sense and antisense strand selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively,
   wherein the oligonucleotide reduces STAT3 mRNA by at least 75%.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide reduces STAT3 mRNA by at least 75%.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide reduces STAT3 mRNA by at least 75%.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide reduces STAT3 mRNA by at least 75%.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide reduces STAT3 mRNA by at least 75%.

In some embodiments, an oligonucleotide for reducing expression of STAT3 mRNA comprises a sense and antisense strand selected from:
(a) SEQ ID NOs: 857 and 947, respectively;
(b) SEQ ID NOs: 858 and 948, respectively;
(c) SEQ ID NOs: 859 and 949, respectively;
(d) SEQ ID NOs: 860 and 950, respectively;
(e) SEQ ID NOs: 862 and 952, respectively;
(f) SEQ ID NOs: 867 and 957, respectively;
(g) SEQ ID NOs: 875 and 965, respectively; and
(h) SEQ ID NOs: 876 and 966, respectively,
   wherein the oligonucleotide is conjugated to a lipid.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a lipid on the sense strand.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a lipid on the sense strand.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a lipid on the sense strand.

In some embodiments, an oligonucleotide for reducing expression of STAT3 mRNA comprises a sense and antisense strand selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively,
   wherein the oligonucleotide is conjugated to a lipid on the sense strand.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a lipid on the sense strand.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a lipid on the sense strand.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a lipid on the sense strand.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a lipid on the sense strand.

In some embodiments, an oligonucleotide for reducing expression of STAT3 mRNA comprises a sense and antisense strand selected from:
(a) SEQ ID NOs: 857 and 947, respectively;
(b) SEQ ID NOs: 858 and 948, respectively;
(c) SEQ ID NOs: 859 and 949, respectively;
(d) SEQ ID NOs: 860 and 950, respectively;
(e) SEQ ID NOs: 862 and 952, respectively;
(f) SEQ ID NOs: 867 and 957, respectively;
(g) SEQ ID NOs: 875 and 965, respectively; and
(h) SEQ ID NOs: 876 and 966, respectively,
   wherein the oligonucleotide is conjugated to a C18 lipid on the sense strand.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a C18 lipid on the sense strand.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a C18 lipid on the sense strand.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a C18 lipid on the sense strand.

In some embodiments, an oligonucleotide for reducing expression of STAT3 mRNA comprises a sense and antisense strand selected from:
(a) SEQ ID NOs: 862 and 952, respectively;
(b) SEQ ID NOs: 875 and 965, respectively;
(c) SEQ ID NOs: 876 and 966, respectively; and
(d) SEQ ID NOs: 920 and 1010, respectively,
   wherein the oligonucleotide is conjugated to a C18 lipid on the sense strand.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a C18 lipid on the sense strand.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a C18 lipid on the sense strand.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a C18 lipid on the sense strand.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a C18 lipid on the sense strand.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide reduces STAT3 mRNA in humans, non-human primates, and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide reduces STAT3 mRNA in humans and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide reduces STAT3 mRNA in humans by at least 75%.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide reduces STAT3 mRNA in humans and non-human primates (i.e. the oligonucleotide is a species cross-reactive oligonucleotide) by at least 75%.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide reduces STAT3 mRNA in humans by at least 75%.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide reduces STAT3 mRNA in humans by at least 75%.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide reduces STAT3 mRNA in humans by at least 75%.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a lipid on the sense strand and reduces STAT3 mRNA in humans, non-human primates, and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a lipid on the sense strand and reduces STAT3 mRNA in humans and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a lipid on the sense strand and reduces STAT3 mRNA in humans.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a lipid on the sense strand and reduces STAT3 mRNA in humans and non-human primates (i.e. the oligonucleotide is a species cross-reactive oligonucleotide).

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a lipid on the sense strand and reduces STAT3 mRNA in humans.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a lipid on the sense strand and reduces STAT3 mRNA in humans.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a lipid on the sense strand and reduces STAT3 mRNA in humans.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a C18 lipid on the sense strand and reduces STAT3 mRNA in humans, non-human primates, and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a C18 lipid on the sense strand and reduces STAT3 mRNA in humans and mice (i.e. the oligonucleotide is a species cross-reactive oligonucleotide).

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a C18 lipid on the sense strand and reduces STAT3 mRNA in humans.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a C18 lipid on the sense strand and reduces STAT3 mRNA in humans and non-human primates (i.e. the oligonucleotide is a species cross-reactive oligonucleotide).

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a C18 on the sense strand lipid and reduces STAT3 mRNA in humans.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a C18 lipid on the sense strand and reduces STAT3 mRNA in humans.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a C18 lipid on the sense strand and reduces STAT3 mRNA in humans.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a lipid on the sense strand and reduces STAT3 mRNA in humans, non-human primates, and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a lipid on the sense strand and reduces STAT3 mRNA in humans and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a lipid on the sense strand and reduces STAT3 mRNA in humans by at least 75%.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a lipid on the sense strand and reduces STAT3 mRNA in humans and non-human primates (i.e. the oligonucleotide is a species cross-reactive oligonucleotide) by at least 75%.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a lipid on the sense strand and reduces STAT3 mRNA in humans by at least 75%.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a lipid on the sense strand and reduces STAT3 mRNA in humans by at least 75%.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a lipid on the sense strand and reduces STAT3 mRNA in humans by at least 75%.

In some embodiments, an oligonucleotide for reducing expression of STAT3 mRNA comprises a sense and antisense strand selected from:
(a) SEQ ID NOs: 901 and 991, respectively;
(b) SEQ ID NOs: 910 and 1000, respectively;
(c) SEQ ID NOs: 899 and 989, respectively;
(d) SEQ ID NOs: 896 and 986, respectively;
(e) SEQ ID NOs: 892 and 982, respectively;
(f) SEQ ID NOs: 890 and 980, respectively; and
(g) SEQ ID NOs: 889 and 979, respectively,
   wherein the oligonucleotide is conjugated to a C18 lipid on the sense strand and reduces STAT3 mRNA in humans, non-human primates, and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 940 and 1030, respectively;
(b) SEQ ID NOs: 937 and 1027, respectively; and
(c) SEQ ID NOs: 939 and 1029, respectively,
   wherein the oligonucleotide is conjugated to a C18 lipid on the sense strand and reduces STAT3 mRNA in humans and mice (i.e. the oligonucleotide is a species cross-reactive oligonucleotide) by at least 75%.

In some embodiments, an oligonucleotide for reducing expression of STAT3 mRNA comprises a sense and antisense strand selected from:
(a) SEQ ID NOs: 915 and 1005, respectively;
(b) SEQ ID NOs: 924 and 1014, respectively;
(c) SEQ ID NOs: 913 and 1003, respectively; and
(d) SEQ ID NOs: 920 and 1010, respectively,
   wherein the oligonucleotide is conjugated to a C18 lipid on the sense strand and reduces STAT3 mRNA in humans by at least 75%.

In some embodiments, an oligonucleotide for reducing expression of STAT3 mRNA comprises the sense strand sequence of SEQ ID NO: 862 and the antisense strand sequence of SEQ ID NO: 952, wherein the oligonucleotide is conjugated to a C18 lipid on the sense strand and reduces STAT3 mRNA in humans and non-human primates (i.e. the oligonucleotide is a species cross-reactive oligonucleotide) by at least 75%.

In some embodiments, an oligonucleotide for reducing expression of STAT3 mRNA comprises the sense strand sequence of SEQ ID NO: 875 and the antisense strand sequence of SEQ ID NO: 965, wherein the oligonucleotide is conjugated to a C18 lipid on the sense strand and reduces STAT3 mRNA in humans by at least 75%.

In some embodiments, an oligonucleotide for reducing expression of STAT3 mRNA comprises the sense strand sequence of SEQ ID NO: 876 and the antisense strand sequence of SEQ ID NO: 966, wherein the oligonucleotide is conjugated to a C18 lipid on the sense strand and reduces STAT3 mRNA in humans by at least 75%.

In some embodiments, an oligonucleotide for reducing expression of STAT3 mRNA comprises the sense strand sequence of SEQ ID NO: 920 and the antisense strand sequence of SEQ ID NO: 1010, wherein the oligonucleotide is conjugated to a C18 lipid on the sense strand and reduces STAT3 mRNA in humans by at least 75%.

### Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide is formulated in buffer solutions such as phosphate buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohol's such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several embodiments are directed to liver-targeted delivery of any of the oligonucleotides herein, targeting of other tissues is also contemplated.

### Methods of Use

### Reducing STAT3 Expression in Cells

The disclosure provides methods for contacting or delivering to a cell or population of cells an effective amount any one of oligonucleotides herein for purposes of reducing STAT3 expression. The methods can include the steps described herein, and these maybe be, but not necessarily, carried out in the sequence as described. Other sequences, however, also are conceivable. Moreover, individual, or multiple steps bay be carried out either in parallel and/or overlapping in time and/or individually or in multiply repeated steps. Furthermore, the methods may include additional, unspecified steps.

Methods herein are useful in any appropriate cell type. In some embodiments, a cell is any cell that expresses mRNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue, and skin). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein are delivered using appropriate nucleic acid delivery methods including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

In some embodiments, reduction of STAT3 expression can be determined by an appropriate assay or technique to evaluate one or more properties or characteristics of a cell or population of cells associated with STAT3 expression (e.g., using an STAT3 expression biomarker) or by an assay or technique that evaluates molecules that are directly indicative of STAT3 expression (e.g., STAT3 mRNA or STAT3 protein). In some embodiments, the extent to which an oligonucleotide herein reduces STAT3 expression is evaluated by comparing STAT3 expression in a cell or population of cells contacted with the oligonucleotide to an appropriate control (e.g., an appropriate cell or population of cells not contacted with the oligonucleotide or contacted with a control oligonucleotide). In some embodiments, an appropriate control level of mRNA expression into protein, after delivery of a RNAi molecule may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide herein results in a reduction in STAT3 expression in a cell or population of cells. In some embodiments, the reduction in STAT3 or STAT3 expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower when compared with an appropriate control level of mRNA. The appropriate control level may be a level of mRNA expression and/or protein translation in a cell or population of cells that has not been contacted with an oligonucleotide herein. In some embodiments, the effect of delivery of an oligonucleotide to a cell according to a method herein is assessed after a finite period. For example, levels of mRNA may be analyzed in a cell at least about 8 hours, about 12 hours, about 18 hours, about 24 hours; or at least about 1, 2, 3, 4, 5, 6, 7 or even up to 14 days after introduction of the oligonucleotide into the cell.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

### Medical Use

The disclosure also provides oligonucleotides for use, or adaptable for use, to treat a subject (e.g., a human having a disease, disorder or condition associated with STAT3 expression) that would benefit from reducing STAT3 expression. In some respects, the disclosure provides oligonucleotides for use, or adapted for use, to treat a subject having a disease, disorder or condition associated with expression of STAT3. The disclosure also provides oligonucleotides for use, or adaptable for use, in the manufacture of a medicament or pharmaceutical composition for treating a disease, disorder or condition associated with STAT3 expression. In some embodiments, the oligonucleotides for use, or adaptable for use, target STAT3 mRNA and reduce STAT3 expression (e.g., via the RNAi pathway). In some embodiments, the oligonucleotides for use, or adaptable for use, target STAT3 mRNA and reduce the amount or level of STAT3 mRNA or STAT3 mRNA, STAT3 protein and/or STAT3 activity.

In addition, the methods below can include selecting a subject having a disease, disorder or condition associated with STAT3 expression or is predisposed to the same. In some instances, the methods can include selecting an individual having a marker for a disease associated with STAT3 expression such as cancer or other chronic lymphoproliferative disorders.

Likewise, and as detailed below, the methods also may include steps such as measuring or obtaining a baseline value for a marker of STAT3 expression, and then comparing such obtained value to one or more other baseline values or values obtained after being administered the oligonucleotide to assess the effectiveness of treatment.

### Methods of Treatment

The disclosure also provides methods of treating a subject having, suspected of having, or at risk of developing a disease, disorder, or condition with an oligonucleotide herein. In some aspects, the disclosure provides methods of treating or attenuating the onset or progression of a disease, disorder or condition associated with STAT3 expression using the oligonucleotides herein. In other aspects, the disclosure provides methods to achieve one or more therapeutic benefits in a subject having a disease, disorder or condition associated with STAT3 expression using the oligonucleotides herein. In some embodiments of the methods herein, the subject is treated by administering a therapeutically effective amount of any one or more of the oligonucleotides herein. In some embodiments, treatment comprises reducing STAT3 expression. In some embodiments, the subject is treated therapeutically. In some embodiments, the subject is treated prophylactically.

In some embodiments of the methods herein, one or more oligonucleotides herein, or a pharmaceutical composition comprising one or more oligonucleotides, is administered to a subject having a disease, disorder or condition associated with STAT3 expression such that STAT3 expression is reduced in the subject, thereby treating the subject. In some embodiments, an amount or level of STAT3 mRNA is reduced in the subject. In some embodiments, an amount or level of STAT3 and/or protein is reduced in the subject

In some embodiments of the methods herein, an oligonucleotide herein, or a pharmaceutical composition comprising the oligonucleotide, is administered to a subject having a disease, disorder or condition associated with STAT3 such that STAT3 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to STAT3 expression prior to administration of one or more oligonucleotides or pharmaceutical composition. In some embodiments, STAT3 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to STAT3 expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with STAT3 expression such that an amount or level of STAT3 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of STAT3 mRNA prior to administration of the oligonucleotide or pharmaceutical composition. In some embodiments, an amount or level of STAT3 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of STAT3 mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with STAT3 expression such that an amount or level of STAT3 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of STAT3 protein prior to administration of the oligonucleotide or pharmaceutical composition. In some embodiments, an amount or level of STAT3 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of STAT3 protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with STAT3 such that an amount or level of STAT3 activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of STAT3 activity prior to administration of the oligonucleotide or pharmaceutical composition. In some embodiments, an amount or level of STAT3 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of STAT3 activity in a subject (e.g., a reference or control subject) not receiving the oligonucleotide or pharmaceutical composition or receiving a control oligonucleotide, pharmaceutical composition or treatment.

Because of their high specificity, the oligonucleotides herein specifically target mRNAs of target genes of diseased cells and tissues. In preventing disease, the target gene may be one which is required for initiation or maintenance of the disease or which has been identified as being associated with a higher risk of contracting the disease. In treating disease, the oligonucleotide can be brought into contact with the cells or tissue exhibiting the disease. For example, an oligonucleotide substantially identical to all or part of a wild-type (i.e., native) or mutated gene associated with a disorder or condition associated with STAT3 expression may be brought into contact with or introduced into a cell or tissue type of interest such as a hepatocyte or other liver cell.

In some embodiments, the target gene may be a target gene from any mammal, such as a human target. Any gene may be silenced according to the method described herein.

Methods described herein are typically involve administering to a subject in an effective amount of an oligonucleotide or oligonucleotides, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

As a non-limiting set of examples, the oligonucleotides herein would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments the oligonucleotides herein are administered alone or in combination. In some embodiments the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Combination Treatment

In some embodiments, the oligonucleotides described herein are used in combination with at least one additional composition or therapeutic agent. In some aspects, the composition or therapeutic agent is selected from the group consisting of: a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, or a combination thereof. In some embodiments, the composition or therapeutic agent targets TGFB, CXCR2, CCR2, ARG1, PTGS2, SOCS1 or PD-L1. In some embodiments, the composition or therapeutic agent targets TGFB. In some embodiments, the composition or therapeutic agent targets CXCR2. In some embodiments, the composition or therapeutic agent targets CCR2. In some embodiments, the composition or therapeutic agent targets ARG1. In some embodiments, the composition or therapeutic agent targets PTGS2. In some embodiments, the composition or therapeutic agent targets SOCS1. In some embodiments, the composition or therapeutic agent targets PD-L1. In some embodiments, the composition or therapeutic agent that targets any of the above targets, is an oligonucleotide (e.g., dsRNAi). In some embodiments, the composition or therapeutic agent that targets any of the above targets, is an antibody or antigen-binding fragment thereof.

### Kits

In some embodiments, the disclosure provides a kit comprising an oligonucleotide herein, and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings. In some embodiments, a kit comprises an oligonucleotide herein, and a pharmaceutically acceptable carrier, or a pharmaceutical composition comprising the oligonucleotide and instructions for treating or delaying progression of a disease, disorder or condition associated with STAT3 expression in a subject in need thereof. In some embodiments, a kit comprises an oligonucleotide herein, and a pharmaceutically acceptable carrier, or a pharmaceutical composition comprising the oligonucleotide and instructions for treating or delaying progression of a cancer in a subject in need thereof.

### EXAMPLES

While the disclosure has been described with reference to the specific embodiments set forth in the following Examples, it should be understood by those skilled in the art that various changes may be made, and equivalents may be substituted without departing from the true spirit and scope of the disclosure. Further, the following Examples are offered by way of illustration and are not intended to limit the scope of the disclosure in any manner. In addition, modifications may be made to adapt to a situation, material, composition of matter, process, process step or steps, to the objective, spirit, and scope of the disclosure. All such modifications are intended to be within the scope of the disclosure. Standard techniques well known in the art or the techniques specifically described below were utilized.

The following examples describe the development of lipid conjugate siRNA delivery mechanism to deliver an RNAi payload to myeloid-derived suppressor cells (MDSCs) to silence genes that mediate immune suppression. Initially a surrogate ALDH2-GalXC lipid conjugate was used to deliver payload to both subtypes of MDSCs in the tumor microenvironment (TME), as well as the MDSCs found in tumor draining lymph nodes (TdLN) to silence ALDH2. Later, a STAT3-GalXC lipid conjugate was constructed to target and silence the STAT3 gene in MDSCs. Targeting STAT3 is considered a promising approach since it is a main transcription factor associated with immunosuppressive activity in myeloid cells. STAT3 activation is known to play an important role in promoting tolerogenic effects in TME. Although STAT3 is expressed by tumor cells, the approach to target the STAT3 signaling in tumor associated myeloid cells in TME and TdLN, without affecting STAT3 signaling in cancer cells, was previously demonstrated to be sufficient to inhibit the tolerogenic effects and induce anti-tumor immunity and inhibit tumor growth of various solid tumors. (Kortylewski et al, NAT MED 2005). As a proof-of-concept target, we demonstrated STAT3 knockdown in both MDSCs in the TME and TdLN. These data suggest that a GalXC-STAT3-lipid conjugate or another target-conjugate combination tailored to an MDSC or TdLN specific target has a potential to sensitize treatment-refractory tumors to immune checkpoint blockade.

In order that the disclosure provided herein may be more fully understood, the following examples are set forth. The examples described in this application are offered to illustrate the methods, compositions, and systems provided herein and are not to be construed in any way as limiting their scope.

### Abbreviations

Ac: acetyl

AcOH: acetic acid
ACN: acetonitrile
Ad: adamantyl
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
BuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium
hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
L-DBTA: dibenzoyl-L-tartaric acid
m-CPBA: meta-chloroperbenzoic acid
M: molar
MeCN: acetonitrile
MeOH: methanol
Me₂S: dimethyl sulfide
MeONa: sodium methylate
Met iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
s or sec: second
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAF: tetrabutylammmonium fluoride
TBAI: tetrabutylammonium iodide
TEA: triethylamine
Tf: trifluoromethanesulfonate
TfAA, TFMSA or Tf₂O: trifluoromethanesulfonic anhydride
TFA: trifluoroacetic acid
TIBSCl: 2,4,6-triisopropylbenzenesulfonyl chloride
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
UPLC: Ultra Performance Liquid Chromatography
wt: weight
Xantphos: 4,5-bis(diphenylphosphino)-9,9-dimethylxanthene

### Example 1: Preparation of Double-Stranded RNAi Oligonucleotides

### General Synthetic Methods

The following examples are intended to illustrate the disclosure and are not to be construed as being limitations thereon. Temperatures are given in degrees centigrade (C). If not mentioned otherwise, all evaporations are performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the nucleic acid or analogues thereof of the present disclosure are either commercially available or can be produced by organic synthesis methods known to one of ordinary skill in the art (METHODS OF ORGANIC SYNTHESIS, Thieme, Volume 21 (Houben-Weyl 4th Ed. 1952)). Further, the nucleic acid or analogues thereof of the present disclosure can be produced by organic synthesis methods known to one of ordinary skill in the art as shown in the following examples.

All reactions are carried out under nitrogen or argon unless otherwise stated.

Proton NMR (¹H NMR) was conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleic acid or analogues thereof were prepared according to the following general procedures. It will be appreciated that, although the general methods depict the synthesis of certain nucleic acid or analogues thereof of the present disclosure, the following general methods, and other methods known to one of ordinary skill in the art, can be applied to all nucleic acid or analogues thereof and subclasses and species of each of these nucleic acid or analogues thereof, as described herein.

### Example 1a: Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

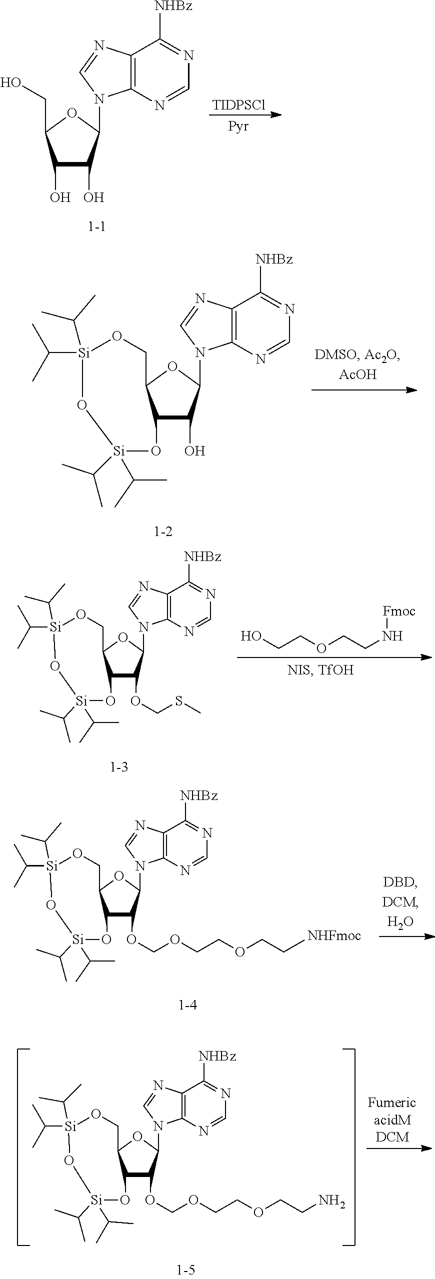

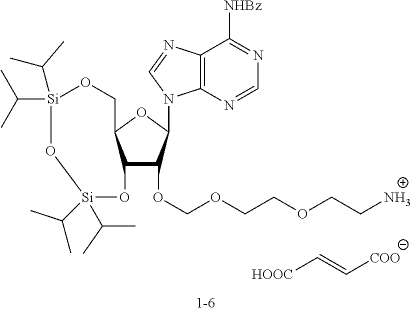

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Example 1b: Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

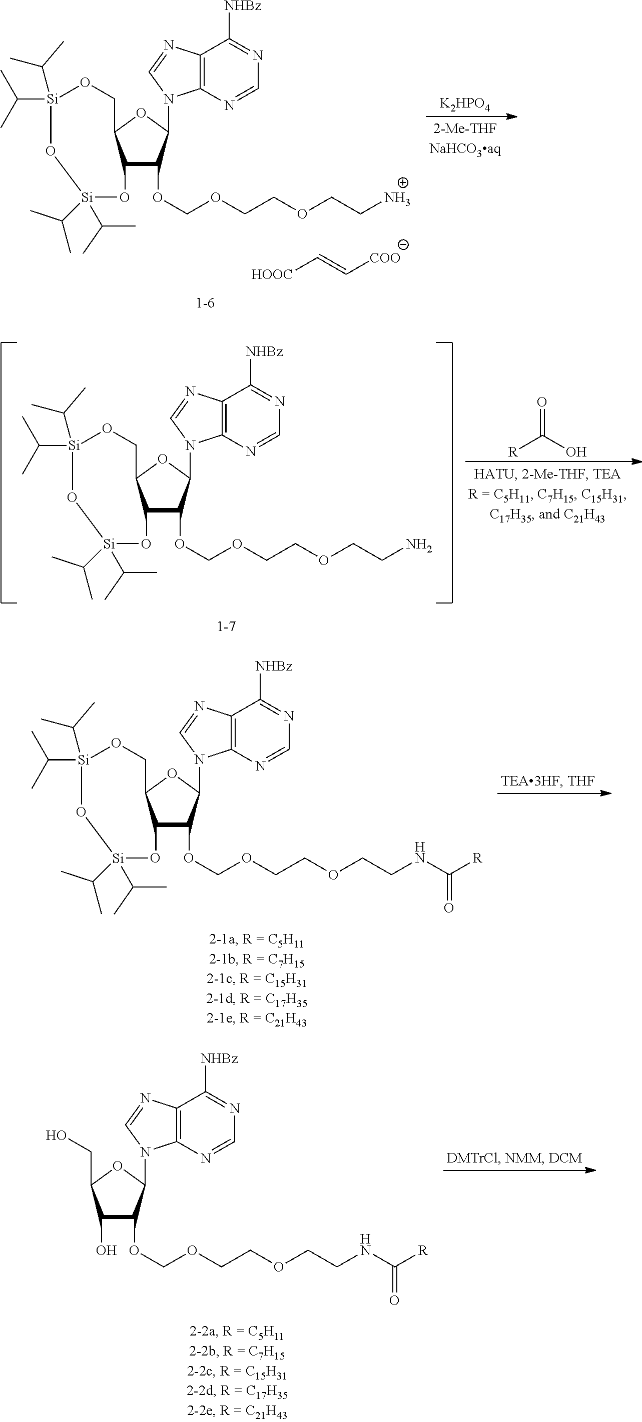

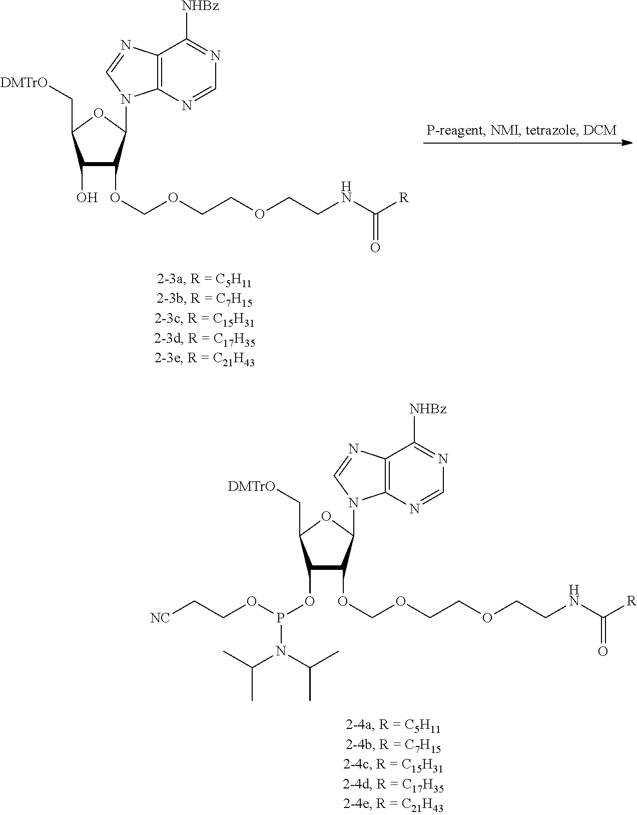

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

### Example 2. Synthesis of GalXC RNAi Oligonucleotide-Lipid Conjugates

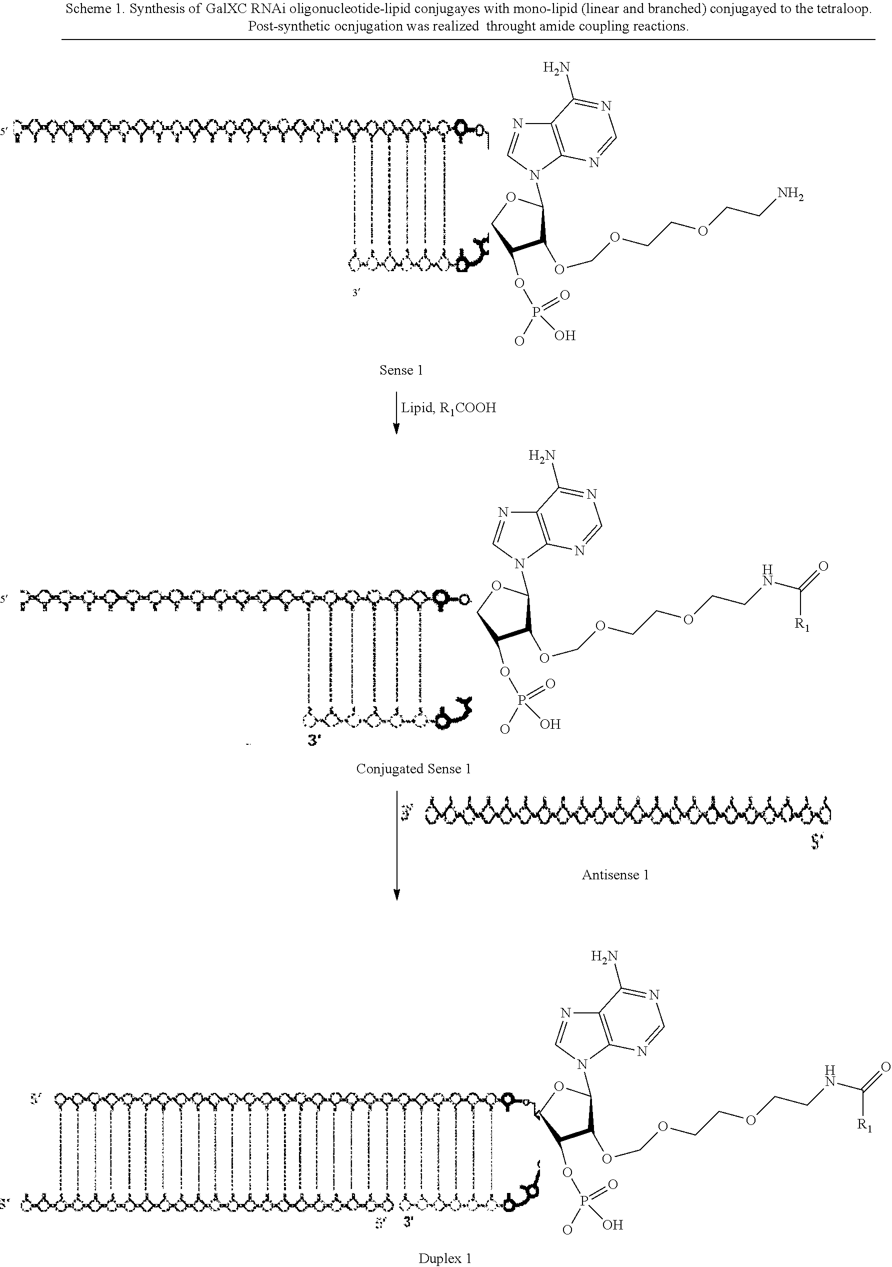

R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1

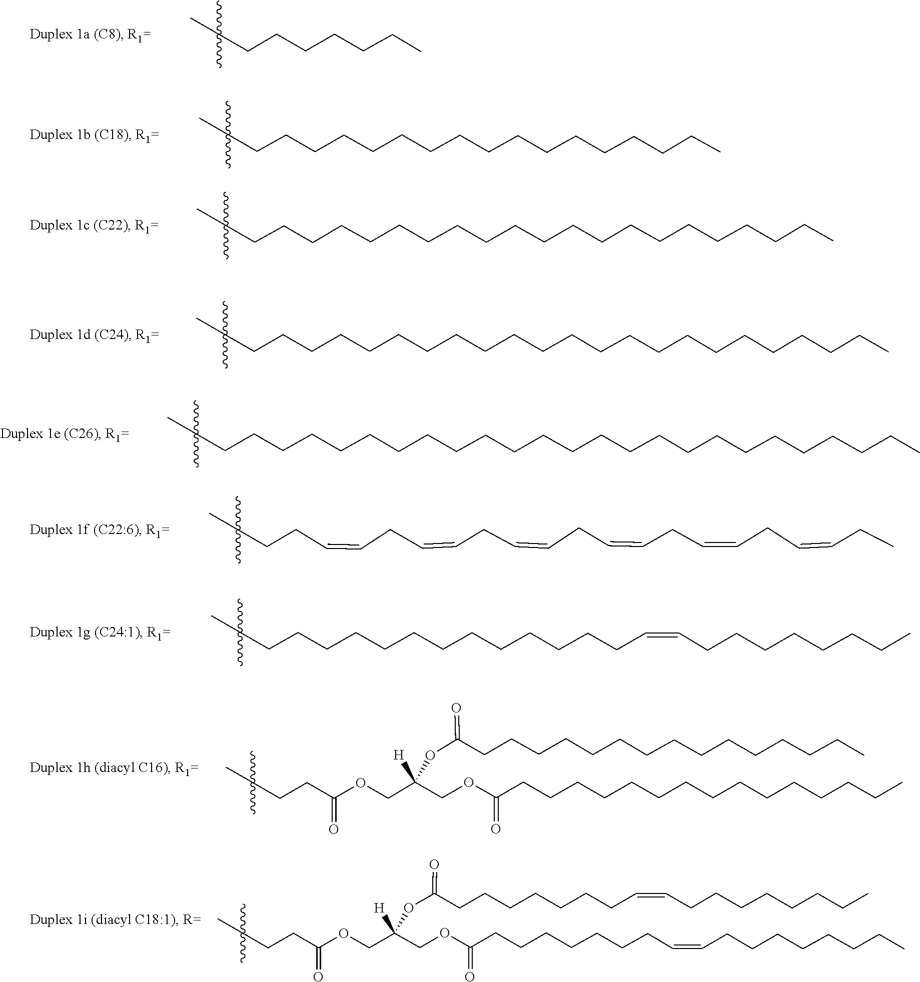

Synthesis Sense 1 and Antisense 1 were prepared by solid-phase synthesis.

Synthesis of Conjugated Sense 1a-1i.

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using Thermomixer at rt. After the reaction was completed indicated by LC-MS analysis, the reaction mixture was diluted with 5 mL of water and purified by revers phase)(Bridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

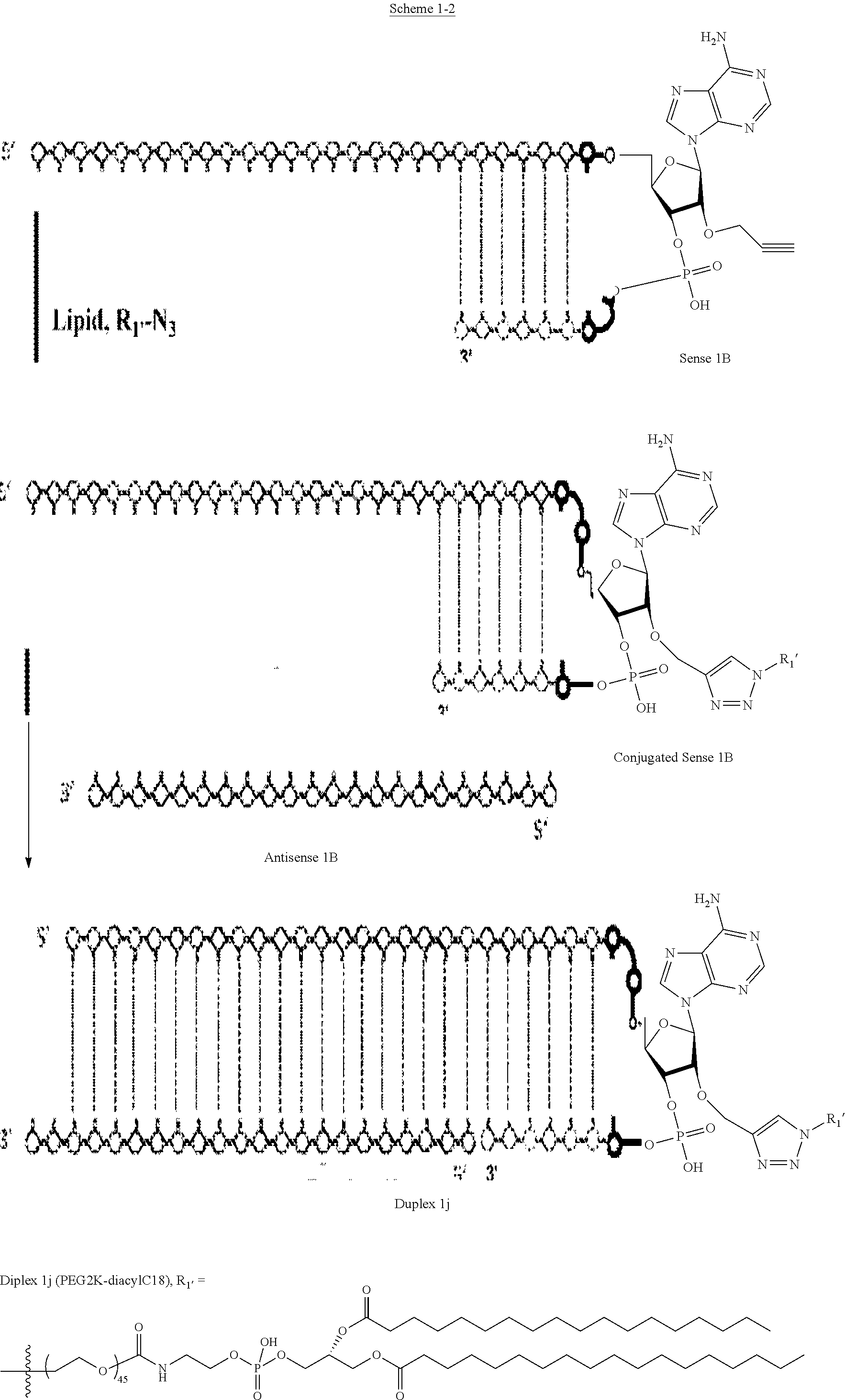

Sense 1B and Antisense 1B were prepared by solid-phase synthesis. Synthesis of Conjugated Sense 1j.

In Eppendorf tube 1, a solution of oligo (10.00 mg, 0.8 umol) in a 3:1 mixture of DMA/H₂O (0.5 mL) was treated with the lipid linker azide (11.26 mg, 4 umol). In Eppendorf tube 2, CuBr dimethyl sulfide (1.64 mg, 8 umol) was dissolved in ACN (0.5 mL). Both solutions were degassed for 10 min by bubbling N₂ through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase)(Bridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

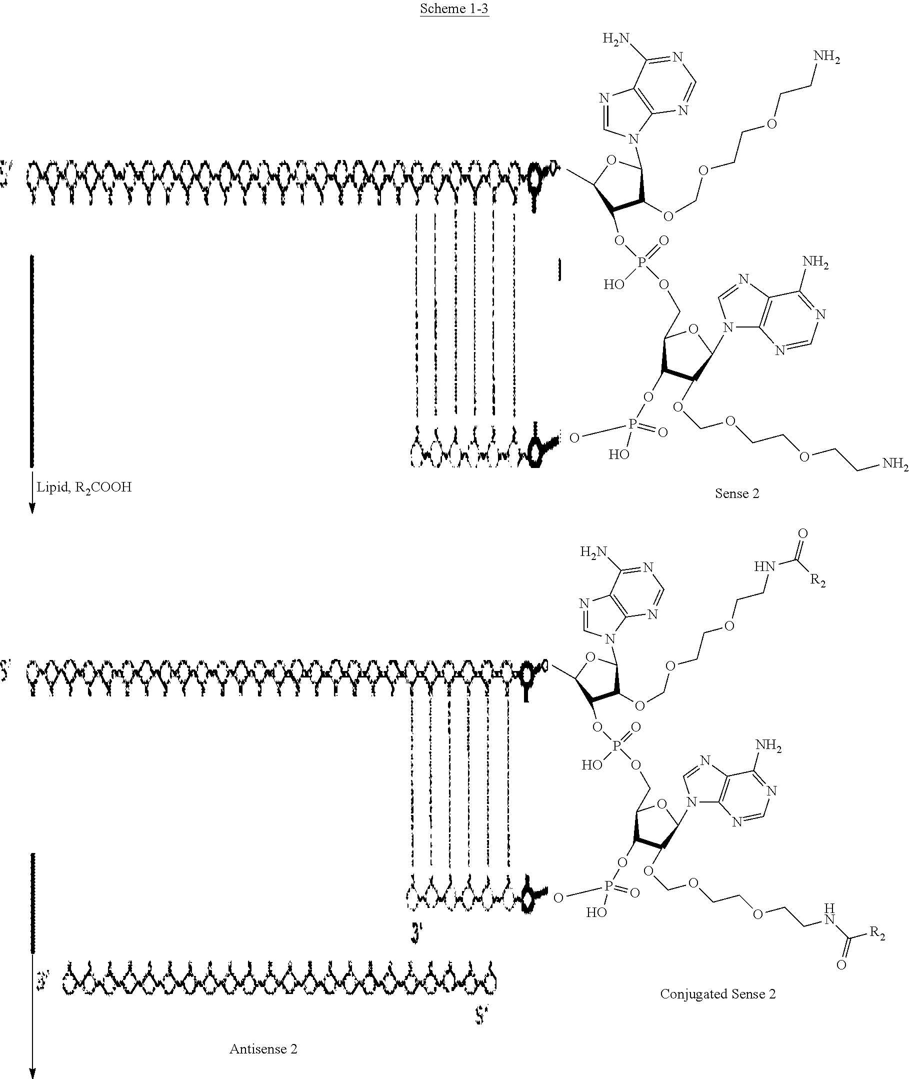

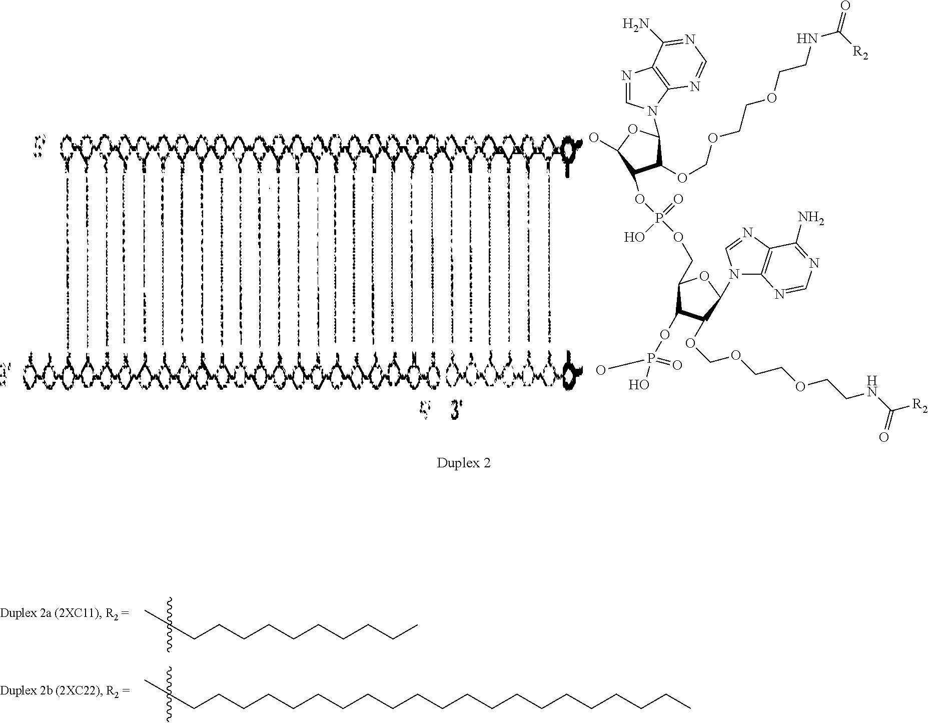

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

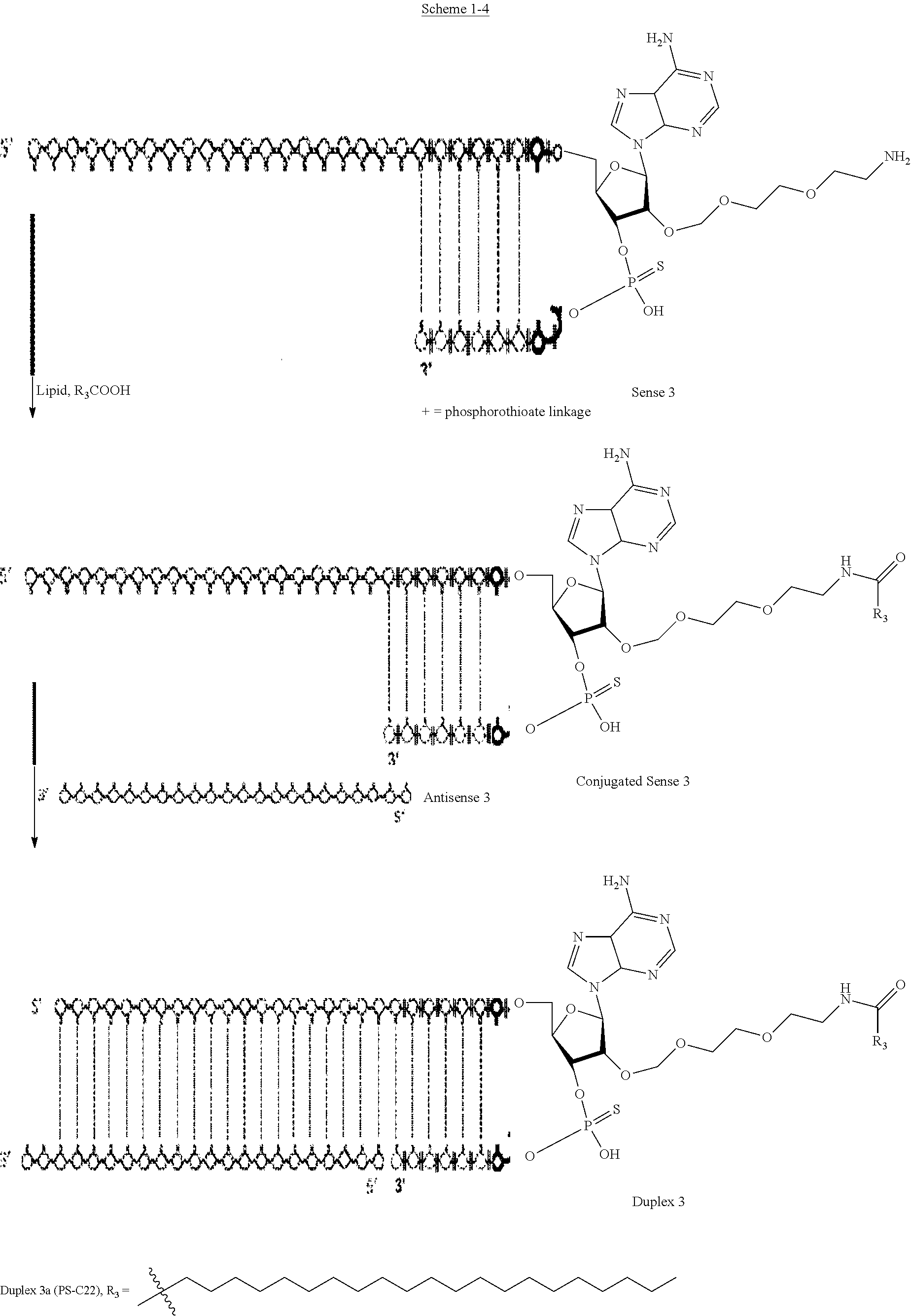

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS-C22) was prepared using the same procedures as described for the annealing of Duplex 1a (C8).

The following Scheme 1-5 depicts the synthesis of GalXC of short sense conjugated with mono-lipid using post-synthetic conjugation approach.

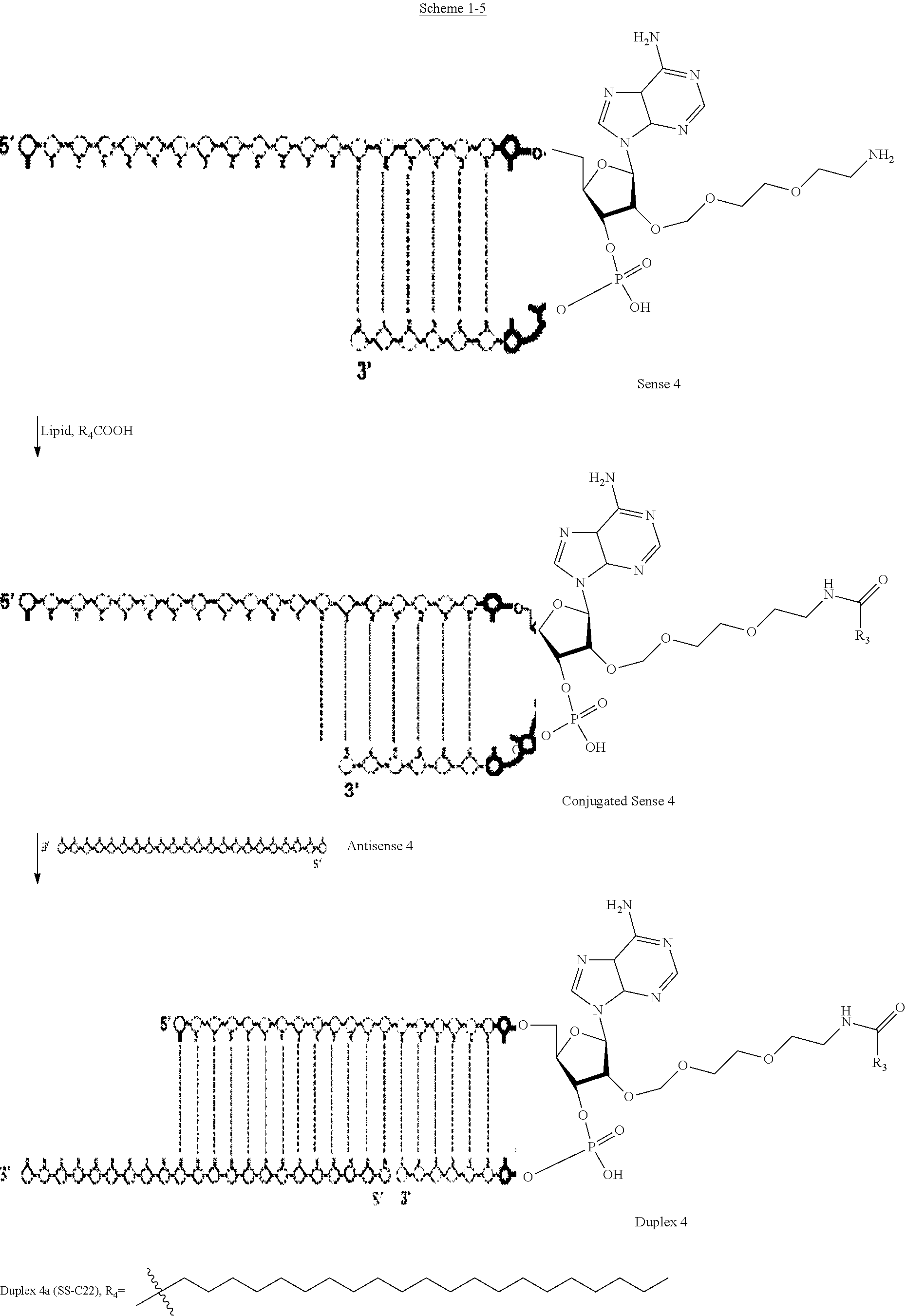

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS-C22) was prepared using the same procedures as described for the annealing of Duplex 1a (C8).

The following Scheme 1-6 depicts the synthesis of Nicked tetraloop GalXC conjugated with tri-adamantane moiety on the loop using post-synthetic conjugation approach.

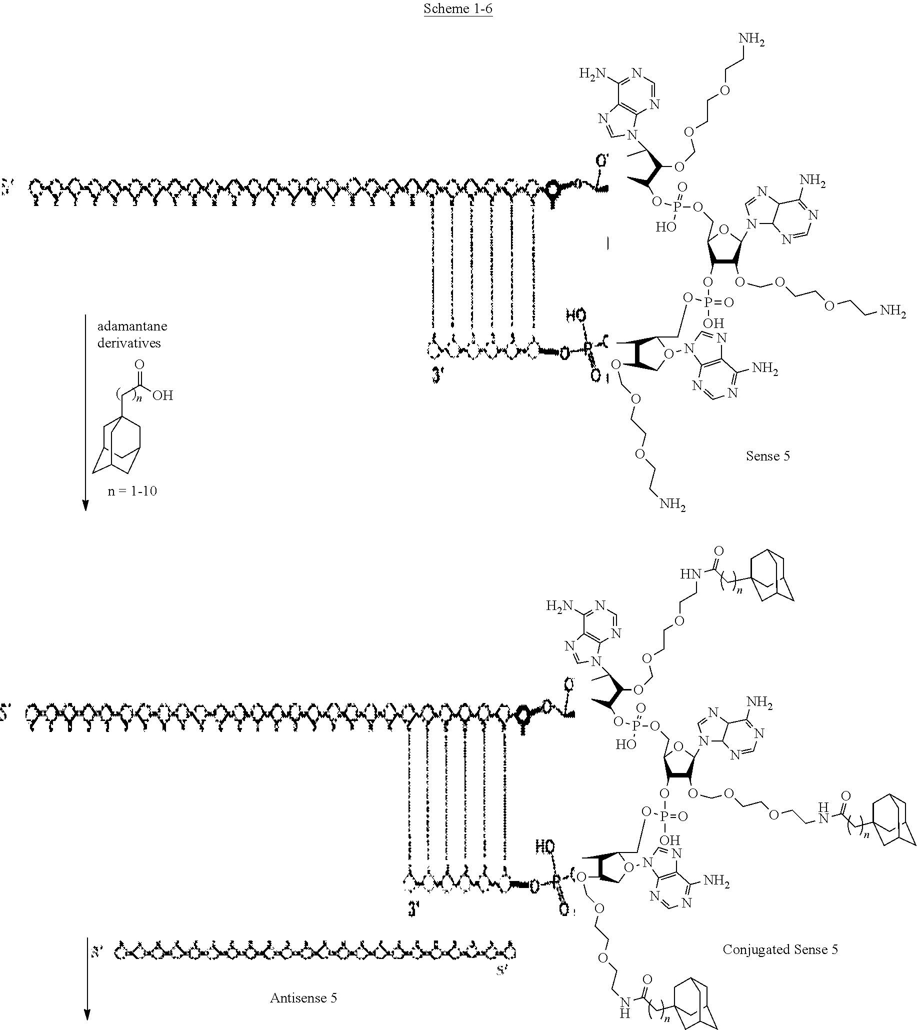

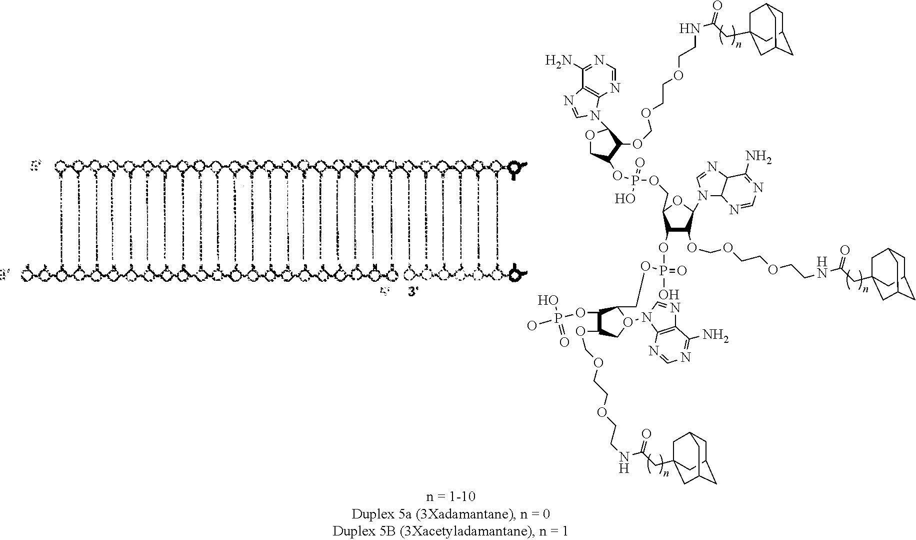

Sense 5 and Antisense 5 were prepared by solid-phase synthesis.

Conjugated Sense 5a and 5b were prepared using similar procedures as described for the synthesis of Conjugated Sense 1a and obtained in 42%-73% yields.

Duplex 5a (3Xadamantane) and Duplex 5b (3Xacetyladamantane) were prepared using the same procedures as described for the annealing of Duplex 1a (C8).

The following scheme 1-7 depicts an example of solid phase synthesis of Nicked tetraloop GalXC conjugated with lipid(s) on the loop.

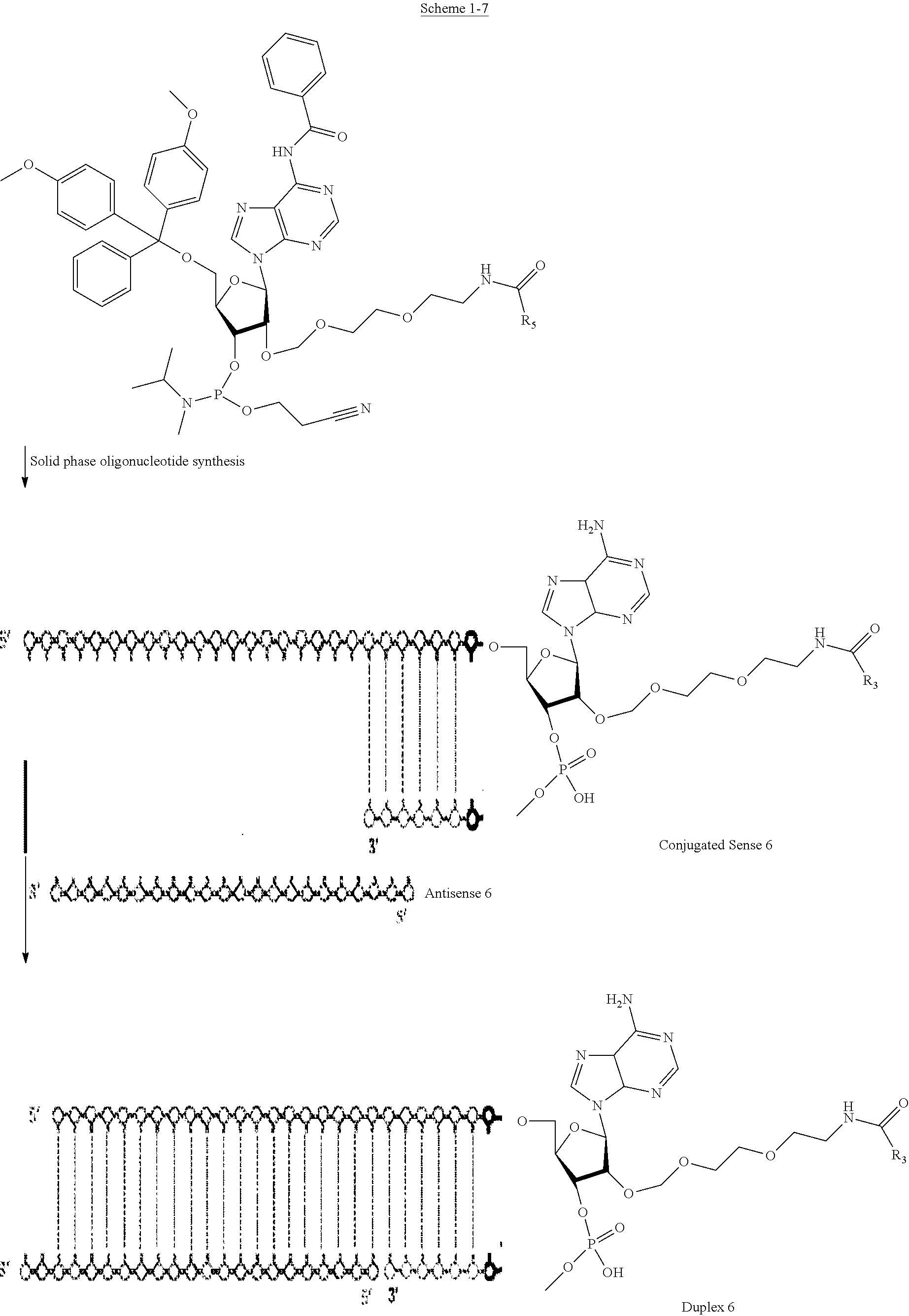

Synthesis of Conjugated Sense 6.

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′ direction using a standard oligonucleotide synthesis protocol. In these efforts, 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed, and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

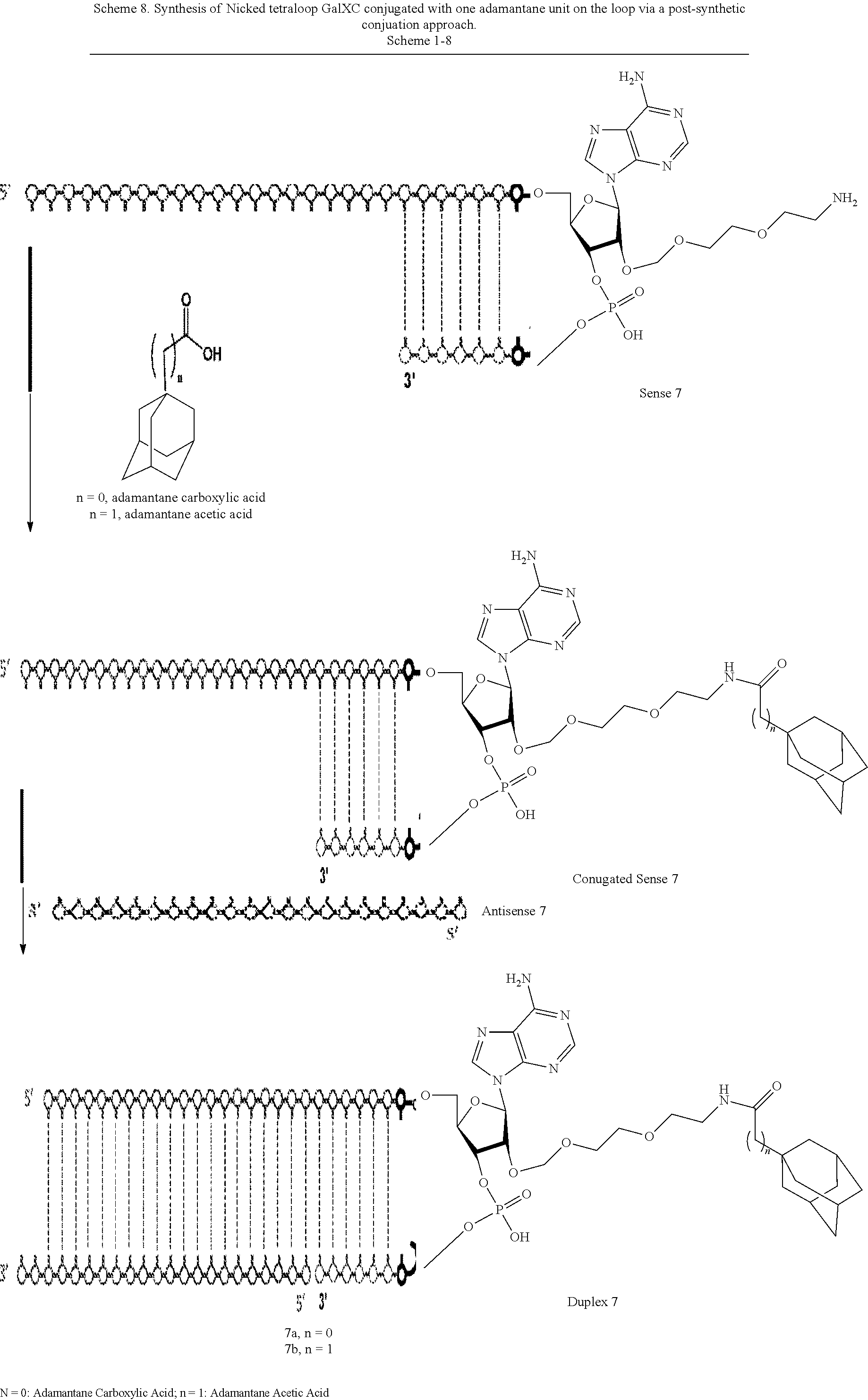

### Synthesis of Conjugated Sense 7a and 7b

Conjugated Sense 7a and Sense 7b were obtained using the same method or a substantially similar method to the synthesis of Conjugated Sense 5.

### Synthesis Example of Duplex 7a and 7b

Duplex 7a and Duplex 7b were obtained using the same method or a substantially similar method to the synthesis of Duplex 5.

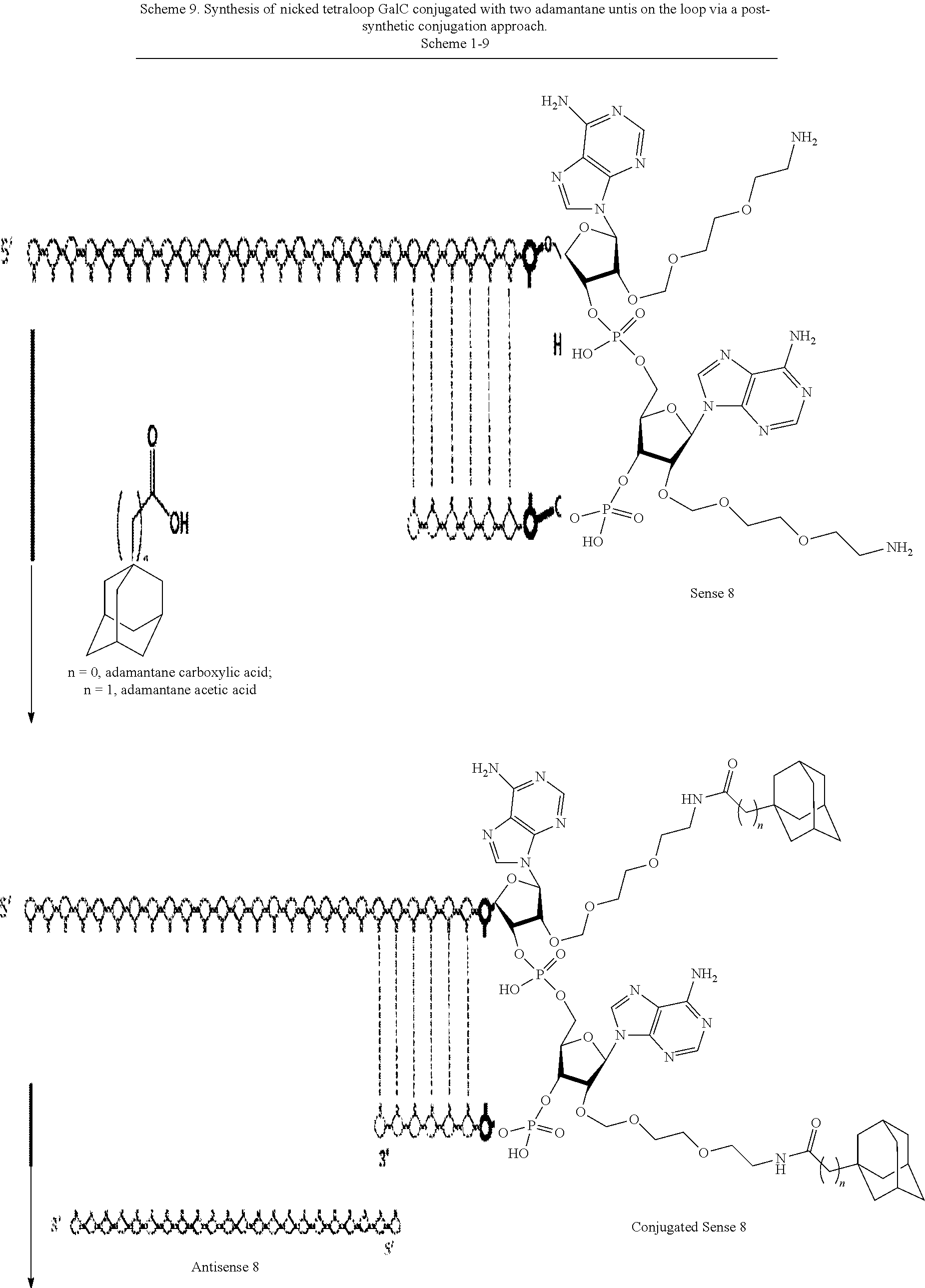

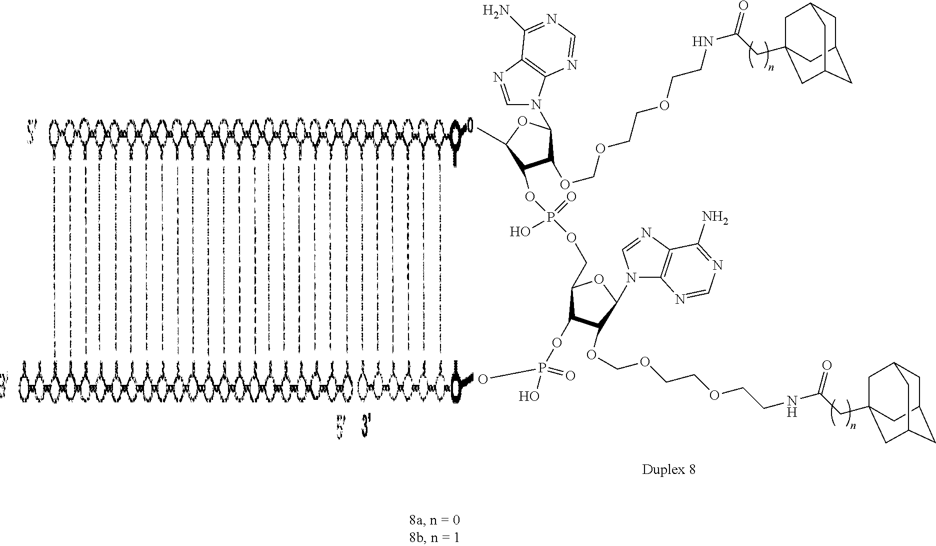

### Synthesis of Conjugated Sense 8a and 8b

Conjugated Sense 8a and Sense 8b were obtained using the same method or a substantially similar method to the synthesis of Conjugated Sense 5.

### Synthesis Example of Duplex 8a and 8b

Duplex 8a and Duplex 8b were obtained using the same method or a substantially similar method to the synthesis of Duplex 5.

The following Scheme 1-10 depicts the synthesis of GalXC of short sense and short stem loop conjugated with mono-lipid using post-synthetic conjugation approach.

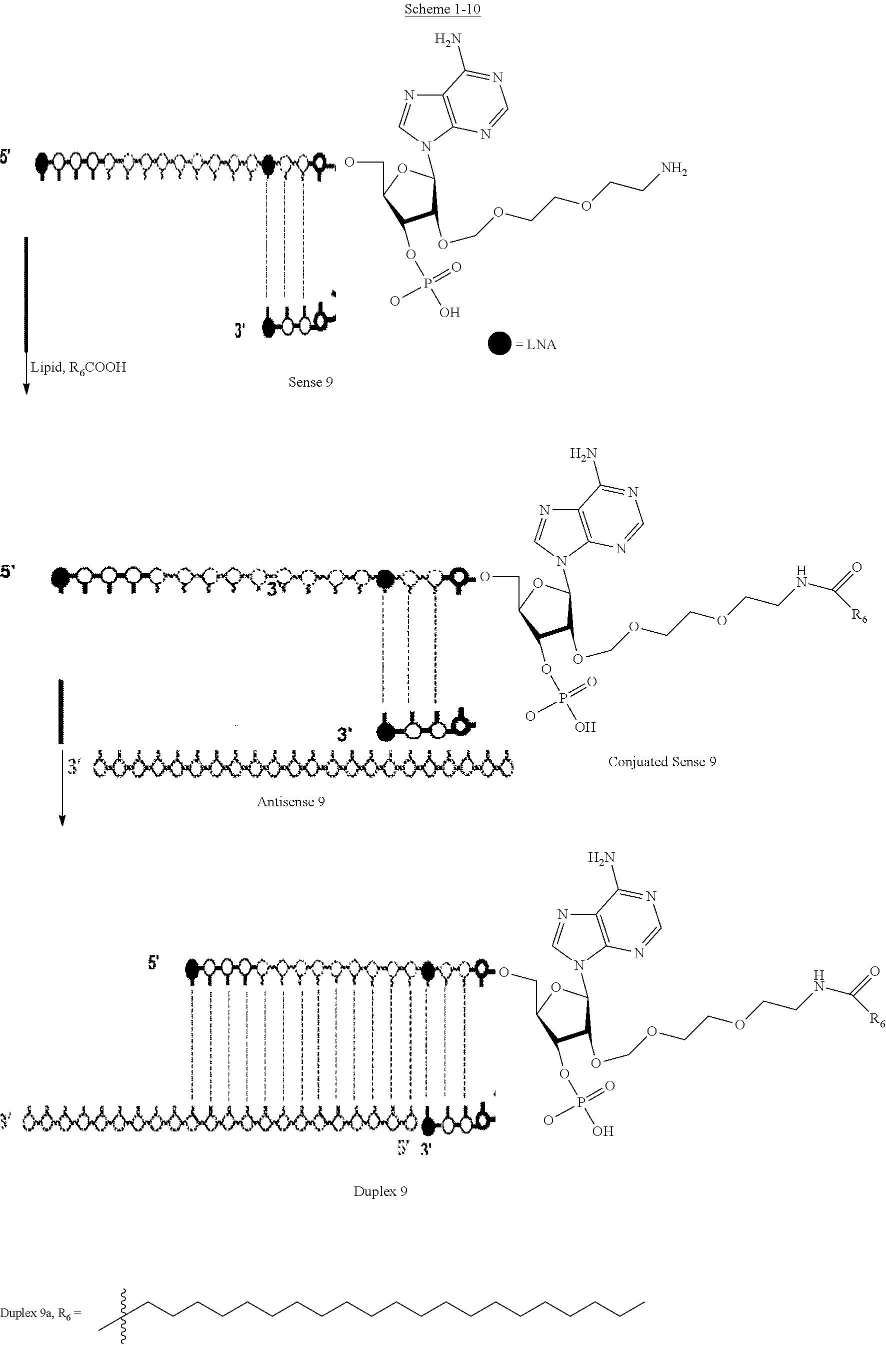

### Synthesis of Sense 9a

Conjugated Sense 9a was obtained using the same method or a substantially similar method to the synthesis of Conjugated Sense 5.

Synthesis example of Duplex 9a

Duplex 9a was obtained using the same method or a substantially similar method to the synthesis of Duplex 5.

The following Scheme 1-11 depicts the synthesis of GalXC conjugated with mono-lipid at 5′-end using post-synthetic conjugation approach.

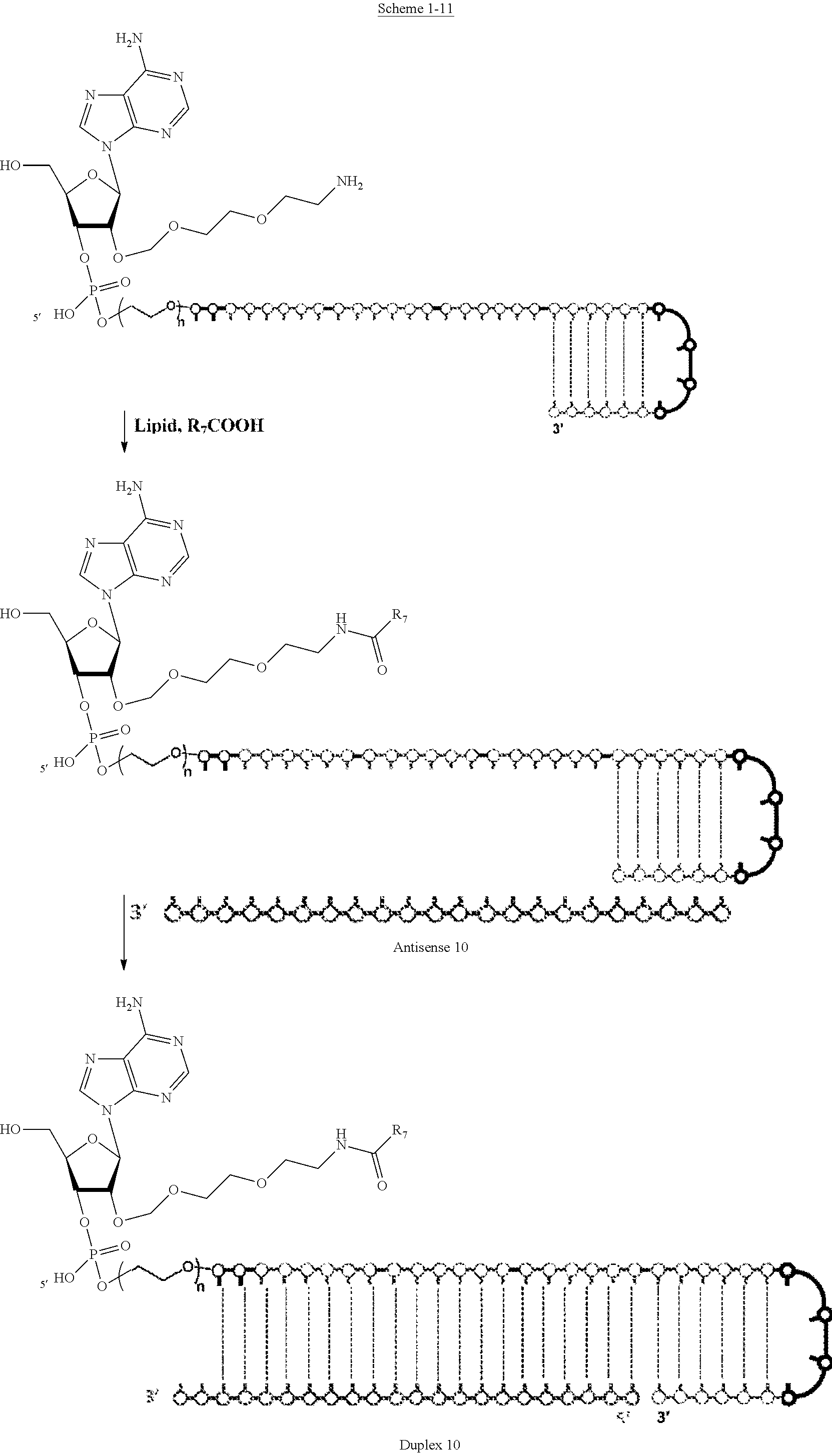

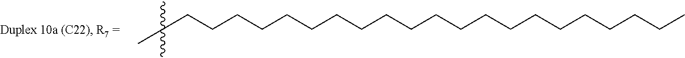 [SMILES: *CCCCCCCCCCCCCCCCCCCCC]

### Synthesis of Conjugated Sense 10a

Conjugated Sense 10a was obtained using the same method or a substantially similar method to the synthesis of Conjugated Sense 5.

### Synthesis Example of Duplex 10a

Duplex 10a was obtained using the same method or a substantially similar method to the synthesis of Duplex 5.

The following Scheme 1-12a and 1-12b depict the synthesis of GalXC with blunt end conjugated with mono-lipid at 3′-end or 5′-end using post-synthetic conjugation approach.

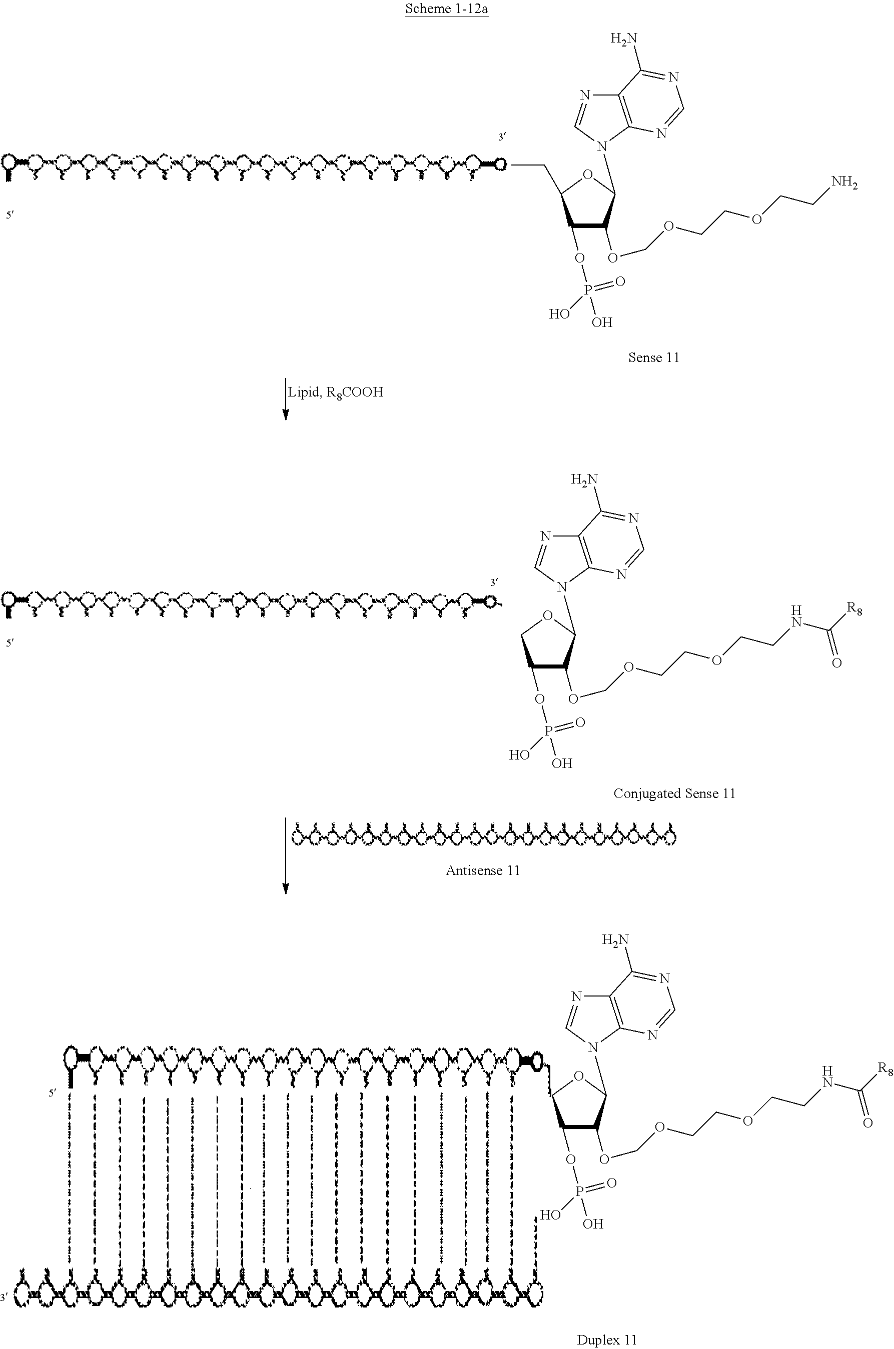

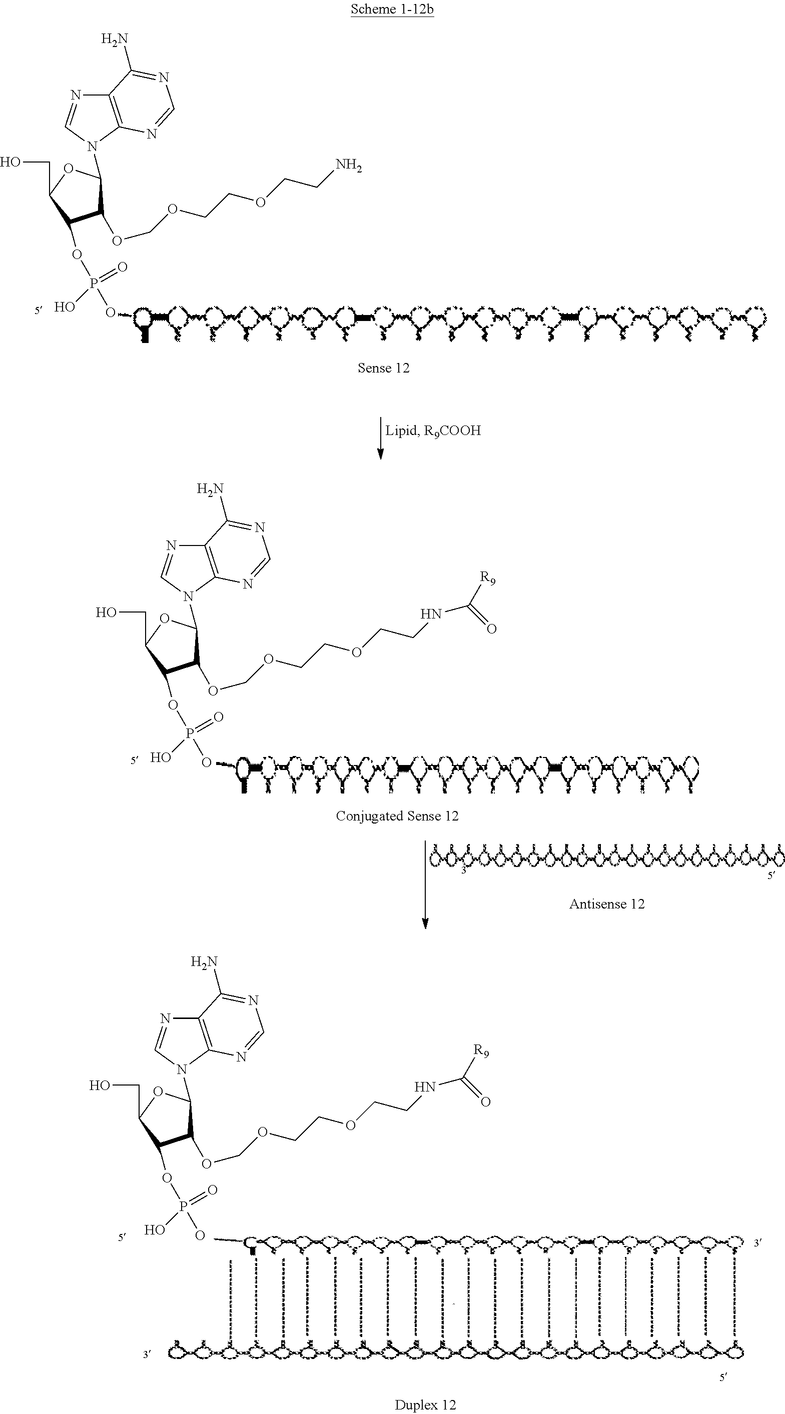

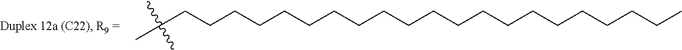 [SMILES: *CCCCCCCCCCCCCCCCCCCCC]

### Synthesis of Conjugated Sense 11a and 12a

Conjugated Sense 11a and 12a were obtained using the same method or a substantially similar method to the synthesis of Conjugated Sense 5.

### Synthesis Example of Duplex 11a and 12a

Duplex 11a and 12a were obtained using the same method or a substantially similar method to the synthesis of Duplex 5.

Conjugates Duplex 8D and Duplex 9D were obtained using the same method or a substantially similar method to the synthesis of Duplex 5.

Later, acyl chains were conjugated to a nucleic acid inhibitor molecule that targets the STAT3 gene, a gene that is expressed in the tissues of interest. A passenger strand with 2′-amine linkers [ademA] was used for post solid phase conjugation. Different types of lipids were conjugated using the same chemistry to generate a series of conjugates (FIGS. 1A and 1B). SAR studies were performed to identify a lipid conjugate that could be used to deliver payloads to the tissues of interest in order to mediate target knockdown.

### Example 3: In Vivo Tumor Models

Briefly, 6-8-week-old immunocompromised (Nude)/Immunocompetent (C57BL/6) mice were injected subcutaneously with 2×10⁶ Pan02 cells (mouse pancreatic cancer cell line), 2×10⁶ B16F10 cells (mouse melanoma cell line) or 5×10⁶ LS411N cells (human colorectal cancer cell line) under the right shoulder. When the tumors reached a volume of 300-500 mm³, they were randomized into different cohorts and subjected to dosing with GalXC lipid conjugates. Each GalXC lipid conjugate was dosed subcutaneously at a total volume of 10 mL/kg. Mouse pancreatic cell line Pan02 was obtained from NCI and mouse melanoma cell line Bl6F10 and human colorectal cell line LS411N were obtained from ATCC (Manassas, VA). All cells were grown in RPMI/DMEM medium supplemented with 10% FBS. Pan02, Bl6F10 and LS411N tumors are known to maintain very suppressive, or cold, tumor microenvironments.

### Example 4: Differential Delivery of GalXC Lipid Conjugates to Different Components of the Tumor Microenvironment

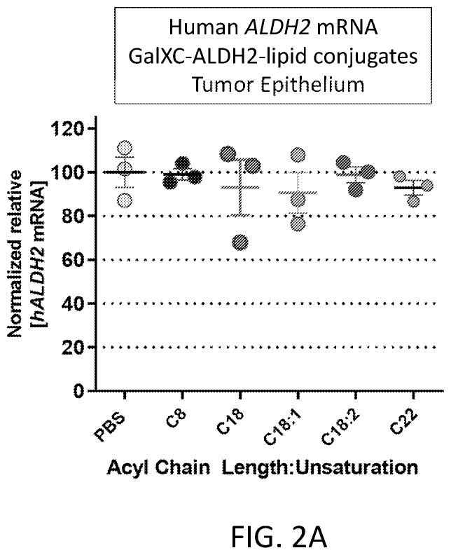

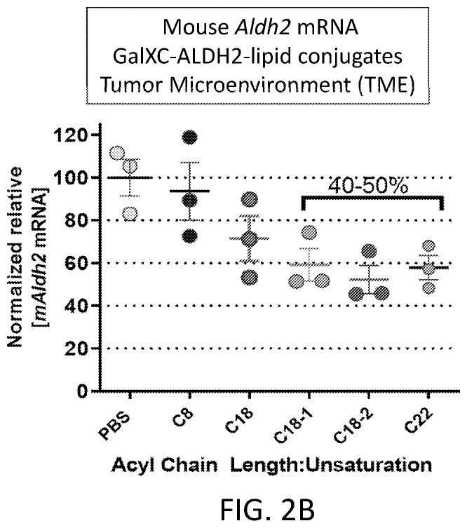

To elucidate differential delivery of GalXC lipid conjugates, human xenograft tumors (LS411N cells) were implanted in nude mice, as described in Example 3. At about two weeks post implant, when tumor volume reached ˜300-400 mm³, mice were randomized into 6 groups (n=3) and treated with a single dose of either Phosphate Buffered Saline (PBS) or an GalXC-ALDH2-lipid conjugate as outlined in Scheme 1 of Example 2 (GalXC-C8, GalXC-C18, GalXC-C18-1, GalXC-C18-2 or GalXC-C22) at 10 mg/kg. Three days post subcutaneous injection, tumors were collected and analyzed by qPCR to determine mRNA levels of human ALDH2 and mouse Aldh2. In bulk tumor tissue, mRNA expression levels of the human ALDH2 gene remained at baseline across all groups, however mouse Aldh2 mRNA levels were decreased by ˜40-50% across all groups treated with GalXC-ALDH2-lipid conjugates, including C18, C18-1, C18-2 and C22, except C8 as compared to the PBS control (FIGS. 2A and 2B). These data suggest that the GalXC-ALDH2-lipid conjugates did not mediate siRNA delivery and target knockdown in human tumor epithelium, but mediated siRNA delivery to components in tumor microenvironment in order to facilitate target knockdown. To further confirm this observation, a follow-up study was run in the same tumor type. LS411N human xenograft tumors were implanted in nude mice, as described above. After randomization into 12 groups, GalXC-ALDH2-C22 conjugate at 10, 25 and 50 mg/kg and PBS control, mice were treated with a single subcutaneous dose of test article accordingly.

TABLE 2
GalXC-lipid conjugate ALDH2 Tool Molecules

| | | | Sense | Antisense | |
|---|---|---|---|---|---|
| | | | Strand | strand | |
| | | Sequence | SEQ | SEQ | |
| Oligo | DP # | Type | ID NO | ID NO | Conjugate |
| GalXC- | DP15543P: | Unmodified | 1 | 2 | C18 |
| ALDH2-C18 | DP11674G | Modified | 3 | 4 | C18 |
| GalXC- | DP15545P: | Unmodified | 5 | 6 | C22 |
| ALDH2-C22 | DP11674G | Modified | 7 | 8 | C22 |

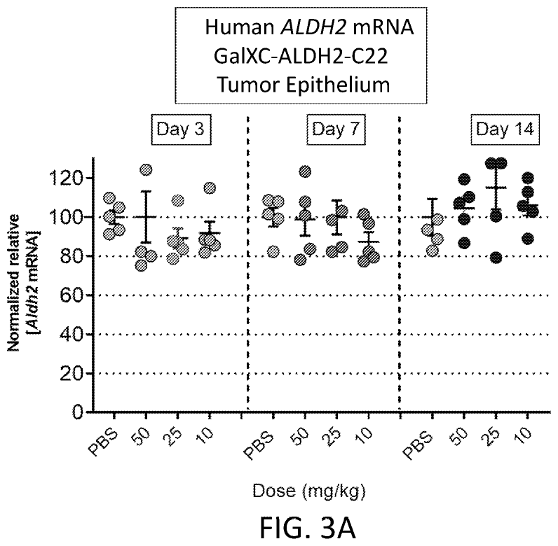

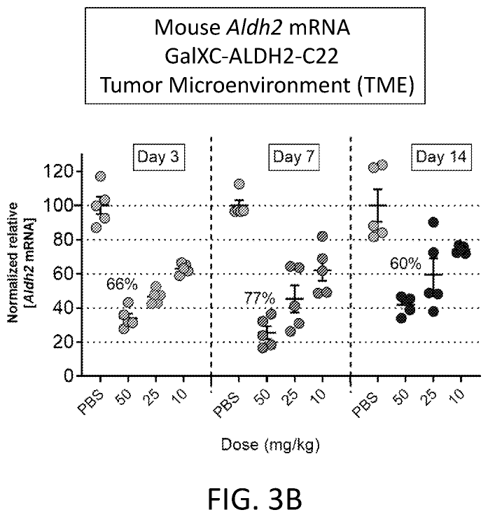

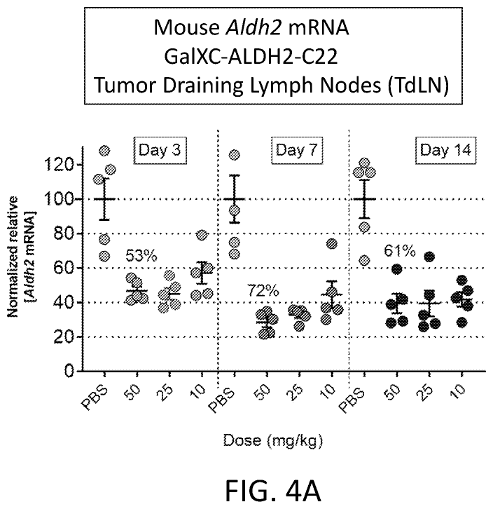

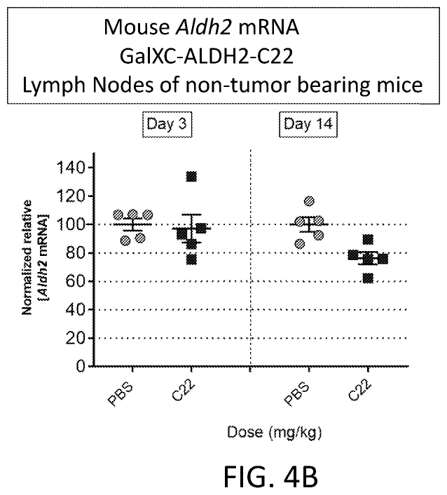

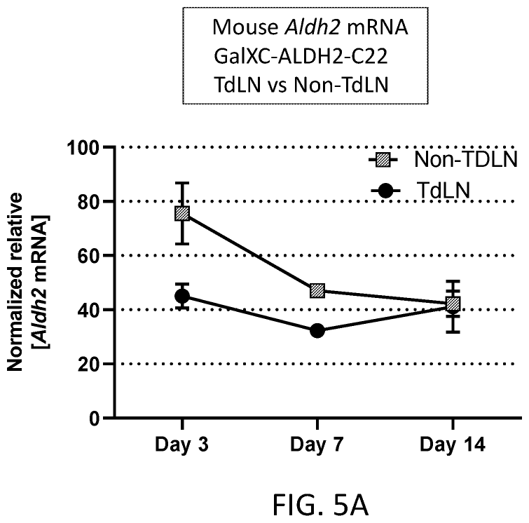

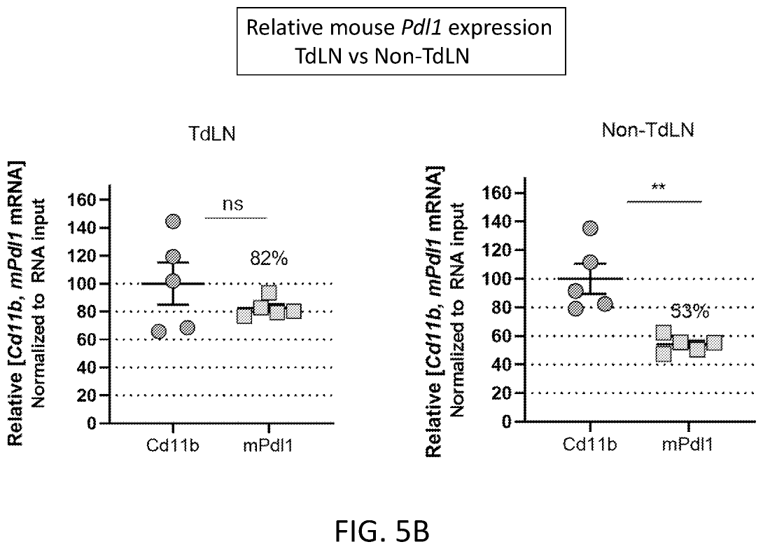

Dose response and duration of activity were determined by measuring the mouse and human Aldh2/ALDH2 mRNA levels on days 3, 7- and 14 post treatment. In parallel, the activity of GalXC-ALDH2-C22 in non-tumor bearing mice was also investigated at 25 mg/kg dose level on days 3 and 14 post treatment (FIG. 4B). As observed previously, no target knockdown was observed in human tumor epithelial parenchyma at any dose level, including the high dose of 50 mg/kg (FIG. 3A). However, robust knockdown of Aldh2 mRNA was observed in mouse host tissue (tumor microenvironment) (FIG. 3B). Nadir for mRNA knockdown in the murine TME was observed at one-week post-dose. ED₅₀ at nadir was observed to be between 10 and 25 mg/kg with the max knockdown was greater than 75%. Robust mRNA knockdown was maintained for at least two weeks post-dose. In the same study, tumor draining lymph nodes (axillary and inguinal) from the mice were also collected and analyzed by qPCR for mRNA levels of mouse Aldh2. As demonstrated in FIG. 4A, potent and durable activity was observed regardless of dose level. The ED₅₀ in tumor draining lymph nodes (TdLN) was determined to be <10 mg/kg. The absence of a dose related response suggests that there was saturation of activity even at the lowest dose level of GalXC-ALDH2-C22. FIG. 4B shows that no target knockdown was observed in the lymph nodes (LNs) of non-tumor bearing mice treated with GalXC-ALDH2-C22. Without being bound by theory, it is possible that lack of activity in control LNs suggests that the activity demonstrated in TdLN is tumor mediated and that GalXC-ALDH-C22 conjugate gained access to the LNs through the tumor lymphatic drainage. To examine whether target knockdown was also observed across different lymph nodes types in tumor bearing mice, the non-draining lymph nodes (LNs on the opposite side of the body to the TdLN), were also collected and analyzed for target mRNA levels at all 3 time points. As shown in FIG. 5A, the target mRNA levels in non-TdLN were reduced 20% on day 3, 50% on day 7 and reached the same level (60%) of knockdown as observed in TdLN on day 14. The level of immune suppressive characteristics of cell populations was assessed by determining the ratio of mRNA markers CD11b and Pdl1 in a given cell population. In this experiment, the murine mRNA ratio of these markers was found to be significantly lower in non-TdLN compared to TdLN on day 14 (FIG. 5B), suggesting that the cell population present in TdLN is more suppressive than the cell population present in Non-TdLN.

### Example 5: GalXC Lipid Conjugates Mediate Target Knockdown in Tumor-Associated Myeloid Cells

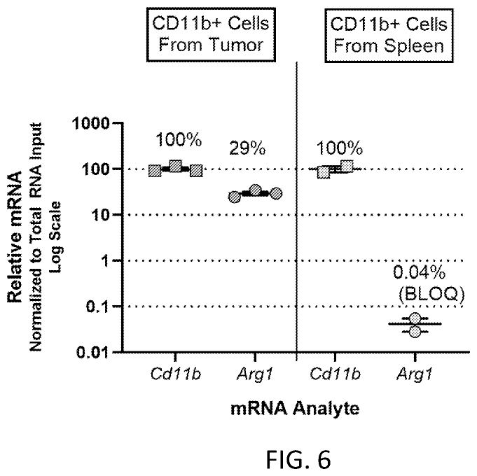

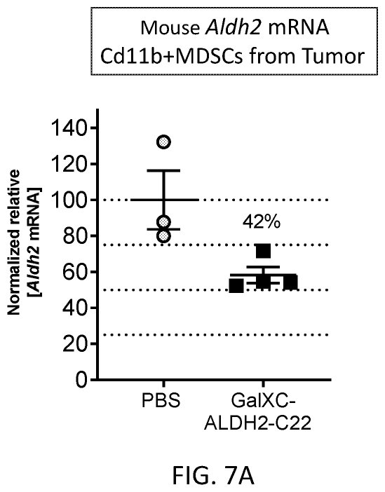

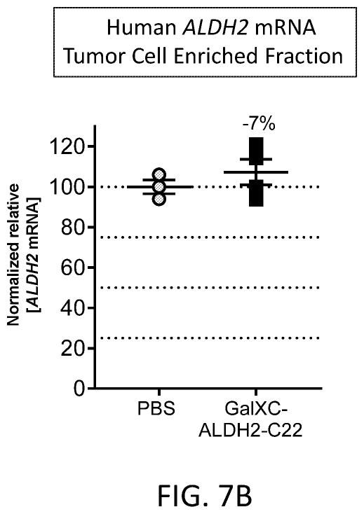

Myeloid-derived suppressor cells (MDSCs) are a heterogeneous population of cells that expand during tumorigenesis and which have the remarkable ability to suppress T-cell responses. Collectively, MDSCs are characterized by the co-expression of cell surface or mRNA markers CD11b (a marker for the myeloid cells of the macrophage lineage) and Gr-1 (a marker for the myeloid lineage differentiation antigen) and denoted as CD11b⁺Gr-1⁺ cells. Gr-1 is further comprised of 2 components Ly6G and Ly6C. MDSCs consist of two subsets: Granulocytic MDSC (G-MDSC), further characterized as CD11b⁺Ly6G⁺Ly6C^{lo}, and monocytic MDSC (M-MDSC) characterized as CD11b⁺Ly6G⁻Ly6C^{hi}. To elucidate the cell populations susceptible to target knockdown mediated by GalXC lipid conjugates in mouse host tissue, specifically to investigate target knockdown in the CD11b⁺ MDSCs of the TME, LS411N human xenograft tumors were implanted in nude mice as described in Example 3. After randomization mice were treated with a single dose of either GalXC-ALDH2-C22 conjugate at 25 mg/kg or PBS. At 3 days post dose, the murine host CD11b⁺ cells (myeloid derived suppressor cells or MDSC) and human tumor cells were isolated from single cell suspensions of tumors through positive and negative magnetic separation methods, respectively, using MACS separation technology (Miltenyi Biotec Inc, Auburn, CA). To isolate the CD11b positive cells, a single cell suspension of tumor was made using gentle MACS dissociator. CD11b positive cells in the single cell suspension were then magnetically labeled with MACS microbeads and enriched by passing through MACS columns and subsequently eluting the retained labeled cells in the column as positively selected fractions (CD11b MicroBeads UltraPure, mouse kit Cat #130-126-725). For tumor cell separation, non-target cells in the cell suspension were magnetically labeled with a cocktail of microbeads and passed through the MACS columns. During this process, the unwanted labeled cells were retained in the column and the unlabeled target cells (tumor cells) were collected in the flow-through as pure fraction. (Tumor Cell Isolation Kit, human Cat #130-108-339). CD11b⁺ cells were also isolated from the single cell suspensions of spleens of normal mice to compare the suppressive activity of the CD11b⁺ populations from different tissue types. Assuming comparable Aldh2 expression across cell types, CD11b+ MDSC preps were shown to be >90% pure. Upon isolation of the immune cell population, CD11b and Arg1 (markers characterizing immune suppression capabilities) mRNA levels were measured in both populations and the relative levels determined. In this analysis, CD11b mRNA was set to 100% in tumor and spleen subpopulations. While Arg1 was highly expressed in isolated MDSCs, it was not expressed (Ct>35) in spleen myeloid cells using the same affinity separation protocol, suggesting that the MDSCs in TME have high immune suppressive capabilities as compared to other myeloid derived cells, as this is one of the mechanisms that MDSCs use to inactivate tumor T-cells to suppress antitumor immune responses (FIG. 6). To determine if the GalXC-ALDH2-C22 mediated target knockdown was observed in the isolated CD11b⁺ cells and/or tumor cells, qPCR was performed, and the Aldh2/ALDH2 mRNA levels were determined. As demonstrated in FIGS. 7A and 7B, there was roughly 42% target knockdown observed in isolated CD11b⁺ cells, however there was no target knockdown observed in the isolated tumor cells. These data confirm the observation previously made in data collected from bulk tumor samples.

### Example 6: Using SAR to Identify a GalXC Lipid Conjugate Favorable for Delivery of siRNA to the Tumor Microenvironment and Tumor Draining Lymph Nodes

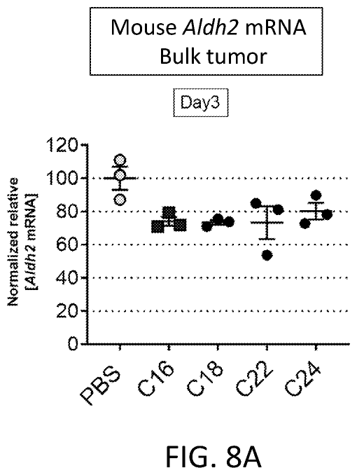

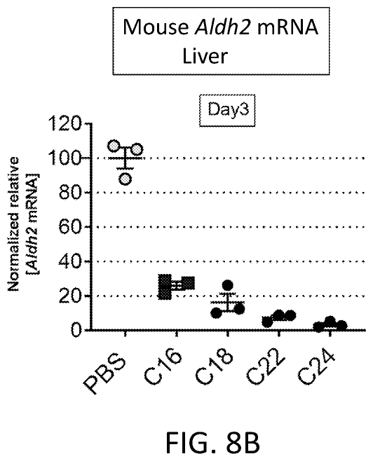

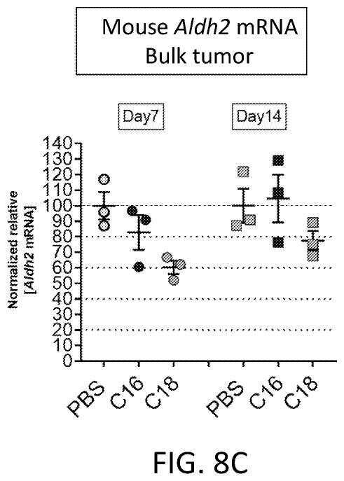

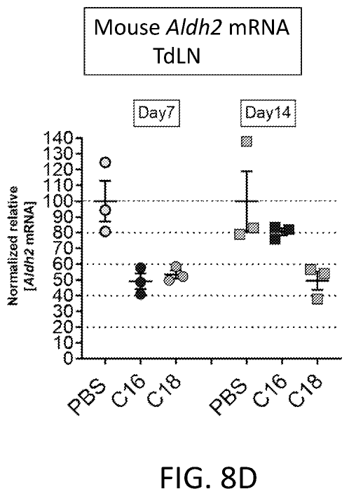

To identify a lipid conjugate with the most favorable properties to deliver payload and mediate target knockdown with the highest selectivity to myeloid cells in TME, a series of GalXC lipid conjugates as demonstrated in Scheme 1 (C16, C18, C22 and C24) were generated. To investigate these test articles, Pan02 murine pancreatic tumor cells were implanted in nude mice. When the tumors reached a volume of 300-400 mm³, the mice were randomized into groups and treated with either a single dose of PBS or a GalXC lipid conjugate (C16, C18, C22 and C24) at 25 mg/kg. Target knockdown was assessed on day 3 in bulk tumor and in liver (FIGS. 8A and 8B) to identify a GalXC lipid a conjugate with selectivity towards the target tissue (MDSCs) as compared to normal liver tissue. On day 3 post dose, Aldh2 mRNA levels in the tumors of all the treatment groups were decreased to a similar degree. There was a trend observed in the Aldh2 levels in livers of GalXC-ALDH2-lipid conjugate groups of a correlation of higher lipid acyl chain length with lower target knockdown (C24>C22>C18>C16), suggesting that these conjugates may use different mechanisms to traffic to TME versus Liver. Since the shorter lipid acyl chain conjugates C16 and C18 seem to be more liver sparing without compromising TME activity, as compared to longer acyl chain conjugates C22 and C24, the C16/C18 lipid conjugates were further explored in a separate study to further characterize their activity. Pan02 tumor bearing mice were treated with a single subcutaneous dose of GalXC-ALDH2-C16 or GalXC-ALDH-C18 at 25 mg/kg, or PBS and activity was monitored in bulk tumor tissue and TdLN on days 7 and 14. As shown in FIGS. 8C and 8D, the C18 conjugate outperformed C16 in target knockdown in bulk tumor at both time points. Although both test articles showed similar activity in TdLN on day 7, the C16 conjugate mediated activity was significantly reduced on day 14 while C18 mediated activity was maintained. Based on these data, the GalXC-ALDH2-C18 conjugate was selected for further studies.

### Example 7: Differences in the Onset of Activity and Dose-Dependence in Myeloid Derived Suppressor Cell Subsets

While it has been demonstrated GalXC-ALDH2-lipid conjugates mediate delivery and silence the Aldh2 gene in CD11b⁺ cells, it is critical to determine whether knockdown is mediated in either of the cell types or in both subsets of cells. Since these cell population subsets use different mechanisms to exert immune suppressive activity, it is important to identify which cell populations the GalXC lipid conjugates show activity toward to identify appropriate therapeutic targets. As demonstrated in the literature, signaling through GM-CSF along with STAT3 or STAT5 plays a key role in recruiting granulocytic-MDSCs (G-MDSCs) to the TME and is heavily involved in their expansion and suppression by increasing the FATP2 receptors (SLC27A2; gene encoding FATP2) on G-MDSC and allowing for efficient uptake of long chain fatty acids, according to recent findings (Veglia et al, NATURE (2019) 569:73-78(2019), one of the fatty acids, arachidonic acid, when metabolized to PGE₂ by COX-2 enzyme (gene encoding COX-2; PTGS2), is involved in T-cell suppression. Monocytic MDSCs (M-MDSCs), on the other hand, are also recruited to the TME from bone marrow where they become suppressive. M-MDSCs are known to have a higher-level expression of lipid trafficking receptors such as SCARB1 and LDLR that are likely to be involving in lipid uptake. Once each of the myeloid cell subsets become suppressive, they heavily express suppression associated markers such as ARG1, TGFβ, IDO, ROS and many others.

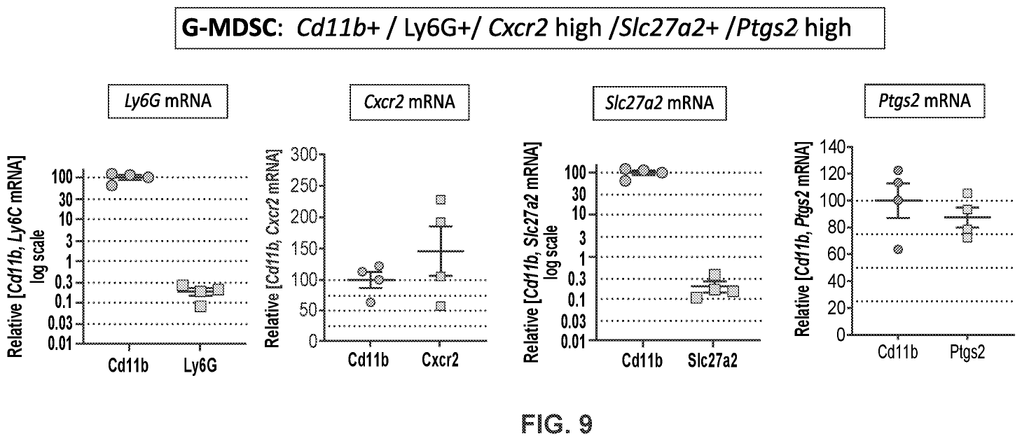

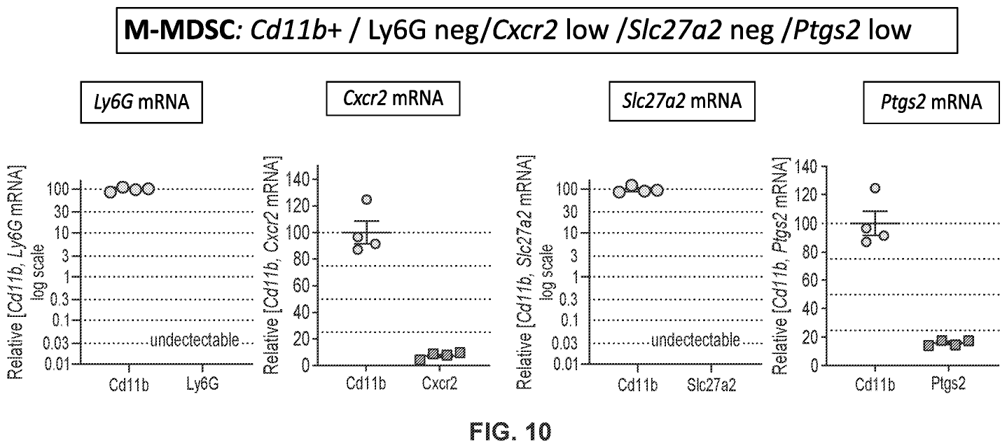

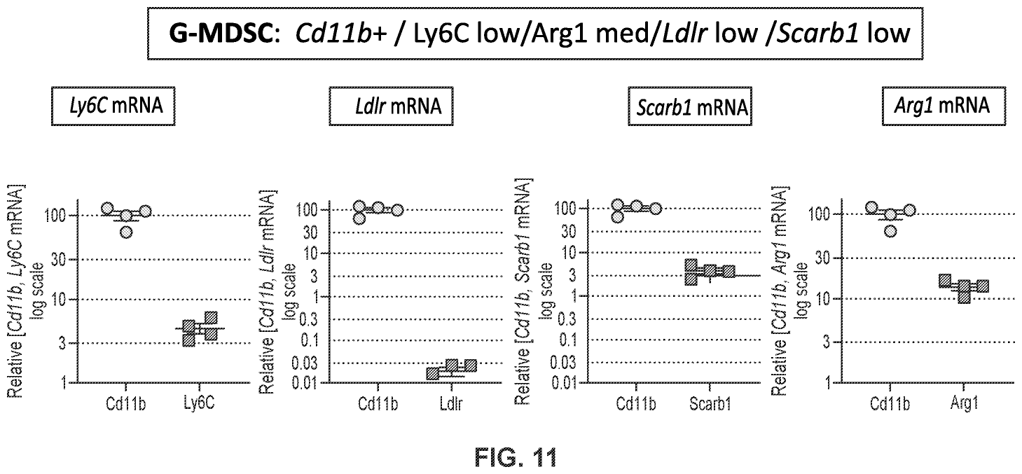

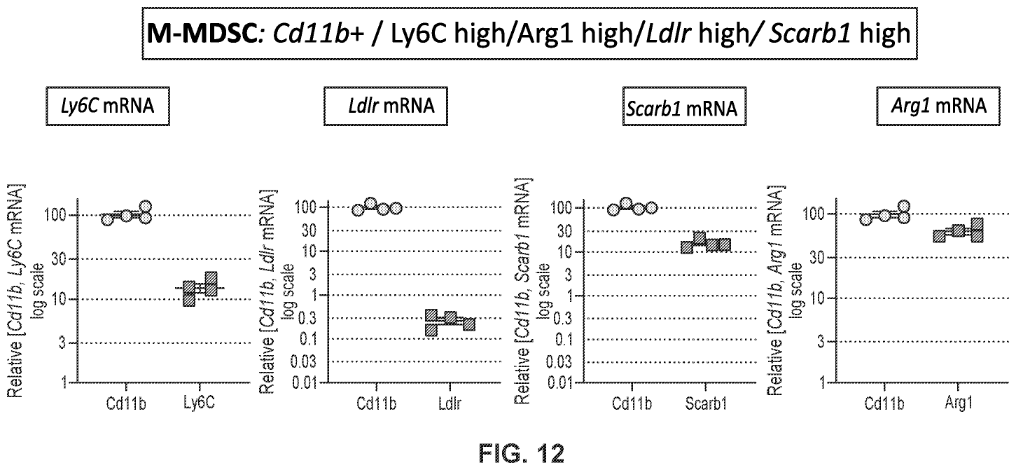

To determine whether the GalXC lipid conjugates mediate knockdown in either G-MDSC or M-MDSC cells or both, the gentle MACS magnetic separation method was used to isolate these cells as outlined for CD11b cell separation. As described above, a single cell suspension of tumor was made using gentle MACS dissociator. The Ly-6G⁺ fraction (or G-MDSC) was then isolated from the single cell suspension by magnetically labeling the Ly6G+ cells with MACS microbeads and passing through MACS columns and subsequently eluting the labeled cells as positively selected fractions. For separation of M-MDSCs (Ly6G⁻Gr-1⁺), the Gr-1⁺ cells present in the remaining flow through after Ly6G separation were magnetically labeled with MACS microbeads and passed through MACS columns to isolate the pure fraction by positive selection (Miltenyi Biotec Inc, Auburn CA, MDSC kit Cat #130-094-538). Through multiple positive and negative selection steps, pure MDSC subpopulations were isolated. These isolated populations were characterized by measuring multiple key markers that are expressed when G-MDSCs are differentiated from M-MDSCs as demonstrated in FIGS. 9 and 10. mRNA markers Ly6G, CxCr2, Slc27a2 and Ptgs2 are preferentially expressed by G-MDSCs and not by M-MDSCs. Expression of specific markers such as CxCr2, Scl27a2 and Ptgs2 suggest the recruitment and suppression activity of G-MDSCs in the TME. Likewise, mRNA markers Ly6C, Scarb1, Ldlr and Arg1 are highly expressed by M-MDSCs (FIGS. 11 and 12) compared to G-MDSCs. Higher expression of lipid trafficking receptors such as Scarb1 and Ldlr in M-MDSCs may play key role in lipid uptake. These mRNA marker profiles of isolated cell subpopulations were found to be consistent with the literature.

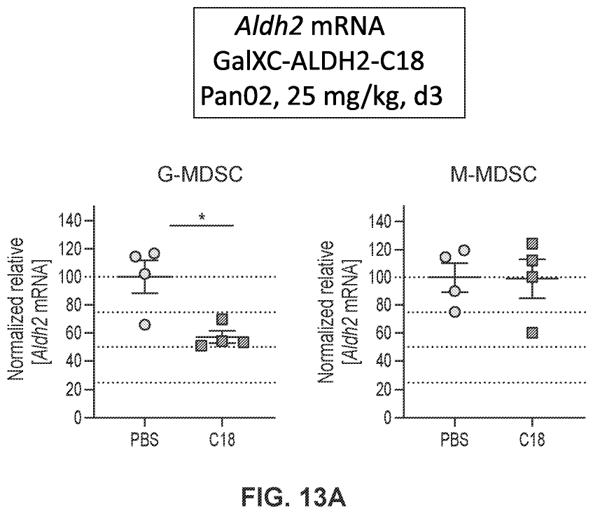

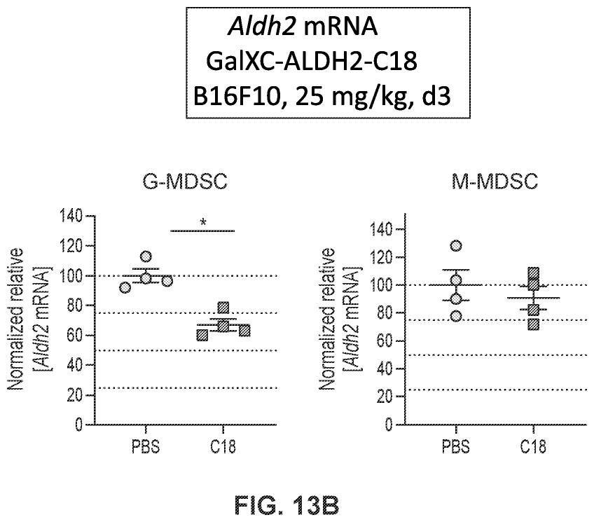

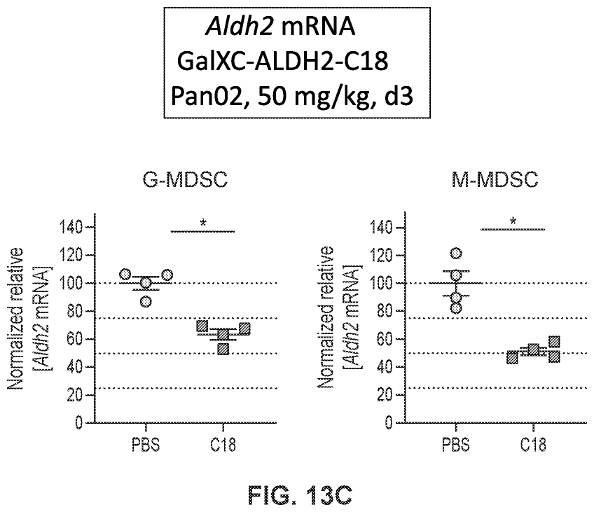

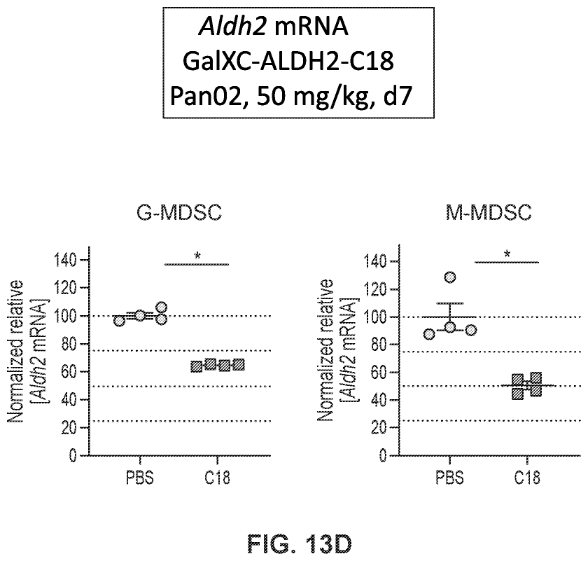

To identify in which cell populations knockdown can be mediated, Pan02 tumors were grown in nude mice as described in Example 3. After randomization into treatment groups mice received a single dose of either with GalXC-ALDH2-C18 at 25 mg/kg or a PBS control. At 3 days post treatment, tumors were collected, and the G-MDSC and M-MDSC populations were isolated. qPCR was used to determine the target mRNA levels. At this dose level, ˜40% Aldh2 mRNA knockdown was observed in only the G-MDSC subset and not in the M-MDSC subset. A follow-up study conducted in the same manner with a different tumor model, Bl6F10 (murine melanoma tumor) was performed to assess target knockdown pattern across tumor types. Bl6F10 tumors were implanted into nude mice as in Example 3 and when the tumors reached a volume of ˜300 mm³ size, the mice were randomized into treatment groups and treated with a single dose of the GalXC-ALDH2-C18 conjugate at 25 mg/kg, or PBS. At 3 days post treatment, mRNA levels were analyzed as described previously. As shown in FIGS. 13A and 13B, Aldh2 knockdown was observed only in G-MDSCs collected from both Pan02 and Bl6F10 tumors. To understand further how the dose level of GalXC lipid conjugate plays a role in delivery, the higher dose of 50 mg/kg was included in Pan02 tumor bearing mice and target knockdown was monitored on days 3 and 7. As shown in FIG. 13C, at a higher dose, the target knockdown in the G-MDSC population remained the same as the knockdown observed with 25 mg/kg. In addition, there was roughly 50% knockdown observed in the M-MDSC subset as well. The activity in each cell subset was maintained for a week post dose (FIG. 13D) suggesting that the delivery could be happening to G-MDSC first, likely through the FATP2 receptors, and once that population is saturated delivery shifts to the M-MDSCs (through Scarb1 and Ldlr) to mediate knockdown in this cell type. This suggests that the onset of activity and dose dependence maybe different between these two MDSC cell subsets.

### Example 8: Tissue Specific Targets in MDSC Cell Populations and Tumor Draining Lymph Nodes

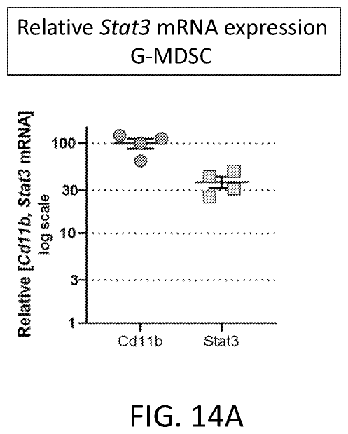

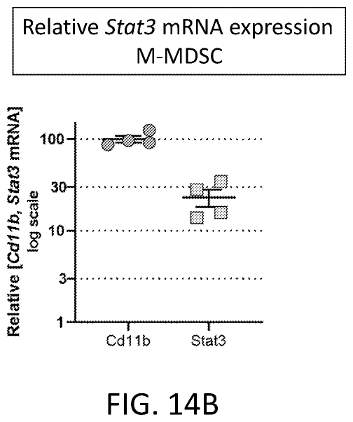

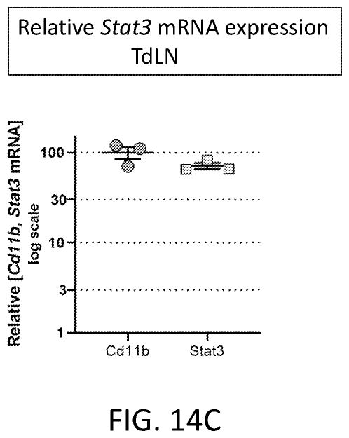

The data above demonstrate that the two MDSC subsets mediate immune suppression through different mechanisms. While CXCR2, SCL27A2 and PTGS2 are identified as specific potential targets on G-MDSCs, and PD-L1 would be a more specific target for cells residing in the TdLN, there are few targets that are expressed on both subsets of MDSC cells in the TME and cell types residing in TdLN. STAT3 is one such target that is expressed in all tissues of interest (i.e., tumor cells and immune cells in the tumor microenvironment). Expression of STAT3 was measured in Pan02 tumors (FIGS. 14A-14C). STAT3 is involved in immune suppression with examples abundantly reported in literature. Targeting STAT3 transcription through an RNAi mechanism could potentially overcome the challenges in the development of pharmacological STAT3 inhibitors. For these reasons STAT3 was selected as a proof-of-concept target to demonstrate tissue specific activity in the tissues of interest. STAT3 sequences were designed in the GalXC format with described modification patterns and screening for target knockdown in liver tissue was performed in normal CD-1 mice. Eighteen STAT3-GalXC conjugates (Table 3) were dosed once subcutaneously at 3 mg/kg.

TABLE 3
GalXC Compound Candidates for Identifying Tool Compounds for
Proof-of-concept Studies in Mice:

| | | | Sense | Antisense | |
|---|---|---|---|---|---|
| | | | strand | strand | |
| | | Sequence | SEQ | SEQ | Con- |
| Oligo | DP # | Type | ID NO | ID NO | jugate |
| GalXC-STAT3- | DP21679P: | Unmodified | 9 | 10 | GalNAc |
| 838 | DP21678G | Modified | 11 | 12 | GalNAc |
| GalXC-STAT3- | DP21697P: | Unmodified | 13 | 14 | GalNAc |
| 1390 | DP21696G | Modified | 15 | 16 | GalNAc |
| GalXC-STAT3- | DP21677P: | Unmodified | 17 | 18 | GalNAc |
| 1394 | DP21676G | Modified | 19 | 20 | GalNAc |
| GalXC-STAT3- | DP21691P: | Unmodified | 21 | 22 | GalNAc |
| 1398 | DP21690G | Modified | 23 | 24 | GalNAc |
| GalXC-STAT3- | DP21671P: | Unmodified | 25 | 26 | GalNAc |
| 1399 | DP21670G | Modified | 27 | 28 | GalNAc |
| GalXC-STAT3- | DP21673P: | Unmodified | 29 | 30 | GalNAc |
| 1400 | DP21672G | Modified | 31 | 32 | GalNAc |
| GalXC-STAT3- | DP21687P: | Unmodified | 33 | 34 | GalNAc |
| 1401 | DP21686G | Modified | 35 | 36 | GalNAc |
| GalXC-STAT3- | DP21675P: | Unmodified | 37 | 38 | GalNAc |
| 1402 | DP21674G | Modified | 39 | 40 | GalNAc |
| GalXC-STAT3- | DP21701P: | Unmodified | 41 | 42 | GalNAc |
| 1759 | DP21700G | Modified | 43 | 44 | GalNAc |
| GalXC-STAT3- | DP21689P: | Unmodified | 45 | 46 | GalNAc |
| 2029 | DP21688G | Modified | 47 | 48 | GalNAc |
| GalXC-STAT3- | DP21693P: | Unmodified | 49 | 50 | GalNAc |
| 2034 | DP21692G | Modified | 51 | 52 | GalNAc |
| GalXC-STAT3- | DP21699P: | Unmodified | 53 | 54 | GalNAc |
| 2448 | DP21698G | Modified | 55 | 56 | GalNAc |
| GalXC-STAT3- | DP21695P: | Unmodified | 57 | 58 | GalNAc |
| 2527 | DP21694G | Modified | 59 | 60 | GalNAc |
| GalXC-STAT3- | DP21683P: | Unmodified | 61 | 62 | GalNAc |
| 4107 | DP21682G | Modified | 63 | 64 | GalNAc |
| GalXC-STAT3- | DP21669P: | Unmodified | 65 | 66 | GalNAc |
| 4110 | DP21668G | Modified | 67 | 68 | GalNAc |
| GalXC-STAT3- | DP21667P: | Unmodified | 69 | 70 | GalNAc |
| 4123 | DP21666G | Modified | 71 | 72 | GalNAc |
| GalXC-STAT3- | DP21685P: | Unmodified | 73 | 74 | GalNAc |
| 4435 | DP21684G | Modified | 75 | 76 | GalNAc |
| GalXC-STAT3- | DP21681P: | Unmodified | 77 | 78 | GalNAc |
| 4474 | DP21680G | Modified | 79 | 80 | GalNAc |

### Modification Key for Table 3

| Symbol | Modification/linkage |
|---|---|
| mX | 2′-O-methyl modified nucleotide |
| fX | 2′-fluoro modified nucleotide |
| -S- | phosphorothioate linkage |
| — | phosphodiester linkage |
| [MePhosphonate-4O-mX] | 4′-O-monomethylphosphonate-2′-O-methyl |
| | modified nucleotide |
| ademX-GalNAc | 2′-aminodiethoxymethanol-nucleotide- |
| | GalNAc (GalNAc-conjugated nucleotide) |

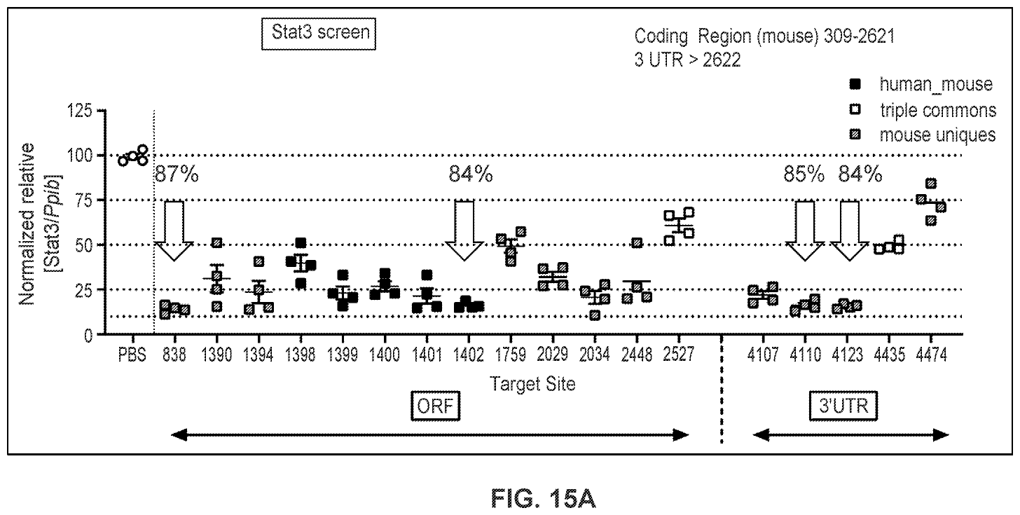

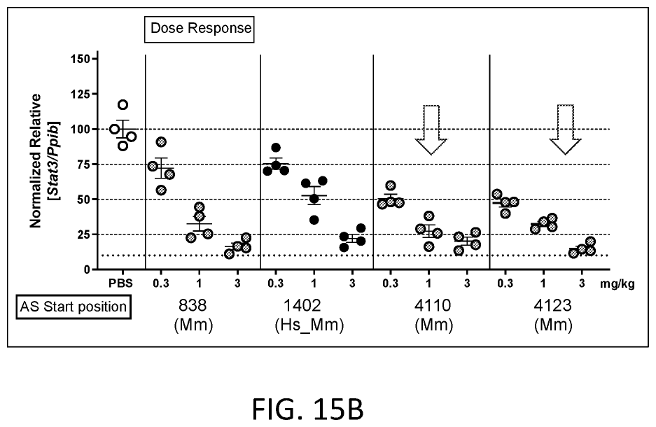

Five days post injection, livers were collected and subjected to mRNA analysis by qPCR. As a result of the screen, four sequences (GalXC-STAT3-838, GalXC-STAT3-1402, GalXC-STAT3-4110 and GalXC-STAT3-4123) that showed >85% target knockdown in liver were selected for further evaluation (FIG. 15A). Of these sequences three were identified as mouse specific and one was identified as human-mouse cross-reactive. These 4 sequences were further screened in CD-1 mice at 3 different doses (0.3, 1 and 3 mg/kg) to assess the dose response. GalXC-STAT3-4110 and 4123 were identified as the most potent sequences after the dose response screen, each with ED₅₀ of 0.3 mg/kg and thus these molecules were selected for further studies (FIG. 15B). C18 lipid conjugation was performed for both GalXC-STAT3-4110 or 4123 for proof-of-concept studies (Table 4).

TABLE 4
GalXC-STAT3 Lipid Conjugates

| SEQ ID | Oligonucleotide | Sequence Type | Ligand |
|---|---|---|---|
| 81 | GalXC-STAT3-4110-C18 | Modified Sense strand | C18 |
| 82 | | Modified Antisense strand | C18 |
| 83 | GalXC-STAT3-4123-C18 | Modified Sense strand | C18 |
| 84 | | Modified Antisense strand | C18 |

TABLE 5
GalXC-STAT3 Lipid Conjugates

| | Sequence | Sense strand | Antisense strand | |
|---|---|---|---|---|
| Oligo | Type | SEQ ID NO | SEQ ID NO | Conjugate |
| GalXC-STAT3- | Unmodified | 65 | 66 | C18 |
| 4110-C18 | Modified | 67 | 68 | C18 |
| GalXC-STAT3- | Unmodified | 69 | 70 | C18 |
| 4123-C18 | Modified | 71 | 72 | C18 |

### Modification Key for Tables 2, 4 and 5

| Symbol | Modification/linkage |
|---|---|
| mX | 2′-O-methyl modified nucleotide |
| fX | 2′-fluoro modified nucleotide |
| -S- | phosphorothioate linkage |
| — | phosphodiester linkage |
| [MePhosphonate-4O-mX] | 4′-O-monomethylphosphonate-2′-O-methyl |
| | modified nucleotide |
| adem X-C# | 2′-aminodiethoxymethanol-nucleotide- |
| | hydrocarbon chain (Lipid conjugate attached |
| | to a nucleotide (e.g. C16 or C18)) |

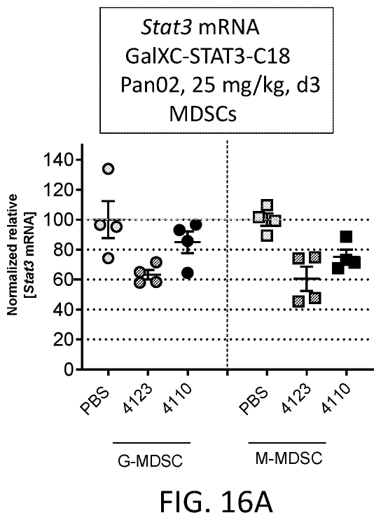

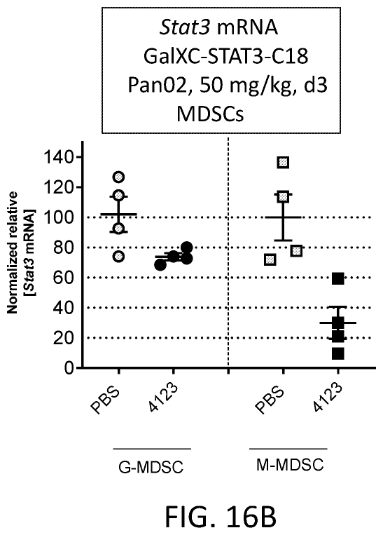

To evaluate the performance of GalXC-STAT3-C18 conjugates, Pan02 tumors were implanted in nude mice and upon reaching sufficient tumor volume mice were subjected to randomization as previously described. Mice received either a single dose of GalXC-STAT3-C18 4110 and 4123 subcutaneously at 25 mg/kg, 50 mg/kg, or PBS. At 3 days post injection, bulk tumors were collected. MDSC subsets were isolated as described in Example 5 and target mRNA was analyzed by qPCR (FIGS. 16A and 16B). Stat3 mRNA levels were reduced by ˜40% in G-MDSC and M-MDSCs by GalXC-STAT3-C18-4123. GalXC-STAT3-C18-4110 reduced the Stat3 mRNA levels only by 20% in both MDSC subsets. It is worth noting that the Aldh2 levels were reduced only in G-MDSC by the GalXC-ALDH2-lipid conjugates at the given dose and time point and the level of knockdown was comparable to the reduction of Stat3 levels in G-MDSC that were observed in the current experiment. Stat3 levels in M-MDSCs were reduced after GalXC-STAT3-C18 as compared to no reduction of Aldh2 levels in M-MDSC after GalXC-ALDH2-lipid conjugate treatment. The higher overall Aldh2 expression levels in M-MDSC compared to Stat3 levels may explain the difference in activity.

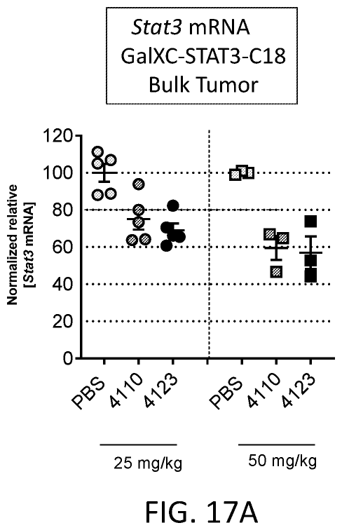

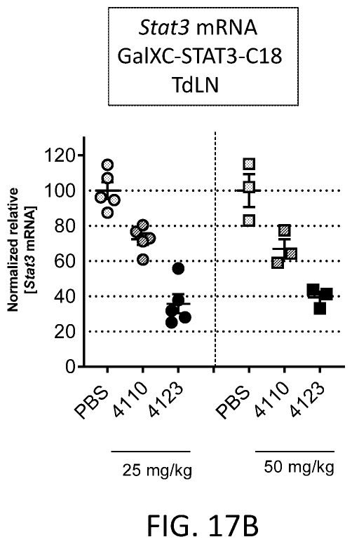

To understand how the dose level of GalXC-STAT3-C18 conjugates plays a role in trafficking of these molecules to different tissues and cell subsets, a follow-up study was performed as previously described with the same tumor model. Pan02 tumor bearing mice were treated with a single subcutaneous dose of either GalXC-STAT3-C18-4123 at 50 mg/kg, or PBS and Stat3 mRNA levels were measured after 3 days. The Stat3 knockdown in G-MDSC was not significantly altered as compared to the knockdown observed at the 25 mg/kg dose, however there was a significant improvement in Stat3 silencing observed in M-MDSC subset at this same dose level. In parallel study performed as previously described, Stat3 knockdown was assessed in bulk tumors and TdLNs on day 7 (FIGS. 17A and 17B). Dose dependent Stat3 mRNA knockdown was observed in bulk tumor with both GalXC-STAT3-C18 sequences. In TdLNs Stat3 mRNA levels were reduced by ˜60-65% by GalXC-STAT3-C18-4123, ˜25-30% by GalXC-STAT3-C18-4110 at both doses suggesting a saturation effect at these dose levels. Based on the data, GalXC-STAT3-C18-4123 was selected for further efficacy evaluations in immunocompetent mice.

### Example 9: STAT3 Inhibition Decreases the PD-L1 Levels in MDSCs and Mediates Acute Tumor Effects

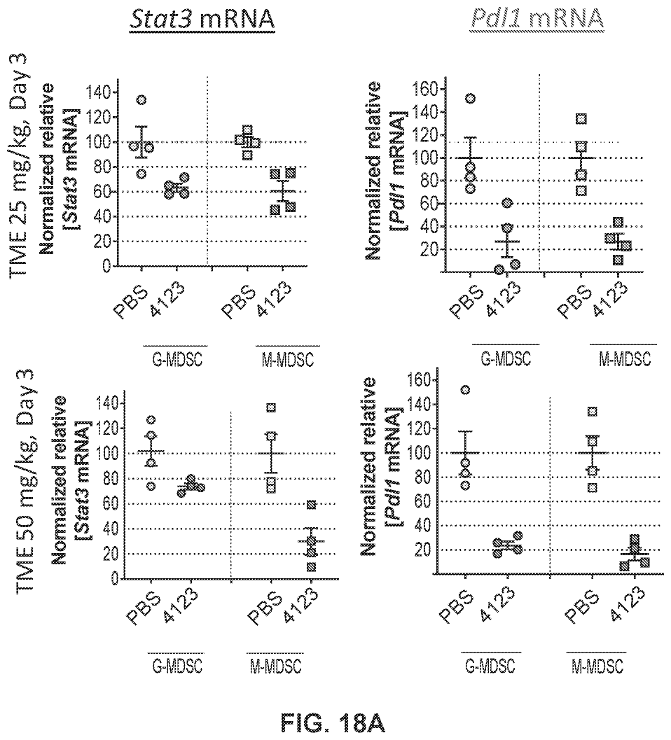

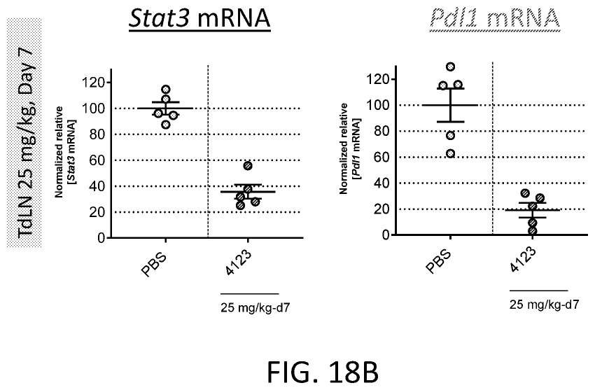

The transcriptional signature of phosphorylated STAT3 has been positively correlated with PD-L1 expression in tumors (Song et al, JOURNAL OF CELL PHYSIOLOGY (2020), Zerdes et al, CANCERS (2019), Song et al, BLOOD (2018). To extrapolate this correlation to STAT3 expressed by MDSCs, isolated populations of MDSCs treated with either PBS or a GalXC-STAT3 conjugate were assayed for Pdl1 mRNA. Pdl1 mRNA levels were decreased by ˜80% in both G-MDSC and M-MDSC populations treated with either 25 or 50 mg/kg of a GalXC-STAT3 (FIG. 18A). The Pdl1 levels were also dramatically reduced in TdLN after treatment with the GalXC-STAT3 conjugate, specifically GalXC-STAT3-C18-4123 (FIG. 18B). These data suggest a potential for downstream immunomodulation of PD-L1 after knockdown of STAT3.

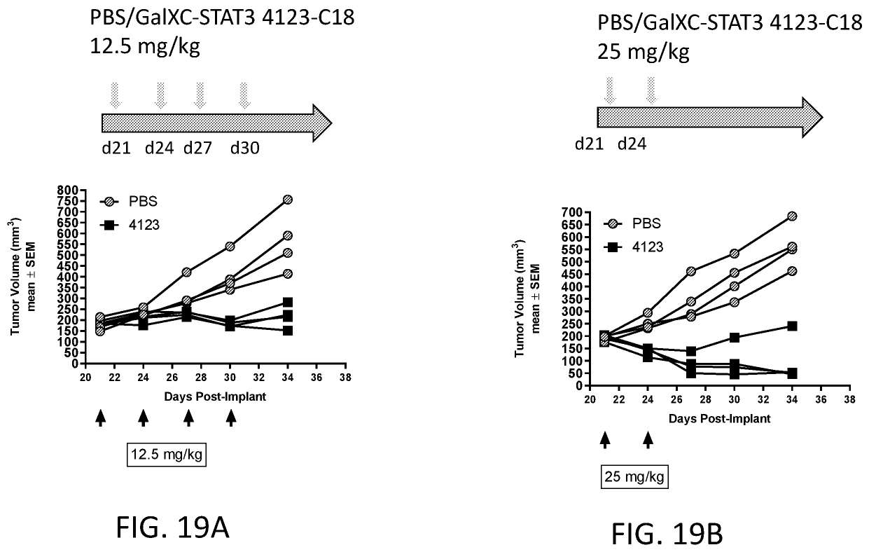

In a separate study, a Pan02 (murine pancreatic syngeneic model) tumor bearing C57BL/6 mice (n=4 per group) were treated subcutaneously with GalXC-STAT3-C18 conjugate following a split dosing model where all animals received a total dose of 50 mg/kg, dosed as either 25 mg/kg×2 doses or 12.5 mg/kg×4 doses. Tumors treated using the 25 mg/kg split dose showed acute tumor regression, even after the first dose (FIG. 19B). After the second dose of 25 mg/kg, tumors from 3 out of 4 mice regressed to sizes that were too small to be collected for further processing. The anti-tumor effect of the GalXC-STAT3 treatment was also observed in mice that received the 12.5 mg/kg split doses (FIG. 19A). These data suggest that STAT3 mediated regulation of PD-L1 results in an acute and dramatic effect on tumor growth in the Pan02 tumor bearing immunocompetent mice.

### Example 10: Preparation of Double-Stranded RNAi Oligonucleotides

### Oligonucleotide Synthesis and Purification

The double-stranded RNAi (dsRNA) oligonucleotides described in the foregoing Examples were chemically synthesized using methods described herein. Generally, dsRNAi oligonucleotides were synthesized using solid phase oligonucleotide synthesis methods as described for 19-23mer siRNAs (see, e.g., Scaringe et al. (1990) NUCLEIC ACIDS RES. 18:5433-41 and Usman et al. (1987) J. AM. CHEM. SOC. 109:p, 7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) COLD SPRING HARE PERSPECT BIOL. 9(1):a023812; Beaucage S. L., Caruthers M. H. Studies on Nucleotide Chemistry V. Deoxynucleoside Phosphoramidites—A New Class of Key Intermediates for Deoxypolynucleotide Synthesis. TETRAHEDRON LETT. 1981; 22:1859-62. doi: 10.1016/S0040-4039(01)90461-7). dsRNAi oligonucleotides having a 19mer core sequence were formatted into constructs having a 25mer sense strand and a 27mer antisense strand to allow for processing by the RNAi machinery. The 19mer core sequence is complementary to a region in the STAT3 mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-2684). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 10011M concentration) in duplex buffer consisting of 100 mM potassium acetate, 30 mM HEPES, pH 7.5. Complementary sense and antisense strands were mixed in equal molar amounts to yield a final solution of, for example, 5011M duplex. Samples were heated to 100° C. for 5′ in RNA buffer (IDT) and were allowed to cool to room temperature before use. The dsRNA oligonucleotides were stored at ˜20° C. Single strand RNA oligomers were stored lyophilized or in nuclease-free water at −80° C.

### Example 11: Generation of STAT3-Targeting Double-Stranded RNAi Oligonucleotides

Identification of STAT3 mRNA Target Sequences

Signal transducer and activator of transcription 3 (STAT3) is a transcription factor involved in several development and disease functions. To generate RNAi oligonucleotide inhibitors of STAT3 expression, a computer-based algorithm was used to computationally identify STAT3 mRNA target sequences suitable for assaying inhibition of STAT3 expression by the RNAi pathway. The algorithm provided RNAi oligonucleotide guide (antisense) strand sequences each having a region of complementarity to a suitable STAT3 target sequence of human STAT3 mRNA (e.g., SEQ ID NO:1217; Table 6). Some of the guide strand sequences identified by the algorithm were also complementary to the corresponding STAT3 target sequence of monkey STAT3 mRNA (SEQ ID NO: 1218 Table 6) and/or mouse STAT3 mRNA. STAT3 RNAi oligonucleotides comprising a region of complementarity to homologous STAT3 mRNA target sequences with nucleotide sequence similarity are predicted to have the ability to target homologous STAT3 mRNAs.

TABLE 6
Sequences of Human and Monkey STAT3 mRNA

| Species | Ref Seq # | SEQ ID NO |
|---|---|---|
| Human (Hs) | NM_139276.3 | 1217 |
| M. Fascicularis (Mf) | XM_005584240.2 | 1218 |
| Mus Musculus (Mm) | NM_213659.3 | 1229 |

RNAi oligonucleotides (formatted as DsiRNA oligonucleotides) were generated as described in Example 10 for evaluation in vitro. Each DsiRNA was generated with the same modification pattern, and each with a unique guide strand having a region of complementarity to a STAT3 target sequence identified by SEQ ID NOs: 89-280. Modifications for the sense and anti-sense DsiRNA included the following (X—any nucleotide; m—2′-O-methyl modified nucleotide; r—ribosyl modified nucleotide):

### Sense Strand:

rXmXrXmXrXrXrXrXrXrXrXrXrXmXrXmXrXrXrXrXrXrXrXXX

### Anti-sense Strand:

mXmXmXmXrXrXrXrXrXrXmXrXmXrXrXrXrXrXrXrXrXrXmXrXmXmXmX

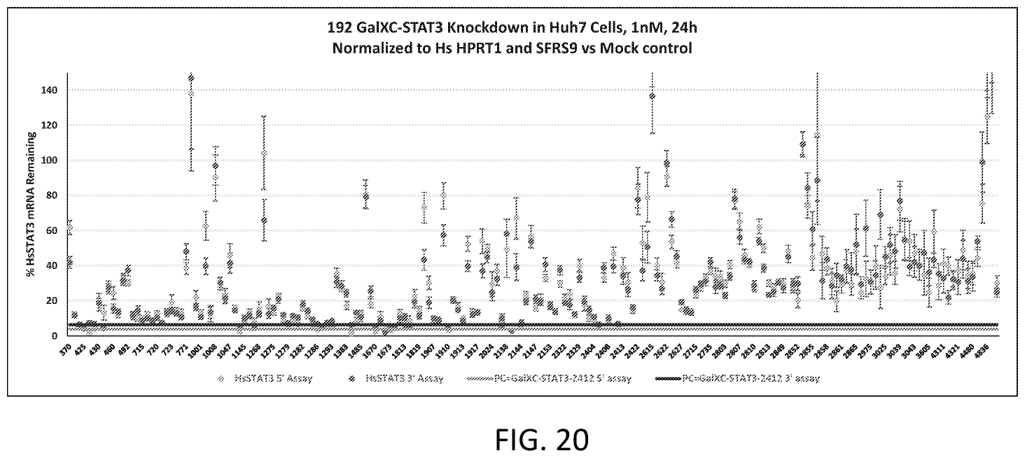

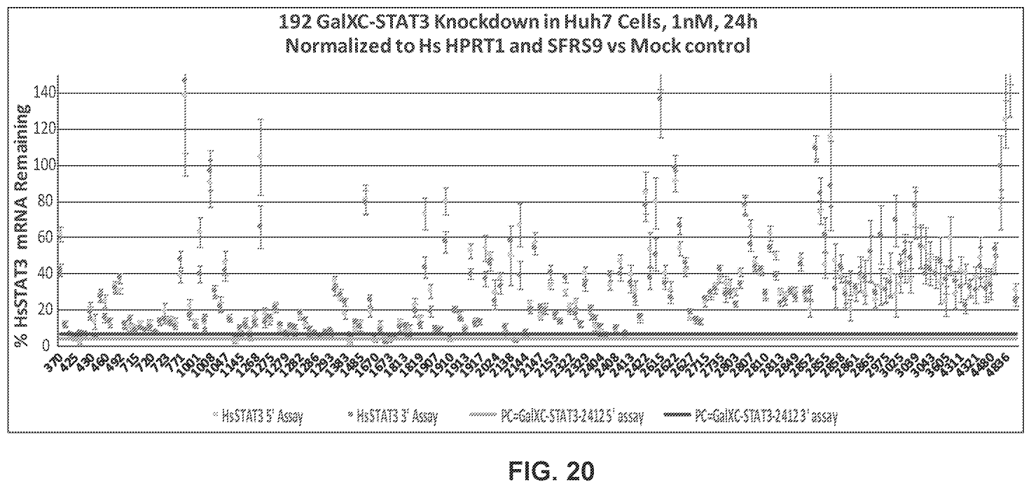

The ability of each of the modified DsiRNA in Table 7 to reduce STAT3 mRNA was measured using in vitro cell-based assays. Briefly, human hepatocyte (Huh7) cells expressing endogenous human STAT3 gene were transfected with each of the DsiRNAs listed in Table 7 at 1 nM in separate wells of a multi-well cell-culture plate. Cells were maintained for 24 hours following transfection with the modified DsiRNA, and then the amount of remaining STAT3 mRNA from the transfected cells was determined using TAQMAN®-based qPCR assays. Two qPCR assays, a 3′ assay and 5′ assay (Forward 1—SEQ ID NO:1219), Reverse 1—SEQ ID NO:1220, Probe 1—SEQ ID NO: 1221; Forward 2—SEQ ID NO: 1222, Reverse 2—SEQ ID NO: 1223, Probe 2—SEQ ID NO: 1224) were used to determine STAT3 mRNA levels as measured using PCR probes conjugated to 6-carboxy-fluorescein (FAM). Each primer pair was assayed for % remaining RNA as shown in Table 7 and FIG. 20. DsiRNAs resulting in less than or equal to 10% STAT3 mRNA remaining in DsiRNA-transfected cells when compared to mock-transfected cells were considered DsiRNA “hits”. The Huh7 cell-based assay evaluating the ability of the DsiRNAs listed in Table 7 to inhibit STAT3 expression identified several candidate DsiRNAs.

Taken together, these results show that DsiRNAs designed to target human STAT3 mRNA inhibit STAT3 expression in cells, as determined by a reduced amount of STAT3 mRNA in DsiRNA-transfected cells relative to control cells. These results demonstrate that the nucleotide sequences comprising the DsiRNA are useful for generating RNAi oligonucleotides to inhibit STAT3 expression. Further, these results demonstrate that multiple STAT3 mRNA target sequences are suitable for the RNAi-mediated inhibition of STAT3 expression.

TABLE 7
Analysis of STAT3 mRNA in Huh7 cells

| SED | SED | | | | | | | |
|---|---|---|---|---|---|---|---|---|
| ID NO | ID NO | | | | | | | |
| (Sense | (Anti-sense | DsiRNA | Average | | STAT3-5′ Assay | | STAT3-3′ Assay | |
| Strand) | Strand) | name | % remaining | SEM | % remaining | SEM | % remaining | SEM |
| 473 | 665 | 370 | 51.9 | 3.7 | 61.8 | 4.0 | 41.9 | 3.3 |
| 474 | 666 | 372 | 12.0 | 1.3 | 12.3 | 1.5 | 11.7 | 1.2 |
| 475 | 667 | 424 | 5.9 | 1.5 | 5.3 | 1.7 | 6.5 | 1.2 |
| 476 | 668 | 425 | 4.4 | 1.0 | 4.7 | 0.8 | 4.2 | 1.2 |
| 477 | 669 | 426 | 4.6 | 1.2 | 2.1 | 1.0 | 7.2 | 1.5 |
| 478 | 670 | 429 | 5.5 | 1.0 | 4.2 | 0.6 | 6.9 | 1.3 |
| 479 | 671 | 430 | 19.0 | 3.9 | 19.3 | 5.0 | 18.7 | 2.7 |
| 480 | 672 | 432 | 8.8 | 2.5 | 13.3 | 4.2 | 4.4 | 0.8 |
| 481 | 673 | 433 | 27.6 | 2.9 | 27.6 | 3.6 | 27.5 | 2.2 |
| 482 | 674 | 460 | 20.1 | 3.1 | 24.5 | 3.7 | 15.6 | 2.5 |
| 483 | 675 | 461 | 12.9 | 1.9 | 12.4 | 2.0 | 13.5 | 1.9 |
| 484 | 676 | 462 | 32.2 | 2.9 | 32.7 | 2.9 | 31.6 | 2.9 |
| 485 | 677 | 492 | 33.8 | 2.3 | 30.3 | 1.6 | 37.3 | 3.0 |
| 486 | 678 | 678 | 11.7 | 2.0 | 11.7 | 2.3 | 11.8 | 1.6 |
| 487 | 679 | 681 | 12.5 | 2.3 | 10.4 | 2.0 | 14.6 | 2.5 |
| 488 | 680 | 715 | 9.5 | 0.8 | 10.4 | 0.9 | 8.7 | 0.7 |
| 489 | 681 | 716 | 11.2 | 1.1 | 12.5 | 1.4 | 9.9 | 0.7 |
| 490 | 682 | 717 | 8.4 | 1.5 | 8.0 | 1.4 | 8.7 | 1.6 |
| 491 | 683 | 720 | 11.4 | 1.7 | 12.4 | 1.8 | 10.4 | 1.5 |
| 492 | 684 | 721 | 7.5 | 0.9 | 7.3 | 0.8 | 7.6 | 0.9 |
| 493 | 685 | 722 | 13.3 | 2.0 | 13.5 | 2.1 | 13.1 | 2.0 |
| 494 | 686 | 723 | 16.7 | 3.2 | 18.9 | 4.5 | 14.4 | 1.9 |
| 495 | 687 | 724 | 13.6 | 1.7 | 14.2 | 2.0 | 12.9 | 1.5 |
| 496 | 688 | 768 | 12.1 | 2.0 | 13.1 | 2.2 | 11.0 | 1.8 |
| 497 | 689 | 771 | 43.2 | 3.9 | 38.4 | 3.3 | 48.0 | 4.6 |
| 498 | 690 | 773 | 142.6 | 42.3 | 138.3 | 44.1 | 146.9 | 40.4 |
| 499 | 691 | 1000 | 19.3 | 2.9 | 22.0 | 3.9 | 16.5 | 2.0 |
| 500 | 692 | 1001 | 12.1 | 1.6 | 13.3 | 1.7 | 11.0 | 1.4 |
| 501 | 693 | 1003 | 51.3 | 6.5 | 62.8 | 8.3 | 39.8 | 4.7 |
| 502 | 694 | 1006 | 13.0 | 3.9 | 12.3 | 4.2 | 13.6 | 3.7 |
| 503 | 695 | 1008 | 93.5 | 12.0 | 90.0 | 13.1 | 96.9 | 11.0 |
| 504 | 696 | 1009 | 30.1 | 3.2 | 29.9 | 3.7 | 30.4 | 2.8 |
| 505 | 697 | 1010 | 22.1 | 3.5 | 22.7 | 4.4 | 21.5 | 2.6 |
| 506 | 698 | 1047 | 43.7 | 6.3 | 45.8 | 6.8 | 41.6 | 5.7 |
| 507 | 699 | 1067 | 15.3 | 1.3 | 16.0 | 1.5 | 14.5 | 1.1 |
| 508 | 700 | 1068 | 3.6 | 0.7 | 2.5 | 0.8 | 4.8 | 0.7 |
| 509 | 701 | 1145 | 9.2 | 2.2 | 8.4 | 2.5 | 9.9 | 1.8 |
| 510 | 702 | 1151 | 12.4 | 2.1 | 13.0 | 2.4 | 11.9 | 1.9 |
| 511 | 703 | 1241 | 6.7 | 1.9 | 8.3 | 1.9 | 5.1 | 1.8 |
| 512 | 704 | 1268 | 14.3 | 3.0 | 15.6 | 3.8 | 13.0 | 2.2 |
| 513 | 705 | 1272 | 85.2 | 16.3 | 104.4 | 20.9 | 66.1 | 11.8 |
| 514 | 706 | 1273 | 15.1 | 3.3 | 17.3 | 3.9 | 12.8 | 2.7 |
| 515 | 707 | 1275 | 14.7 | 1.7 | 13.7 | 1.8 | 15.8 | 1.7 |
| 516 | 708 | 1277 | 21.7 | 2.0 | 22.5 | 1.7 | 20.9 | 2.3 |
| 517 | 709 | 1278 | 10.8 | 1.4 | 9.4 | 1.9 | 12.1 | 0.9 |
| 518 | 710 | 1279 | 6.8 | 0.7 | 6.3 | 0.7 | 7.3 | 0.8 |
| 519 | 711 | 1280 | 9.9 | 1.0 | 8.2 | 1.0 | 11.5 | 1.0 |
| 520 | 712 | 1281 | 8.6 | 1.1 | 6.7 | 0.9 | 10.5 | 1.4 |
| 521 | 713 | 1282 | 17.0 | 1.9 | 15.8 | 1.6 | 18.1 | 2.1 |
| 522 | 714 | 1283 | 12.8 | 1.5 | 11.3 | 1.4 | 14.2 | 1.7 |
| 523 | 715 | 1284 | 7.8 | 1.0 | 6.2 | 0.8 | 9.4 | 1.3 |
| 524 | 716 | 1286 | 5.5 | 0.4 | 3.9 | 0.5 | 7.0 | 0.4 |
| 525 | 717 | 1287 | 5.1 | 0.6 | 4.6 | 0.9 | 5.6 | 0.3 |
| 526 | 718 | 1292 | 6.4 | 0.8 | 5.3 | 0.6 | 7.6 | 1.1 |
| 527 | 719 | 1293 | 7.3 | 0.8 | 5.9 | 0.9 | 8.7 | 0.6 |
| 528 | 720 | 1299 | 33.4 | 3.0 | 35.8 | 2.7 | 30.9 | 3.2 |
| 529 | 721 | 1305 | 27.5 | 1.9 | 26.7 | 0.6 | 28.3 | 3.1 |
| 530 | 722 | 1383 | 20.8 | 2.2 | 17.4 | 2.3 | 24.3 | 2.1 |
| 531 | 723 | 1388 | 4.0 | 0.8 | 1.6 | 0.6 | 6.3 | 0.9 |
| 532 | 724 | 1427 | 11.0 | 1.5 | 8.6 | 2.0 | 13.3 | 1.0 |
| 533 | 725 | 1485 | 11.6 | 2.3 | 12.4 | 2.1 | 10.8 | 2.6 |
| 534 | 726 | 1584 | 80.0 | 7.3 | 80.7 | 8.2 | 79.4 | 6.5 |
| 535 | 727 | 1586 | 22.0 | 2.8 | 18.6 | 2.6 | 25.4 | 3.0 |
| 536 | 728 | 1670 | 4.0 | 0.5 | 2.6 | 0.4 | 5.4 | 0.6 |
| 537 | 729 | 1671 | 9.9 | 2.6 | 10.8 | 3.1 | 8.9 | 2.1 |
| 538 | 730 | 1672 | 2.8 | 0.8 | 3.6 | 1.2 | 2.1 | 0.5 |
| 539 | 731 | 1673 | 3.7 | 0.9 | 3.1 | 1.0 | 4.2 | 0.9 |
| 540 | 732 | 1674 | 5.2 | 1.5 | 5.0 | 1.7 | 5.4 | 1.3 |
| 541 | 733 | 1676 | 11.5 | 2.3 | 13.0 | 2.1 | 10.1 | 2.4 |
| 542 | 734 | 1813 | 8.8 | 2.1 | 6.9 | 2.2 | 10.7 | 2.0 |
| 543 | 735 | 1815 | 7.0 | 1.9 | 8.9 | 2.7 | 5.0 | 1.1 |
| 544 | 736 | 1817 | 21.2 | 3.5 | 22.8 | 3.6 | 19.6 | 3.5 |
| 545 | 737 | 1819 | 13.3 | 1.9 | 15.0 | 1.9 | 11.5 | 1.8 |
| 546 | 738 | 1904 | 58.3 | 7.3 | 73.2 | 8.7 | 43.4 | 5.9 |
| 547 | 739 | 1906 | 24.6 | 3.5 | 30.2 | 3.8 | 18.9 | 3.2 |
| 548 | 740 | 1907 | 9.7 | 1.4 | 9.4 | 1.9 | 9.9 | 0.9 |
| 549 | 741 | 1908 | 9.0 | 1.4 | 9.2 | 1.5 | 8.9 | 1.3 |
| 550 | 742 | 1909 | 68.6 | 6.7 | 79.9 | 7.5 | 57.4 | 6.0 |
| 551 | 743 | 1910 | 4.3 | 0.6 | 3.3 | 0.6 | 5.4 | 0.6 |
| 552 | 744 | 1911 | 20.4 | 1.6 | 20.6 | 1.7 | 20.2 | 1.6 |
| 553 | 745 | 1912 | 15.6 | 1.6 | 16.6 | 2.4 | 14.7 | 0.8 |
| 554 | 746 | 1913 | 9.4 | 1.0 | 10.1 | 0.9 | 8.8 | 1.1 |
| 555 | 747 | 1914 | 46.2 | 3.6 | 52.5 | 4.2 | 39.8 | 3.0 |
| 556 | 748 | 1916 | 12.9 | 2.0 | 13.3 | 2.2 | 12.4 | 1.7 |
| 557 | 749 | 1917 | 13.3 | 1.4 | 13.4 | 1.5 | 13.3 | 1.3 |
| 558 | 750 | 1919 | 45.6 | 5.5 | 54.0 | 7.0 | 37.1 | 4.0 |
| 559 | 751 | 1920 | 47.5 | 2.8 | 49.9 | 2.3 | 45.1 | 3.4 |
| 560 | 752 | 2024 | 27.1 | 5.9 | 29.5 | 7.1 | 24.7 | 4.6 |
| 561 | 753 | 2135 | 35.1 | 3.7 | 37.4 | 3.4 | 32.8 | 3.9 |
| 562 | 754 | 2136 | 8.6 | 2.1 | 6.9 | 2.0 | 10.3 | 2.2 |
| 563 | 755 | 2138 | 54.0 | 12.5 | 49.8 | 16.5 | 58.1 | 8.5 |
| 564 | 756 | 2139 | 2.9 | 0.6 | 2.8 | 0.7 | 3.1 | 0.6 |
| 565 | 757 | 2143 | 53.2 | 9.7 | 67.0 | 11.8 | 39.3 | 7.7 |
| 566 | 758 | 2144 | 6.2 | 1.6 | 5.1 | 1.3 | 7.2 | 1.9 |
| 567 | 759 | 2145 | 21.4 | 2.1 | 23.1 | 2.2 | 19.8 | 2.0 |
| 568 | 760 | 2146 | 55.3 | 5.0 | 56.7 | 6.3 | 54.0 | 3.7 |
| 569 | 761 | 2147 | 18.2 | 1.9 | 15.6 | 1.4 | 20.8 | 2.4 |
| 570 | 762 | 2148 | 20.2 | 2.5 | 20.7 | 3.1 | 19.8 | 1.9 |
| 571 | 763 | 2151 | 36.9 | 3.0 | 33.2 | 2.0 | 40.7 | 3.9 |
| 572 | 764 | 2153 | 17.1 | 1.9 | 17.3 | 2.2 | 17.0 | 1.6 |
| 573 | 765 | 2154 | 13.7 | 1.3 | 13.9 | 1.6 | 13.6 | 0.9 |
| 574 | 766 | 2159 | 33.6 | 2.2 | 29.7 | 1.9 | 37.5 | 2.6 |
| 575 | 767 | 2322 | 20.1 | 1.8 | 21.3 | 2.5 | 18.8 | 1.2 |
| 576 | 768 | 2325 | 20.6 | 2.6 | 23.7 | 2.7 | 17.5 | 2.5 |
| 577 | 769 | 2327 | 12.1 | 1.4 | 11.8 | 1.4 | 12.4 | 1.4 |
| 578 | 770 | 2329 | 36.8 | 3.0 | 40.3 | 3.3 | 33.4 | 2.8 |
| 579 | 771 | 2333 | 18.9 | 3.1 | 18.5 | 4.2 | 19.4 | 2.0 |
| 580 | 772 | 2335 | 12.5 | 1.9 | 10.1 | 1.8 | 14.9 | 2.1 |
| 581 | 773 | 2404 | 9.8 | 2.2 | 8.7 | 3.0 | 10.8 | 1.3 |
| 582 | 774 | 2405 | 6.1 | 1.3 | 5.9 | 1.1 | 6.4 | 1.4 |
| 583 | 775 | 2407 | 36.0 | 2.7 | 33.2 | 2.6 | 38.9 | 2.9 |
| 584 | 776 | 2408 | 9.3 | 2.0 | 8.6 | 1.9 | 10.0 | 2.0 |
| 585 | 777 | 2411 | 43.2 | 3.7 | 46.9 | 3.7 | 39.6 | 3.6 |
| 586 | 778 | 2412 | 6.1 | 1.2 | 5.3 | 1.4 | 7.0 | 1.0 |
| 587 | 779 | 2413 | 36.9 | 5.5 | 39.0 | 5.8 | 34.8 | 5.3 |
| 588 | 780 | 2416 | 28.6 | 4.9 | 30.4 | 5.6 | 26.7 | 4.2 |
| 589 | 781 | 2418 | 15.5 | 1.9 | 15.0 | 2.1 | 16.0 | 1.7 |
| 590 | 782 | 2422 | 81.2 | 10.1 | 84.5 | 11.5 | 77.9 | 8.8 |
| 591 | 783 | 2427 | 45.3 | 7.7 | 53.2 | 9.4 | 37.3 | 5.9 |
| 592 | 784 | 2612 | 64.9 | 11.5 | 79.1 | 14.0 | 50.6 | 9.0 |
| 593 | 785 | 2615 | 153.3 | 24.5 | 170.0 | 27.8 | 136.6 | 21.1 |
| 594 | 786 | 2616 | 37.3 | 3.8 | 40.0 | 4.5 | 34.5 | 3.1 |
| 595 | 787 | 2617 | 28.9 | 4.1 | 30.8 | 4.8 | 27.0 | 3.3 |
| 596 | 788 | 2622 | 94.8 | 6.4 | 91.1 | 5.7 | 98.5 | 7.1 |
| 597 | 789 | 2625 | 60.0 | 4.2 | 53.6 | 3.9 | 66.4 | 4.4 |
| 598 | 790 | 2626 | 43.4 | 2.9 | 41.3 | 2.6 | 45.5 | 3.1 |
| 599 | 791 | 2627 | 17.1 | 1.0 | 15.0 | 0.6 | 19.2 | 1.4 |
| 600 | 792 | 2692 | 14.2 | 1.9 | 14.0 | 1.6 | 14.3 | 2.1 |
| 601 | 793 | 2693 | 13.6 | 1.4 | 14.0 | 1.4 | 13.2 | 1.5 |
| 602 | 794 | 2715 | 24.9 | 1.8 | 23.5 | 1.9 | 26.2 | 1.8 |
| 603 | 795 | 2719 | 28.7 | 2.3 | 28.2 | 2.6 | 29.3 | 2.0 |
| 604 | 796 | 2721 | 32.2 | 2.3 | 33.2 | 2.0 | 31.1 | 2.6 |
| 605 | 797 | 2735 | 39.4 | 2.2 | 36.7 | 1.7 | 42.0 | 2.6 |
| 606 | 798 | 2741 | 31.3 | 3.9 | 34.6 | 4.1 | 28.1 | 3.8 |
| 607 | 799 | 2801 | 31.4 | 2.7 | 33.7 | 3.3 | 29.0 | 2.1 |
| 608 | 800 | 2803 | 26.5 | 1.9 | 29.8 | 2.1 | 23.1 | 1.7 |
| 609 | 801 | 2804 | 37.3 | 2.2 | 40.7 | 2.4 | 33.9 | 2.1 |
| 610 | 802 | 2806 | 77.7 | 5.2 | 77.1 | 5.0 | 78.2 | 5.3 |
| 611 | 803 | 2807 | 60.9 | 4.2 | 65.4 | 4.7 | 56.3 | 3.8 |
| 612 | 804 | 2808 | 44.7 | 2.9 | 45.9 | 3.5 | 43.5 | 2.4 |
| 613 | 805 | 2809 | 41.7 | 1.9 | 41.0 | 1.9 | 42.3 | 1.8 |
| 614 | 806 | 2810 | 28.6 | 2.9 | 28.3 | 3.1 | 28.8 | 2.6 |
| 615 | 807 | 2811 | 58.2 | 3.1 | 62.4 | 4.1 | 54.0 | 2.1 |
| 616 | 808 | 2812 | 44.4 | 2.3 | 50.1 | 2.4 | 38.7 | 2.2 |
| 617 | 809 | 2813 | 26.7 | 1.6 | 30.0 | 1.8 | 23.5 | 1.3 |
| 618 | 810 | 2846 | 26.4 | 2.3 | 27.8 | 2.1 | 25.0 | 2.5 |
| 619 | 811 | 2848 | 30.9 | 1.4 | 31.3 | 1.4 | 30.5 | 1.5 |
| 620 | 812 | 2849 | 28.5 | 2.8 | 29.6 | 3.0 | 27.4 | 2.7 |
| 621 | 813 | 2850 | 46.7 | 3.4 | 48.2 | 3.5 | 45.2 | 3.4 |
| 622 | 814 | 2851 | 28.7 | 3.3 | 28.0 | 3.3 | 29.4 | 3.3 |
| 623 | 815 | 2852 | 25.0 | 4.1 | 20.3 | 4.2 | 29.8 | 3.9 |
| 624 | 816 | 2853 | 109.6 | 6.9 | 109.9 | 6.6 | 109.2 | 7.1 |
| 625 | 817 | 2854 | 79.0 | 7.6 | 73.6 | 6.4 | 84.3 | 8.7 |
| 626 | 818 | 2855 | 53.0 | 8.6 | 44.8 | 7.4 | 61.1 | 9.8 |
| 627 | 819 | 2856 | 101.8 | 31.5 | 115.1 | 38.1 | 88.4 | 24.9 |
| 628 | 820 | 2857 | 39.3 | 10.0 | 47.1 | 9.7 | 31.6 | 10.3 |
| 629 | 821 | 2858 | 41.4 | 5.1 | 38.8 | 4.0 | 44.0 | 6.2 |
| 630 | 822 | 2859 | 29.8 | 7.4 | 31.1 | 7.5 | 28.5 | 7.3 |
| 631 | 823 | 2860 | 27.2 | 6.4 | 19.8 | 5.9 | 34.6 | 6.9 |
| 632 | 824 | 2861 | 30.8 | 3.8 | 29.5 | 5.0 | 32.1 | 2.6 |
| 633 | 825 | 2862 | 38.3 | 8.0 | 37.1 | 6.5 | 39.6 | 9.6 |
| 634 | 826 | 2863 | 33.5 | 8.0 | 29.4 | 6.2 | 37.6 | 9.8 |
| 635 | 827 | 2865 | 50.2 | 15.0 | 48.2 | 12.7 | 52.1 | 17.2 |
| 636 | 828 | 2867 | 27.3 | 4.0 | 25.0 | 3.8 | 29.6 | 4.1 |
| 637 | 829 | 2868 | 47.0 | 13.0 | 32.6 | 10.1 | 61.4 | 16.0 |
| 638 | 830 | 2975 | 30.7 | 6.7 | 30.6 | 6.7 | 30.9 | 6.8 |
| 639 | 831 | 2979 | 37.2 | 9.9 | 39.7 | 11.8 | 34.8 | 8.1 |
| 640 | 832 | 2985 | 48.7 | 13.2 | 28.0 | 12.3 | 69.3 | 14.2 |
| 641 | 833 | 3025 | 39.6 | 5.1 | 33.9 | 4.6 | 45.3 | 5.6 |
| 642 | 834 | 3037 | 49.0 | 10.8 | 46.3 | 11.5 | 51.7 | 10.1 |
| 643 | 835 | 3038 | 42.1 | 8.1 | 36.0 | 6.6 | 48.2 | 9.6 |
| 644 | 836 | 3039 | 74.7 | 12.0 | 72.4 | 13.0 | 77.0 | 11.0 |
| 645 | 837 | 3041 | 54.7 | 11.6 | 54.4 | 11.0 | 54.9 | 12.1 |
| 646 | 838 | 3042 | 46.9 | 8.2 | 54.3 | 11.3 | 39.6 | 5.1 |
| 647 | 839 | 3043 | 44.9 | 9.5 | 47.5 | 10.3 | 42.2 | 8.8 |
| 648 | 840 | 3225 | 40.3 | 8.4 | 40.7 | 8.8 | 39.9 | 8.0 |
| 649 | 841 | 3226 | 41.0 | 12.2 | 34.7 | 11.5 | 47.2 | 12.9 |
| 650 | 842 | 3605 | 30.6 | 8.1 | 24.7 | 8.3 | 36.5 | 7.9 |
| 651 | 843 | 3611 | 51.3 | 8.2 | 59.5 | 12.2 | 43.1 | 4.1 |
| 652 | 844 | 3906 | 32.1 | 6.8 | 28.6 | 7.9 | 35.5 | 5.6 |
| 653 | 845 | 4311 | 37.2 | 8.0 | 41.7 | 7.8 | 32.6 | 8.2 |
| 654 | 846 | 4314 | 31.0 | 4.5 | 39.9 | 5.2 | 22.0 | 3.8 |
| 655 | 847 | 4317 | 32.1 | 4.8 | 31.9 | 5.3 | 32.3 | 4.3 |
| 656 | 848 | 4321 | 34.1 | 6.7 | 37.3 | 6.2 | 30.9 | 7.2 |
| 657 | 849 | 4465 | 46.3 | 11.0 | 48.9 | 11.3 | 43.8 | 10.8 |
| 658 | 850 | 4479 | 33.1 | 7.5 | 34.8 | 7.8 | 31.4 | 7.1 |
| 659 | 851 | 4480 | 34.7 | 7.3 | 36.0 | 6.7 | 33.5 | 7.9 |
| 660 | 852 | 4831 | 49.1 | 4.0 | 44.4 | 4.9 | 53.7 | 3.2 |
| 661 | 853 | 4833 | 87.3 | 14.1 | 75.5 | 11.0 | 99.1 | 17.2 |
| 662 | 854 | 4836 | 139.9 | 17.1 | 124.8 | 15.2 | 154.9 | 19.1 |
| 663 | 855 | 4837 | 175.2 | 39.6 | 185.9 | 41.5 | 164.5 | 37.7 |
| 664 | 856 | 4909 | 27.6 | 3.2 | 30.6 | 3.8 | 24.7 | 2.6 |
| | | PC | 5.2 | 0.7 | 3.9 | 0.7 | 6.4 | 0.7 |
| | | (2412) | | | | | | |

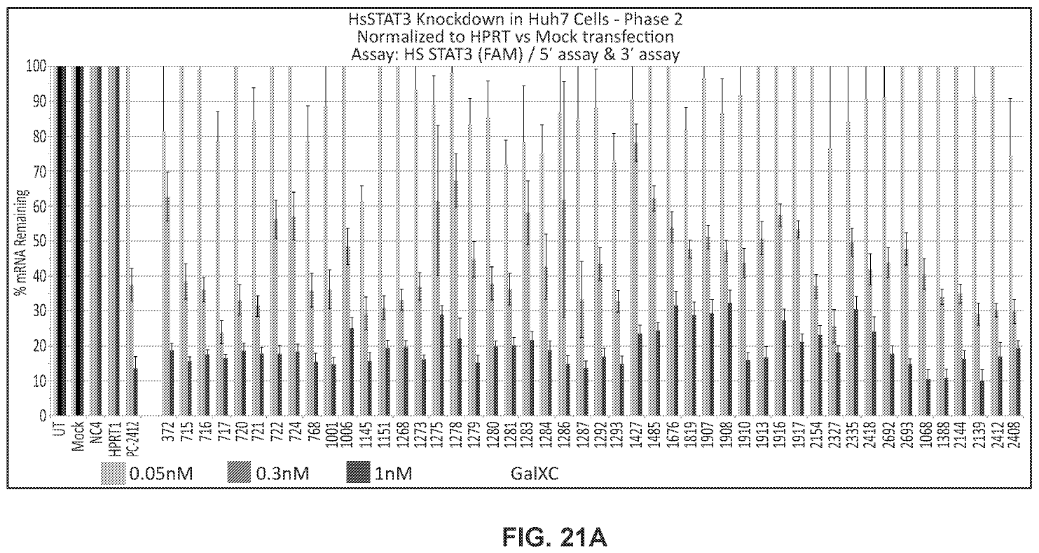

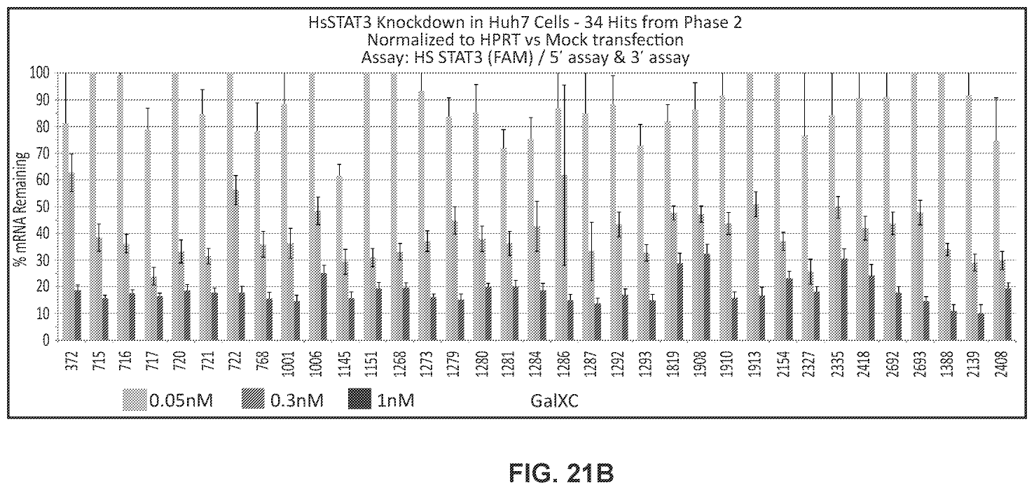

Following the initial in vitro screen, 48 constructs were selected for dosing studies. Huh7 cells were treated for 24 hours with 0.05 nM, 0.3 nM, or 1 nM of oligonucleotide. mRNA was isolated and measured to determine a potent dose (FIG. 21A). Of the tested oligonucleotides, 34 sequences were selected for further testing in vivo (Table 8 and FIG. 21B).

TABLE 8
Analysis of STAT3 mRNA in Huh7 Dosing Study

| | 1 nM | | 0.3 nM | | 0.05 nM | |
|---|---|---|---|---|---|---|
| | % Remaining | Standard | % Remaining | Standard | % Remaining | Standard |
| | mRNA | Deviation | mRNA | Deviation | mRNA | Deviation |
| STAT3-372 | 18.7 | 2.0 | 62.7 | 7.0 | 81.3 | 20.0 |
| STAT3-715 | 15.7 | 1.2 | 38.4 | 5.0 | 106.5 | 11.5 |
| STAT3-716 | 17.6 | 1.3 | 36.1 | 3.4 | 99.3 | 10.2 |
| STAT3-717 | 16.6 | 1.0 | 23.9 | 3.3 | 78.8 | 8.1 |
| STAT3-720 | 18.6 | 2.3 | 33.2 | 4.3 | 111.2 | 9.0 |
| STAT3-721 | 17.8 | 1.8 | 31.4 | 2.9 | 84.6 | 9.2 |
| STAT3-722 | 17.8 | 2.4 | 56.3 | 5.4 | 109.4 | 11.7 |
| STAT3-724 | 18.5 | 2.1 | 57.2 | 6.8 | 119.7 | 11.1 |
| STAT3-768 | 15.6 | 2.3 | 36.0 | 4.8 | 78.4 | 10.4 |
| STAT3-1001 | 14.7 | 2.1 | 36.3 | 5.6 | 88.5 | 13.2 |
| STAT3-1006 | 25.2 | 3.0 | 48.5 | 5.2 | 105.4 | 14.0 |
| STAT3-1068 | 10.5 | 2.7 | 40.5 | 4.5 | 144.0 | 37.7 |
| STAT3-1145 | 15.7 | 2.4 | 29.3 | 4.6 | 61.6 | 4.3 |
| STAT3-1151 | 19.4 | 2.2 | 31.0 | 3.3 | 103.5 | 7.8 |
| STAT3-1268 | 19.7 | 1.8 | 33.1 | 3.1 | 101.6 | 10.4 |
| STAT3-1273 | 16.2 | 1.1 | 37.1 | 3.9 | 93.4 | 9.3 |
| STAT3-1275 | 29.1 | 2.5 | 61.6 | 21.5 | 89.1 | 8.3 |
| STAT3-1278 | 22.2 | 5.7 | 67.4 | 7.6 | 98.0 | 8.8 |
| STAT3-1279 | 15.3 | 2.0 | 44.9 | 5.1 | 83.6 | 7.1 |
| STAT3-1280 | 19.8 | 1.5 | 37.9 | 4.7 | 85.3 | 10.4 |
| STAT3-1281 | 20.2 | 2.2 | 36.3 | 4.5 | 71.9 | 7.0 |
| STAT3-1283 | 21.8 | 2.4 | 58.1 | 9.1 | 78.3 | 16.1 |
| STAT3-1284 | 18.8 | 2.6 | 42.7 | 9.3 | 75.2 | 8.0 |
| STAT3-1286 | 15.0 | 2.2 | 61.9 | 33.7 | 86.9 | 19.8 |
| STAT3-1287 | 13.7 | 2.0 | 33.3 | 10.9 | 85.0 | 36.0 |
| STAT3-1292 | 17.0 | 2.3 | 43.4 | 4.7 | 88.3 | 10.9 |
| STAT3-1293 | 15.0 | 2.1 | 32.8 | 3.1 | 72.9 | 7.9 |
| STAT3-1388 | 11.0 | 2.3 | 34.1 | 2.2 | 111.9 | 28.3 |
| STAT3-1427 | 23.5 | 2.3 | 78.1 | 5.4 | 90.6 | 15.0 |
| STAT3-1485 | 24.4 | 2.1 | 62.2 | 3.5 | 114.1 | 12.6 |
| STAT3-1676 | 31.5 | 4.2 | 54.1 | 4.4 | 102.3 | 9.4 |
| STAT3-1819 | 28.9 | 3.6 | 47.8 | 2.6 | 82.0 | 6.2 |
| STAT3-1907 | 29.5 | 3.8 | 51.2 | 3.4 | 96.7 | 13.5 |
| STAT3-1908 | 32.4 | 3.6 | 47.2 | 3.0 | 86.4 | 10.0 |
| STAT3-1910 | 15.9 | 2.2 | 43.8 | 4.1 | 91.6 | 19.2 |
| STAT3-1913 | 16.8 | 3.1 | 50.9 | 4.7 | 106.2 | 20.7 |
| STAT3-1916 | 27.4 | 3.2 | 57.4 | 3.2 | 153.0 | 18.1 |
| STAT3-1917 | 21.2 | 2.3 | 53.3 | 2.4 | 117.9 | 27.1 |
| STAT3-2139 | 9.9 | 3.3 | 29.1 | 3.2 | 91.8 | 15.7 |
| STAT3-2144 | 16.3 | 2.3 | 34.9 | 2.8 | 105.9 | 37.8 |
| STAT3-2154 | 23.2 | 2.6 | 37.1 | 3.4 | 113.4 | 24.6 |
| STAT3-2327 | 18.2 | 1.9 | 25.7 | 4.7 | 76.6 | 31.2 |
| STAT3-2335 | 30.5 | 3.6 | 49.7 | 4.0 | 84.3 | 28.4 |
| STAT3-2408 | 19.4 | 2.0 | 29.8 | 3.4 | 74.6 | 16.2 |
| STAT3-2412 | 17.0 | 4.1 | 30.3 | 1.9 | 105.7 | 29.5 |
| STAT3-2418 | 24.2 | 4.2 | 42.0 | 4.5 | 90.7 | 28.0 |
| STAT3-2692 | 17.8 | 2.3 | 43.8 | 4.2 | 91.1 | 19.3 |
| STAT3-2693 | 14.8 | 1.5 | 47.8 | 4.6 | 124.5 | 25.5 |

### Example 12: RNAi Oligonucleotide Inhibition of STAT3 In Vivo

The in vitro screening assay in Example 11 validated the ability of STAT3-targeting DsiRNAs to knock-down target mRNA. To confirm the ability of the RNAi oligonucleotides to knockdown STAT3 in vivo, an HDI mouse model was used. A subset of the DsiRNAs identified in Example 11 were used to generate corresponding double-stranded RNAi oligonucleotides comprising a nicked tetraloop GalNAc-conjugated structure (referred to herein as “GalNAc-conjugated STAT3 oligonucleotides” or “GalNAc-STAT3 oligonucleotides”) having a 36-mer passenger strand and a 22-mer guide strand (Table 10 and Table 11). Further, the nucleotide sequences comprising the passenger strand and guide strand have a distinct pattern of modified nucleotides and phosphorothioate linkages. Three of the nucleotides comprising the tetraloop were each conjugated to a GalNAc moiety (CAS #14131-60-3). The modification patterns used are illustrated below:

### Pattern 1

Sense Strand: 5′ mX-S-mX-mX-mX-mX-mX-mX-fX-fX-fX-fX[-mX-]₁₆-[ademX-GalNAc]-[ademX-GalNAc]-[ademX-GalNAc]-mX-mX-mX-mX-mX-mX 3′.

### Hybridized to:

### Antisense Strand: 5′ [MePhosphonate-4O-mX]-S-fX-S-fX-fX-fX-mX-fX-mX-mX-fX-mX-mX-mX-fX-mX-mX-mX-mX-mX-mX-S-mX-S-mX 3′

Or, represented as:
Sense Strand: [mXs][mX][mX][mX][mX][mX][mX][fX][fX][fX][fX][mX][mX][mX][mX][mX][mX][mX][mX][mX][mX][mX][mX][mX][mX][mX][mX][ademA-GalNAc][ademA-GalNAc][ademA-GalNAc][mX][mX][mX][mX][mX][mX]

### Hybridized to:

Antisense Strand: [MePhosphonate-4O-mXs][fXs][fX][fX][fX][mX][fX][mX][mX][fX][mX][mX][mX][fX][mX][mX][mX][mX][mX][mXs][mXs][mX]

### Pattern 2

Sense Strand: 5′ mX-S-mX-mX-mX-mX-mX-mX-fX-fX-fX-fX[-mX-]₁₆-[ademX-GalNAc]-[ademX-GalNAc]-[ademX-GalNAc]-mX-mX-mX-mX-mX-mX 3′.

### Hybridized to:

### Antisense Strand: 5′ [MePhosphonate-4O-mX]-S-fX-S-fX-S-fX-fX-mX-fX-mX-mX-fX-mX-mX-mX-fX-mX-mX-mX-mX-mX-mX-S-mX-S-mX 3′

Or, represented as:
Sense Strand: [mXs][mX][mX][mX][mX][mX][mX][fX][fX][fX][fX][mX][mX][mX][mX][mX][mX][mX][mX][mX][mX][mX][mX][mX][mX][mX][mX][ademA-GalNAc][ademA-GalNAc][ademA-GalNAc][mX][mX][mX][mX][mX][mX]

### Hybridized to:

Antisense Strand: [MePhosphonate-4O-mXs][fXs][fXs][fX][fX][mX][fX][mX][mX][fX][mX][mX][mX][fX][mX][mX][mX][mX][mX][mXs][mXs][mX]

### (Modification key: Table 9).

| | |
|---|---|
| Symbol | Modification/linkage |

Key 1

| | |
|---|---|
| mX | 2′-O-methyl modified nucleotide |
| fX | 2′-fluoro modified nucleotide |
| -S- | phosphorothioate linkage |
| — | phosphodiester linkage |
| [MePhosphonate-4O-mX] | 4′-O-monomethylphosphonate-2′-O-methyl |
| | modified nucleotide |
| ademA-GalNAc | 2′-aminodiethoxymethanol-adenine-GalNAc |
| | (GalNAc attached to an adenine nucleotide) |

Key 2

| | |
|---|---|
| [mXs] | 2′-O-methyl modified nucleotide with a |
| | phosphorothioate linkage to the |
| | neighboring nucleotide |
| [fXs] | 2′-fluoro modified nucleotide with a |
| | phosphorothioate linkage to the |
| | neighboring nucleotide |
| [mX] | 2′-O-methyl modified nucleotide with |
| | phosphodiester linkages to |
| | neighboring nucleotides |
| [fX] | 2′-fluoro modified nucleotide with |
| | phosphodiester linkages to |
| | neighboring nucleotides |

Oligonucleotides in Table 10 and Table 11 were evaluated in mice engineered to transiently express human STAT3 mRNA in hepatocytes of the mouse liver. Briefly, 6-8-week-old female CD-1 mice (n=4-5) were subcutaneously administered the indicated GalNAc-conjugated STAT3 oligonucleotides at a dose of 1 mg/kg formulated in PBS. A control group of mice (n=3-4) were administered only PBS. Three days later (72 hours), the mice were hydrodynamically injected (HDI) with a DNA plasmid encoding the full human STAT3 gene (25 μg) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine STAT3 mRNA levels as described in Example 11. mRNA levels were measured for human mRNA. The values were normalized for transfection efficiency using the NeoR gene included on the DNA plasmid. A benchmark control (STAT3-1388) comprising a different modification pattern, was used for both assays (Sense Strand SEQ ID NO: 1100; Antisense Strand SEQ ID NO: 1190).

TABLE 10
GalNAc-Conjugated STAT3 RNAi Oligonucleotides for HDI screen

| | Unmodified | Unmodified | Modified | Modified |
|---|---|---|---|---|
| | Sense | Antisense | Sense | Antisense |
| | Strand | strand | Strand | strand |
| STAT3-372 | 861 | 951 | 1041 | 1131 |
| STAT3-715 | 857 | 947 | 1037 | 1127 |
| STAT3-716 | 858 | 948 | 1038 | 1128 |
| STAT3-717 | 859 | 949 | 1039 | 1129 |
| STAT3-720 | 860 | 950 | 1040 | 1130 |
| STAT3-721 | 862 | 952 | 1042 | 1132 |
| STAT3-722 | 863 | 953 | 1043 | 1133 |
| STAT3-768 | 864 | 954 | 1044 | 1134 |
| STAT3-1001 | 865 | 955 | 1045 | 1135 |
| STAT3-1006 | 866 | 956 | 1046 | 1136 |
| STAT3-1145 | 867 | 957 | 1047 | 1137 |
| STAT3-1151 | 868 | 958 | 1048 | 1138 |
| STAT3-1268 | 869 | 959 | 1049 | 1139 |
| STAT3-1273 | 870 | 960 | 1050 | 1140 |
| STAT3-1279 | 871 | 961 | 1051 | 1141 |
| STAT3-1280 | 872 | 962 | 1052 | 1142 |
| STAT3-1281 | 873 | 963 | 1053 | 1143 |
| STAT3-1388 | 920 | 1010 | 1100 | 1190 |

TABLE 11
GalNAc-Conjugated STAT3 RNAi Oligonucleotides for HDI screen

| | Unmodified | Unmodified | Modified | Modified |
|---|---|---|---|---|
| | Sense | Antisense | Sense | Antisense |
| | Strand | strand | Strand | strand |
| STAT3-1284 | 874 | 964 | 1054 | 1144 |
| STAT3-1286 | 875 | 965 | 1055 | 1145 |
| STAT3-1287 | 876 | 966 | 1056 | 1146 |
| STAT3-1292 | 877 | 967 | 1057 | 1147 |
| STAT3-1293 | 878 | 968 | 1058 | 1148 |
| STAT3-1819 | 879 | 969 | 1059 | 1149 |
| STAT3-1908 | 880 | 970 | 1060 | 1150 |
| STAT3-1910 | 881 | 971 | 1061 | 1151 |
| STAT3-1913 | 882 | 972 | 1062 | 1152 |
| STAT3-2154 | 883 | 973 | 1063 | 1153 |
| STAT3-2327 | 884 | 974 | 1064 | 1154 |
| STAT3-2335 | 885 | 975 | 1065 | 1155 |
| STAT3-2418 | 886 | 976 | 1066 | 1156 |
| STAT3-2692 | 887 | 977 | 1067 | 1157 |
| STAT3-2693 | 888 | 978 | 1068 | 1158 |
| STAT3-2139 | 940 | 1030 | 1120 | 1210 |
| STAT3-2408 | 896 | 986 | 1076 | 1166 |
| STAT3-1388 | 920 | 1010 | 1100 | 1190 |

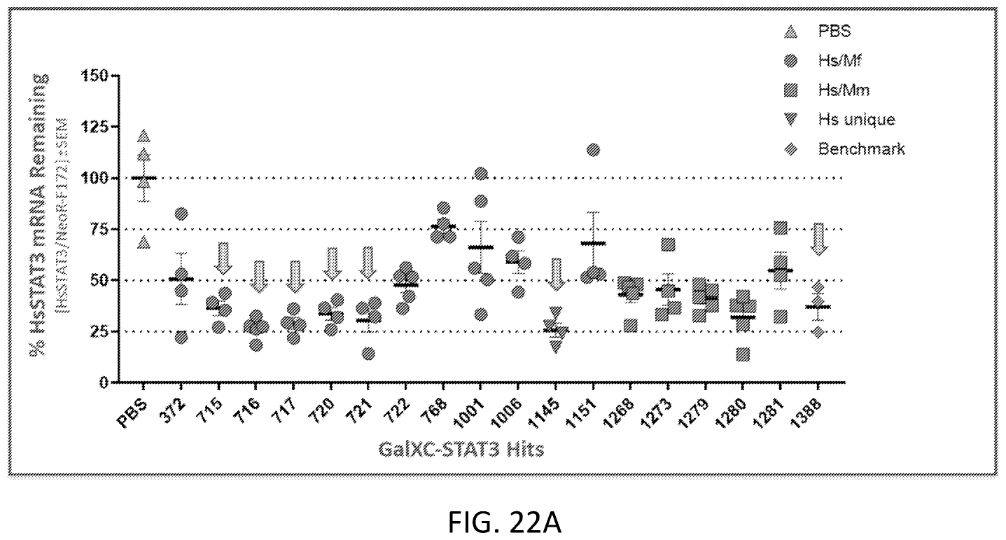

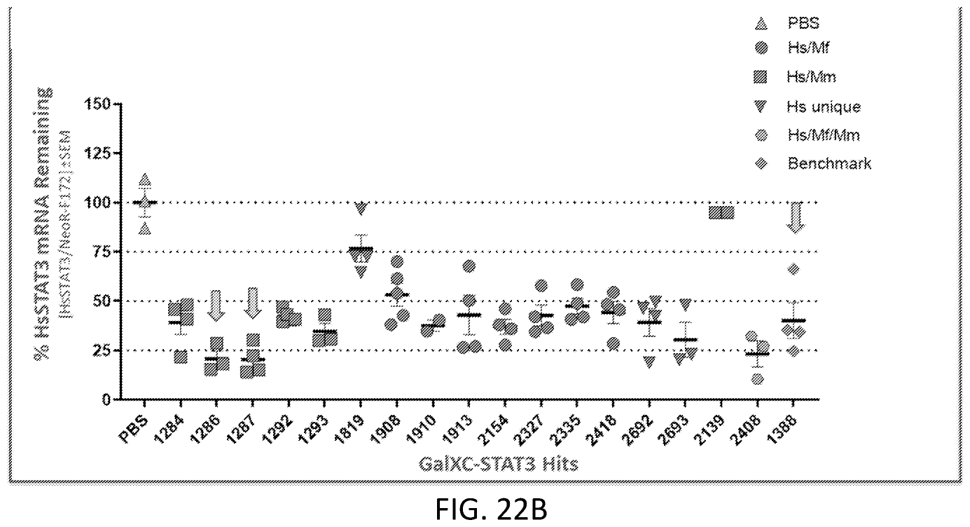

The results in FIGS. 22A and 22B demonstrate that GalNAc-conjugated STAT3 oligonucleotides designed to target human STAT3 mRNA inhibited human STAT3 mRNA expression in HDI mice, as determined by a reduction in the amount of human STAT3 mRNA expression in liver samples from HDI mice treated with GalNAc-conjugated STAT3 oligonucleotides relative to control HDI mice treated with only PBS.

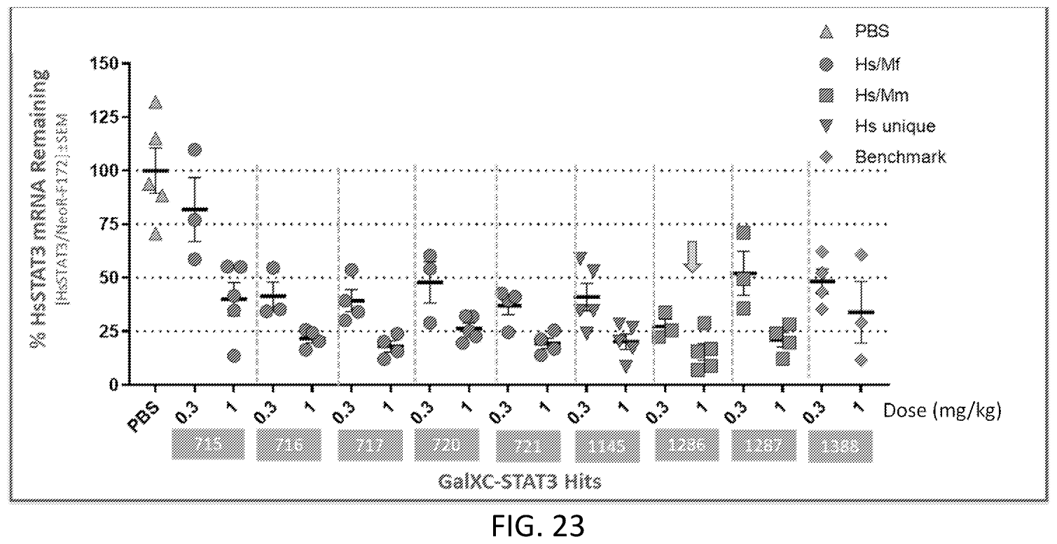

A subset of the GalNAc-conjugated STAT3 oligonucleotides tested in FIGS. 22A and 22B were further validated in a dosing study. Specifically, dosing studies were carried out using nine GalNAc-conjugated STAT3 oligonucleotides (STAT3-715, STAT3-716, STAT3-717, STAT3-720, STAT3-721, STAT3-1145, STAT3-1286, STAT3-1286, and STAT3-1287). Mice were hydrodynamically injected as described above and treated with 0.1 mg/kg, 0.3 mg/kg, or 1 mg/kg of oligonucleotide. Livers were collected after one day, and STAT3 expression was measured to determine a potent dose (FIG. 23). All GalNAc-conjugated STAT3 oligonucleotides were able to reduce STAT3 expression at a 1 mg/kg dose and STAT3-1286 was able to reduce expression at a 0.3 mg/kg dose. Overall, the HDI studies identified several potential GalNAc-conjugated STAT3 oligonucleotides for inhibiting STAT3 expression in liver.

### Example 13: Species Specific RNAi Oligonucleotide Inhibition of STAT3 In Vivo

To confirm the ability of RNAi oligonucleotides to knockdown STAT3 in vivo, several cross species and species specific GalNAc-conjugated STAT3 oligonucleotides were generated. Specifically, triple common (targeting human, non-human primate, and mouse; Hs/Mf/Mm), human/mouse (Hs/Mm), and human specific (Hs) oligonucleotides were evaluated.

### Hs/Mf/Mm and Hs/Mm Commons

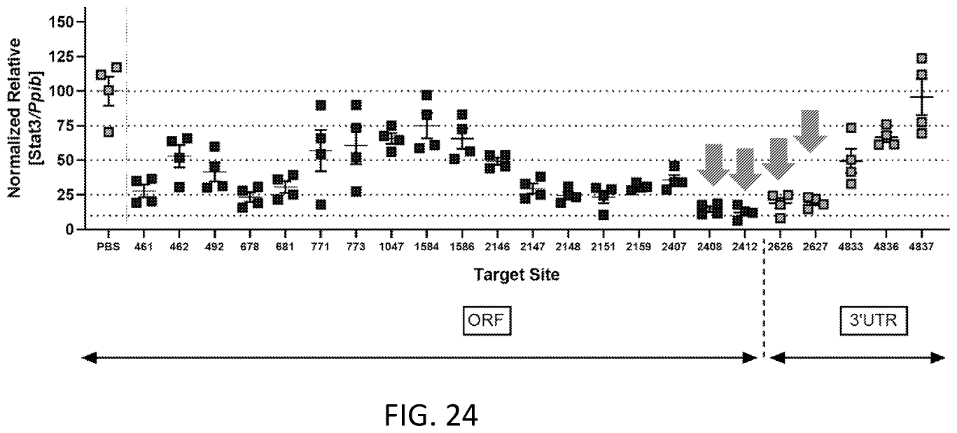

Mice expressing endogenous mouse STAT3 in the liver were subcutaneously injected at a dose of 3 mg/kg with the GalNAc-conjugated STAT3 oligonucleotides set forth in Table 12. Livers were collected after five days, and STAT3 expression was measured. Overall, the study identified several potential Hs/Mf/Mm GalNAc-conjugated STAT3 oligonucleotides for inhibiting STAT3 expression in liver (FIG. 24).

TABLE 12
GalNAc-Conjugated Human/Monkey/Mouse STAT3 RNAi
Oligonucleotides for Endogenous STAT3 screen.

| | Unmodified | Unmodified | Modified | Modified |
|---|---|---|---|---|
| | Sense | Antisense | Sense | Antisense |
| | Strand | strand | Strand | strand |
| STAT3-461 | 901 | 991 | 1081 | 1171 |
| STAT3-462 | 906 | 996 | 1086 | 1176 |
| STAT3-492 | 905 | 995 | 1085 | 1175 |
| STAT3-678 | 910 | 1000 | 1090 | 1180 |
| STAT3-681 | 909 | 999 | 1089 | 1179 |
| STAT3-771 | 908 | 998 | 1088 | 1178 |
| STAT3-773 | 904 | 994 | 1084 | 1174 |
| STAT3-1047 | 903 | 993 | 1083 | 1173 |
| STAT3-1584 | 902 | 992 | 1082 | 1172 |
| STAT3-1586 | 907 | 997 | 1087 | 1177 |
| STAT3-2146 | 898 | 988 | 1078 | 1168 |
| STAT3-2147 | 900 | 990 | 1080 | 1170 |
| STAT3-2148 | 899 | 989 | 1079 | 1169 |
| STAT3-2151 | 893 | 983 | 1073 | 1163 |
| STAT3-2159 | 897 | 987 | 1077 | 1167 |
| STAT3-2407 | 891 | 981 | 1071 | 1161 |
| STAT3-2408 | 896 | 986 | 1076 | 1166 |
| STAT3-2412 | 892 | 982 | 1072 | 1162 |
| STAT3-2626 | 890 | 980 | 1070 | 1160 |
| STAT3-2627 | 889 | 979 | 1069 | 1159 |
| STAT3-4833 | 912 | 1002 | 1092 | 1182 |
| STAT3-4836 | 895 | 985 | 1075 | 1165 |
| STAT3-4837 | 911 | 1001 | 1091 | 1181 |

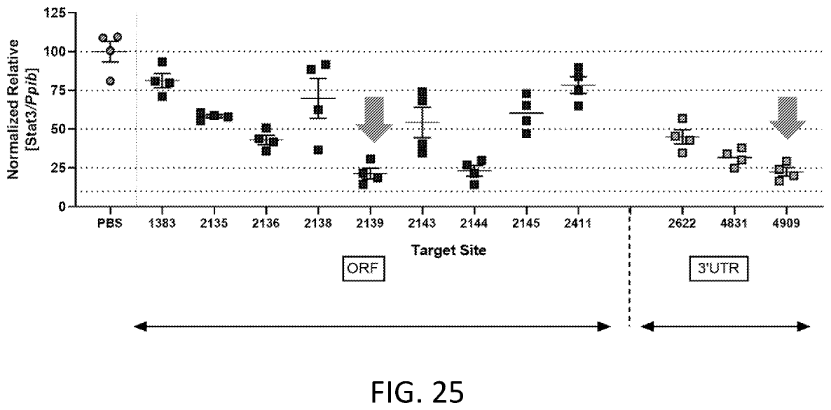

Human/Mouse GalNAc-conjugated STAT3 oligonucleotides set forth in Table 13 were tested in mice endogenously expressing mouse STAT3. As described above, mice were subcutaneously injected at a dose of 3 mg/kg with oligonucleotide. Livers were collected after five days, and mouse STAT3 expression was measured. Overall, the study identified several potential Hs/Mm GalNAc-conjugated STAT3 oligonucleotides for inhibiting STAT3 expression in liver (FIG. 25).

TABLE 13
GalNAc-Conjugated Human/Mouse STAT3 RNAi
Oligonucleotides for Endogenous STAT3 Screen.

| | Unmodified | Unmodified | Modified | Modified |
|---|---|---|---|---|
| | Sense | Antisense | Sense | Antisense |
| | Strand | strand | Strand | strand |
| STAT3-1383 | 946 | 1036 | 1126 | 1216 |
| STAT3-2135 | 945 | 1035 | 1125 | 1206 |
| STAT3-2136 | 935 | 1025 | 1115 | 1205 |
| STAT3-2138 | 938 | 1028 | 1118 | 1208 |
| STAT3-2139 | 940 | 1030 | 1120 | 1210 |
| STAT3-2143 | 936 | 1026 | 1116 | 1206 |
| STAT3-2144 | 937 | 1027 | 1117 | 1207 |
| STAT3-2145 | 942 | 1032 | 1122 | 1212 |
| STAT3-2411 | 941 | 1031 | 1121 | 1211 |
| STAT3-2622 | 944 | 1034 | 1124 | 1214 |
| STAT3-4831 | 943 | 1033 | 1123 | 1213 |
| STAT3-4909 | 939 | 1029 | 1119 | 1209 |

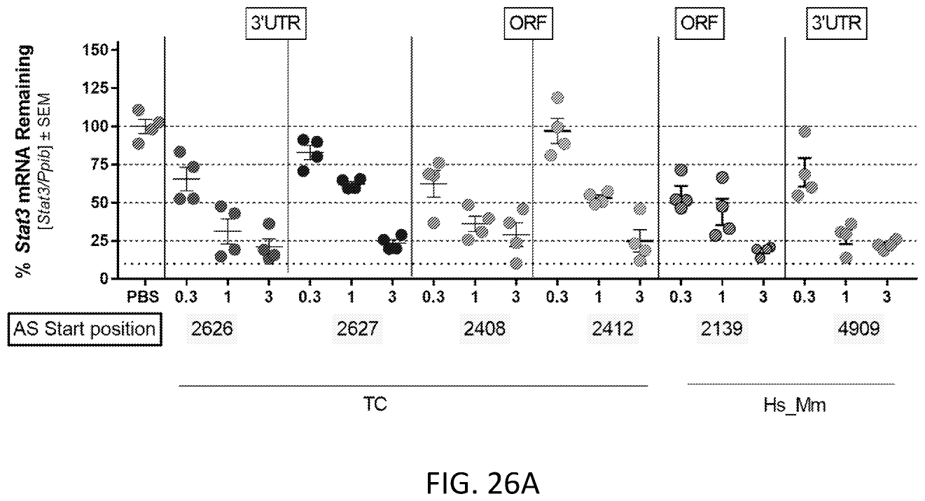

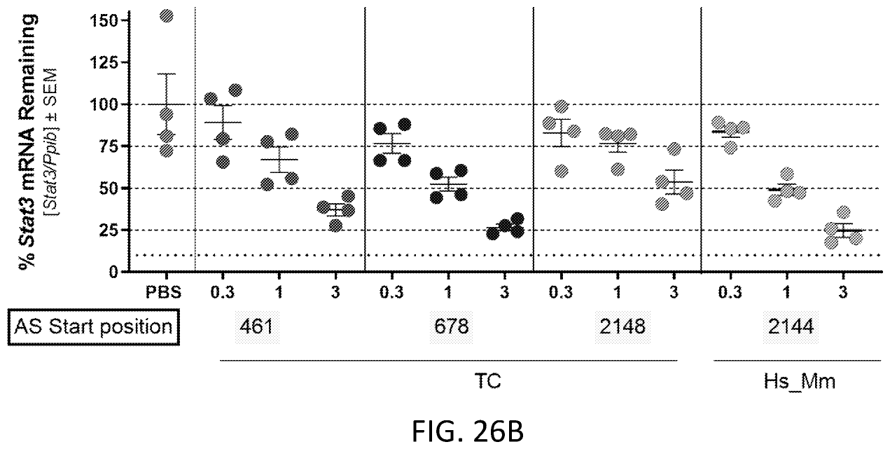

A subset of the GalNAc-conjugated STAT3 oligonucleotides tested in FIGS. 24 and 25 were further validated in a dosing study. Specifically, dosing studies were carried out using ten GalNAc-conjugated STAT3 oligonucleotides (STAT3-2626, STAT3-2627, STAT3-2408, STAT3-2412, STAT3-2139, STAT3-4909, STAT3-461, STAT3-678, STAT3-2148, and STAT3-2144). Mice endogenously expressing mouse STAT3 were subcutaneously injected with 0.3 mg/kg, 1 mg/kg, or 3 mg/kg oligonucleotide. Livers were collected after five days, and mouse STAT3 expression was measured to determine a potent dose (FIGS. 26A and 26B). Overall, the endogenous mouse STAT3 expression studies identified several potential GalNAc-conjugated STAT3 oligonucleotides for inhibiting mouse STAT3 expression in liver.

### Hs Specific

Using the HDI model described in Example 12, human specific GalNAc-conjugated STAT3 oligonucleotides were evaluated. Specifically, 6-8-week-old female CD-1 mice (n=4-5) were subcutaneously administered the indicated GalNAc-conjugated STAT3 oligonucleotides (Table 14) at a dose of 1 mg/kg formulated in PBS. A control group of mice (n=3-4) were administered only PBS. Three days later (72 hours), the mice were hydrodynamically injected (HDI) with a DNA plasmid encoding the full human STAT3 gene (25 μg) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine STAT3 mRNA levels.

TABLE 14
GalNAc-Conjugated Human STAT3 RNAi Oligonucleotides
for Exogenous STAT3Screen.

| | Unmodified | Unmodified | Modified | Modified |
|---|---|---|---|---|
| | Sense | Antisense | Sense | Antisense |
| | Strand | strand | Strand | strand |
| STAT3-424 | 926 | 1016 | 1106 | 1196 |
| STAT3-425 | 932 | 1022 | 1112 | 1202 |
| STAT3-426 | 915 | 1005 | 1095 | 1185 |
| STAT3-429 | 921 | 1011 | 1101 | 1191 |
| STAT3-430 | 923 | 1013 | 1103 | 1193 |
| STAT3-432 | 924 | 1014 | 1104 | 1194 |
| STAT3-433 | 918 | 1008 | 1098 | 1188 |
| STAT3-1067 | 917 | 1007 | 1097 | 1187 |
| STAT3-1670 | 919 | 1009 | 1099 | 1189 |
| STAT3-1241 | 930 | 1020 | 1110 | 1200 |
| STAT3-1388 | 920 | 1010 | 1100 | 1190 |
| STAT3-1671 | 934 | 1024 | 1114 | 1204 |
| STAT3-1672 | 931 | 1021 | 1111 | 1201 |
| STAT3-1673 | 914 | 1004 | 1094 | 1184 |
| STAT3-1674 | 929 | 1019 | 1109 | 1199 |
| STAT3-1813 | 928 | 1018 | 1108 | 1198 |
| STAT3-1815 | 925 | 1015 | 1105 | 1195 |
| STAT3-1817 | 933 | 1023 | 1113 | 1203 |
| STAT3-2024 | 927 | 1017 | 1107 | 1197 |
| STAT3-2404 | 916 | 1006 | 1096 | 1186 |
| STAT3-2405 | 922 | 1012 | 1102 | 1192 |

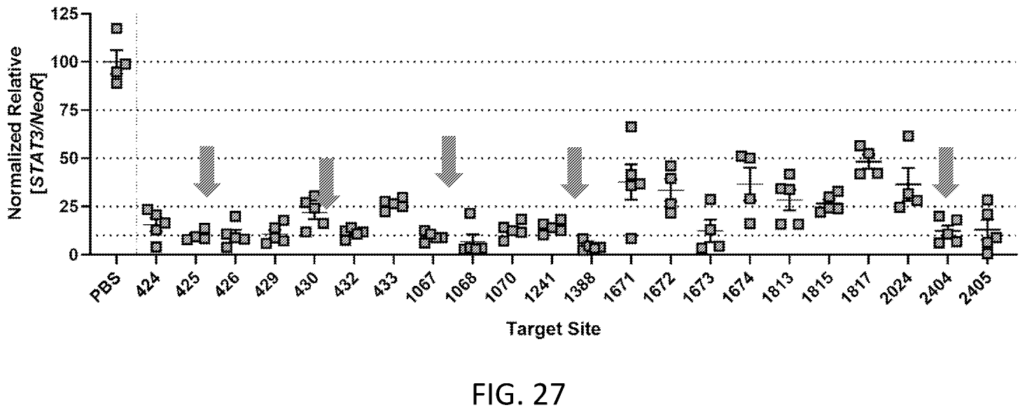

The results in FIG. 27 demonstrate that GalNAc-conjugated STAT3 oligonucleotides designed to target human STAT3 mRNA inhibited human STAT3 mRNA expression in HDI mice, as determined by a reduction in the amount of human STAT3 mRNA expression in liver samples from HDI mice treated with GalNAc-conjugated STAT3 oligonucleotides relative to control HDI mice treated with only PBS.

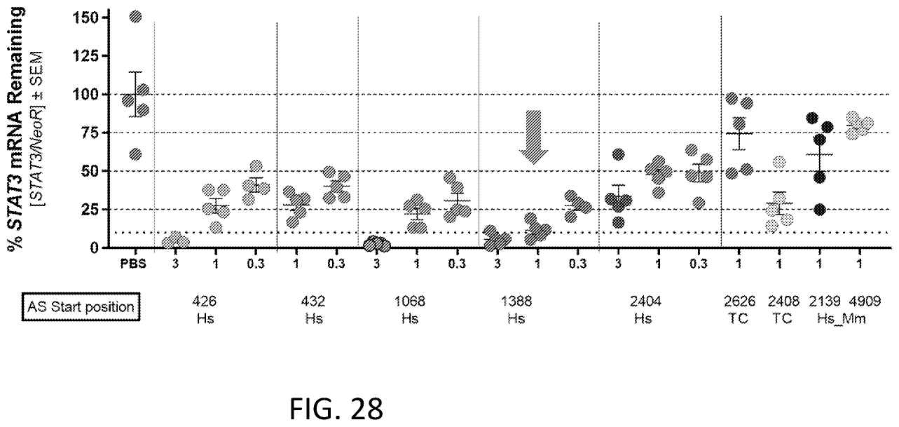

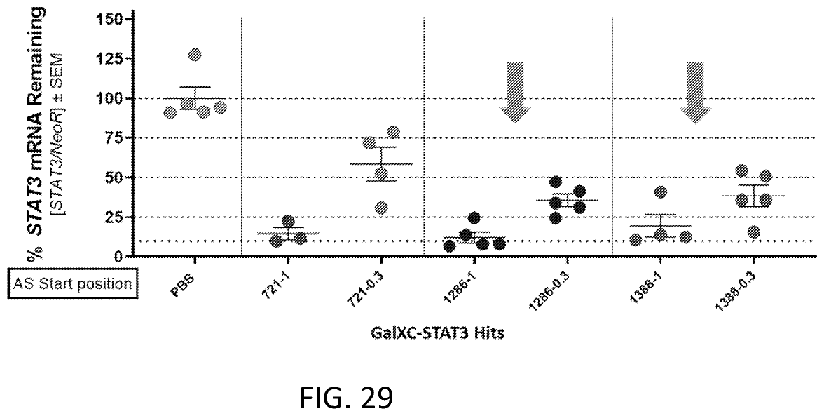

A subset of the GalNAc-conjugated STAT3 oligonucleotides tested in FIG. 27 were further validated in a dosing study. Specifically, dosing studies were carried out using five GalNAc-conjugated STAT3 oligonucleotides (STAT3-426, STAT3-432, STAT3-1068, STAT3-1388, and STAT3-2404). Mice were hydrodynamically injected as described above and treated with 0.3 mg/kg, 1 mg/kg, or 3 mg/kg of oligonucleotide. Livers were collected after one day, and human STAT3 expression was measured to determine a potent dose (FIG. 28). A dose of 1 mg/kg was capable of reducing STAT3 mRNA by about 75%, thereby identifying several potential GalNAc-conjugated STAT3 oligonucleotides for inhibiting STAT3 expression in liver. The best 2 sequences from FIG. 23 and the best sequence from FIG. 28 are tested in the final HDI screen (FIG. 29).

### Example 14: Specific STAT3 Inhibition by GalNAc-Conjugated STAT3 Oligonucleotides

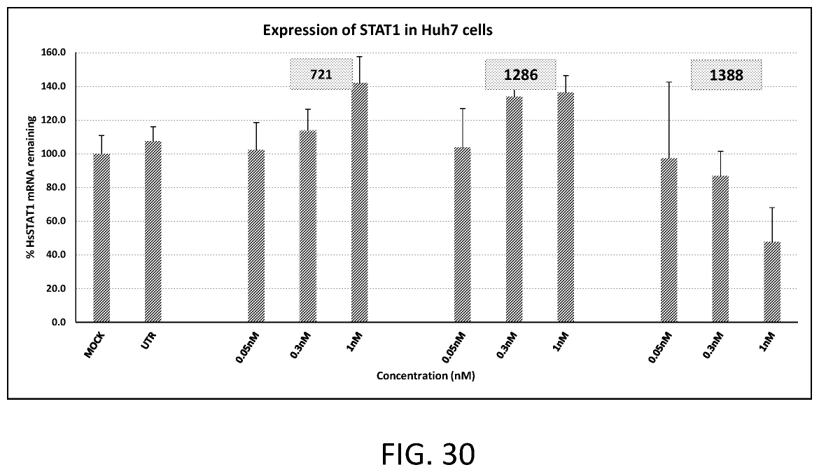

The specificity of the GalNAc-conjugated STAT3 oligonucleotides to inhibit STAT3 rather than a family member (e.g. STAT1) was measured. Specifically, Huh7 cells expressing endogenous STAT1 were treated for 24 hours with 0.05 nM, 0.3 nM, or 1 nM of a GalNAc-conjugated STAT3 oligonucleotide (STAT3-721, STAT3-1286, and STAT3-1388) using lipofectamine as transfection agent. The percent (%) remaining mRNA was measured compared to a mock control (PBS; no lipofectamine or siRNA) and UTR (un-transfected; treated with lipofectamine but no siRNA) (Table 15 and FIG. 30). STAT3 721 and 1286 did not downregulate human STAT1 but STAT3 1388 did (Table 15). Oligonucleotides did not downregulate STAT1 expression demonstrating a specificity for STAT3 with limited off-target effects for STAT1.

TABLE 15
STAT1 Expression

| Sample | Concentration | | % Expression | SEM |
|---|---|---|---|---|
| Mock | | | 100.0 | 10.8 |
| UTR | | | 107.5 | 8.4 |
| STAT3-721 | 0.05 | nM | 102.3 | 16.2 |
| | 0.3 | nM | 113.6 | 12.8 |
| | 1 | nM | 142.0 | 15.6 |
| STAT3-1286 | 0.05 | nM | 103.7 | 23.0 |
| | 0.3 | nM | 133.8 | 9.6 |
| | 1 | nM | 136.3 | 10.0 |
| STAT3-1388 | 0.05 | nM | 97.3 | 45.2 |
| | 0.3 | nM | 86.8 | 14.6 |
| | 1 | nM | 47.7 | 20.3 |

SEQUENCE LISTING

| | | | | SEQ |
|---|---|---|---|---|
| Name | Description | Species | Sequence | ID NO |
| GalXC- | Unmodified | | GGUGGAUGAAACUCAGUUUAGCAGCCG | 1 |
| ALDH2- | 36 mer | | AAAGGCUGC | |
| C18 | | | | |
| GalXC- | Unmodified | | UAAACUGAGUUUCAUCCACCGG | 2 |
| ALDH2- | 22 mer | | | |
| C18 | | | | |
| GalXC- | Modified | | [mGs][mG][fU][mG][fG][mA][mU][fG][mA] | 3 |
| ALDH2- | 36 mer | | [fA][mA][fC][fU][mC][fA][mG][fU][mU][mU] | |
| C18 | | | [mA][mG][mC][mA][mG][mC][mC][mG][ademA- | |
| | | | C18][mA][mA][mG][mG][mC] | |
| | | | [mU][mG][mC] | |
| GalXC- | Modified | | [MePhosphonate-4O- | 4 |
| ALDH2- | 22 mer | | mUs][fAs][fA][fA][fC][mU][fG][mA][mG][fU] | |
| C18 | | | [mU][mU][mC][fA][mU][fC][mC][mA][fC][mCs] | |
| | | | [mGs][mG] | |
| GalXC- | Unmodified | | GGUGGAUGAAACUCAGUUUAGCAGCCG | 5 |
| ALDH2- | 36 mer | | AAAGGCUGC | |
| C22 | | | | |
| GalXC- | Unmodified | | UAAACUGAGUUUCAUCCACCGG | 6 |
| ALDH2- | 22 mer | | | |
| C22 | | | | |
| GalXC- | Modified | | [mGs][mG][fU][mG][fG][mA][mU][fG][mA] | 7 |
| ALDH2- | 36 mer | | [fA][mA][fC][fU][mC][fA][mG][fU][mU][mU] | |
| C22 | | | [mA][mG][mC][mA][mG][mC][mC][mG][ademA- | |
| | | | C22][mA][mA][mG][mG][mC] | |
| | | | [mU][mG][mC] | |
| GalXC- | Modified | | [MePhosphonate-4O- | 8 |
| ALDH2- | 22 mer | | mUs][fAs][fA][fA][fC][mU][fG][mA][mG][fU] | |
| C22 | | | [mU][mU][mC][fA][mU][fC][mC][mA][fC][mCs] | |
| | | | [mGs][mG] | |
| GalXC- | Unmodified | | AGGACGACUUUGAUUUCAAAGCAGCCG | 9 |
| STAT3- | 36 mer | | AAAGGCUGC | |
| 838 | | | | |
| GalXC- | Unmodified | | UUUGAAAUCAAAGUCGUCCUGG | 10 |
| STAT3- | 22 mer | | | |
| 838 | | | | |
| GalXC- | Modified | | [mAs][mG][mG][mA][mC][mG][mA][fC][fU] | 11 |
| STAT3- | 36 mer | | [fU][fU][mG][mA][mU][mU][mU][mC][mA][mA] | |
| 838 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 12 |
| STAT3- | 22 mer | | mUs][fUs][fU][fG][fA][mA][fA][mU][mC][fA] | |
| 838 | | | [mA][mA][mG][fU][mC][mG][mU][mC][mC] | |
| | | | [mUs][mGs][mG] | |
| GalXC- | Unmodified | | UCAAAUUUCCUGAGUUGAAAGCAGCCG | 13 |
| STAT3- | 36 mer | | AAAGGCUGC | |
| 1390 | | | | |
| GalXC- | Unmodified | | UUUCAACUCAG | 14 |
| STAT3- | 22 mer | | GAAUUUGAGG | |
| 1390 | | | | |
| GalXC- | Modified | | [mUs][mC][mA][mA][mA][mU][mU][fU][fC] | 15 |
| STAT3- | 36 mer | | [fC][fU][mG][mA][mG][mU][mU][mG][mA][mA] | |
| 1390 | | | [mA][mG][mC][mA][mG][mC][mC][mG][ademA- | |
| | | | GalNAc][ademA-GalNAc][ademA- | |
| | | | GalNAc][mG\|mG\|mC\|mU][mG\|mC] | |
| GalXC- | Modified | | [MePhosphonate-4O- | 16 |
| STAT3- | 22 mer | | mUs][fUs][fU][fC][fA][mA][fC][mU][mC][fA] | |
| 1390 | | | [mG][mG][mA][fA][mA][mU][mU][mU][mG] | |
| | | | [mAs][mGs][mG] | |
| GalXC- | Unmodified | | AUUUCCUGAGUUGAAUUAUAGCAGCCG | 17 |
| STAT3- | 36 mer | | AAAGGCUGC | |
| 1394 | | | | |
| GalXC- | Unmodified | | UAUAAUUCAACUCAGGAAAUGG | 18 |
| STAT3- | 22 mer | | | |
| 1394 | | | | |
| GalXC- | Modified | | [mAs][mU][mU][mU][mC][mC][mU][fG][fA] | 19 |
| STAT3- | 36 mer | | [fG][fU][mU][mG][mA][mA][mU][mU][mA] | |
| 1394 | | | [mU][mA][mG][mC][mA][mG][mC][mC][mG][ademA- | |
| | | | GalNAc][ademA-GalNAc][ademA- | |
| | | | GalNac][mG][mG][mC][mU][mG][mC] | |
| GalXC- | Modified | | [MePhosphonate-4O- | 20 |
| STAT3- | 22 mer | | mUs][fAs][fU][fA][fA][mU][fU][mC][mA][fA] | |
| 1394 | | | [mC][mU][mC][fA][mG][mG][mA][mA][mA] | |
| | | | [mUs][mGs][mG] | |
| GalXC- | Unmodified | | CCUGAGUUGAAUUAUCAGCAGCAGCCG | 21 |
| STAT3- | 36 mer | | AAAGGCUGC | |
| 1398 | | | | |
| GalXC- | Unmodified | | UGCUGAUAAUUCAACUCAGGGG | 22 |
| STAT3- | 22 mer | | | |
| 1398 | | | | |
| GalXC- | Modified | | [mCs][mC][mU][mG][mA][mG][mu][fU][fG][fA] | |
| STAT3- | 36 mer | | [fA][mU][mU][mA][mU][mC][mA][mG][mC] | 23 |
| 1398 | | | [mA][mG][mC][mA][mG][mC][mC][mG][ademA- | |
| | | | GalNAc][ademA-GalNAc][ademA- | |
| | | | GalNac][mG][mG][mC][mU][mG][mC] | |
| GalXC- | Modified | | [MePhosphonate-4O- | 24 |
| STAT3- | 22 mer | | mUs][fGs][fC][fU][fG][mA][fU][mA][mA][fU] | |
| 1398 | | | [mU][mC][mA][fA][mC][mU][mC][mA][mG] | |
| | | | [mGs][mGs][mG] | |
| GalXC- | Unmodified | | CUGAGUUGAAUUAUCAGCUAGCAGCCG | 25 |
| STAT3- | 36 mer | | AAAGGCUGC | |
| 1399 | | | | |
| GalXC- | Unmodified | | UAGCUGAUAAUUCAACUCAGGG | 26 |
| STAT3- | 22 mer | | | |
| 1399 | | | | |
| GalXC- | Modified | | [mCs][mU][mG][mA][mG][mU][mU][fG][fA] | 27 |
| STAT3- | 36 mer | | [fA][fU][mU][mA][mU][mC][mA][mG][mC][mU] | |
| 1399 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 28 |
| STAT3- | 22 mer | | mUs][fAs][fG][fC][fU][mG][fA][mU][mA][fA] | |
| 1399 | | | [mU][mU][mC][fA][mA][mC][mU][mC][mA] | |
| | | | [mGs][mGs][mG] | |
| GalXC- | Unmodified | | UGAGUUGAAUUAUCAGCUUAGCAGCCG | 29 |
| STAT3- | 36 mer | | AAAGGCUGC | |
| 1400 | | | | |
| GalXC- | Unmodified | | UAAGCUGAUAAUUCAACUCAGG | 30 |
| STAT3- | 22 mer | | | |
| 1400 | | | | |
| GalXC- | Modified | | [mUs][mG][mA][mG][mU][mU][mG][fA][fA][fU] | 31 |
| STAT3- | 36 mer | | [fU][mA][mU][mC][mA][mG][mC][mU][mU] | |
| 1400 | | | [mA][mG][mC][mA][mG][mC][mC][mG][ademA- | |
| | | | GalNAc][ademA-GalNAc][ademA- | |
| | | | GalNac][mG][mG][mC][mU][mG][mC] | |
| GalXC- | Modified | | [MePhosphonate-4O- | 32 |
| STAT3- | 22 mer | | mUs][fAs][fA][fG][fC][mU][fG][mA][mU][fA] | |
| 1400 | | | [mA][mU][mU][fC][mA][mA][mC][mU][mC] | |
| | | | [mAs][mGs][mG] | |
| GalXC- | Unmodified | | GAGUUGAAUUAUCAGCUUAAGCAGCCG | 33 |
| STAT3- | 36 mer | | AAAGGCUGC | |
| 1401 | | | | |
| GalXC- | Unmodified | | UUAAGCUGAUAAUUCAACUCGG | 34 |
| STAT3- | 22 mer | | | |
| 1401 | | | | |
| GalXC- | Modified | | [mGs][mA][mG][mU][mU][mG][mA][fA][fU] | 35 |
| STAT3- | 36 mer | | [fU][fA][mU][mC][mA][mG][mC][mU][mU][mA] | |
| 1401 | | | mA][mG][mC][mA][mG][mC][mC][mG][ademA- | |
| | | | GalNAc][ademA-GalNAc][ademA- | |
| | | | GalNAc][mG][mG][mC\|mU][mG\|mC] | |
| GalXC- | Modified | | [MePhosphonate-4O- | 36 |
| STAT3- | 22 mer | | mUs][fUs][fA][fA][fG][mC][fU][mG][mA][fU] | |
| 1401 | | | [mA][mA][mU][fU][mC][mA][mA][mC][mU][mCs] | |
| | | | [mGs][mG] | |
| GalXC- | Unmodified | | AGUUGAAUUAUCAGCUUAAAGCAGCCG | 37 |
| STAT3- | 36 mer | | AAAGGCUGC | |
| 1402 | | | | |
| GalXC- | Unmodified | | UUUAAGCUGAUAAUUCAACUGG | 38 |
| STAT3- | 22 mer | | | |
| 1402 | | | | |
| GalXC- | Modified | | [mAs][mG][mU][mU][mG][mA][mA][fU][fU][fA] | 39 |
| STAT3- | 36 mer | | [fU][mC][mA][mG][mC][mU][mU][mA][mA] | |
| 1402 | | | [mA][mG][mC][mA][mG][mC][mC][mG][ademA- | |
| | | | GalNAc][ademA-GalNAc][ademA- | |
| | | | GalNAc][mG][mG][mC\|mU][mG\|mC] | |
| GalXC- | Modified | | [MePhosphonate-4O- | 40 |
| STAT3- | 22 mer | | mUs][fUs][fU][fA][fA][mG][fC][mU][mG][fA] | |
| 1402 | | | [mU][mA][mA][fU][mU][mC][mA][mA][mC] | |
| | | | [mUs][mGs][mG] | |
| GalXC- | Unmodified | | CAAUCCUGUGGUAUAACAUAGCAGCCG | 41 |
| STAT3- | 36 mer | | AAAGGCUGC | |
| 1759 | | | | |
| GalXC- | Unmodified | | UAUGUUAUACCACAGGAUUGGG | 42 |
| STAT3- | 22 mer | | | |
| 1759 | | | | |
| GalXC- | Modified | | [mCs][mA][mA][mU][mC][mC][mU][fG][fU] | 43 |
| STAT3- | 36 mer | | [fG][fG][mU][mA][mU][mA][mA][mC][mA][mU] | |
| 1759 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 44 |
| STAT3- | 22 mer | | mUs][fAs][fU][fG][fU][mU][fA][mU][mA][fC] | |
| 1759 | | | [mC][mA][mC][fA][mG][mG][mA][mU][mU][mGs] | |
| | | | [mGs][mG] | |
| GalXC- | Unmodified | | ACAAUAUCAUCGACCUUGUAGCAGCCG | 45 |
| STAT3- | 36 mer | | AAAGGCUGC | |
| 2029 | | | | |
| GalXC- | Unmodified | | UACAAGGUCGAUGAUAUUGUGG | 46 |
| STAT3- | 22 mer | | | |
| 2029 | | | | |
| GalXC- | Modified | | [mAs][mC][mA][mA][mU][mA][mU][fC][A][fU] | 47 |
| STAT3- | 36 mer | | [fC][mG][mA][mC][mC][mU][mU][mG][mU] | |
| 2029 | | | [mA][mG][mC][mA][mG][mC][mC][mG][ademA- | |
| | | | GalNAc][ademA-GalNAc][ademA- | |
| | | | GalNAc][mG][G][mC][mU][mG][mC] | |
| GalXC- | Modified | | [MePhosphonate-4O- | 48 |
| STAT3- | 22 mer | | mUs][fAs][fC][fA][fA][mG][fG][mU][mC][fG] | |
| 2029 | | | [mA][mU][mG][fA][mU][mA][mU][mU][mG] | |
| | | | [mUs][mGs][mG] | |
| GalXC- | Unmodified | | AUCAUCGACCUUGUGAAAAAGCAGCCG | 49 |
| STAT3- | 36 mer | | AAAGGCUGC | |
| 2034 | | | | |
| GalXC- | Unmodified | | UUUUUCACAAGGUCGAUGAUGG | 50 |
| STAT3- | 22 mer | | | |
| 2034 | | | | |
| GalXC- | Modified | | [mAs][mU][mC][mA][mU][mC][mG][fA][fC][fC] | 51 |
| STAT3- | 36 mer | | [fU][mU][mG][mU][mG][mA][mA][mA][mA] | |
| 2034 | | | [mA][mG][mC][mA][mG][mC][mC][mG][ademA- | |
| | | | GalNAc][ademA-GalNAc][ademA- | |
| | | | GalNac][mG][mG][mC][mU][mg][mC] | |
| GalXC- | Modified | | [MePhosphonate-4O- | 52 |
| STAT3- | 22 mer | | mUs][fUs][fU][fU][fU][mC][fA][mC][mA][fA] | |
| 2034 | | | [mG][mG][mU][fC][mG][mA][mU][mG][mA] | |
| | | | [mUs][mGs][mG] | |
| GalXC- | Unmodified | | CUGAAGACCAAGUUCAUCUAGCAGCCG | 53 |
| STAT3- | 36 mer | | AAAGGCUGC | |
| 2448 | | | | |
| GalXC- | Unmodified | | UAGAUGAACUU | 54 |
| STAT3- | 22 mer | | GGUCUUCAGGG | |
| 2448 | | | | |
| GalXC- | Modified | | [mCs][mU][mG][mA][mA][mG][mA][fC][fC][fA] | 55 |
| STAT3- | 36 mer | | [fA][mG][mU][mU][mC][mA][mU][mC][mU] | |
| 2448 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 56 |
| STAT3- | 22 mer | | mUs][fAs][fG][fA][fU][mG][fA][mA][mC][fU] | |
| 2448 | | | [mU][mG][mG][fU][mC][mU][mU][mC][mA][mGs] | |
| | | | [mGs][mG] | |
| GalXC- | Unmodified | | AUUCAUUGAUGCAGUUUGGAGCAGCCG | 57 |
| STAT3- | 36 mer | | AAAGGCUGC | |
| 2527 | | | | |
| GalXC- | Unmodified | | UCCAAACUGCAUCAAUGAAUGG | 58 |
| STAT3- | 22 mer | | | |
| 2527 | | | | |
| GalXC- | Modified | | [mAs][mU][mU][mC][mA][mU][mU][fG][fA][fU] | 59 |
| STAT3- | 36 mer | | [fG][mC][mA][mG][mU][mU][mU][mG][mG] | |
| 2527 | | | [mA][mG][mC][mA][mG][mC][mC][mG][ademA- | |
| | | | GalNAc][ademA-GalNAc][ademA- | |
| | | | GalNAc][mG][mG][mC\|mU][mG][mC] | |
| GalXC- | Modified | | [MePhosphonate-4O- | 60 |
| STAT3- | 22 mer | | mUs][fCs][fC][fA][fA][mA][fC][mU][mG][fC] | |
| 2527 | | | [mA][mU][mC][fA][mA][mU][mG][mA][mA] | |
| | | | [mUs][mGs][mG] | |
| GalXC- | Unmodified | | CCCAUCAAUGUUCUUUAGUAGCAGCCG | 61 |
| STAT3- | 36 mer | | AAAGGCUGC | |
| 4107 | | | | |
| GalXC- | Unmodified | | UACUAAAGAACAUUGAUGGGGG | 62 |
| STAT3- | 22 mer | | | |
| 4107 | | | | |
| GalXC- | Modified | | [mCs][mC][mC][mA][mU][mC][mA][fA][fU][fG] | 63 |
| STAT3- | 36 mer | | [fU][mU][mC][mU][mU][mU][mA][mG][mU] | |
| 4107 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 64 |
| STAT3- | 22 mer | | mUs][fAs][fC][fU][fA][mA][fA][mG][mA][fA] | |
| 4107 | | | [mC][mA][mU][fU][mG][mA][mU][mG][mG] | |
| | | | [mGs][mGs][mG] | |
| GalXC- | Unmodified | | AUCAAUGUUCUUUAGUUAUAGCAGCCG | 65 |
| STAT3- | 36 mer | | AAAGGCUGC | |
| 4110 | | | | |
| GalXC- | Unmodified | | UAUAACUAAAGAACAUUGAUGG | 66 |
| STAT3- | 22 mer | | | |
| 4110 | | | | |
| GalXC- | Modified | | [mAs][mU][mC][mA][mA][mU][mG][fU][fU][fC] | 67 |
| STAT3- | 36 mer | | [fU][mU][mU][mA][mG][mU][mU][mA][mU] | |
| 4110 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 68 |
| STAT3- | 22 mer | | mUs][fAs][fU][fA][fA][mC][fU][mA][mA][fA] | |
| 4110 | | | [mG][mA][mA][fC][mA][mU][mU][mG][mA] | |
| | | | [mUs][mGs][mG] | |
| GalXC- | Unmodified | | AGUUAUACAAUAAGCUGAAAGCAGCCG | 69 |
| STAT3- | 36 mer | | AAAGGCUGC | |
| 4123 | | | | |
| GalXC- | Unmodified | | UUUCAGCUUAUUGUAUAACUGG | 70 |
| STAT3- | 22 mer | | | |
| 4123 | | | | |
| GalXC- | Modified | | [mAs][mG][mU][mU][mA][mU][mA][fC][fA][fA] | 71 |
| STAT3- | 36 mer | | [fU][mA][mA][mG][mC][mU][mG][mA][mA] | |
| 4123 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 72 |
| STAT3- | 22 mer | | mUs][fUs][fU][fC][fA][mG][fC][mU][mU][fA] | |
| 4123 | | | [mU][mU][mG][fU][mA][mU][mA][mA][mC] | |
| | | | [mUs][mGs][mG] | |
| GalXC- | Unmodified | | AGUGUAAAAAUUUAUAUUAAGCAGCCG | 73 |
| STAT3- | 36 mer | | AAAGGCUGC | |
| 4435 | | | | |
| GalXC- | Unmodified | | UUAAUAUAAAUUUUUACACUGG | 74 |
| STAT3- | 22 mer | | | |
| 4435 | | | | |
| GalXC- | Modified | | [mAs][mG][mU][mG][mU][mA][mA][fA][fA][fA] | 75 |
| STAT3- | 36 mer | | [fU][mU][mU][mA][mU][mA][mU][mU][mA] | |
| 4435 | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| GalXC- | Modified | | [MePhosphonate-4O- | 76 |
| STAT3- | 22 mer | | mUs][fUs][fA][fA][fU][mA][fU][mA][mA][fA] | |
| 4435 | | | [mU][mU][mU][fU][mU][mA][mC][mA][mC] | |
| | | | [mUs][mGs][mG] | |
| GalXC- | Unmodified | | UUGUUUGUUUUUGUAUAUUAGCAGCCG | 77 |
| STAT3- | 36 mer | | AAAGGCUGC | |
| 4474 | | | | |
| GalXC- | Unmodified | | UUAAUAUAAAUUUUUACACUGG | 78 |
| STAT3- | 22 mer | | | |
| 4474 | | | | |
| GalXC- | Modified | | [mUs][mU][mG][mU][mU][mU][mG][fU][fU][fU] | 79 |
| STAT3- | 36 mer | | [fU][mU][mG][mU][mA][mU][mA][mU][mU] | |
| 4474 | | | [mA][mG][mC][mA][mG][mC][mC][mG][ademA- | |
| | | | GalNAc][ademA-GalNAc][ademA- | |
| | | | GalNac][mG][mG][mC][mU][mG][mC] | |
| GalXC- | Modified | | [MePhosphonate-4O- | 80 |
| STAT3- | 22 mer | | mUs][fAs][fA][fU][fA][mU][fA][mC][mA][fA] | |
| 4474 | | | [mA][mA][mA][fC][mA][mA][mA][mC][mA][mAs] | |
| | | | [mGs][mG] | |
| GalXC- | Modified | | [mAs][mU][mC][mA][mA][mU][mG][fU][fU][fC] | 81 |
| STAT3- | 36 mer | | [fU][mU][mU][mA][mG][mU][mU][mA][mU] | |
| 4110- | | | [mA][mG][mC][mA][mG][mC][mC][mG][ademA- | |
| C18 | | | C18][mA][mA]\|mG\|mG][mC][ mU][mG\|[mC] | |
| GalXC- | Modified | | [MePhosphonate-4O- | 82 |
| STAT3- | 22 mer | | mUs][fAs][fU][fA][fA][mC][fU][mA][mA][fA] | |
| 4110- | | | [mG][mA][mA][fC][mA][mU][mU][mG][mA] | |
| C18 | | | [mUs][mGs][mG] | |
| GalXC- | Modified | | [mAs][mG][mU][mU][mA][mU][mA][fC][fA][fA] | 83 |
| STAT3- | 36 mer | | [fU][mA][mA][mG][mC][mU][mG][mA][mA] | |
| 4123- | | | [mA][mG\|mC][mA][mG][mC][mC][mG][ademA- | |
| C18 | | | C18][mA][mA][mG][mG][mC][mU][mG][mC] | |
| GalXC- | Modified | | [MePhosphonate-4O- | 84 |
| STAT3- | 22 mer | | mUs][fUs][fU][fC][fA][mG][fC][mU][mU][fA] | |
| 4123- | | | [mU][mU][mG][fU][mA][mU][mA][mA][mC] | |
| C18 | | | [mUs][mGs][mG] | |
| | STAT3 | | GTCGCAGCCGAGGGAACAAGCCCCAACC | 85 |
| | Human (Hs) | | GGATCCTGGACAGGCACCCCGGCTTGGC | |
| | NM_001369512.1 | | GCTGTCTCTCCCCCTCGGCTCGGAGAGGC | |
| | (Genbank | | CCTTCGGCCTGAGGGAGCCTCGCCGCCC | |
| | RefSeq #) | | GTCCCCGGCACACGCGCAGCCCCGGCCT | |
| | | | CTCGGCCTCTGCCGGAGAAACAGGATGG | |
| | | | CCCAATGGAATCAGCTACAGCAGCTTGA | |
| | | | CACACGGTACCTGGAGCAGCTCCATCAG | |
| | | | CTCTACAGTGACAGCTTCCCAATGGAGCT | |
| | | | GCGGCAGTTTCTGGCCCCTTGGATTGAGA | |
| | | | GTCAAGATTGGGCATATGCGGCCAGCAA | |
| | | | AGAATCACATGCCACTTTGGTGTTTCATA | |
| | | | ATCTCCTGGGAGAGATTGACCAGCAGTA | |
| | | | TAGCCGCTTCCTGCAAGAGTCGAATGTTC | |
| | | | TCTATCAGCACAATCTACGAAGAATCAA | |
| | | | GCAGTTTCTTCAGAGCAGGTATCTTGAGA | |
| | | | AGCCAATGGAGATTGCCCGGATTGTGGC | |
| | | | CCGGTGCCTGTGGGAAGAATCACGCCTT | |
| | | | CTACAGACTGCAGCCACTGCGGCCCAGC | |
| | | | AAGGGGGCCAGGCCAACCACCCCACAGC | |
| | | | AGCCGTGGTGACGGAGAAGCAGCAGATG | |
| | | | CTGGAGCAGCACCTTCAGGATGTCCGGA | |
| | | | AGAGAGTGCAGGATCTAGAACAGAAAAT | |
| | | | GAAAGTGGTAGAGAATCTCCAGGATGAC | |
| | | | TTTGATTTCAACTATAAAACCCTCAAGAG | |
| | | | TCAAGGAGACATGCAAGATCTGAATGGA | |
| | | | AACAACCAGTCAGTGACCAGGCAGAAGA | |
| | | | TGCAGCAGCTGGAACAGATGCTCACTGC | |
| | | | GCTGGACCAGATGCGGAGAAGCATCGTG | |
| | | | AGTGAGCTGGCGGGGCTTTTGTCAGCGA | |
| | | | TGGAGTACGTGCAGAAAACTCTCACGGA | |
| | | | CGAGGAGCTGGCTGACTGGAAGAGGCGG | |
| | | | CAACAGATTGCCTGCATTGGAGGCCCGC | |
| | | | CCAACATCTGCCTAGATCGGCTAGAAAA | |
| | | | CTGGATAACGTCATTAGCAGAATCTCAA | |
| | | | CTTCAGACCCGTCAACAAATTAAGAAAC | |
| | | | TGGAGGAGTTGCAGCAAAAAGTTTCCTA | |
| | | | CAAAGGGGACCCCATTGTACAGCACCGG | |
| | | | CCGATGCTGGAGGAGAGAATCGTGGAGC | |
| | | | TGTTTAGAAACTTAATGAAAAGTGCCTTT | |
| | | | GTGGTGGAGCGGCAGCCCTGCATGCCCA | |
| | | | TGCATCCTGACCGGCCCCTCGTCATCAAG | |
| | | | ACCGGCGTCCAGTTCACTACTAAAGTCA | |
| | | | GGTTGCTGGTCAAATTCCCTGAGTTGAAT | |
| | | | TATCAGCTTAAAATTAAAGTGTGCATTGA | |
| | | | CAAAGACTCTGGGGACGTTGCAGCTCTC | |
| | | | AGAGGATCCCGGAAATTTAACATTCTGG | |
| | | | GCACAAACACAAAAGTGATGAACATGGA | |
| | | | AGAATCCAACAACGGCAGCCTCTCTGCA | |
| | | | GAATTCAAACACTTGACCCTGAGGGAGC | |
| | | | AGAGATGTGGGAATGGGGGCCGAGCCAA | |
| | | | TTGTGATGCTTCCCTGATTGTGACTGAGG | |
| | | | AGCTGCACCTGATCACCTTTGAGACCGA | |
| | | | GGTGTATCACCAAGGCCTCAAGATTGAC | |
| | | | CTAGAGACCCACTCCT | |
| | | | TGCCAGTTGTGGTGATCTCCAACATCTGT | |
| | | | CAGATGCCAAATGCCTGGGCGTCCATCCT | |
| | | | GTGGTACAACATGCTGACCAACAATCCC | |
| | | | AAGAATGTAAACTTTTTTACCAAGCCCCC | |
| | | | AATTGGAACCTGGGATCAAGTGGCCGAG | |
| | | | GTCCTGAGCTGGCAGTTCTCCTCCACCAC | |
| | | | CAAGCGAGGACTGAGCATCGAGCAGCTG | |
| | | | ACTACACTGGCAGAGAAACTCTTGGGAC | |
| | | | CTGGTGTGAATTATTCAGGGTGTCAGATC | |
| | | | ACATGGGCTAAATTTTGCAAAGAAAACA | |
| | | | TGGCTGGCAAGGGCTTCTCCTTCTGGGTC | |
| | | | TGGCTGGACAATATCATTGACCTTGTGAA | |
| | | | AAAGTACATCCTGGCCCTTTGGAACGAA | |
| | | | GGGTACATCATGGGCTTTATCAGTAAGG | |
| | | | AGCGGGAGCGGGCCATCTTGAGCACTAA | |
| | | | GCCTCCAGGCACCTTCCTGCTAAGATTCA | |
| | | | GTGAAAGCAGCAAAGAAGGAGGCGTCAC | |
| | | | TTTCACTTGGGTGGAGAAGGACATCAGC | |
| | | | GGTAAGACCCAGATCCAGTCCGTGGAAC | |
| | | | CATACACAAAGCAGCAGCTGAACAACAT | |
| | | | GTCATTTGCTGAAATCATCATGGGCTATA | |
| | | | AGATCATGGATGCTACCAATATCCTGGTG | |
| | | | TCTCCACTGGTCTATCTCTATCCTGACAT | |
| | | | TCCCAAGGAGGAGGCATTCGGAAAGTAT | |
| | | | TGTCGGCCAGAGAGCCAGGAGCATCCTG | |
| | | | AAGCTGACCCAGGTAGCGCTGCCCCATA | |
| | | | CCTGAAGACCAAGTTTATCTGTGTGACAC | |
| | | | CA | |
| | | | ACGACCTGCAGCAATACCATTGACCTGC | |
| | | | CGATGTCCCCCCGCACTTTAGATTCATTG | |
| | | | ATGCAGTTTGGAAATAATGGTGAAGGTG | |
| | | | CTGAACCCTCAGCAGGAGGGCAGTTTGA | |
| | | | GTCCCTCACCTTTGACATGGAGTTGACCT | |
| | | | CGGAGTGCGCTACCTCCCCCATGTGAGG | |
| | | | AGCTGAGAACGGAAGCTGCAGAAAGATA | |
| | | | CGACTGAGGCGCCTACCTGCATTCTGCCA | |
| | | | CCCCTCACACAGCCAAACCCCAGATCAT | |
| | | | CTGAAACTACTAACTTTGTGGTTCCAGAT | |
| | | | TTTTTTTAATCTCCTACTTCTGCTATCTTT | |
| | | | GAGCAATCTGGGCACTTTTAAAAATAGA | |
| | | | GAAATGAGTGAATGTGGGTGATCTGCTTT | |
| | | | TATCTAAATGCAAATAAGGATGTGTTCTC | |
| | | | TGAGACCCATGATCAGGGGATGTGGCGG | |
| | | | GGGGTGGCTAGAGGGAGAAAAAGGAAA | |
| | | | TGTCTTGTGTTGTTTTGTTCCCCTGCCCTC | |
| | | | CTTTCTCAGCAGCTTTTTGTTATTGTTGTT | |
| | | | GTTGTTCTTAGACAAGTGCCTCCTGGTGC | |
| | | | CTGCGGCATCCTTCTGCCTGTTTCTGTAA | |
| | | | GCAAATGCCACAGGCCACCTATAGCTAC | |
| | | | ATACTCCTGGCATTGCACTTTTTAACCTT | |
| | | | GCTGACATCCAAATAGAAGATAGGACTA | |
| | | | TCTAAGCCCTAGGTTTCTTTTTAAATTAA | |
| | | | GAAATAATAACAATTAAAGGGCAAAAAA | |
| | | | CACTGTATCAGCATAGCCTTTCTGTATTT | |
| | | | AAGAAACTTAAGCAGCCGGGCATGGTGG | |
| | | | CTCACGCCTGTAATCCCAGCACTTTGGGA | |
| | | | GGCCGAGGCGGATCATAAGGTCAGGAGA | |
| | | | TCAAGACCATCCTGGCTAACACGGTGAA | |
| | | | ACCCCGTCTCTACTAAAAGTACAAAAAA | |
| | | | TTAGCTGGGTGTGGTGGTGGGCGCC | |
| | | | TGTAGTCCCAGCTACTCGGGAGGCTGAG | |
| | | | GCAGGAGAATCGCTTGAACCTGAGAGGC | |
| | | | GGAGGTTGCAGTGAGCCAAAATTGCACC | |
| | | | ACTGCACACTGCACTCCATCCTGGGCGAC | |
| | | | AGTCTGAGACTCTGTCTCAAAAAAAAAA | |
| | | | AAAAAAAAAAGAAACTTCAGTTAACAGC | |
| | | | CTCCTTGGTGCTTTAAGCATTCAGCTTCC | |
| | | | TTCAGGCTGGTAATTTATATAATCCCTGA | |
| | | | AACGGGCTTCAGGTCAAACCCTTAAGAC | |
| | | | ATCTGAAGCTGCAACCTGGCCTTTGGTGT | |
| | | | TGAAATAGGAAGGTTTAAGGAGAATCTA | |
| | | | AGCATTTTAGACTTTTTTTTATAAATAGA | |
| | | | CTTATTTTCCTTTGTAATGTATTGGCCTTT | |
| | | | TAGTGAGTAAGGCTGGGCAGAGGGTGCT | |
| | | | TACAACCTTGACTCCCTTTCTCCCTGGAC | |
| | | | TTGATCTGCTGTTTCAGAGGCTAGGTTGT | |
| | | | TTCTGTGGGTGCCTTATCAGGGCTGGGAT | |
| | | | ACTTCTGATTCTGGCTTCCTTCCTGCCCC | |
| | | | ACCCTCCCGACCCCAGTCCCCCTGATCCT | |
| | | | GCTAGAGGCATGTCTCCTTGCGTGTCTAA | |
| | | | AGGTCCCTCATCCTGTTTGTTTTAGGAAT | |
| | | | CCTGGTCTCAGGACCTCATGGAAGAAGA | |
| | | | GGGGGAGAGAGTTACAGGTTGGACATGA | |
| | | | TGCACACTATGGGGCCCCAGCGACGTGT | |
| | | | CTGGTTGAGCTCAGGGAATATGGTTCTTA | |
| | | | GCCAGTTTCTTGGTGATATCCAGTGGCAC | |
| | | | TTGTAATGGCGTCTTCATTCAGTTCA | |
| | | | TGCAGGGCAAAGGCTTACTGATAAACTT | |
| | | | GAGTCTGCCCTCGTATGAGGGTGTATACC | |
| | | | TGGCCTCCCTCTGAGGCTGGTGACTCCTC | |
| | | | CCTGCTGGGGCCCCACAGGTGAGGCAGA | |
| | | | ACAGCTAGAGGGCCTCCCCGCCTGCCCG | |
| | | | CCTTGGCTGGCTAGCTCGCCTCTCCTGTG | |
| | | | CGTATGGGAACACCTAGCACGTGCTGGA | |
| | | | TGGGCTGCCTCTGACTCAGAGGCATGGC | |
| | | | CGGATTTGGCAACTCAAAACCACCTTGCC | |
| | | | TCAGCTGATCAGAGTTTCTGTGGAATTCT | |
| | | | GTTTGTTAAATCAAATTAGCTGGTCTCTG | |
| | | | AATTAAGGGGGAGACGACCTTCTCTAAG | |
| | | | ATGAACAGGGTTCGCCCCAGTCCTCCTGC | |
| | | | CTGGAGACAGTTGATGTGTCATGCAGAG | |
| | | | CTCTTACTTCTCCAGCAACACTCTTCAGT | |
| | | | ACATAATAAGCTTAACTGATAAACAGAA | |
| | | | TATTTAGAAAGGTGAGACTTGGGCTTACC | |
| | | | ATTGGGTTTAAATCATAGGGACCTAGGG | |
| | | | CGAGGGTTCAGGGCTTCTCTGGAGCAGA | |
| | | | TATTGTCAAGTTCATGGCCTTAGGTAGCA | |
| | | | TGTATCTGGTCTTAACTCTGATTGTAGCA | |
| | | | AAAGTTCTGAGAGGAGCTGAGCCCTGTT | |
| | | | GTGGCCCATTAAAGAACAGGGTCCTCAG | |
| | | | GCCCTGCCCGCTTCCTGTCCACTGCCCCC | |
| | | | TCCCCATCCCCAGCCCAGCCGAGGGAAT | |
| | | | CCCGTGGGTTGCTTACCTACCTATAAGGT | |
| | | | GGTTTATAAGCTGCTGTCCTGGCCACTGC | |
| | | | ATTCAAATTCCAATGTGTACTTCATAGTG | |
| | | | TAAAAATTTATATTATTGTGAGGTTTTTT | |
| | | | GTCTTTTTTTTTTTTTTTTTTTTTTGGTATA | |
| | | | TTGCTGTATCTACTTTAACTTCCAGAAAT | |
| | | | AAACGTTATATAGGAACCGTC | |
| | Stem Loop | | GCAGCCGAAAGGCUGC | 86 |
| GalXC- | Modified | | [mAs][mU][mC][mA][mA][mU][mG][fU][fU][fC] | 87 |
| STAT3- | 36 mer | | [fU][mU][mU][mA][mG][mU][mU][mA][mU] | |
| 2029 | | | [mA][mG][mC][mA][mG][mC][mC][mG][ademA- | |
| | | | C18][mA][mA\|mG\|mG][mC\|mU][mG\|mC] | |
| STAT3- | Modified | | [mAs][mG][mU][mU][mA][mU][mA][fC][fA][fA] | 88 |
| 4123- | 36 mer | | [fU][mA][mA][mG][mC][mU][mG][mA][mA] | |
| C18 | | | [mA][mG][mC][mA][mG][mC][mC][mG][ademA- | |
| | | | C18][mA][mA][mG][mG][mC][mU][mG][mC] | |
| STAT3- | Sense | | CACUUUGGUGUUUCAUAAU | 89 |
| 370 | 19 mer | | | |
| STAT3- | Sense | | CUUUGGUGUUUCAUAAUCU | 90 |
| 372 | 19 mer | | | |
| STAT3- | Sense | | CCUGCAAGAGUCGAAUGUU | 91 |
| 424 | 19 mer | | | |
| STAT3- | Sense | | CUGCAAGAGUCGAAUGUUC | 92 |
| 425 | 19 mer | | | |
| STAT3- | Sense | | UGCAAGAGUCGAAUGUUCU | 93 |
| 426 | 19 mer | | | |
| STAT3- | Sense | | AAGAGUCGAAUGUUCUCUA | 94 |
| 429 | 19 mer | | | |
| STAT3- | Sense | | AGAGUCGAAUGUUCUCUAU | 95 |
| 430 | 19 mer | | | |
| STAT3- | Sense | | AGUCGAAUGUUCUCUAUCA | 96 |
| 432 | 19 mer | | | |
| STAT3- | Sense | | GUCGAAUGUUCUCUAUCAG | 97 |
| 433 | 19 mer | | | |
| STAT3- | Sense | | ACGAAGAAUCAAGCAGUUU | 98 |
| 460 | 19 mer | | | |
| STAT3- | Sense | | CGAAGAAUCAAGCAGUUUC | 99 |
| 461 | 19 mer | | | |
| STAT3- | Sense | | GAAGAAUCAAGCAGUUUCU | 100 |
| 462 | 19 mer | | | |
| STAT3- | Sense | | AUCUUGAGAAGCCAAUGGA | 101 |
| 492 | 19 mer | | | |
| STAT3- | Sense | | AGGAUCUAGAACAGAAAAU | 102 |
| 678 | 19 mer | | | |
| STAT3- | Sense | | AUCUAGAACAGAAAAUGAA | 103 |
| 681 | 19 mer | | | |
| STAT3- | Sense | | CCAGGAUGACUUUGAUUUC | 104 |
| 715 | 19 mer | | | |
| STAT3- | Sense | | CAGGAUGACUUUGAUUUCA | 105 |
| 716 | 19 mer | | | |
| STAT3- | Sense | | AGGAUGACUUUGAUUUCAA | 106 |
| 717 | 19 mer | | | |
| STAT3- | Sense | | AUGACUUUGAUUUCAACUA | 107 |
| 720 | 19 mer | | | |
| STAT3- | Sense | | UGACUUUGAUUUCAACUAU | 108 |
| 721 | 19 mer | | | |
| STAT3- | Sense | | GACUUUGAUUUCAACUAUA | 109 |
| 722 | 19 mer | | | |
| STAT3- | Sense | | ACUUUGAUUUCAACUAUAA | 110 |
| 723 | 19 mer | | | |
| STAT3- | Sense | | CUUUGAUUUCAACUAUAAA | 111 |
| 724 | 19 mer | | | |
| STAT3- | Sense | | AAGAUCUGAAUGGAAACAA | 112 |
| 768 | 19 mer | | | |
| STAT3- | Sense | | AUCUGAAUGGAAACAACCA | 113 |
| 771 | 19 mer | | | |
| STAT3- | Sense | | CUGAAUGGAAACAACCAGU | 114 |
| 773 | 19 mer | | | |
| STAT3- | Sense | | AGAAAACUGGAUAACGUCA | 115 |
| 1000 | 19 mer | | | |
| STAT3- | Sense | | GAAAACUGGAUAACGUCAU | 116 |
| 1001 | 19 mer | | | |
| STAT3- | Sense | | AAACUGGAUAACGUCAUUA | 117 |
| 1003 | 19 mer | | | |
| STAT3- | Sense | | CUGGAUAACGUCAUUAGCA | 118 |
| 1006 | 19 mer | | | |
| STAT3- | Sense | | GGAUAACGUCAUUAGCAGA | 119 |
| 1008 | 19 mer | | | |
| STAT3- | Sense | | GAUAACGUCAUUAGCAGAA | 120 |
| 1009 | 19 mer | | | |
| STAT3- | Sense | | AUAACGUCAUUAGCAGAAU | 121 |
| 1010 | 19 mer | | | |
| STAT3- | Sense | | AACAAAUUAAGAAACUGGA | 122 |
| 1047 | 19 mer | | | |
| STAT3- | Sense | | GAGUUGCAGCAAAAAGUUU | 123 |
| 1067 | 19 mer | | | |
| STAT3- | Sense | | AGUUGCAGCAAAAAGUUUC | 124 |
| 1068 | 19 mer | | | |
| STAT3- | Sense | | CUGUUUAGAAACUUAAUGA | 125 |
| 1145 | 19 mer | | | |
| STAT3- | Sense | | AGAAACUUAAUGAAAAGUG | 126 |
| 1151 | 19 mer | | | |
| STAT3- | Sense | | CAGUUCACUACUAAAGUCA | 127 |
| 1241 | 19 mer | | | |
| STAT3- | Sense | | GUCAAAUUCCCUGAGUUGA | 128 |
| 1268 | 19 mer | | | |
| STAT3- | Sense | | AAUUCCCUGAGUUGAAUUA | 129 |
| 1272 | 19 mer | | | |
| STAT3- | Sense | | AUUCCCUGAGUUGAAUUAU | 130 |
| 1273 | 19 mer | | | |
| STAT3- | Sense | | UCCCUGAGUUGAAUUAUCA | 131 |
| 1275 | 19 mer | | | |
| STAT3- | Sense | | CCUGAGUUGAAUUAUCAGC | 132 |
| 1277 | 19 mer | | | |
| STAT3- | Sense | | CUGAGUUGAAUUAUCAGCU | 133 |
| 1278 | 19 mer | | | |
| STAT3- | Sense | | UGAGUUGAAUUAUCAGCUU | 134 |
| 1279 | 19 mer | | | |
| STAT3- | Sense | | GAGUUGAAUUAUCAGCUUA | 135 |
| 1280 | 19 mer | | | |
| STAT3- | Sense | | AGUUGAAUUAUCAGCUUAA | 136 |
| 1281 | 19 mer | | | |
| STAT3- | Sense | | GUUGAAUUAUCAGCUUAAA | 137 |
| 1282 | 19 mer | | | |
| STAT3- | Sense | | UUGAAUUAUCAGCUUAAAA | 138 |
| 1283 | 19 mer | | | |
| STAT3- | Sense | | UGAAUUAUCAGCUUAAAAU | 139 |
| 1284 | 19 mer | | | |
| STAT3- | Sense | | AAUUAUCAGCUUAAAAUUA | 140 |
| 1286 | 19 mer | | | |
| STAT3- | Sense | | AUUAUCAGCUUAAAAUUAA | 141 |
| 1287 | 19 mer | | | |
| STAT3- | Sense | | CAGCUUAAAAUUAAAGUGU | 142 |
| 1292 | 19 mer | | | |
| STAT3- | Sense | | AGCUUAAAAUUAAAGUGUG | 143 |
| 1293 | 19 mer | | | |
| STAT3- | Sense | | AAAUUAAAGUGUGCAUUGA | 144 |
| 1299 | 19 mer | | | |
| STAT3- | Sense | | AAGUGUGCAUUGACAAAGA | 145 |
| 1305 | 19 mer | | | |
| STAT3- | Sense | | CAAAAGUGAUGAACAUGGA | 146 |
| 1383 | 19 mer | | | |
| STAT3- | Sense | | GUGAUGAACAUGGAAGAAU | 147 |
| 1388 | 19 mer | | | |
| STAT3- | Sense | | GCAGAAUUCAAACACUUGA | 148 |
| 1427 | 19 mer | | | |
| STAT3- | Sense | | AUUGUGAUGCUUCCCUGAU | 149 |
| 1485 | 19 mer | | | |
| STAT3- | Sense | | CCUUGCCAGUUGUGGUGAU | 150 |
| 1584 | 19 mer | | | |
| STAT3- | Sense | | UUGCCAGUUGUGGUGAUCU | 151 |
| 1586 | 19 mer | | | |
| STAT3- | Sense | | CCCAAGAAUGUAAACUUUU | 152 |
| 1670 | 19 mer | | | |
| STAT3- | Sense | | CCAAGAAUGUAAACUUUUU | 153 |
| 1671 | 19 mer | | | |
| STAT3- | Sense | | CAAGAAUGUAAACUUUUUU | 154 |
| 1672 | 19 mer | | | |
| STAT3- | Sense | | AAGAAUGUAAACUUUUUUA | 155 |
| 1673 | 19 mer | | | |
| STAT3- | Sense | | AGAAUGUAAACUUUUUUAC | 156 |
| 1674 | 19 mer | | | |
| STAT3- | Sense | | AAUGUAAACUUUUUUACCA | 157 |
| 1676 | 19 mer | | | |
| STAT3- | Sense | | ACCUGGUGUGAAUUAUUCA | 158 |
| 1813 | 19 mer | | | |
| STAT3- | Sense | | CUGGUGUGAAUUAUUCAGG | 159 |
| 1815 | 19 mer | | | |
| STAT3- | Sense | | GGUGUGAAUUAUUCAGGGU | 160 |
| 1817 | 19 mer | | | |
| STAT3- | Sense | | UGUGAAUUAUUCAGGGUGU | 161 |
| 1819 | 19 mer | | | |
| STAT3- | Sense | | CUGGACAAUAUCAUUGACC | 162 |
| 1904 | 19 mer | | | |
| STAT3- | Sense | | GGACAAUAUCAUUGACCUU | 163 |
| 1906 | 19 mer | | | |
| STAT3- | Sense | | GACAAUAUCAUUGACCUUG | 164 |
| 1907 | 19 mer | | | |
| STAT3- | Sense | | ACAAUAUCAUUGACCUUGU | 165 |
| 1908 | 19 mer | | | |
| STAT3- | Sense | | CAAUAUCAUUGACCUUGUG | 166 |
| 1909 | 19 mer | | | |
| STAT3- | Sense | | AAUAUCAUUGACCUUGUGA | 167 |
| 1910 | 19 mer | | | |
| STAT3- | Sense | | AUAUCAUUGACCUUGUGAA | 168 |
| 1911 | 19 mer | | | |
| STAT3- | Sense | | UAUCAUUGACCUUGUGAAA | 169 |
| 1912 | 19 mer | | | |
| STAT3- | Sense | | AUCAUUGACCUUGUGAAAA | 170 |
| 1913 | 19 mer | | | |
| STAT3- | Sense | | UCAUUGACCUUGUGAAAAA | 171 |
| 1914 | 19 mer | | | |
| STAT3- | Sense | | AUUGACCUUGUGAAAAAGU | 172 |
| 1916 | 19 mer | | | |
| STAT3- | Sense | | UUGACCUUGUGAAAAAGUA | 173 |
| 1917 | 19 mer | | | |
| STAT3- | Sense | | GACCUUGUGAAAAAGUACA | 174 |
| 1919 | 19 mer | | | |
| STAT3- | Sense | | ACCUUGUGAAAAAGUACAU | 175 |
| 1920 | 19 mer | | | |
| STAT3- | Sense | | ACCUUCCUGCUAAGAUUCA | 176 |
| 2024 | 19 mer | | | |
| STAT3- | Sense | | AAGCAGCAGCUGAACAACA | 177 |
| 2135 | 19 mer | | | |
| STAT3- | Sense | | AGCAGCAGCUGAACAACAU | 178 |
| 2136 | 19 mer | | | |
| STAT3- | Sense | | CAGCAGCUGAACAACAUGU | 179 |
| 2138 | 19 mer | | | |
| STAT3- | Sense | | AGCAGCUGAACAACAUGUC | 180 |
| 2139 | 19 mer | | | |
| STAT3- | Sense | | GCUGAACAACAUGUCAUUU | 181 |
| 2143 | 19 mer | | | |
| STAT3- | Sense | | CUGAACAACAUGUCAUUUG | 182 |
| 2144 | 19 mer | | | |
| STAT3- | Sense | | UGAACAACAUGUCAUUUGC | 183 |
| 2145 | 19 mer | | | |
| STAT3- | Sense | | GAACAACAUGUCAUUUGCU | 184 |
| 2146 | 19 mer | | | |
| STAT3- | Sense | | AACAACAUGUCAUUUGCUG | 185 |
| 2147 | 19 mer | | | |
| STAT3- | Sense | | ACAACAUGUCAUUUGCUGA | 186 |
| 2148 | 19 mer | | | |
| STAT3- | Sense | | ACAUGUCAUUUGCUGAAAU | 187 |
| 2151 | 19 mer | | | |
| STAT3- | Sense | | AUGUCAUUUGCUGAAAUCA | 188 |
| 2153 | 19 mer | | | |
| STAT3- | Sense | | UGUCAUUUGCUGAAAUCAU | 189 |
| 2154 | 19 mer | | | |
| STAT3- | Sense | | UUUGCUGAAAUCAUCAUGG | 190 |
| 2159 | 19 mer | | | |
| STAT3- | Sense | | CAUACCUGAAGACCAAGUU | 191 |
| 2322 | 19 mer | | | |
| STAT3- | Sense | | ACCUGAAGACCAAGUUUAU | 192 |
| 2325 | 19 mer | | | |
| STAT3- | Sense | | CUGAAGACCAAGUUUAUCU | 193 |
| 2327 | 19 mer | | | |
| STAT3- | Sense | | GAAGACCAAGUUUAUCUGU | 194 |
| 2329 | 19 mer | | | |
| STAT3- | Sense | | ACCAAGUUUAUCUGUGUGA | 195 |
| 2333 | 19 mer | | | |
| STAT3- | Sense | | CAAGUUUAUCUGUGUGACA | 196 |
| 2335 | 19 mer | | | |
| STAT3- | Sense | | AGAUUCAUUGAUGCAGUUU | 197 |
| 2404 | 19 mer | | | |
| STAT3- | Sense | | GAUUCAUUGAUGCAGUUUG | 198 |
| 2405 | 19 mer | | | |
| STAT3- | Sense | | UUCAUUGAUGCAGUUUGGA | 199 |
| 2407 | 19 mer | | | |
| STAT3- | Sense | | UCAUUGAUGCAGUUUGGAA | 200 |
| 2408 | 19 mer | | | |
| STAT3- | Sense | | UUGAUGCAGUUUGGAAAUA | 201 |
| 2411 | 19 mer | | | |
| STAT3- | Sense | | UGAUGCAGUUUGGAAAUAA | 202 |
| 2412 | 19 mer | | | |
| STAT3- | Sense | | GAUGCAGUUUGGAAAUAAU | 203 |
| 2413 | 19 mer | | | |
| STAT3- | Sense | | GCAGUUUGGAAAUAAUGGU | 204 |
| 2416 | 19 mer | | | |
| STAT3- | Sense | | AGUUUGGAAAUAAUGGUGA | 205 |
| 2418 | 19 mer | | | |
| STAT3- | Sense | | UGGAAAUAAUGGUGAAGGU | 206 |
| 2422 | 19 mer | | | |
| STAT3- | Sense | | AUAAUGGUGAAGGUGCUGA | 207 |
| 2427 | 19 mer | | | |
| STAT3- | Sense | | CUGAAACUACUAACUUUGU | 208 |
| 2612 | 19 mer | | | |
| STAT3- | Sense | | AAACUACUAACUUUGUGGU | 209 |
| 2615 | 19 mer | | | |
| STAT3- | Sense | | AACUACUAACUUUGUGGUU | 210 |
| 2616 | 19 mer | | | |
| STAT3- | Sense | | ACUACUAACUUUGUGGUUC | 211 |
| 2617 | 19 mer | | | |
| STAT3- | Sense | | UAACUUUGUGGUUCCAGAU | 212 |
| 2622 | 19 mer | | | |
| STAT3- | Sense | | CUUUGUGGUUCCAGAUUUU | 213 |
| 2625 | 19 mer | | | |
| STAT3- | Sense | | UUUGUGGUUCCAGAUUUUU | 214 |
| 2626 | 19 mer | | | |
| STAT3- | Sense | | UUGUGGUUCCAGAUUUUUU | 215 |
| 2627 | 19 mer | | | |
| STAT3- | Sense | | AAAUAGAGAAAUGAGUGAA | 216 |
| 2692 | 19 mer | | | |
| STAT3- | Sense | | AAUAGAGAAAUGAGUGAAU | 217 |
| 2693 | 19 mer | | | |
| STAT3- | Sense | | GGUGAUCUGCUUUUAUCUA | 218 |
| 2715 | 19 mer | | | |
| STAT3- | Sense | | AUCUGCUUUUAUCUAAAUG | 219 |
| 2719 | 19 mer | | | |
| STAT3- | Sense | | CUGCUUUUAUCUAAAUGCA | 220 |
| 2721 | 19 mer | | | |
| STAT3- | Sense | | AUGCAAAUAAGGAUGUGUU | 221 |
| 2735 | 19 mer | | | |
| STAT3- | Sense | | AUAAGGAUGUGUUCUCUGA | 222 |
| 2741 | 19 mer | | | |
| STAT3- | Sense | | GAAAAAGGAAAUGUCUUGU | 223 |
| 2801 | 19 mer | | | |
| STAT3- | Sense | | AAAAGGAAAUGUCUUGUGU | 224 |
| 2803 | 19 mer | | | |
| STAT3- | Sense | | AAAGGAAAUGUCUUGUGUU | 225 |
| 2804 | 19 mer | | | |
| STAT3- | Sense | | AGGAAAUGUCUUGUGUUGU | 226 |
| 2806 | 19 mer | | | |
| STAT3- | Sense | | GGAAAUGUCUUGUGUUGUU | 227 |
| 2807 | 19 mer | | | |
| STAT3- | Sense | | GAAAUGUCUUGUGUUGUUU | 228 |
| 2808 | 19 mer | | | |
| STAT3- | Sense | | AAAUGUCUUGUGUUGUUUU | 229 |
| 2809 | 19 mer | | | |
| STAT3- | Sense | | AAUGUCUUGUGUUGUUUUG | 230 |
| 2810 | 19 mer | | | |
| STAT3- | Sense | | AUGUCUUGUGUUGUUUUGU | 231 |
| 2811 | 19 mer | | | |
| STAT3- | Sense | | UGUCUUGUGUUGUUUUGUU | 232 |
| 2812 | 19 mer | | | |
| STAT3- | Sense | | GUCUUGUGUUGUUUUGUUC | 233 |
| 2813 | 19 mer | | | |
| STAT3- | Sense | | CUCAGCAGCUUUUUGUUAU | 234 |
| 2846 | 19 mer | | | |
| STAT3- | Sense | | CAGCAGCUUUUUGUUAUUG | 235 |
| 2848 | 19 mer | | | |
| STAT3- | Sense | | AGCAGCUUUUUGUUAUUGU | 236 |
| 2849 | 19 mer | | | |
| STAT3- | Sense | | GCAGCUUUUUGUUAUUGUU | 237 |
| 2850 | 19 mer | | | |
| STAT3- | Sense | | CAGCUUUUUGUUAUUGUUG | 238 |
| 2851 | 19 mer | | | |
| STAT3- | Sense | | AGCUUUUUGUUAUUGUUGU | 239 |
| 2852 | 19 mer | | | |
| STAT3- | Sense | | GCUUUUUGUUAUUGUUGUU | 240 |
| 2853 | 19 mer | | | |
| STAT3- | Sense | | CUUUUUGUUAUUGUUGUUG | 241 |
| 2854 | 19 mer | | | |
| STAT3- | Sense | | UUUUUGUUAUUGUUGUUGU | 242 |
| 2855 | 19 mer | | | |
| STAT3- | Sense | | UUUUGUUAUUGUUGUUGUU | 243 |
| 2856 | 19 mer | | | |
| STAT3- | Sense | | UUUGUUAUUGUUGUUGUUG | 244 |
| 2857 | 19 mer | | | |
| STAT3- | Sense | | UUGUUAUUGUUGUUGUUGU | 245 |
| 2858 | 19 mer | | | |
| STAT3- | Sense | | UGUUAUUGUUGUUGUUGUU | 246 |
| 2859 | 19 mer | | | |
| STAT3- | Sense | | GUUAUUGUUGUUGUUGUUC | 247 |
| 2860 | 19 mer | | | |
| STAT3- | Sense | | UUAUUGUUGUUGUUGUUCU | 248 |
| 2861 | 19 mer | | | |
| STAT3- | Sense | | UAUUGUUGUUGUUGUUCUU | 249 |
| 2862 | 19 mer | | | |
| STAT3- | Sense | | AUUGUUGUUGUUGUUCUUA | 250 |
| 2863 | 19 mer | | | |
| STAT3- | Sense | | UGUUGUUGUUGUUCUUAGA | 251 |
| 2865 | 19 mer | | | |
| STAT3- | Sense | | UUGUUGUUGUUCUUAGACA | 252 |
| 2867 | 19 mer | | | |
| STAT3- | Sense | | UGUUGUUGUUCUUAGACAA | 253 |
| 2868 | 19 mer | | | |
| STAT3- | Sense | | CUUUUUAACCUUGCUGACA | 254 |
| 2975 | 19 mer | | | |
| STAT3- | Sense | | UUAACCUUGCUGACAUCCA | 255 |
| 2979 | 19 mer | | | |
| STAT3- | Sense | | UUGCUGACAUCCAAAUAGA | 256 |
| 2985 | 19 mer | | | |
| STAT3- | Sense | | AGGUUUCUUUUUAAAUUAA | 257 |
| 3025 | 19 mer | | | |
| STAT3- | Sense | | AAAUUAAGAAAUAAUAACA | 258 |
| 3037 | 19 mer | | | |
| STAT3- | Sense | | AAUUAAGAAAUAAUAACAA | 259 |
| 3038 | 19 mer | | | |
| STAT3- | Sense | | AUUAAGAAAUAAUAACAAU | 260 |
| 3039 | 19 mer | | | |
| STAT3- | Sense | | UAAGAAAUAAUAACAAUUA | 26 |
| 3041 | 19 mer | | | |
| STAT3- | Sense | | AAGAAAUAAUAACAAUUAA | 262 |
| 3042 | 19 mer | | | |
| STAT3- | Sense | | AGAAAUAAUAACAAUUAAA | 263 |
| 3043 | 19 mer | | | |
| STAT3- | Sense | | ACUAAAAGUACAAAAAAUU | 264 |
| 3225 | 19 mer | | | |
| STAT3- | Sense | | CUAAAAGUACAAAAAAUUA | 265 |
| 3226 | 19 mer | | | |
| STAT3- | Sense | | AGACUUAUUUUCCUUUGUA | 266 |
| 3605 | 19 mer | | | |
| STAT3- | Sense | | AUUUUCCUUUGUAAUGUAU | 267 |
| 3611 | 19 mer | | | |
| STAT3- | Sense | | AGUUACAGGUUGGACAUGA | 268 |
| 3906 | 19 mer | | | |
| STAT3- | Sense | | UGUGGAAUUCUGUUUGUUA | 269 |
| 4311 | 19 mer | | | |
| STAT3- | Sense | | GGAAUUCUGUUUGUUAAAU | 270 |
| 4314 | 19 mer | | | |
| STAT3- | Sense | | AUUCUGUUUGUUAAAUCAA | 271 |
| 4317 | 19 mer | | | |
| STAT3- | Sense | | UGUUUGUUAAAUCAAAUUA | 272 |
| 4321 | 19 mer | | | |
| STAT3- | Sense | | ACAUAAUAAGCUUAACUGA | 273 |
| 4465 | 19 mer | | | |
| STAT3- | Sense | | ACUGAUAAACAGAAUAUUU | 274 |
| 4479 | 19 mer | | | |
| STAT3- | Sense | | CUGAUAAACAGAAUAUUUA | 275 |
| 4480 | 19 mer | | | |
| STAT3- | Sense | | UAGUGUAAAAAUUUAUAUU | 276 |
| 4831 | 19 mer | | | |
| STAT3- | Sense | | GUGUAAAAAUUUAUAUUAU | 277 |
| 4833 | 19 mer | | | |
| STAT3- | Sense | | UAAAAAUUUAUAUUAUUGU | 278 |
| 4836 | 19 mer | | | |
| STAT3- | Sense | | AAAAAUUUAUAUUAUUGUG | 279 |
| 4837 | 19 mer | | | |
| STAT3- | Sense | | UUUAACUUCCAGAAAUAAA | 280 |
| 4909 | 19 mer | | | |
| STAT3- | Antisense | | AUUAUGAAACACCAAAGUG | 281 |
| 370 | 19 mer | | | |
| STAT3- | Antisense | | AGAUUAUGAAACACCAAAG | 282 |
| 372 | 19 mer | | | |
| STAT3- | Antisense | | AACAUUCGACUCUUGCAGG | 283 |
| 424 | 19 mer | | | |
| STAT3- | Antisense | | GAACAUUCGACUCUUGCAG | 284 |
| 425 | 19 mer | | | |
| STAT3- | Antisense | | AGAACAUUCGACUCUUGCA | 285 |
| 426 | 19 mer | | | |
| STAT3- | Antisense | | UAGAGAACAUUCGACUCUU | 286 |
| 429 | 19 mer | | | |
| STAT3- | Antisense | | AUAGAGAACAUUCGACUCU | 287 |
| 430 | 19 mer | | | |
| STAT3- | Antisense | | UGAUAGAGAACAUUCGACU | 288 |
| 432 | 19 mer | | | |
| STAT3- | Antisense | | CUGAUAGAGAACAUUCGAC | 289 |
| 433 | 19 mer | | | |
| STAT3- | Antisense | | AAACUGCUUGAUUCUUCGU | 290 |
| 460 | 19 mer | | | |
| STAT3- | Antisense | | GAAACUGCUUGAUUCUUCG | 291 |
| 461 | 19 mer | | | |
| STAT3- | Antisense | | AGAAACUGCUUGAUUCUUC | 292 |
| 462 | 19 mer | | | |
| STAT3- | Antisense | | UCCAUUGGCUUCUCAAGAU | 293 |
| 492 | 19 mer | | | |
| STAT3- | Antisense | | AUUUUCUGUUCUAGAUCCU | 294 |
| 678 | 19 mer | | | |
| STAT3- | Antisense | | UUCAUUUUCUGUUCUAGAU | 295 |
| 681 | 19 mer | | | |
| STAT3- | Antisense | | GAAAUCAAAGUCAUCCUGG | 296 |
| 715 | 19 mer | | | |
| STAT3- | Antisense | | UGAAAUCAAAGUCAUCCUG | 297 |
| 716 | 19 mer | | | |
| STAT3- | Antisense | | UUGAAAUCAAAGUCAUCCU | 298 |
| 717 | 19 mer | | | |
| STAT3- | Antisense | | UAGUUGAAAUCAAAGUCAU | 299 |
| 720 | 19 mer | | | |
| STAT3- | Antisense | | AUAGUUGAAAUCAAAGUCA | 300 |
| 721 | 19 mer | | | |
| STAT3- | Antisense | | UAUAGUUGAAAUCAAAGUC | 301 |
| 722 | 19 mer | | | |
| STAT3- | Antisense | | UUAUAGUUGAAAUCAAAGU | 302 |
| 723 | 19 mer | | | |
| STAT3- | Antisense | | UUUAUAGUUGAAAUCAAAG | 303 |
| 724 | 19 mer | | | |
| STAT3- | Antisense | | UUGUUUCCAUUCAGAUCUU | 304 |
| 768 | 19 mer | | | |
| STAT3- | Antisense | | UGGUUGUUUCCAUUCAGAU | 305 |
| 771 | 19 mer | | | |
| STAT3- | Antisense | | ACUGGUUGUUUCCAUUCAG | 306 |
| 773 | 19 mer | | | |
| STAT3- | Antisense | | UGACGUUAUCCAGUUUUCU | 307 |
| 1000 | 19 mer | | | |
| STAT3- | Antisense | | AUGACGUUAUCCAGUUUUC | 308 |
| 1001 | 19 mer | | | |
| STAT3- | Antisense | | UAAUGACGUUAUCCAGUUU | 309 |
| 1003 | 19 mer | | | |
| STAT3- | Antisense | | UGCUAAUGACGUUAUCCAG | 310 |
| 1006 | 19 mer | | | |
| STAT3- | Antisense | | UCUGCUAAUGACGUUAUCC | 311 |
| 1008 | 19 mer | | | |
| STAT3- | Antisense | | UUCUGCUAAUGACGUUAUC | 312 |
| 1009 | 19 mer | | | |
| STAT3- | Antisense | | AUUCUGCUAAUGACGUUAU | 313 |
| 1010 | 19 mer | | | |
| STAT3- | Antisense | | UCCAGUUUCUUAAUUUGUU | 314 |
| 1047 | 19 mer | | | |
| STAT3- | Antisense | | AAACUUUUUGCUGCAACUC | 315 |
| 1067 | 19 mer | | | |
| STAT3- | Antisense | | GAAACUUUUUGCUGCAACU | 316 |
| 1068 | 19 mer | | | |
| STAT3- | Antisense | | UCAUUAAGUUUCUAAACAG | 317 |
| 1145 | 19 mer | | | |
| STAT3- | Antisense | | CACUUUUCAUUAAGUUUCU | 318 |
| 1151 | 19 mer | | | |
| STAT3- | Antisense | | UGACUUUAGUAGUGAACUG | 319 |
| 1241 | 19 mer | | | |
| STAT3- | Antisense | | UCAACUCAGGGAAUUUGAC | 320 |
| 1268 | 19 mer | | | |
| STAT3- | Antisense | | UAAUUCAACUCAGGGAAUU | 321 |
| 1272 | 19 mer | | | |
| STAT3- | Antisense | | AUAAUUCAACUCAGGGAAU | 322 |
| 1273 | 19 mer | | | |
| STAT3- | Antisense | | UGAUAAUUCAACUCAGGGA | 323 |
| 1275 | 19 mer | | | |
| STAT3- | Antisense | | GCUGAUAAUUCAACUCAGG | 324 |
| 1277 | 19 mer | | | |
| STAT3- | Antisense | | AGCUGAUAAUUCAACUCAG | 325 |
| 1278 | 19 mer | | | |
| STAT3- | Antisense | | AAGCUGAUAAUUCAACUCA | 326 |
| 1279 | 19 mer | | | |
| STAT3- | Antisense | | UAAGCUGAUAAUUCAACUC | 327 |
| 1280 | 19 mer | | | |
| STAT3- | Antisense | | UUAAGCUGAUAAUUCAACU | 328 |
| 1281 | 19 mer | | | |
| STAT3- | Antisense | | UUUAAGCUGAUAAUUCAAC | 329 |
| 1282 | 19 mer | | | |
| STAT3- | Antisense | | UUUUAAGCUGAUAAUUCAA | 330 |
| 1283 | 19 mer | | | |
| STAT3- | Antisense | | AUUUUAAGCUGAUAAUUCA | 331 |
| 1284 | 19 mer | | | |
| STAT3- | Antisense | | UAAUUUUAAGCUGAUAAUU | 332 |
| 1286 | 19 mer | | | |
| STAT3- | Antisense | | UUAAUUUUAAGCUGAUAAU | 333 |
| 1287 | 19 mer | | | |
| STAT3- | Antisense | | ACACUUUAAUUUUAAGCUG | 334 |
| 1292 | 19 mer | | | |
| STAT3- | Antisense | | CACACUUUAAUUUUAAGCU | 335 |
| 1293 | 19 mer | | | |
| STAT3- | Antisense | | UCAAUGCACACUUUAAUUU | 336 |
| 1299 | 19 mer | | | |
| STAT3- | Antisense | | UCUUUGUCAAUGCACACUU | 337 |
| 1305 | 19 mer | | | |
| STAT3- | Antisense | | UCCAUGUUCAUCACUUUUG | 338 |
| 1383 | 19 mer | | | |
| STAT3- | Antisense | | AUUCUUCCAUGUUCAUCAC | 339 |
| 1388 | 19 mer | | | |
| STAT3- | Antisense | | UCAAGUGUUUGAAUUCUGC | 340 |
| 1427 | 19 mer | | | |
| STAT3- | Antisense | | AUCAGGGAAGCAUCACAAU | 341 |
| 1485 | 19 mer | | | |
| STAT3- | Antisense | | AUCACCACAACUGGCAAGG | 342 |
| 1584 | 19 mer | | | |
| STAT3- | Antisense | | AGAUCACCACAACUGGCAA | 343 |
| 1586 | 19 mer | | | |
| STAT3- | Antisense | | AAAAGUUUACAUUCUUGGG | 344 |
| 1670 | 19 mer | | | |
| STAT3- | Antisense | | AAAAAGUUUACAUUCUUGG | 345 |
| 1671 | 19 mer | | | |
| STAT3- | Antisense | | AAAAAAGUUUACAUUCUUG | 346 |
| 1672 | 19 mer | | | |
| STAT3- | Antisense | | UAAAAAAGUUUACAUUCUU | 347 |
| 1673 | 19 mer | | | |
| STAT3- | Antisense | | GUAAAAAAGUUUACAUUCU | 348 |
| 1674 | 19 mer | | | |
| STAT3- | Antisense | | UGGUAAAAAAGUUUACAUU | 349 |
| 1676 | 19 mer | | | |
| STAT3- | Antisense | | UGAAUAAUUCACACCAGGU | 350 |
| 1813 | 19 mer | | | |
| STAT3- | Antisense | | CCUGAAUAAUUCACACCAG | 351 |
| 1815 | 19 mer | | | |
| STAT3- | Antisense | | ACCCUGAAUAAUUCACACC | 352 |
| 1817 | 19 mer | | | |
| STAT3- | Antisense | | ACACCCUGAAUAAUUCACA | 353 |
| 1819 | 19 mer | | | |
| STAT3- | Antisense | | GGUCAAUGAUAUUGUCCAG | 354 |
| 1904 | 19 mer | | | |
| STAT3- | Antisense | | AAGGUCAAUGAUAUUGUCC | 355 |
| 1906 | 19 mer | | | |
| STAT3- | Antisense | | CAAGGUCAAUGAUAUUGUC | 356 |
| 1907 | 19 mer | | | |
| STAT3- | Antisense | | ACAAGGUCAAUGAUAUUGU | 357 |
| 1908 | 19 mer | | | |
| STAT3- | Antisense | | CACAAGGUCAAUGAUAUUG | 358 |
| 1909 | 19 mer | | | |
| STAT3- | Antisense | | UCACAAGGUCAAUGAUAUU | 359 |
| 1910 | 19 mer | | | |
| STAT3- | Antisense | | UUCACAAGGUCAAUGAUAU | 360 |
| 1911 | 19 mer | | | |
| STAT3- | Antisense | | UUUCACAAGGUCAAUGAUA | 361 |
| 1912 | 19 mer | | | |
| STAT3- | Antisense | | UUUUCACAAGGUCAAUGAU | 362 |
| 1913 | 19 mer | | | |
| STAT3- | Antisense | | UUUUUCACAAGGUCAAUGA | 363 |
| 1914 | 19 mer | | | |
| STAT3- | Antisense | | ACUUUUUCACAAGGUCAAU | 364 |
| 1916 | 19 mer | | | |
| STAT3- | Antisense | | UACUUUUUCACAAGGUCAA | 365 |
| 1917 | 19 mer | | | |
| STAT3- | Antisense | | UGUACUUUUUCACAAGGUC | 366 |
| 1919 | 19 mer | | | |
| STAT3- | Antisense | | AUGUACUUUUUCACAAGGU | 367 |
| 1920 | 19 mer | | | |
| STAT3- | Antisense | | UGAAUCUUAGCAGGAAGGU | 368 |
| 2024 | 19 mer | | | |
| STAT3- | Antisense | | UGUUGUUCAGCUGCUGCUU | 369 |
| 2135 | 19 mer | | | |
| STAT3- | Antisense | | AUGUUGUUCAGCUGCUGCU | 370 |
| 2136 | 19 mer | | | |
| STAT3- | Antisense | | ACAUGUUGUUCAGCUGCUG | 371 |
| 2138 | 19 mer | | | |
| STAT3- | Antisense | | GACAUGUUGUUCAGCUGCU | 372 |
| 2139 | 19 mer | | | |
| STAT3- | Antisense | | AAAUGACAUGUUGUUCAGC | 373 |
| 2143 | 19 mer | | | |
| STAT3- | Antisense | | CAAAUGACAUGUUGUUCAG | 374 |
| 2144 | 19 mer | | | |
| STAT3- | Antisense | | GCAAAUGACAUGUUGUUCA | 375 |
| 2145 | 19 mer | | | |
| STAT3- | Antisense | | AGCAAAUGACAUGUUGUUC | 376 |
| 2146 | 19 mer | | | |
| STAT3- | Antisense | | CAGCAAAUGACAUGUUGUU | 377 |
| 2147 | 19 mer | | | |
| STAT3- | Antisense | | UCAGCAAAUGACAUGUUGU | 378 |
| 2148 | 19 mer | | | |
| STAT3- | Antisense | | AUUUCAGCAAAUGACAUGU | 379 |
| 2151 | 19 mer | | | |
| STAT3- | Antisense | | UGAUUUCAGCAAAUGACAU | 380 |
| 2153 | 19 mer | | | |
| STAT3- | Antisense | | AUGAUUUCAGCAAAUGACA | 381 |
| 2154 | 19 mer | | | |
| STAT3- | Antisense | | CCAUGAUGAUUUCAGCAAA | 382 |
| 2159 | 19 mer | | | |
| STAT3- | Antisense | | AACUUGGUCUUCAGGUAUG | 383 |
| 2322 | 19 mer | | | |
| STAT3- | Antisense | | AUAAACUUGGUCUUCAGGU | 384 |
| 2325 | 19 mer | | | |
| STAT3- | Antisense | | AGAUAAACUUGGUCUUCAG | 385 |
| 2327 | 19 mer | | | |
| STAT3- | Antisense | | ACAGAUAAACUUGGUCUUC | 386 |
| 2329 | 19 mer | | | |
| STAT3- | Antisense | | UCACACAGAUAAACUUGGU | 387 |
| 2333 | 19 mer | | | |
| STAT3- | Antisense | | UGUCACACAGAUAAACUUG | 388 |
| 2335 | 19 mer | | | |
| STAT3- | Antisense | | AAACUGCAUCAAUGAAUCU | 389 |
| 2404 | 19 mer | | | |
| STAT3- | Antisense | | CAAACUGCAUCAAUGAAUC | 390 |
| 2405 | 19 mer | | | |
| STAT3- | Antisense | | UCCAAACUGCAUCAAUGAA | 391 |
| 2407 | 19 mer | | | |
| STAT3- | Antisense | | UUCCAAACUGCAUCAAUGA | 392 |
| 2408 | 19 mer | | | |
| STAT3- | Antisense | | UAUUUCCAAACUGCAUCAA | 393 |
| 2411 | 19 mer | | | |
| STAT3- | Antisense | | UUAUUUCCAAACUGCAUCA | 394 |
| 2412 | 19 mer | | | |
| STAT3- | Antisense | | AUUAUUUCCAAACUGCAUC | 395 |
| 2413 | 19 mer | | | |
| STAT3- | Antisense | | ACCAUUAUUUCCAAACUGC | 396 |
| 2416 | 19 mer | | | |
| STAT3- | Antisense | | UCACCAUUAUUUCCAAACU | 397 |
| 2418 | 19 mer | | | |
| STAT3- | Antisense | | ACCUUCACCAUUAUUUCCA | 398 |
| 2422 | 19 mer | | | |
| STAT3- | Antisense | | UCAGCACCUUCACCAUUAU | 399 |
| 2427 | 19 mer | | | |
| STAT3- | Antisense | | ACAAAGUUAGUAGUUUCAG | 400 |
| 2612 | 19 mer | | | |
| STAT3- | Antisense | | ACCACAAAGUUAGUAGUUU | 401 |
| 2615 | 19 mer | | | |
| STAT3- | Antisense | | AACCACAAAGUUAGUAGUU | 402 |
| 2616 | 19 mer | | | |
| STAT3- | Antisense | | GAACCACAAAGUUAGUAGU | 403 |
| 2617 | 19 mer | | | |
| STAT3- | Antisense | | AUCUGGAACCACAAAGUUA | 404 |
| 2622 | 19 mer | | | |
| STAT3- | Antisense | | AAAAUCUGGAACCACAAAG | 405 |
| 2625 | 19 mer | | | |
| STAT3- | Antisense | | AAAAAUCUGGAACCACAAA | 406 |
| 2626 | 19 mer | | | |
| STAT3- | Antisense | | AAAAAAUCUGGAACCACAA | 407 |
| 2627 | 19 mer | | | |
| STAT3- | Antisense | | UUCACUCAUUUCUCUAUUU | 408 |
| 2692 | 19 mer | | | |
| STAT3- | Antisense | | AUUCACUCAUUUCUCUAUU | 409 |
| 2693 | 19 mer | | | |
| STAT3- | Antisense | | UAGAUAAAAGCAGAUCACC | 410 |
| 2715 | 19 mer | | | |
| STAT3- | Antisense | | CAUUUAGAUAAAAGCAGAU | 411 |
| 2719 | 19 mer | | | |
| STAT3- | Antisense | | UGCAUUUAGAUAAAAGCAG | 412 |
| 2721 | 19 mer | | | |
| STAT3- | Antisense | | AACACAUCCUUAUUUGCAU | 413 |
| 2735 | 19 mer | | | |
| STAT3- | Antisense | | UCAGAGAACACAUCCUUAU | 414 |
| 2741 | 19 mer | | | |
| STAT3- | Antisense | | ACAAGACAUUUCCUUUUUC | 415 |
| 2801 | 19 mer | | | |
| STAT3- | Antisense | | ACACAAGACAUUUCCUUUU | 416 |
| 2803 | 19 mer | | | |
| STAT3- | Antisense | | AACACAAGACAUUUCCUUU | 417 |
| 2804 | 19 mer | | | |
| STAT3- | Antisense | | ACAACACAAGACAUUUCCU | 418 |
| 2806 | 19 mer | | | |
| STAT3- | Antisense | | AACAACACAAGACAUUUCC | 419 |
| 2807 | 19 mer | | | |
| STAT3- | Antisense | | AAACAACACAAGACAUUUC | 420 |
| 2808 | 19 mer | | | |
| STAT3- | Antisense | | AAAACAACACAAGACAUUU | 421 |
| 2809 | 19 mer | | | |
| STAT3- | Antisense | | CAAAACAACACAAGACAUU | 422 |
| 2810 | 19 mer | | | |
| STAT3- | Antisense | | ACAAAACAACACAAGACAU | 423 |
| 2811 | 19 mer | | | |
| STAT3- | Antisense | | AACAAAACAACACAAGACA | 424 |
| 2812 | 19 mer | | | |
| STAT3- | Antisense | | GAACAAAACAACACAAGAC | 425 |
| 2813 | 19 mer | | | |
| STAT3- | Antisense | | AUAACAAAAAGCUGCUGAG | 426 |
| 2846 | 19 mer | | | |
| STAT3- | Antisense | | CAAUAACAAAAAGCUGCUG | 427 |
| 2848 | 19 mer | | | |
| STAT3- | Antisense | | ACAAUAACAAAAAGCUGCU | 428 |
| 2849 | 19 mer | | | |
| STAT3- | Antisense | | AACAAUAACAAAAAGCUGC | 429 |
| 2850 | 19 mer | | | |
| STAT3- | Antisense | | CAACAAUAACAAAAAGCUG | 430 |
| 2851 | 19 mer | | | |
| STAT3- | Antisense | | ACAACAAUAACAAAAAGCU | 431 |
| 2852 | 19 mer | | | |
| STAT3- | Antisense | | AACAACAAUAACAAAAAGC | 432 |
| 2853 | 19 mer | | | |
| STAT3- | Antisense | | CAACAACAAUAACAAAAAG | 433 |
| 2854 | 19 mer | | | |
| STAT3- | Antisense | | ACAACAACAAUAACAAAAA | 434 |
| 2855 | 19 mer | | | |
| STAT3- | Antisense | | AACAACAACAAUAACAAAA | 435 |
| 2856 | 19 mer | | | |
| STAT3- | Antisense | | CAACAACAACAAUAACAAA | 436 |
| 2857 | 19 mer | | | |
| STAT3- | Antisense | | ACAACAACAACAAUAACAA | 437 |
| 2858 | 19 mer | | | |
| STAT3- | Antisense | | AACAACAACAACAAUAACA | 438 |
| 2859 | 19 mer | | | |
| STAT3- | Antisense | | GAACAACAACAACAAUAAC | 439 |
| 2860 | 19 mer | | | |
| STAT3- | Antisense | | AGAACAACAACAACAAUAA | 440 |
| 2861 | 19 mer | | | |
| STAT3- | Antisense | | AAGAACAACAACAACAAUA | 441 |
| 2862 | 19 mer | | | |
| STAT3- | Antisense | | UAAGAACAACAACAACAAU | 442 |
| 2863 | 19 mer | | | |
| STAT3- | Antisense | | UCUAAGAACAACAACAACA | 443 |
| 2865 | 19 mer | | | |
| STAT3- | Antisense | | UGUCUAAGAACAACAACAA | 444 |
| 2867 | 19 mer | | | |
| STAT3- | Antisense | | UUGUCUAAGAACAACAACA | 445 |
| 2868 | 19 mer | | | |
| STAT3- | Antisense | | UGUCAGCAAGGUUAAAAAG | 446 |
| 2975 | 19 mer | | | |
| STAT3- | Antisense | | UGGAUGUCAGCAAGGUUAA | 447 |
| 2979 | 19 mer | | | |
| STAT3- | Antisense | | UCUAUUUGGAUGUCAGCAA | 448 |
| 2985 | 19 mer | | | |
| STAT3- | Antisense | | UUAAUUUAAAAAGAAACCU | 449 |
| 3025 | 19 mer | | | |
| STAT3- | Antisense | | UGUUAUUAUUUCUUAAUUU | 450 |
| 3037 | 19 mer | | | |
| STAT3- | Antisense | | UUGUUAUUAUUUCUUAAUU | 451 |
| 3038 | 19 mer | | | |
| STAT3- | Antisense | | AUUGUUAUUAUUUCUUAAU | 452 |
| 3039 | 19 mer | | | |
| STAT3- | Antisense | | UAAUUGUUAUUAUUUCUUA | 453 |
| 3041 | 19 mer | | | |
| STAT3- | Antisense | | UUAAUUGUUAUUAUUUCUU | 454 |
| 3042 | 19 mer | | | |
| STAT3- | Antisense | | UUUAAUUGUUAUUAUUUCU | 455 |
| 3043 | 19 mer | | | |
| STAT3- | Antisense | | AAUUUUUUGUACUUUUAGU | 456 |
| 3225 | 19 mer | | | |
| STAT3- | Antisense | | UAAUUUUUUGUACUUUUAG | 457 |
| 3226 | 19 mer | | | |
| STAT3- | Antisense | | UACAAAGGAAAAUAAGUCU | 458 |
| 3605 | 19 mer | | | |
| STAT3- | Antisense | | AUACAUUACAAAGGAAAAU | 459 |
| 3611 | 19 mer | | | |
| STAT3- | Antisense | | UCAUGUCCAACCUGUAACU | 460 |
| 3906 | 19 mer | | | |
| STAT3- | Antisense | | UAACAAACAGAAUUCCACA | 461 |
| 4311 | 19 mer | | | |
| STAT3- | Antisense | | AUUUAACAAACAGAAUUCC | 462 |
| 4314 | 19 mer | | | |
| STAT3- | Antisense | | UUGAUUUAACAAACAGAAU | 463 |
| 4317 | 19 mer | | | |
| STAT3- | Antisense | | UAAUUUGAUUUAACAAACA | 464 |
| 4321 | 19 mer | | | |
| STAT3- | Antisense | | UCAGUUAAGCUUAUUAUGU | 465 |
| 4465 | 19 mer | | | |
| STAT3- | Antisense | | AAAUAUUCUGUUUAUCAGU | 466 |
| 4479 | 19 mer | | | |
| STAT3- | Antisense | | UAAAUAUUCUGUUUAUCAG | 467 |
| 4480 | 19 mer | | | |
| STAT3- | Antisense | | AAUAUAAAUUUUUACACUA | 468 |
| 4831 | 19 mer | | | |
| STAT3- | Antisense | | AUAAUAUAAAUUUUUACAC | 469 |
| 4833 | 19 mer | | | |
| STAT3- | Antisense | | ACAAUAAUAUAAAUUUUUA | 470 |
| 4836 | 19 mer | | | |
| STAT3- | Antisense | | CACAAUAAUAUAAAUUUUU | 471 |
| 4837 | 19 mer | | | |
| STAT3- | Antisense | | UUUAUUUCUGGAAGUUAAA | 472 |
| 4909 | 19 mer | | | |
| STAT3- | 25 mer | | CACUUUGGUGUUUCAUAAUAGCAGC | 473 |
| 370 | Sense | | | |
| | Strand | | | |
| STAT3- | 25 mer | | CUUUGGUGUUUCAUAAUCUAGCAGC | 474 |
| 372 | Sense | | | |
| | Strand | | | |
| STAT3- | 25 mer | | CCUGCAAGAGUCGAAUGUUAGCAGC | 475 |
| 424 | Sense | | | |
| | Strand | | | |
| STAT3- | 25 mer | | CUGCAAGAGUCGAAUGUUCAGCAGC | 476 |
| 425 | Sense | | | |
| | Strand | | | |
| STAT3- | 25 mer | | UGCAAGAGUCGAAUGUUCUAGCAGC | 477 |
| 426 | Sense | | | |
| | Strand | | | |
| STAT3- | 25 mer | | AAGAGUCGAAUGUUCUCUAAGCAGC | 478 |
| 429 | Sense | | | |
| | Strand | | | |
| STAT3- | 25 mer | | AGAGUCGAAUGUUCUCUAUAGCAGC | 479 |
| 430 | Sense | | | |
| | Strand | | | |
| STAT3- | 25 mer | | AGUCGAAUGUUCUCUAUCAAGCAGC | 480 |
| 432 | Sense | | | |
| | Strand | | | |
| STAT3- | 25 mer | | GUCGAAUGUUCUCUAUCAGAGCAGC | 481 |
| 433 | Sense | | | |
| | Strand | | | |
| STAT3- | 25 mer | | ACGAAGAAUCAAGCAGUUUAGCAGC | 482 |
| 460 | Sense | | | |
| | Strand | | | |
| STAT3- | 25 mer | | CGAAGAAUCAAGCAGUUUCAGCAGC | 483 |
| 461 | Sense | | | |
| | Strand | | | |
| STAT3- | 25 mer | | GAAGAAUCAAGCAGUUUCUAGCAGC | 484 |
| 462 | Sense | | | |
| | Strand | | | |
| STAT3- | 25 mer | | AUCUUGAGAAGCCAAUGGAAGCAGC | 485 |
| 492 | Sense | | | |
| | Strand | | | |
| STAT3- | 25 mer | | AGGAUCUAGAACAGAAAAUAGCAGC | 486 |
| 678 | Sense | | | |
| | Strand | | | |
| STAT3- | 25 mer | | AUCUAGAACAGAAAAUGAAAGCAGC | 487 |
| 681 | Sense | | | |
| | Strand | | | |
| STAT3- | 25 mer | | CCAGGAUGACUUUGAUUUCAGCAGC | 488 |
| 715 | Sense | | | |
| | Strand | | | |
| STAT3- | 25 mer | | CAGGAUGACUUUGAUUUCAAGCAGC | 489 |
| 716 | Sense | | | |
| | Strand | | | |
| STAT3- | 25 mer | | AGGAUGACUUUGAUUUCAAAGCAGC | 490 |
| 717 | Sense | | | |
| | Strand | | | |
| STAT3- | 25 mer | | AUGACUUUGAUUUCAACUAAGCAGC | 491 |
| 720 | Sense | | | |
| | Strand | | | |
| STAT3- | 25 mer | | UGACUUUGAUUUCAACUAUAGCAGC | 492 |
| 721 | Sense | | | |
| | Strand | | | |
| STAT3- | 25 mer | | GACUUUGAUUUCAACUAUAAGCAGC | 493 |
| 722 | Sense | | | |
| | Strand | | | |
| STAT3- | 25 mer | | ACUUUGAUUUCAACUAUAAAGCAGC | 494 |
| 723 | Sense | | | |
| | Strand | | | |
| STAT3- | 25 mer | | CUUUGAUUUCAACUAUAAAAGCAGC | 495 |
| 724 | Sense | | | |
| | Strand | | | |
| STAT3- | 25 mer | | AAGAUCUGAAUGGAAACAAAGCAGC | 496 |
| 768 | Sense | | | |
| | Strand | | | |
| STAT3- | 25 mer | | AUCUGAAUGGAAACAACCAAGCAGC | 497 |
| 771 | Sense | | | |
| | Strand | | | |
| STAT3- | 25 mer | | CUGAAUGGAAACAACCAGUAGCAGC | 498 |
| 773 | Sense | | | |
| | Strand | | | |
| STAT3- | 25 mer | | AGAAAACUGGAUAACGUCAAGCAGC | 499 |
| 1000 | Sense | | | |
| | Strand | | | |
| STAT3- | 25 mer | | GAAAACUGGAUAACGUCAUAGCAGC | 500 |
| 1001 | Sense | | | |
| | Strand | | | |
| STAT3- | 25 mer | | AAACUGGAUAACGUCAUUAAGCAGC | 501 |
| 1003 | Sense | | | |
| | Strand | | | |
| STAT3- | 25 mer | | CUGGAUAACGUCAUUAGCAAGCAGC | 502 |
| 1006 | Sense | | | |
| | Strand | | | |
| STAT3- | 25 mer | | GGAUAACGUCAUUAGCAGAAGCAGC | 503 |
| 1008 | Sense | | | |
| | Strand | | | |
| STAT3- | 25 mer | | GAUAACGUCAUUAGCAGAAAGCAGC | 504 |
| 1009 | Sense | | | |
| | Strand | | | |
| STAT3- | 25 mer | | AUAACGUCAUUAGCAGAAUAGCAGC | 505 |
| 1010 | Sense | | | |
| | Strand | | | |
| STAT3- | 25 mer | | AACAAAUUAAGAAACUGGAAGCAGC | 506 |
| 1047 | Sense | | | |
| | Strand | | | |
| STAT3- | 25 mer | | GAGUUGCAGCAAAAAGUUUAGCAGC | 507 |
| 1067 | Sense | | | |
| | Strand | | | |
| STAT3- | 25 mer | | AGUUGCAGCAAAAAGUUUCAGCAGC | 508 |
| 1068 | Sense | | | |
| | Strand | | | |
| STAT3- | 25 mer | | CUGUUUAGAAACUUAAUGAAGCAGC | 509 |
| 1145 | Sense | | | |
| | Strand | | | |
| STAT3- | 25 mer | | AGAAACUUAAUGAAAAGUGAGCAGC | 510 |
| 1151 | Sense | | | |
| | Strand | | | |
| STAT3- | 25 mer | | CAGUUCACUACUAAAGUCAAGCAGC | 511 |
| 1241 | Sense | | | |
| | Strand | | | |
| STAT3- | 25 mer | | GUCAAAUUCCCUGAGUUGAAGCAGC | 512 |
| 1268 | Sense | | | |
| | Strand | | | |
| STAT3- | 25 mer | | AAUUCCCUGAGUUGAAUUAAGCAGC | 513 |
| 1272 | Sense | | | |
| | Strand | | | |
| STAT3- | 25 mer | | AUUCCCUGAGUUGAAUUAUAGCAGC | 514 |
| 1273 | Sense | | | |
| | Strand | | | |
| STAT3- | 25 mer | | UCCCUGAGUUGAAUUAUCAAGCAGC | 515 |
| 1275 | Sense | | | |
| | Strand | | | |
| STAT3- | 25 mer | | CCUGAGUUGAAUUAUCAGCAGCAGC | 516 |
| 1277 | Sense | | | |
| | Strand | | | |
| STAT3- | 25 mer | | CUGAGUUGAAUUAUCAGCUAGCAGC | 517 |
| 1278 | Sense | | | |
| | Strand | | | |
| STAT3- | 25 mer | | UGAGUUGAAUUAUCAGCUUAGCAGC | 518 |
| 1279 | Sense | | | |
| | Strand | | | |
| STAT3- | 25 mer | | GAGUUGAAUUAUCAGCUUAAGCAGC | 519 |
| 1280 | Sense | | | |
| | Strand | | | |
| STAT3- | 25 mer | | AGUUGAAUUAUCAGCUUAAAGCAGC | 520 |
| 1281 | Sense | | | |
| | Strand | | | |
| STAT3- | 25 mer | | GUUGAAUUAUCAGCUUAAAAGCAGC | 521 |
| 1282 | Sense | | | |
| | Strand | | | |
| STAT3- | 25 mer | | UUGAAUUAUCAGCUUAAAAAGCAGC | 522 |
| 1283 | Sense | | | |
| | Strand | | | |
| STAT3- | 25 mer | | UGAAUUAUCAGCUUAAAAUAGCAGC | 523 |
| 1284 | Sense | | | |
| | Strand | | | |
| STAT3- | 25 mer | | AAUUAUCAGCUUAAAAUUAAGCAGC | 524 |
| 1286 | Sense | | | |
| | Strand | | | |
| STAT3- | 25 mer | | AUUAUCAGCUUAAAAUUAAAGCAGC | 525 |
| 1287 | Sense | | | |
| | Strand | | | |
| STAT3- | 25 mer | | CAGCUUAAAAUUAAAGUGUAGCAGC | 526 |
| 1292 | Sense | | | |
| | Strand | | | |
| STAT3- | 25 mer | | AGCUUAAAAUUAAAGUGUGAGCAGC | 527 |
| 1293 | Sense | | | |
| | Strand | | | |
| STAT3- | 25 mer | | AAAUUAAAGUGUGCAUUGAAGCAGC | 528 |
| 1299 | Sense | | | |
| | Strand | | | |
| STAT3- | 25 mer | | AAGUGUGCAUUGACAAAGAAGCAGC | 529 |
| 1305 | Sense | | | |
| | Strand | | | |
| STAT3- | 25 mer | | CAAAAGUGAUGAACAUGGAAGCAGC | 530 |
| 1383 | Sense | | | |
| | Strand | | | |
| STAT3- | 25 mer | | GUGAUGAACAUGGAAGAAUAGCAGC | 531 |
| 1388 | Sense | | | |
| | Strand | | | |
| STAT3- | 25 mer | | GCAGAAUUCAAACACUUGAAGCAGC | 532 |
| 1427 | Sense | | | |
| | Strand | | | |
| STAT3- | 25 mer | | AUUGUGAUGCUUCCCUGAUAGCAGC | 533 |
| 1485 | Sense | | | |
| | Strand | | | |
| STAT3- | 25 mer | | CCUUGCCAGUUGUGGUGAUAGCAGC | 534 |
| 1584 | Sense | | | |
| | Strand | | | |
| STAT3- | 25 mer | | UUGCCAGUUGUGGUGAUCUAGCAGC | 535 |
| 1586 | Sense | | | |
| | Strand | | | |
| STAT3- | 25 mer | | CCCAAGAAUGUAAACUUUUAGCAGC | 536 |
| 1670 | Sense | | | |
| | Strand | | | |
| STAT3- | 25 mer | | CCAAGAAUGUAAACUUUUUAGCAGC | 537 |
| 1671 | Sense | | | |
| | Strand | | | |
| STAT3- | 25 mer | | CAAGAAUGUAAACUUUUUUAGCAGC | 538 |
| 1672 | Sense | | | |
| | Strand | | | |
| STAT3- | 25 mer | | AAGAAUGUAAACUUUUUUAAGCAGC | 539 |
| 1673 | Sense | | | |
| | Strand | | | |
| STAT3- | 25 mer | | AGAAUGUAAACUUUUUUACAGCAGC | 540 |
| 1674 | Sense | | | |
| | Strand | | | |
| STAT3- | 25 mer | | AAUGUAAACUUUUUUACCAAGCAGC | 541 |
| 1676 | Sense | | | |
| | Strand | | | |
| STAT3- | 25 mer | | ACCUGGUGUGAAUUAUUCAAGCAGC | 542 |
| 1813 | Sense | | | |
| | Strand | | | |
| STAT3- | 25 mer | | CUGGUGUGAAUUAUUCAGGAGCAGC | 543 |
| 1815 | Sense | | | |
| | Strand | | | |
| STAT3- | 25 mer | | GGUGUGAAUUAUUCAGGGUAGCAGC | 544 |
| 1817 | Sense | | | |
| | Strand | | | |
| STAT3- | 25 mer | | UGUGAAUUAUUCAGGGUGUAGCAGC | 545 |
| 1819 | Sense | | | |
| | Strand | | | |
| STAT3- | 25 mer | | CUGGACAAUAUCAUUGACCAGCAGC | 546 |
| 1904 | Sense | | | |
| | Strand | | | |
| STAT3- | 25 mer | | GGACAAUAUCAUUGACCUUAGCAGC | 547 |
| 1906 | Sense | | | |
| | Strand | | | |
| STAT3- | 25 mer | | GACAAUAUCAUUGACCUUGAGCAGC | 548 |
| 1907 | Sense | | | |
| | Strand | | | |
| STAT3- | 25 mer | | ACAAUAUCAUUGACCUUGUAGCAGC | 549 |
| 1908 | Sense | | | |
| | Strand | | | |
| STAT3- | 25 mer | | CAAUAUCAUUGACCUUGUGAGCAGC | 550 |
| 1909 | Sense | | | |
| | Strand | | | |
| STAT3- | 25 mer | | AAUAUCAUUGACCUUGUGAAGCAGC | 551 |
| 1910 | Sense | | | |
| | Strand | | | |
| STAT3- | 25 mer | | AUAUCAUUGACCUUGUGAAAGCAGC | 552 |
| 1911 | Sense | | | |
| | Strand | | | |
| STAT3- | 25 mer | | UAUCAUUGACCUUGUGAAAAGCAGC | 553 |
| 1912 | Sense | | | |
| | Strand | | | |
| STAT3- | 25 mer | | AUCAUUGACCUUGUGAAAAAGCAGC | 554 |
| 1913 | Sense | | | |
| | Strand | | | |
| STAT3- | 25 mer | | UCAUUGACCUUGUGAAAAAAGCAGC | 555 |
| 1914 | Sense | | | |
| | Strand | | | |
| STAT3- | 25 mer | | AUUGACCUUGUGAAAAAGUAGCAGC | 556 |
| 1916 | Sense | | | |
| | Strand | | | |
| STAT3- | 25 mer | | UUGACCUUGUGAAAAAGUAAGCAGC | 557 |
| 1917 | Sense | | | |
| | Strand | | | |
| STAT3- | 25 mer | | GACCUUGUGAAAAAGUACAAGCAGC | 558 |
| 1919 | Sense | | | |
| | Strand | | | |
| STAT3- | 25 mer | | ACCUUGUGAAAAAGUACAUAGCAGC | 559 |
| 1920 | Sense | | | |
| | Strand | | | |
| STAT3- | 25 mer | | ACCUUCCUGCUAAGAUUCAAGCAGC | 560 |
| 2024 | Sense | | | |
| | Strand | | | |
| STAT3- | 25 mer | | AAGCAGCAGCUGAACAACAAGCAGC | 561 |
| 2135 | Sense | | | |
| | Strand | | | |
| STAT3- | 25 mer | | AGCAGCAGCUGAACAACAUAGCAGC | 562 |
| 2136 | Sense | | | |
| | Strand | | | |
| STAT3- | 25 mer | | CAGCAGCUGAACAACAUGUAGCAGC | 563 |
| 2138 | Sense | | | |
| | Strand | | | |
| STAT3- | 25 mer | | AGCAGCUGAACAACAUGUCAGCAGC | 564 |
| 2139 | Sense | | | |
| | Strand | | | |
| STAT3- | 25 mer | | GCUGAACAACAUGUCAUUUAGCAGC | 565 |
| 2143 | Sense | | | |
| | Strand | | | |
| STAT3- | 25 mer | | CUGAACAACAUGUCAUUUGAGCAGC | 566 |
| 2144 | Sense | | | |
| | Strand | | | |
| STAT3- | 25 mer | | UGAACAACAUGUCAUUUGCAGCAGC | 567 |
| 2145 | Sense | | | |
| | Strand | | | |
| STAT3- | 25 mer | | GAACAACAUGUCAUUUGCUAGCAGC | 568 |
| 2146 | Sense | | | |
| | Strand | | | |
| STAT3- | 25 mer | | AACAACAUGUCAUUUGCUGAGCAGC | 569 |
| 2147 | Sense | | | |
| | Strand | | | |
| STAT3- | 25 mer | | ACAACAUGUCAUUUGCUGAAGCAGC | 570 |
| 2148 | Sense | | | |
| | Strand | | | |
| STAT3- | 25 mer | | ACAUGUCAUUUGCUGAAAUAGCAGC | 571 |
| 2151 | Sense | | | |
| | Strand | | | |
| STAT3- | 25 mer | | AUGUCAUUUGCUGAAAUCAAGCAGC | 572 |
| 2153 | Sense | | | |
| | Strand | | | |
| STAT3- | 25 mer | | UGUCAUUUGCUGAAAUCAUAGCAGC | 573 |
| 2154 | Sense | | | |
| | Strand | | | |
| STAT3- | 25 mer | | UUUGCUGAAAUCAUCAUGGAGCAGC | 574 |
| 2159 | Sense | | | |
| | Strand | | | |
| STAT3- | 25 mer | | CAUACCUGAAGACCAAGUUAGCAGC | 575 |
| 2322 | Sense | | | |
| | Strand | | | |
| STAT3- | 25 mer | | ACCUGAAGACCAAGUUUAUAGCAGC | 576 |
| 2325 | Sense | | | |
| | Strand | | | |
| STAT3- | 25 mer | | CUGAAGACCAAGUUUAUCUAGCAGC | 577 |
| 2327 | Sense | | | |
| | Strand | | | |
| STAT3- | 25 mer | | GAAGACCAAGUUUAUCUGUAGCAGC | 578 |
| 2329 | Sense | | | |
| | Strand | | | |
| STAT3- | 25 mer | | ACCAAGUUUAUCUGUGUGAAGCAGC | 579 |
| 2333 | Sense | | | |
| | Strand | | | |
| STAT3- | 25 mer | | CAAGUUUAUCUGUGUGACAAGCAGC | 580 |
| 2335 | Sense | | | |
| | Strand | | | |
| STAT3- | 25 mer | | AGAUUCAUUGAUGCAGUUUAGCAGC | 581 |
| 2404 | Sense | | | |
| | Strand | | | |
| STAT3- | 25 mer | | GAUUCAUUGAUGCAGUUUGAGCAGC | 582 |
| 2405 | Sense | | | |
| | Strand | | | |
| STAT3- | 25 mer | | UUCAUUGAUGCAGUUUGGAAGCAGC | 583 |
| 2407 | Sense | | | |
| | Strand | | | |
| STAT3- | 25 mer | | UCAUUGAUGCAGUUUGGAAAGCAGC | 584 |
| 2408 | Sense | | | |
| | Strand | | | |
| STAT3- | 25 mer | | UUGAUGCAGUUUGGAAAUAAGCAGC | 585 |
| 2411 | Sense | | | |
| | Strand | | | |
| STAT3- | 25 mer | | UGAUGCAGUUUGGAAAUAAAGCAGC | 586 |
| 2412 | Sense | | | |
| | Strand | | | |
| STAT3- | 25 mer | | GAUGCAGUUUGGAAAUAAUAGCAGC | 587 |
| 2413 | Sense | | | |
| | Strand | | | |
| STAT3- | 25 mer | | GCAGUUUGGAAAUAAUGGUAGCAGC | 588 |
| 2416 | Sense | | | |
| | Strand | | | |
| STAT3- | 25 mer | | AGUUUGGAAAUAAUGGUGAAGCAGC | 589 |
| 2418 | Sense | | | |
| | Strand | | | |
| STAT3- | 25 mer | | UGGAAAUAAUGGUGAAGGUAGCAGC | 590 |
| 2422 | Sense | | | |
| | Strand | | | |
| STAT3- | 25 mer | | AUAAUGGUGAAGGUGCUGAAGCAGC | 591 |
| 2427 | Sense | | | |
| | Strand | | | |
| STAT3- | 25 mer | | CUGAAACUACUAACUUUGUAGCAGC | 592 |
| 2612 | Sense | | | |
| | Strand | | | |
| STAT3- | 25 mer | | AAACUACUAACUUUGUGGUAGCAGC | 593 |
| 2615 | Sense | | | |
| | Strand | | | |
| STAT3- | 25 mer | | AACUACUAACUUUGUGGUUAGCAGC | 594 |
| 2616 | Sense | | | |
| | Strand | | | |
| STAT3- | 25 mer | | ACUACUAACUUUGUGGUUCAGCAGC | 595 |
| 2617 | Sense | | | |
| | Strand | | | |
| STAT3- | 25 mer | | UAACUUUGUGGUUCCAGAUAGCAGC | 596 |
| 2622 | Sense | | | |
| | Strand | | | |
| STAT3- | 25 mer | | CUUUGUGGUUCCAGAUUUUAGCAGC | 597 |
| 2625 | Sense | | | |
| | Strand | | | |
| STAT3- | 25 mer | | UUUGUGGUUCCAGAUUUUUAGCAGC | 598 |
| 2626 | Sense | | | |
| | Strand | | | |
| STAT3- | 25 mer | | UUGUGGUUCCAGAUUUUUUAGCAGC | 599 |
| 2627 | Sense | | | |
| | Strand | | | |
| STAT3- | 25 mer | | AAAUAGAGAAAUGAGUGAAAGCAGC | 600 |
| 2692 | Sense | | | |
| | Strand | | | |
| STAT3- | 25 mer | | AAUAGAGAAAUGAGUGAAUAGCAGC | 601 |
| 2693 | Sense | | | |
| | Strand | | | |
| STAT3- | 25 mer | | GGUGAUCUGCUUUUAUCUAAGCAGC | 602 |
| 2715 | Sense | | | |
| | Strand | | | |
| STAT3- | 25 mer | | AUCUGCUUUUAUCUAAAUGAGCAGC | 603 |
| 2719 | Sense | | | |
| | Strand | | | |
| STAT3- | 25 mer | | CUGCUUUUAUCUAAAUGCAAGCAGC | 604 |
| 2721 | Sense | | | |
| | Strand | | | |
| STAT3- | 25 mer | | AUGCAAAUAAGGAUGUGUUAGCAGC | 605 |
| 2735 | Sense | | | |
| | Strand | | | |
| STAT3- | 25 mer | | AUAAGGAUGUGUUCUCUGAAGCAGC | 606 |
| 2741 | Sense | | | |
| | Strand | | | |
| STAT3- | 25 mer | | GAAAAAGGAAAUGUCUUGUAGCAGC | 607 |
| 2801 | Sense | | | |
| | Strand | | | |
| STAT3- | 25 mer | | AAAAGGAAAUGUCUUGUGUAGCAGC | 608 |
| 2803 | Sense | | | |
| | Strand | | | |
| STAT3- | 25 mer | | AAAGGAAAUGUCUUGUGUUAGCAGC | 609 |
| 2804 | Sense | | | |
| | Strand | | | |
| STAT3- | 25 mer | | AGGAAAUGUCUUGUGUUGUAGCAGC | 610 |
| 2806 | Sense | | | |
| | Strand | | | |
| STAT3- | 25 mer | | GGAAAUGUCUUGUGUUGUUAGCAGC | 611 |
| 2807 | Sense | | | |
| | Strand | | | |
| STAT3- | 25 mer | | GAAAUGUCUUGUGUUGUUUAGCAGC | 612 |
| 2808 | Sense | | | |
| | Strand | | | |
| STAT3- | 25 mer | | AAAUGUCUUGUGUUGUUUUAGCAGC | 613 |
| 2809 | Sense | | | |
| | Strand | | | |
| STAT3- | 25 mer | | AAUGUCUUGUGUUGUUUUGAGCAGC | 614 |
| 2810 | Sense | | | |
| | Strand | | | |
| STAT3- | 25 mer | | AUGUCUUGUGUUGUUUUGUAGCAGC | 615 |
| 2811 | Sense | | | |
| | Strand | | | |
| STAT3- | 25 mer | | UGUCUUGUGUUGUUUUGUUAGCAGC | 616 |
| 2812 | Sense | | | |
| | Strand | | | |
| STAT3- | 25 mer | | GUCUUGUGUUGUUUUGUUCAGCAGC | 617 |
| 2813 | Sense | | | |
| | Strand | | | |
| STAT3- | 25 mer | | CUCAGCAGCUUUUUGUUAUAGCAGC | 618 |
| 2846 | Sense | | | |
| | Strand | | | |
| STAT3- | 25 mer | | CAGCAGCUUUUUGUUAUUGAGCAGC | 619 |
| 2848 | Sense | | | |
| | Strand | | | |
| STAT3- | 25 mer | | AGCAGCUUUUUGUUAUUGUAGCAGC | 620 |
| 2849 | Sense | | | |
| | Strand | | | |
| STAT3- | 25 mer | | GCAGCUUUUUGUUAUUGUUAGCAGC | 621 |
| 2850 | Sense | | | |
| | Strand | | | |
| STAT3- | 25 mer | | CAGCUUUUUGUUAUUGUUGAGCAGC | 622 |
| 2851 | Sense | | | |
| | Strand | | | |
| STAT3- | 25 mer | | AGCUUUUUGUUAUUGUUGUAGCAGC | 623 |
| 2852 | Sense | | | |
| | Strand | | | |
| STAT3- | 25 mer | | GCUUUUUGUUAUUGUUGUUAGCAGC | 624 |
| 2853 | Sense | | | |
| | Strand | | | |
| STAT3- | 25 mer | | CUUUUUGUUAUUGUUGUUGAGCAGC | 625 |
| 2854 | Sense | | | |
| | Strand | | | |
| STAT3- | 25 mer | | UUUUUGUUAUUGUUGUUGUAGCAGC | 626 |
| 2855 | Sense | | | |
| | Strand | | | |
| STAT3- | 25 mer | | UUUUGUUAUUGUUGUUGUUAGCAGC | 627 |
| 2856 | Sense | | | |
| | Strand | | | |
| STAT3- | 25 mer | | UUUGUUAUUGUUGUUGUUGAGCAGC | 628 |
| 2857 | Sense | | | |
| | Strand | | | |
| STAT3- | 25 mer | | UUGUUAUUGUUGUUGUUGUAGCAGC | 629 |
| 2858 | Sense | | | |
| | Strand | | | |
| STAT3- | 25 mer | | UGUUAUUGUUGUUGUUGUUAGCAGC | 630 |
| 2859 | Sense | | | |
| | Strand | | | |
| STAT3- | 25 mer | | GUUAUUGUUGUUGUUGUUCAGCAGC | 631 |
| 2860 | Sense | | | |
| | Strand | | | |
| STAT3- | 25 mer | | UUAUUGUUGUUGUUGUUCUAGCAGC | 632 |
| 2861 | Sense | | | |
| | Strand | | | |
| STAT3- | 25 mer | | UAUUGUUGUUGUUGUUCUUAGCAGC | 633 |
| 2862 | Sense | | | |
| | Strand | | | |
| STAT3- | 25 mer | | AUUGUUGUUGUUGUUCUUAAGCAGC | 634 |
| 2863 | Sense | | | |
| | Strand | | | |
| STAT3- | 25 mer | | UGUUGUUGUUGUUCUUAGAAGCAGC | 635 |
| 2865 | Sense | | | |
| | Strand | | | |
| STAT3- | 25 mer | | UUGUUGUUGUUCUUAGACAAGCAGC | 636 |
| 2867 | Sense | | | |
| | Strand | | | |
| STAT3- | 25 mer | | UGUUGUUGUUCUUAGACAAAGCAGC | 637 |
| 2868 | Sense | | | |
| | Strand | | | |
| STAT3- | 25 mer | | CUUUUUAACCUUGCUGACAAGCAGC | 638 |
| 2975 | Sense | | | |
| | Strand | | | |
| STAT3- | 25 mer | | UUAACCUUGCUGACAUCCAAGCAGC | 639 |
| 2979 | Sense | | | |
| | Strand | | | |
| STAT3- | 25 mer | | UUGCUGACAUCCAAAUAGAAGCAGC | 640 |
| 2985 | Sense | | | |
| | Strand | | | |
| STAT3- | 25 mer | | AGGUUUCUUUUUAAAUUAAAGCAGC | 641 |
| 3025 | Sense | | | |
| | Strand | | | |
| STAT3- | 25 mer | | AAAUUAAGAAAUAAUAACAAGCAGC | 642 |
| 3037 | Sense | | | |
| | Strand | | | |
| STAT3- | 25 mer | | AAUUAAGAAAUAAUAACAAAGCAGC | 643 |
| 3038 | Sense | | | |
| | Strand | | | |
| STAT3- | 25 mer | | AUUAAGAAAUAAUAACAAUAGCAGC | 644 |
| 3039 | Sense | | | |
| | Strand | | | |
| STAT3- | 25 mer | | UAAGAAAUAAUAACAAUUAAGCAGC | 645 |
| 3041 | Sense | | | |
| | Strand | | | |
| STAT3- | 25 mer | | AAGAAAUAAUAACAAUUAAAGCAGC | 646 |
| 3042 | Sense | | | |
| | Strand | | | |
| STAT3- | 25 mer | | AGAAAUAAUAACAAUUAAAAGCAGC | 647 |
| 3043 | Sense | | | |
| | Strand | | | |
| STAT3- | 25 mer | | ACUAAAAGUACAAAAAAUUAGCAGC | 648 |
| 3225 | Sense | | | |
| | Strand | | | |
| STAT3- | 25 mer | | CUAAAAGUACAAAAAAUUAAGCAGC | 649 |
| 3226 | Sense | | | |
| | Strand | | | |
| STAT3- | 25 mer | | AGACUUAUUUUCCUUUGUAAGCAGC | 650 |
| 3605 | Sense | | | |
| | Strand | | | |
| STAT3- | 25 mer | | AUUUUCCUUUGUAAUGUAUAGCAGC | 651 |
| 3611 | Sense | | | |
| | Strand | | | |
| STAT3- | 25 mer | | AGUUACAGGUUGGACAUGAAGCAGC | 652 |
| 3906 | Sense | | | |
| | Strand | | | |
| STAT3- | 25 mer | | UGUGGAAUUCUGUUUGUUAAGCAGC | 653 |
| 4311 | Sense | | | |
| | Strand | | | |
| STAT3- | 25 mer | | GGAAUUCUGUUUGUUAAAUAGCAGC | 654 |
| 4314 | Sense | | | |
| | Strand | | | |
| STAT3- | 25 mer | | AUUCUGUUUGUUAAAUCAAAGCAGC | 655 |
| 4317 | Sense | | | |
| | Strand | | | |
| STAT3- | 25 mer | | UGUUUGUUAAAUCAAAUUAAGCAGC | 656 |
| 4321 | Sense | | | |
| | Strand | | | |
| STAT3- | 25 mer | | ACAUAAUAAGCUUAACUGAAGCAGC | 657 |
| 4465 | Sense | | | |
| | Strand | | | |
| STAT3- | 25 mer | | ACUGAUAAACAGAAUAUUUAGCAGC | 658 |
| 4479 | Sense | | | |
| | Strand | | | |
| STAT3- | 25 mer | | CUGAUAAACAGAAUAUUUAAGCAGC | 659 |
| 4480 | Sense | | | |
| | Strand | | | |
| STAT3- | 25 mer | | UAGUGUAAAAAUUUAUAUUAGCAGC | 660 |
| 4831 | Sense | | | |
| | Strand | | | |
| STAT3- | 25 mer | | GUGUAAAAAUUUAUAUUAUAGCAGC | 661 |
| 4833 | Sense | | | |
| | Strand | | | |
| STAT3- | 25 mer | | UAAAAAUUUAUAUUAUUGUAGCAGC | 662 |
| 4836 | Sense | | | |
| | Strand | | | |
| STAT3- | 25 mer | | AAAAAUUUAUAUUAUUGUGAGCAGC | 663 |
| 4837 | Sense | | | |
| | Strand | | | |
| STAT3- | 25 mer | | UUUAACUUCCAGAAAUAAAAGCAGC | 664 |
| 4909 | Sense | | | |
| | Strand | | | |
| STAT3- | 27 mer | | GCUGCUAUUAUGAAACACCAAAGUGGG | 665 |
| 370 | Antisense | | | |
| | Strand | | | |
| STAT3- | 27 mer | | GCUGCUAGAUUAUGAAACACCAAAGGG | 666 |
| 372 | Antisense | | | |
| | Strand | | | |
| STAT3- | 27 mer | | GCUGCUAACAUUCGACUCUUGCAGGGG | 667 |
| 424 | Antisense | | | |
| | Strand | | | |
| STAT3- | 27 mer | | GCUGCUGAACAUUCGACUCUUGCAGGG | 668 |
| 425 | Antisense | | | |
| | Strand | | | |
| STAT3- | 27 mer | | GCUGCUAGAACAUUCGACUCUUGCAGG | 669 |
| 426 | Antisense | | | |
| | Strand | | | |
| STAT3- | 27 mer | | GCUGCUUAGAGAACAUUCGACUCUUGG | 670 |
| 429 | Antisense | | | |
| | Strand | | | |
| STAT3- | 27 mer | | GCUGCUAUAGAGAACAUUCGACUCUGG | 671 |
| 430 | Antisense | | | |
| | Strand | | | |
| STAT3- | 27 mer | | GCUGCUUGAUAGAGAACAUUCGACUGG | 672 |
| 432 | Antisense | | | |
| | Strand | | | |
| STAT3- | 27 mer | | GCUGCUCUGAUAGAGAACAUUCGACGG | 673 |
| 433 | Antisense | | | |
| | Strand | | | |
| STAT3- | 27 mer | | GCUGCUAAACUGCUUGAUUCUUCGUGG | 674 |
| 460 | Antisense | | | |
| | Strand | | | |
| STAT3- | 27 mer | | GCUGCUGAAACUGCUUGAUUCUUCGGG | 675 |
| 461 | Antisense | | | |
| | Strand | | | |
| STAT3- | 27 mer | | GCUGCUAGAAACUGCUUGAUUCUUCGG | 676 |
| 462 | Antisense | | | |
| | Strand | | | |
| STAT3- | 27 mer | | GCUGCUUCCAUUGGCUUCUCAAGAUGG | 677 |
| 492 | Antisense | | | |
| | Strand | | | |
| STAT3- | 27 mer | | GCUGCUAUUUUCUGUUCUAGAUCCUGG | 678 |
| 678 | Antisense | | | |
| | Strand | | | |
| STAT3- | 27 mer | | GCUGCUUUCAUUUUCUGUUCUAGAUGG | 679 |
| 681 | Antisense | | | |
| | Strand | | | |
| STAT3- | 27 mer | | GCUGCUGAAAUCAAAGUCAUCCUGGGG | 680 |
| 715 | Antisense | | | |
| | Strand | | | |
| STAT3- | 27 mer | | GCUGCUUGAAAUCAAAGUCAUCCUGGG | 681 |
| 716 | Antisense | | | |
| | Strand | | | |
| STAT3- | 27 mer | | GCUGCUUUGAAAUCAAAGUCAUCCUGG | 682 |
| 717 | Antisense | | | |
| | Strand | | | |
| STAT3- | 27 mer | | GCUGCUUAGUUGAAAUCAAAGUCAUGG | 683 |
| 720 | Antisense | | | |
| | Strand | | | |
| STAT3- | 27 mer | | GCUGCUAUAGUUGAAAUCAAAGUCAGG | 684 |
| 721 | Antisense | | | |
| | Strand | | | |
| STAT3- | 27 mer | | GCUGCUUAUAGUUGAAAUCAAAGUCGG | 685 |
| 722 | Antisense | | | |
| | Strand | | | |
| STAT3- | 27 mer | | GCUGCUUUAUAGUUGAAAUCAAAGUGG | 686 |
| 723 | Antisense | | | |
| | Strand | | | |
| STAT3- | 27 mer | | GCUGCUUUUAUAGUUGAAAUCAAAGGG | 687 |
| 724 | Antisense | | | |
| | Strand | | | |
| STAT3- | 27 mer | | GCUGCUUUGUUUCCAUUCAGAUCUUGG | 688 |
| 768 | Antisense | | | |
| | Strand | | | |
| STAT3- | 27 mer | | GCUGCUUGGUUGUUUCCAUUCAGAUGG | 689 |
| 771 | Antisense | | | |
| | Strand | | | |
| STAT3- | 27 mer | | GCUGCUACUGGUUGUUUCCAUUCAGGG | 690 |
| 773 | Antisense | | | |
| | Strand | | | |
| STAT3- | 27 mer | | GCUGCUUGACGUUAUCCAGUUUUCUGG | 691 |
| 1000 | Antisense | | | |
| | Strand | | | |
| STAT3- | 27 mer | | GCUGCUAUGACGUUAUCCAGUUUUCGG | 692 |
| 1001 | Antisense | | | |
| | Strand | | | |
| STAT3- | 27 mer | | GCUGCUUAAUGACGUUAUCCAGUUUGG | 693 |
| 1003 | Antisense | | | |
| | Strand | | | |
| STAT3- | 27 mer | | GCUGCUUGCUAAUGACGUUAUCCAGGG | 694 |
| 1006 | Antisense | | | |
| | Strand | | | |
| STAT3- | 27 mer | | GCUGCUUCUGCUAAUGACGUUAUCCGG | 695 |
| 1008 | Antisense | | | |
| | Strand | | | |
| STAT3- | 27 mer | | GCUGCUUUCUGCUAAUGACGUUAUCGG | 696 |
| 1009 | Antisense | | | |
| | Strand | | | |
| STAT3- | 27 mer | | GCUGCUAUUCUGCUAAUGACGUUAUGG | 697 |
| 1010 | Antisense | | | |
| | Strand | | | |
| STAT3- | 27 mer | | GCUGCUUCCAGUUUCUUAAUUUGUUGG | 698 |
| 1047 | Antisense | | | |
| | Strand | | | |
| STAT3- | 27 mer | | GCUGCUAAACUUUUUGCUGCAACUCGG | 699 |
| 1067 | Antisense | | | |
| | Strand | | | |
| STAT3- | 27 mer | | GCUGCUGAAACUUUUUGCUGCAACUGG | 700 |
| 1068 | Antisense | | | |
| | Strand | | | |
| STAT3- | 27 mer | | GCUGCUUCAUUAAGUUUCUAAACAGGG | 701 |
| 1145 | Antisense | | | |
| | Strand | | | |
| STAT3- | 27 mer | | GCUGCUCACUUUUCAUUAAGUUUCUGG | 702 |
| 1151 | Antisense | | | |
| | Strand | | | |
| STAT3- | 27 mer | | GCUGCUUGACUUUAGUAGUGAACUGGG | 703 |
| 1241 | Antisense | | | |
| | Strand | | | |
| STAT3- | 27 mer | | GCUGCUUCAACUCAGGGAAUUUGACGG | 704 |
| 1268 | Antisense | | | |
| | Strand | | | |
| STAT3- | 27 mer | | GCUGCUUAAUUCAACUCAGGGAAUUGG | 705 |
| 1272 | Antisense | | | |
| | Strand | | | |
| STAT3- | 27 mer | | GCUGCUAUAAUUCAACUCAGGGAAUGG | 706 |
| 1273 | Antisense | | | |
| | Strand | | | |
| STAT3- | 27 mer | | GCUGCUUGAUAAUUCAACUCAGGGAGG | 707 |
| 1275 | Antisense | | | |
| | Strand | | | |
| STAT3- | 27 mer | | GCUGCUGCUGAUAAUUCAACUCAGGGG | 708 |
| 1277 | Antisense | | | |
| | Strand | | | |
| STAT3- | 27 mer | | GCUGCUAGCUGAUAAUUCAACUCAGGG | 709 |
| 1278 | Antisense | | | |
| | Strand | | | |
| STAT3- | 27 mer | | GCUGCUAAGCUGAUAAUUCAACUCAGG | 710 |
| 1279 | Antisense | | | |
| | Strand | | | |
| STAT3- | 27 mer | | GCUGCUUAAGCUGAUAAUUCAACUCGG | 711 |
| 1280 | Antisense | | | |
| | Strand | | | |
| STAT3- | 27 mer | | GCUGCUUUAAGCUGAUAAUUCAACUGG | 712 |
| 1281 | Antisense | | | |
| | Strand | | | |
| STAT3- | 27 mer | | GCUGCUUUUAAGCUGAUAAUUCAACGG | 713 |
| 1282 | Antisense | | | |
| | Strand | | | |
| STAT3- | 27 mer | | GCUGCUUUUUAAGCUGAUAAUUCAAGG | 714 |
| 1283 | Antisense | | | |
| | Strand | | | |
| STAT3- | 27 mer | | GCUGCUAUUUUAAGCUGAUAAUUCAGG | 715 |
| 1284 | Antisense | | | |
| | Strand | | | |
| STAT3- | 27 mer | | GCUGCUUAAUUUUAAGCUGAUAAUUGG | 716 |
| 1286 | Antisense | | | |
| | Strand | | | |
| STAT3- | 27 mer | | GCUGCUUUAAUUUUAAGCUGAUAAUGG | 717 |
| 1287 | Antisense | | | |
| | Strand | | | |
| STAT3- | 27 mer | | GCUGCUACACUUUAAUUUUAAGCUGGG | 718 |
| 1292 | Antisense | | | |
| | Strand | | | |
| STAT3- | 27 mer | | GCUGCUCACACUUUAAUUUUAAGCUGG | 719 |
| 1293 | Antisense | | | |
| | Strand | | | |
| STAT3- | 27 mer | | GCUGCUUCAAUGCACACUUUAAUUUGG | 720 |
| 1299 | Antisense | | | |
| | Strand | | | |
| STAT3- | 27 mer | | GCUGCUUCUUUGUCAAUGCACACUUGG | 721 |
| 1305 | Antisense | | | |
| | Strand | | | |
| STAT3- | 27 mer | | GCUGCUUCCAUGUUCAUCACUUUUGGG | 722 |
| 1383 | Antisense | | | |
| | Strand | | | |
| STAT3- | 27 mer | | GCUGCUAUUCUUCCAUGUUCAUCACGG | 723 |
| 1388 | Antisense | | | |
| | Strand | | | |
| STAT3- | 27 mer | | GCUGCUUCAAGUGUUUGAAUUCUGCGG | 724 |
| 1427 | Antisense | | | |
| | Strand | | | |
| STAT3- | 27 mer | | GCUGCUAUCAGGGAAGCAUCACAAUGG | 725 |
| 1485 | Antisense | | | |
| | Strand | | | |
| STAT3- | 27 mer | | GCUGCUAUCACCACAACUGGCAAGGGG | 726 |
| 1584 | Antisense | | | |
| | Strand | | | |
| STAT3- | 27 mer | | GCUGCUAGAUCACCACAACUGGCAAGG | 727 |
| 1586 | Antisense | | | |
| | Strand | | | |
| STAT3- | 27 mer | | GCUGCUAAAAGUUUACAUUCUUGGGGG | 728 |
| 1670 | Antisense | | | |
| | Strand | | | |
| STAT3- | 27 mer | | GCUGCUAAAAAGUUUACAUUCUUGGGG | 729 |
| 1671 | Antisense | | | |
| | Strand | | | |
| STAT3- | 27 mer | | GCUGCUAAAAAAGUUUACAUUCUUGGG | 730 |
| 1672 | Antisense | | | |
| | Strand | | | |
| STAT3- | 27 mer | | GCUGCUUAAAAAAGUUUACAUUCUUGG | 731 |
| 1673 | Antisense | | | |
| | Strand | | | |
| STAT3- | 27 mer | | GCUGCUGUAAAAAAGUUUACAUUCUGG | 732 |
| 1674 | Antisense | | | |
| | Strand | | | |
| STAT3- | 27 mer | | GCUGCUUGGUAAAAAAGUUUACAUUGG | 733 |
| 1676 | Antisense | | | |
| | Strand | | | |
| STAT3- | 27 mer | | GCUGCUUGAAUAAUUCACACCAGGUGG | 734 |
| 1813 | Antisense | | | |
| | Strand | | | |
| STAT3- | 27 mer | | GCUGCUCCUGAAUAAUUCACACCAGGG | 735 |
| 1815 | Antisense | | | |
| | Strand | | | |
| STAT3- | 27 mer | | GCUGCUACCCUGAAUAAUUCACACCGG | 736 |
| 1817 | Antisense | | | |
| | Strand | | | |
| STAT3- | 27 mer | | GCUGCUACACCCUGAAUAAUUCACAGG | 737 |
| 1819 | Antisense | | | |
| | Strand | | | |
| STAT3- | 27 mer | | GCUGCUGGUCAAUGAUAUUGUCCAGGG | 738 |
| 1904 | Antisense | | | |
| | Strand | | | |
| STAT3- | 27 mer | | GCUGCUAAGGUCAAUGAUAUUGUCCGG | 739 |
| 1906 | Antisense | | | |
| | Strand | | | |
| STAT3- | 27 mer | | GCUGCUCAAGGUCAAUGAUAUUGUCGG | 740 |
| 1907 | Antisense | | | |
| | Strand | | | |
| STAT3- | 27 mer | | GCUGCUACAAGGUCAAUGAUAUUGUGG | 741 |
| 1908 | Antisense | | | |
| | Strand | | | |
| STAT3- | 27 mer | | GCUGCUCACAAGGUCAAUGAUAUUGGG | 742 |
| 1909 | Antisense | | | |
| | Strand | | | |
| STAT3- | 27 mer | | GCUGCUUCACAAGGUCAAUGAUAUUGG | 743 |
| 1910 | Antisense | | | |
| | Strand | | | |
| STAT3- | 27 mer | | GCUGCUUUCACAAGGUCAAUGAUAUGG | 744 |
| 1911 | Antisense | | | |
| | Strand | | | |
| STAT3- | 27 mer | | GCUGCUUUUCACAAGGUCAAUGAUAGG | 745 |
| 1912 | Antisense | | | |
| | Strand | | | |
| STAT3- | 27 mer | | GCUGCUUUUUCACAAGGUCAAUGAUGG | 746 |
| 1913 | Antisense | | | |
| | Strand | | | |
| STAT3- | 27 mer | | GCUGCUUUUUUCACAAGGUCAAUGAGG | 747 |
| 1914 | Antisense | | | |
| | Strand | | | |
| STAT3- | 27 mer | | GCUGCUACUUUUUCACAAGGUCAAUGG | 748 |
| 1916 | Antisense | | | |
| | Strand | | | |
| STAT3- | 27 mer | | GCUGCUUACUUUUUCACAAGGUCAAGG | 749 |
| 1917 | Antisense | | | |
| | Strand | | | |
| STAT3- | 27 mer | | GCUGCUUGUACUUUUUCACAAGGUCGG | 750 |
| 1919 | Antisense | | | |
| | Strand | | | |
| STAT3- | 27 mer | | GCUGCUAUGUACUUUUUCACAAGGUGG | 751 |
| 1920 | Antisense | | | |
| | Strand | | | |
| STAT3- | 27 mer | | GCUGCUUGAAUCUUAGCAGGAAGGUGG | 752 |
| 2024 | Antisense | | | |
| | Strand | | | |
| STAT3- | 27 mer | | GCUGCUUGUUGUUCAGCUGCUGCUUGG | 753 |
| 2135 | Antisense | | | |
| | Strand | | | |
| STAT3- | 27 mer | | GCUGCUAUGUUGUUCAGCUGCUGCUGG | 754 |
| 2136 | Antisense | | | |
| | Strand | | | |
| STAT3- | 27 mer | | GCUGCUACAUGUUGUUCAGCUGCUGGG | 755 |
| 2138 | Antisense | | | |
| | Strand | | | |
| STAT3- | 27 mer | | GCUGCUGACAUGUUGUUCAGCUGCUGG | 756 |
| 2139 | Antisense | | | |
| | Strand | | | |
| STAT3- | 27 mer | | GCUGCUAAAUGACAUGUUGUUCAGCGG | 757 |
| 2143 | Antisense | | | |
| | Strand | | | |
| STAT3- | 27 mer | | GCUGCUCAAAUGACAUGUUGUUCAGGG | 758 |
| 2144 | Antisense | | | |
| | Strand | | | |
| STAT3- | 27 mer | | GCUGCUGCAAAUGACAUGUUGUUCAGG | 759 |
| 2145 | Antisense | | | |
| | Strand | | | |
| STAT3- | 27 mer | | GCUGCUAGCAAAUGACAUGUUGUUCGG | 760 |
| 2146 | Antisense | | | |
| | Strand | | | |
| STAT3- | 27 mer | | GCUGCUCAGCAAAUGACAUGUUGUUGG | 761 |
| 2147 | Antisense | | | |
| | Strand | | | |
| STAT3- | 27 mer | | GCUGCUUCAGCAAAUGACAUGUUGUGG | 762 |
| 2148 | Antisense | | | |
| | Strand | | | |
| STAT3- | 27 mer | | GCUGCUAUUUCAGCAAAUGACAUGUGG | 763 |
| 2151 | Antisense | | | |
| | Strand | | | |
| STAT3- | 27 mer | | GCUGCUUGAUUUCAGCAAAUGACAUGG | 764 |
| 2153 | Antisense | | | |
| | Strand | | | |
| STAT3- | 27 mer | | GCUGCUAUGAUUUCAGCAAAUGACAGG | 765 |
| 2154 | Antisense | | | |
| | Strand | | | |
| STAT3- | 27 mer | | GCUGCUCCAUGAUGAUUUCAGCAAAGG | 766 |
| 2159 | Antisense | | | |
| | Strand | | | |
| STAT3- | 27 mer | | GCUGCUAACUUGGUCUUCAGGUAUGGG | 767 |
| 2322 | Antisense | | | |
| | Strand | | | |
| STAT3- | 27 mer | | GCUGCUAUAAACUUGGUCUUCAGGUGG | 768 |
| 2325 | Antisense | | | |
| | Strand | | | |
| STAT3- | 27 mer | | GCUGCUAGAUAAACUUGGUCUUCAGGG | 769 |
| 2327 | Antisense | | | |
| | Strand | | | |
| STAT3- | 27 mer | | GCUGCUACAGAUAAACUUGGUCUUCGG | 770 |
| 2329 | Antisense | | | |
| | Strand | | | |
| STAT3- | 27 mer | | GCUGCUUCACACAGAUAAACUUGGUGG | 771 |
| 2333 | Antisense | | | |
| | Strand | | | |
| STAT3- | 27 mer | | GCUGCUUGUCACACAGAUAAACUUGGG | 772 |
| 2335 | Antisense | | | |
| | Strand | | | |
| STAT3- | 27 mer | | GCUGCUAAACUGCAUCAAUGAAUCUGG | 773 |
| 2404 | Antisense | | | |
| | Strand | | | |
| STAT3- | 27 mer | | GCUGCUCAAACUGCAUCAAUGAAUCGG | 774 |
| 2405 | Antisense | | | |
| | Strand | | | |
| STAT3- | 27 mer | | GCUGCUUCCAAACUGCAUCAAUGAAGG | 775 |
| 2407 | Antisense | | | |
| | Strand | | | |
| STAT3- | 27 mer | | GCUGCUUUCCAAACUGCAUCAAUGAGG | 776 |
| 2408 | Antisense | | | |
| | Strand | | | |
| STAT3- | 27 mer | | GCUGCUUAUUUCCAAACUGCAUCAAGG | 777 |
| 2411 | Antisense | | | |
| | Strand | | | |
| STAT3- | 27 mer | | GCUGCUUUAUUUCCAAACUGCAUCAGG | 778 |
| 2412 | Antisense | | | |
| | Strand | | | |
| STAT3- | 27 mer | | GCUGCUAUUAUUUCCAAACUGCAUCGG | 779 |
| 2413 | Antisense | | | |
| | Strand | | | |
| STAT3- | 27 mer | | GCUGCUACCAUUAUUUCCAAACUGCGG | 780 |
| 2416 | Antisense | | | |
| | Strand | | | |
| STAT3- | 27 mer | | GCUGCUUCACCAUUAUUUCCAAACUGG | 781 |
| 2418 | Antisense | | | |
| | Strand | | | |
| STAT3- | 27 mer | | GCUGCUACCUUCACCAUUAUUUCCAGG | 782 |
| 2422 | Antisense | | | |
| | Strand | | | |
| STAT3- | 27 mer | | GCUGCUUCAGCACCUUCACCAUUAUGG | 783 |
| 2427 | Antisense | | | |
| | Strand | | | |
| STAT3- | 27 mer | | GCUGCUACAAAGUUAGUAGUUUCAGGG | 784 |
| 2612 | Antisense | | | |
| | Strand | | | |
| STAT3- | 27 mer | | GCUGCUACCACAAAGUUAGUAGUUUGG | 785 |
| 2615 | Antisense | | | |
| | Strand | | | |
| STAT3- | 27 mer | | GCUGCUAACCACAAAGUUAGUAGUUGG | 786 |
| 2616 | Antisense | | | |
| | Strand | | | |
| STAT3- | 27 mer | | GCUGCUGAACCACAAAGUUAGUAGUGG | 787 |
| 2617 | Antisense | | | |
| | Strand | | | |
| STAT3- | 27 mer | | GCUGCUAUCUGGAACCACAAAGUUAGG | 788 |
| 2622 | Antisense | | | |
| | Strand | | | |
| STAT3- | 27 mer | | GCUGCUAAAAUCUGGAACCACAAAGGG | 789 |
| 2625 | Antisense | | | |
| | Strand | | | |
| STAT3- | 27 mer | | GCUGCUAAAAAUCUGGAACCACAAAGG | 790 |
| 2626 | Antisense | | | |
| | Strand | | | |
| STAT3- | 27 mer | | GCUGCUAAAAAAUCUGGAACCACAAGG | 791 |
| 2627 | Antisense | | | |
| | Strand | | | |
| STAT3- | 27 mer | | GCUGCUUUCACUCAUUUCUCUAUUUGG | 792 |
| 2692 | Antisense | | | |
| | Strand | | | |
| STAT3- | 27 mer | | GCUGCUAUUCACUCAUUUCUCUAUUGG | 793 |
| 2693 | Antisense | | | |
| | Strand | | | |
| STAT3- | 27 mer | | GCUGCUUAGAUAAAAGCAGAUCACCGG | 794 |
| 2715 | Antisense | | | |
| | Strand | | | |
| STAT3- | 27 mer | | GCUGCUCAUUUAGAUAAAAGCAGAUGG | 795 |
| 2719 | Antisense | | | |
| | Strand | | | |
| STAT3- | 27 mer | | GCUGCUUGCAUUUAGAUAAAAGCAGGG | 796 |
| 2721 | Antisense | | | |
| | Strand | | | |
| STAT3- | 27 mer | | GCUGCUAACACAUCCUUAUUUGCAUGG | 797 |
| 2735 | Antisense | | | |
| | Strand | | | |
| STAT3- | 27 mer | | GCUGCUUCAGAGAACACAUCCUUAUGG | 798 |
| 2741 | Antisense | | | |
| | Strand | | | |
| STAT3- | 27 mer | | GCUGCUACAAGACAUUUCCUUUUUCGG | 799 |
| 2801 | Antisense | | | |
| | Strand | | | |
| STAT3- | 27 mer | | GCUGCUACACAAGACAUUUCCUUUUGG | 800 |
| 2803 | Antisense | | | |
| | Strand | | | |
| STAT3- | 27 mer | | GCUGCUAACACAAGACAUUUCCUUUGG | 801 |
| 2804 | Antisense | | | |
| | Strand | | | |
| STAT3- | 27 mer | | GCUGCUACAACACAAGACAUUUCCUGG | 802 |
| 2806 | Antisense | | | |
| | Strand | | | |
| STAT3- | 27 mer | | GCUGCUAACAACACAAGACAUUUCCGG | 803 |
| 2807 | Antisense | | | |
| | Strand | | | |
| STAT3- | 27 mer | | GCUGCUAAACAACACAAGACAUUUCGG | 804 |
| 2808 | Antisense | | | |
| | Strand | | | |
| STAT3- | 27 mer | | GCUGCUAAAACAACACAAGACAUUUGG | 805 |
| 2809 | Antisense | | | |
| | Strand | | | |
| STAT3- | 27 mer | | GCUGCUCAAAACAACACAAGACAUUGG | 806 |
| 2810 | Antisense | | | |
| | Strand | | | |
| STAT3- | 27 mer | | GCUGCUACAAAACAACACAAGACAUGG | 807 |
| 2811 | Antisense | | | |
| | Strand | | | |
| STAT3- | 27 mer | | GCUGCUAACAAAACAACACAAGACAGG | 808 |
| 2812 | Antisense | | | |
| | Strand | | | |
| STAT3- | 27 mer | | GCUGCUGAACAAAACAACACAAGACGG | 809 |
| 2813 | Antisense | | | |
| | Strand | | | |
| STAT3- | 27 mer | | GCUGCUAUAACAAAAAGCUGCUGAGGG | 810 |
| 2846 | Antisense | | | |
| | Strand | | | |
| STAT3- | 27 mer | | GCUGCUCAAUAACAAAAAGCUGCUGGG | 811 |
| 2848 | Antisense | | | |
| | Strand | | | |
| STAT3- | 27 mer | | GCUGCUACAAUAACAAAAAGCUGCUGG | 812 |
| 2849 | Antisense | | | |
| | Strand | | | |
| STAT3- | 27 mer | | GCUGCUAACAAUAACAAAAAGCUGCGG | 813 |
| 2850 | Antisense | | | |
| | Strand | | | |
| STAT3- | 27 mer | | GCUGCUCAACAAUAACAAAAAGCUGGG | 814 |
| 2851 | Antisense | | | |
| | Strand | | | |
| STAT3- | 27 mer | | GCUGCUACAACAAUAACAAAAAGCUGG | 815 |
| 2852 | Antisense | | | |
| | Strand | | | |
| STAT3- | 27 mer | | GCUGCUAACAACAAUAACAAAAAGCGG | 816 |
| 2853 | Antisense | | | |
| | Strand | | | |
| STAT3- | 27 mer | | GCUGCUCAACAACAAUAACAAAAAGGG | 817 |
| 2854 | Antisense | | | |
| | Strand | | | |
| STAT3- | 27 mer | | GCUGCUACAACAACAAUAACAAAAAGG | 818 |
| 2855 | Antisense | | | |
| | Strand | | | |
| STAT3- | 27 mer | | GCUGCUAACAACAACAAUAACAAAAGG | 819 |
| 2856 | Antisense | | | |
| | Strand | | | |
| STAT3- | 27 mer | | GCUGCUCAACAACAACAAUAACAAAGG | 820 |
| 2857 | Antisense | | | |
| | Strand | | | |
| STAT3- | 27 mer | | GCUGCUACAACAACAACAAUAACAAGG | 821 |
| 2858 | Antisense | | | |
| | Strand | | | |
| STAT3- | 27 mer | | GCUGCUAACAACAACAACAAUAACAGG | 822 |
| 2859 | Antisense | | | |
| | Strand | | | |
| STAT3- | 27 mer | | GCUGCUGAACAACAACAACAAUAACGG | 823 |
| 2860 | Antisense | | | |
| | Strand | | | |
| STAT3- | 27 mer | | GCUGCUAGAACAACAACAACAAUAAGG | 824 |
| 2861 | Antisense | | | |
| | Strand | | | |
| STAT3- | 27 mer | | GCUGCUAAGAACAACAACAACAAUAGG | 825 |
| 2862 | Antisense | | | |
| | Strand | | | |
| STAT3- | 27 mer | | GCUGCUUAAGAACAACAACAACAAUGG | 826 |
| 2863 | Antisense | | | |
| | Strand | | | |
| STAT3- | 27 mer | | GCUGCUUCUAAGAACAACAACAACAGG | 827 |
| 2865 | Antisense | | | |
| | Strand | | | |
| STAT3- | 27 mer | | GCUGCUUGUCUAAGAACAACAACAAGG | 828 |
| 2867 | Antisense | | | |
| | Strand | | | |
| STAT3- | 27 mer | | GCUGCUUUGUCUAAGAACAACAACAGG | 829 |
| 2868 | Antisense | | | |
| | Strand | | | |
| STAT3- | 27 mer | | GCUGCUUGUCAGCAAGGUUAAAAAGGG | 830 |
| 2975 | Antisense | | | |
| | Strand | | | |
| STAT3- | 27 mer | | GCUGCUUGGAUGUCAGCAAGGUUAAGG | 831 |
| 2979 | Antisense | | | |
| | Strand | | | |
| STAT3- | 27 mer | | GCUGCUUCUAUUUGGAUGUCAGCAAGG | 832 |
| 2985 | Antisense | | | |
| | Strand | | | |
| STAT3- | 27 mer | | GCUGCUUUAAUUUAAAAAGAAACCUGG | 833 |
| 3025 | Antisense | | | |
| | Strand | | | |
| STAT3- | 27 mer | | GCUGCUUGUUAUUAUUUCUUAAUUUGG | 834 |
| 3037 | Antisense | | | |
| | Strand | | | |
| STAT3- | 27 mer | | GCUGCUUUGUUAUUAUUUCUUAAUUGG | 835 |
| 3038 | Antisense | | | |
| | Strand | | | |
| STAT3- | 27 mer | | GCUGCUAUUGUUAUUAUUUCUUAAUGG | 836 |
| 3039 | Antisense | | | |
| | Strand | | | |
| STAT3- | 27 mer | | GCUGCUUAAUUGUUAUUAUUUCUUAGG | 837 |
| 3041 | Antisense | | | |
| | Strand | | | |
| STAT3- | 27 mer | | GCUGCUUUAAUUGUUAUUAUUUCUUGG | 838 |
| 3042 | Antisense | | | |
| | Strand | | | |
| STAT3- | 27 mer | | GCUGCUUUUAAUUGUUAUUAUUUCUGG | 839 |
| 3043 | Antisense | | | |
| | Strand | | | |
| STAT3- | 27 mer | | GCUGCUAAUUUUUUGUACUUUUAGUGG | 840 |
| 3225 | Antisense | | | |
| | Strand | | | |
| STAT3- | 27 mer | | GCUGCUUAAUUUUUUGUACUUUUAGGG | 841 |
| 3226 | Antisense | | | |
| | Strand | | | |
| STAT3- | 27 mer | | GCUGCUUACAAAGGAAAAUAAGUCUGG | 842 |
| 3605 | Antisense | | | |
| | Strand | | | |
| STAT3- | 27 mer | | GCUGCUAUACAUUACAAAGGAAAAUGG | 843 |
| 3611 | Antisense | | | |
| | Strand | | | |
| STAT3- | 27 mer | | GCUGCUUCAUGUCCAACCUGUAACUGG | 844 |
| 3906 | Antisense | | | |
| | Strand | | | |
| STAT3- | 27 mer | | GCUGCUUAACAAACAGAAUUCCACAGG | 845 |
| 4311 | Antisense | | | |
| | Strand | | | |
| STAT3- | 27 mer | | GCUGCUAUUUAACAAACAGAAUUCCGG | 846 |
| 4314 | Antisense | | | |
| | Strand | | | |
| STAT3- | 27 mer | | GCUGCUUUGAUUUAACAAACAGAAUGG | 847 |
| 4317 | Antisense | | | |
| | Strand | | | |
| STAT3- | 27 mer | | GCUGCUUAAUUUGAUUUAACAAACAGG | 848 |
| 4321 | Antisense | | | |
| | Strand | | | |
| STAT3- | 27 mer | | GCUGCUUCAGUUAAGCUUAUUAUGUGG | 849 |
| 4465 | Antisense | | | |
| | Strand | | | |
| STAT3- | 27 mer | | GCUGCUAAAUAUUCUGUUUAUCAGUGG | 850 |
| 4479 | Antisense | | | |
| | Strand | | | |
| STAT3- | 27 mer | | GCUGCUUAAAUAUUCUGUUUAUCAGGG | 851 |
| 4480 | Antisense | | | |
| | Strand | | | |
| STAT3- | 27 mer | | GCUGCUAAUAUAAAUUUUUACACUAGG | 852 |
| 4831 | Antisense | | | |
| | Strand | | | |
| STAT3- | 27 mer | | GCUGCUAUAAUAUAAAUUUUUACACGG | 853 |
| 4833 | Antisense | | | |
| | Strand | | | |
| STAT3- | 27 mer | | GCUGCUACAAUAAUAUAAAUUUUUAGG | 854 |
| 4836 | Antisense | | | |
| | Strand | | | |
| STAT3- | 27 mer | | GCUGCUCACAAUAAUAUAAAUUUUUGG | 855 |
| 4837 | Antisense | | | |
| | Strand | | | |
| STAT3- | 27 mer | | GCUGCUUUUAUUUCUGGAAGUUAAAGG | 856 |
| 4909 | Antisense | | | |
| | Strand | | | |
| STAT3- | Unmodified | | CCAGGAUGACUUUGAUUUCAGCAGCCG | |
| 715 | 36 mer | | AAAGGCUGC | 857 |
| STAT3- | Unmodified | | CAGGAUGACUUUGAUUUCAAGCAGCCG | |
| 716 | 36 mer | | AAAGGCUGC | 858 |
| STAT3- | Unmodified | | AGGAUGACUUUGAUUUCAAAGCAGCCG | |
| 717 | 36 mer | | AAAGGCUGC | 859 |
| STAT3- | Unmodified | | AUGACUUUGAUUUCAACUAAGCAGCCG | |
| 720 | 36 mer | | AAAGGCUGC | 860 |
| STAT3- | Unmodified | | CUUUGGUGUUUCAUAAUCUAGCAGCCG | |
| 372 | 36 mer | | AAAGGCUGC | 861 |
| STAT3- | Unmodified | | UGACUUUGAUUUCAACUAUAGCAGCCG | |
| 721 | 36 mer | | AAAGGCUGC | 862 |
| STAT3- | Unmodified | | GACUUUGAUUUCAACUAUAAGCAGCCG | 863 |
| 722 | 36 mer | | AAAGGCUGC | |
| STAT3- | Unmodified | | AAGAUCUGAAUGGAAACAAAGCAGCCG | 864 |
| 768 | 36 mer | | AAAGGCUGC | |
| STAT3- | Unmodified | | GAAAACUGGAUAACGUCAUAGCAGCCG | 865 |
| 1001 | 36 mer | | AAAGGCUGC | |
| STAT3- | Unmodified | | CUGGAUAACGUCAUUAGCAAGCAGCCG | 866 |
| 1006 | 36 mer | | AAAGGCUGC | |
| STAT3- | Unmodified | | CUGUUUAGAAACUUAAUGAAGCAGCCG | 867 |
| 1145 | 36 mer | | AAAGGCUGC | |
| STAT3- | Unmodified | | AGAAACUUAAUGAAAAGUGAGCAGCCG | 868 |
| 1151 | 36 mer | | AAAGGCUGC | |
| STAT3- | Unmodified | | GUCAAAUUCCCUGAGUUGAAGCAGCCG | 869 |
| 1268 | 36 mer | | AAAGGCUGC | |
| STAT3- | Unmodified | | AUUCCCUGAGUUGAAUUAUAGCAGCCG | 870 |
| 1273 | 36 mer | | AAAGGCUGC | |
| STAT3- | Unmodified | | UGAGUUGAAUUAUCAGCUUAGCAGCCG | 871 |
| 1279 | 36 mer | | AAAGGCUGC | |
| STAT3- | Unmodified | | GAGUUGAAUUAUCAGCUUAAGCAGCCG | 872 |
| 1280 | 36 mer | | AAAGGCUGC | |
| STAT3- | Unmodified | | GAGUUGAAUUAUCAGCUUAAGCAGCCG | 873 |
| 1281 | 36 mer | | AAAGGCUGC | |
| STAT3- | Unmodified | | UGAAUUAUCAGCUUAAAAUAGCAGCCG | 874 |
| 1284 | 36 mer | | AAAGGCUGC | |
| STAT3- | Unmodified | | AAUUAUCAGCUUAAAAUUAAGCAGCCG | 875 |
| 1286 | 36 mer | | AAAGGCUGC | |
| STAT3- | Unmodified | | AUUAUCAGCUUAAAAUUAAAGCAGCCG | 876 |
| 1287 | 36 mer | | AAAGGCUGC | |
| STAT3- | Unmodified | | CAGCUUAAAAUUAAAGUGUAGCAGCCG | 877 |
| 1292 | 36 mer | | AAAGGCUGC | |
| STAT3- | Unmodified | | AGCUUAAAAUUAAAGUGUGAGCAGCCG | 878 |
| 1293 | 36 mer | | AAAGGCUGC | |
| STAT3- | Unmodified | | UGUGAAUUAUUCAGGGUGUAGCAGCCG | 879 |
| 1819 | 36 mer | | AAAGGCUGC | |
| STAT3- | Unmodified | | ACAAUAUCAUUGACCUUGUAGCAGCCG | 880 |
| 1908 | 36 mer | | AAAGGCUGC | |
| STAT3- | Unmodified | | AAUAUCAUUGACCUUGUGAAGCAGCCG | 881 |
| 1910 | 36 mer | | AAAGGCUGC | |
| STAT3- | Unmodified | | AUCAUUGACCUUGUGAAAAAGCAGCCG | 882 |
| 1913 | 36 mer | | AAAGGCUGC | |
| STAT3- | Unmodified | | UGUCAUUUGCUGAAAUCAUAGCAGCCG | 883 |
| 2154 | 36 mer | | AAAGGCUGC | |
| STAT3- | Unmodified | | CUGAAGACCAAGUUUAUCUAGCAGCCG | 884 |
| 2327 | 36 mer | | AAAGGCUGC | |
| STAT3- | Unmodified | | CAAGUUUAUCUGUGUGACAAGCAGCCG | 885 |
| 2335 | 36 mer | | AAAGGCUGC | |
| STAT3- | Unmodified | | AGUUUGGAAAUAAUGGUGAAGCAGCCG | 886 |
| 2418 | 36 mer | | AAAGGCUGC | |
| STAT3- | Unmodified | | AAAUAGAGAAAUGAGUGAAAGCAGCCG | 887 |
| 2692 | 36 mer | | AAAGGCUGC | |
| STAT3- | Unmodified | | AAUAGAGAAAUGAGUGAAUAGCAGCCG | 888 |
| 2693 | 36 mer | | AAAGGCUGC | |
| STAT3- | Unmodified | Hs-Mf- | UUGUGGUUCCAGAUUUUUUAGCAGCCG | 889 |
| 2627 | 36 mer | Mm | AAAGGCUGC | |
| STAT3- | Unmodified | Hs-Mf- | UUUGUGGUUCCAGAUUUUUAGCAGCCG | 890 |
| 2626 | 36 mer | Mm | AAAGGCUGC | |
| STAT3- | Unmodified | Hs-Mf- | UUCAUUGAUGCAGUUUGGAAGCAGCCG | 891 |
| 2407 | 36 mer | Mm | AAAGGCUGC | |
| STAT3- | Unmodified | Hs-Mf- | UGAUGCAGUUUGGAAAUAAAGCAGCCG | 892 |
| 2412 | 36 mer | Mm | AAAGGCUGC | |
| STAT3- | Unmodified | Hs-Mf- | ACAUGUCAUUUGCUGAAAUAGCAGCCG | 893 |
| 2151 | 36 mer | Mm | AAAGGCUGC | |
| STAT3- | Unmodified | Hs-Mf- | CUUUGUGGUUCCAGAUUUUAGCAGCCG | 894 |
| 2625 | 36 mer | Mm | AAAGGCUGC | |
| STAT3- | Unmodified | Hs-Mf- | UAAAAAUUUAUAUUAUUGUAGCAGCCG | 895 |
| 4836 | 36 mer | Mm | AAAGGCUGC | |
| STAT3- | Unmodified | Hs-Mf- | UCAUUGAUGCAGUUUGGAAAGCAGCCG | 896 |
| 2408 | 36 mer | Mm | AAAGGCUGC | |
| STAT3- | Unmodified | Hs-Mf- | UUUGCUGAAAUCAUCAUGGAGCAGCCG | 897 |
| 2159 | 36 mer | Mm | AAAGGCUGC | |
| STAT3- | Unmodified | Hs-Mf- | GAACAACAUGUCAUUUGCUAGCAGCCG | 898 |
| 2146 | 36 mer | Mm | AAAGGCUGC | |
| STAT3- | Unmodified | Hs-Mf- | ACAACAUGUCAUUUGCUGAAGCAGCCG | 899 |
| 2148 | 36 mer | Mm | AAAGGCUGC | |
| STAT3- | Unmodified | Hs-Mf- | AACAACAUGUCAUUUGCUGAGCAGCCG | 900 |
| 2147 | 36 mer | Mm | AAAGGCUGC | |
| STAT3- | Unmodified | Hs-Mf- | CGAAGAAUCAAGCAGUUUCAGCAGCCG | 901 |
| 0461 | 36 mer | Mm | AAAGGCUGC | |
| STAT3- | Unmodified | Hs-Mf- | CCUUGCCAGUUGUGGUGAUAGCAGCCG | 902 |
| 1584 | 36 mer | Mm | AAAGGCUGC | |
| STAT3- | Unmodified | Hs-Mf- | AACAAAUUAAGAAACUGGAAGCAGCCG | 903 |
| 1047 | 36 mer | Mm | AAAGGCUGC | |
| STAT3- | Unmodified | Hs-Mf- | CUGAAUGGAAACAACCAGUAGCAGCCG | 904 |
| 0773 | 36 mer | Mm | AAAGGCUGC | |
| STAT3- | Unmodified | Hs-Mf- | AUCUUGAGAAGCCAAUGGAAGCAGCCG | 905 |
| 0492 | 36 mer | Mm | AAAGGCUGC | |
| STAT3- | Unmodified | Hs-Mf- | GAAGAAUCAAGCAGUUUCUAGCAGCCG | 906 |
| 0462 | 36 mer | Mm | AAAGGCUGC | |
| STAT3- | Unmodified | Hs-Mf- | UUGCCAGUUGUGGUGAUCUAGCAGCCG | 907 |
| 1586 | 36 mer | Mm | AAAGGCUGC | |
| STAT3- | Unmodified | Hs-Mf- | AUCUGAAUGGAAACAACCAAGCAGCCG | 908 |
| 0771 | 36 mer | Mm | AAAGGCUGC | |
| STAT3- | Unmodified | Hs-Mf- | AUCUAGAACAGAAAAUGAAAGCAGCCG | 909 |
| 0681 | 36 mer | Mm | AAAGGCUGC | |
| STAT3- | Unmodified | Hs-Mf- | AGGAUCUAGAACAGAAAAUAGCAGCCG | 910 |
| 0678 | 36 mer | Mm | AAAGGCUGC | |
| STAT3- | Unmodified | Hs-Mf- | AAAAAUUUAUAUUAUUGUGAGCAGCCG | 911 |
| 4837 | 36 mer | Mm | AAAGGCUGC | |
| STAT3- | Unmodified | Hs-Mf- | GUGUAAAAAUUUAUAUUAUAGCAGCCG | 912 |
| 4833 | 36 mer | Mm | AAAGGCUGC | |
| STAT3- | Unmodified | Hs | AGUUGCAGCAAAAAGUUUCAGCAGCCG | 913 |
| 1068 | 36 mer | | AAAGGCUGC | |
| STAT3- | Unmodified | Hs | AAGAAUGUAAACUUUUUUAAGCAGCCG | 914 |
| 1673 | 36 mer | | AAAGGCUGC | |
| STAT3- | Unmodified | Hs | UGCAAGAGUCGAAUGUUCUAGCAGCCG | 915 |
| 0426 | 36 mer | | AAAGGCUGC | |
| STAT3- | Unmodified | Hs | AGAUUCAUUGAUGCAGUUUAGCAGCCG | 916 |
| 2404 | 36 mer | | AAAGGCUGC | |
| STAT3- | Unmodified | Hs | GAGUUGCAGCAAAAAGUUUAGCAGCCG | 917 |
| 1067 | 36 mer | | AAAGGCUGC | |
| STAT3- | Unmodified | Hs | GUCGAAUGUUCUCUAUCAGAGCAGCCG | 918 |
| 0433 | 36 mer | | AAAGGCUGC | |
| STAT3- | Unmodified | Hs | CCCAAGAAUGUAAACUUUUAGCAGCCG | 919 |
| 1670 | 36 mer | | AAAGGCUGC | |
| STAT3- | Unmodified | Hs | GUGAUGAACAUGGAAGAAUAGCAGCCG | 920 |
| 1388 | 36 mer | | AAAGGCUGC | |
| STAT3- | Unmodified | Hs | AAGAGUCGAAUGUUCUCUAAGCAGCCG | 921 |
| 0429 | 36 mer | | AAAGGCUGC | |
| STAT3- | Unmodified | Hs | GAUUCAUUGAUGCAGUUUGAGCAGCCG | 922 |
| 2405 | 36 mer | | AAAGGCUGC | |
| STAT3- | Unmodified | Hs | AGAGUCGAAUGUUCUCUAUAGCAGCCG | 923 |
| 0430 | 36 mer | | AAAGGCUGC | |
| STAT3- | Unmodified | Hs | AGUCGAAUGUUCUCUAUCAAGCAGCCG | 924 |
| 0432 | 36 mer | | AAAGGCUGC | |
| STAT3- | Unmodified | Hs | CUGGUGUGAAUUAUUCAGGAGCAGCCG | 925 |
| 1815 | 36 mer | | AAAGGCUGC | |
| STAT3- | Unmodified | Hs | CCUGCAAGAGUCGAAUGUUAGCAGCCG | 926 |
| 0424 | 36 mer | | AAAGGCUGC | |
| STAT3- | Unmodified | Hs | ACCUUCCUGCUAAGAUUCAAGCAGCCGA | 927 |
| 2024 | 36 mer | | AAGGCUGC | |
| STAT3- | Unmodified | Hs | ACCUGGUGUGAAUUAUUCAAGCAGCCG | 928 |
| 1813 | 36 mer | | AAAGGCUGC | |
| STAT3- | Unmodified | Hs | AGAAUGUAAACUUUUUUACAGCAGCCG | 929 |
| 1674 | 36 mer | | AAAGGCUGC | |
| STAT3- | Unmodified | Hs | CAGUUCACUACUAAAGUCAAGCAGCCG | 930 |
| 1241 | 36 mer | | AAAGGCUGC | |
| STAT3- | Unmodified | Hs | CAAGAAUGUAAACUUUUUUAGCAGCCG | 931 |
| 1672 | 36 mer | | AAAGGCUGC | |
| STAT3- | Unmodified | Hs | CUGCAAGAGUCGAAUGUUCAGCAGCCG | 932 |
| 0425 | 36 mer | | AAAGGCUGC | |
| STAT3- | Unmodified | Hs | GGUGUGAAUUAUUCAGGGUAGCAGCCG | 933 |
| 1817 | 36 mer | | AAAGGCUGC | |
| STAT3- | Unmodified | Hs | CCAAGAAUGUAAACUUUUUAGCAGCCG | 934 |
| 1671 | 36 mer | | AAAGGCUGC | |
| STAT3- | Unmodified | Hs-Mm | AGCAGCAGCUGAACAACAUAGCAGCCG | 935 |
| 2136 | 36 mer | | AAAGGCUGC | |
| STAT3- | Unmodified | Hs-Mm | GCUGAACAACAUGUCAUUUAGCAGCCG | 936 |
| 2143 | 36 mer | | AAAGGCUGC | |
| STAT3- | Unmodified | Hs-Mm | CUGAACAACAUGUCAUUUGAGCAGCCG | 937 |
| 2144 | 36 mer | | AAAGGCUGC | |
| STAT3- | Unmodified | Hs-Mm | CAGCAGCUGAACAACAUGUAGCAGCCG | 938 |
| 2138 | 36 mer | | AAAGGCUGC | |
| STAT3- | Unmodified | Hs-Mm | UUUAACUUCCAGAAAUAAAAGCAGCCG | 939 |
| 4909 | 36 mer | | AAAGGCUGC | |
| STAT3- | Unmodified | Hs-Mm | AGCAGCUGAACAACAUGUCAGCAGCCG | 940 |
| 2139 | 36 mer | | AAAGGCUGC | |
| STAT3- | Unmodified | Hs-Mm | UUGAUGCAGUUUGGAAAUAAGCAGCCG | 941 |
| 2411 | 36 mer | | AAAGGCUGC | |
| STAT3- | Unmodified | Hs-Mm | UGAACAACAUGUCAUUUGCAGCAGCCG | 942 |
| 2145 | 36 mer | | AAAGGCUGC | |
| STAT3- | Unmodified | Hs-Mm | UAGUGUAAAAAUUUAUAUUAGCAGCCG | 943 |
| 4831 | 36 mer | | AAAGGCUGC | |
| STAT3- | Unmodified | Hs-Mm | UAACUUUGUGGUUCCAGAUAGCAGCCG | 944 |
| 2622 | 36 mer | | AAAGGCUGC | |
| STAT3- | Unmodified | Hs-Mm | AAGCAGCAGCUGAACAACAAGCAGCCG | 945 |
| 2135 | 36 mer | | AAAGGCUGC | |
| STAT3- | Unmodified | Hs-Mm | CAAAAGUGAUGAACAUGGAAGCAGCCG | 946 |
| 1383 | 36 mer | | AAAGGCUGC | |
| STAT3- | Unmodified | | UGAAAUCAAAGUCAUCCUGGGG | 947 |
| 715 | 22 mer | | | |
| STAT3- | Unmodified | | UUGAAAUCAAAGUCAUCCUGGG | 948 |
| 716 | 22 mer | | | |
| STAT3- | Unmodified | | UUUGAAAUCAAAGUCAUCCUGG | 949 |
| 717 | 22 mer | | | |
| STAT3- | Unmodified | | UUAGUUGAAAUCAAAGUCAUGG | 950 |
| 720 | 22 mer | | | |
| STAT3- | Unmodified | | UAGAUUAUGAAACACCAAAGGG | 951 |
| 372 | 22 mer | | | |
| STAT3- | Unmodified | | UAUAGUUGAAAUCAAAGUCAGG | 952 |
| 721 | 22 mer | | | |
| STAT3- | Unmodified | | UUAUAGUUGAAAUCAAAGUCGG | 953 |
| 722 | 22 mer | | | |
| STAT3- | Unmodified | | UUUGUUUCCAUUCAGAUCUUGG | 954 |
| 768 | 22 mer | | | |
| STAT3- | Unmodified | | UAUGACGUUAUCCAGUUUUCGG | 955 |
| 1001 | 22 mer | | | |
| STAT3- | Unmodified | | UUGCUAAUGACGUUAUCCAGGG | 956 |
| 1006 | 22 mer | | | |
| STAT3- | Unmodified | | UUCAUUAAGUUUCUAAACAGGG | 957 |
| 1145 | 22 mer | | | |
| STAT3- | Unmodified | | UCACUUUUCAUUAAGUUUCUGG | 958 |
| 1151 | 22 mer | | | |
| STAT3- | Unmodified | | UUCAACUCAGGGAAUUUGACGG | 959 |
| 1268 | 22 mer | | | |
| STAT3- | Unmodified | | UAUAAUUCAACUCAGGGAAUGG | 960 |
| 1273 | 22 mer | | | |
| STAT3- | Unmodified | | UAAGCUGAUAAUUCAACUCAGG | 961 |
| 1279 | 22 mer | | | |
| STAT3- | Unmodified | | UUAAGCUGAUAAUUCAACUCGG | 962 |
| 1280 | 22 mer | | | |
| STAT3- | Unmodified | | UUUAAGCUGAUAAUUCAACUGG | 963 |
| 1281 | 22 mer | | | |
| STAT3- | Unmodified | | UAUUUUAAGCUGAUAAUUCAGG | 964 |
| 1284 | 22 mer | | | |
| STAT3- | Unmodified | | UUAAUUUUAAGCUGAUAAUUGG | 965 |
| 1286 | 22 mer | | | |
| STAT3- | Unmodified | | UUUAAUUUUAAGCUGAUAAUGG | 966 |
| 1287 | 22 mer | | | |
| STAT3- | Unmodified | | UACACUUUAAUUUUAAGCUGGG | 967 |
| 1292 | 22 mer | | | |
| STAT3- | Unmodified | | UCACACUUUAAUUUUAAGCUGG | 968 |
| 1293 | 22 mer | | | |
| STAT3- | Unmodified | | UACACCCUGAAUAAUUCACAGG | 969 |
| 1819 | 22 mer | | | |
| STAT3- | Unmodified | | UACAAGGUCAAUGAUAUUGUGG | 970 |
| 1908 | 22 mer | | | |
| STAT3- | Unmodified | | UUCACAAGGUCAAUGAUAUUGG | 971 |
| 1910 | 22 mer | | | |
| STAT3- | Unmodified | | UUUUUCACAAGGUCAAUGAUGG | 972 |
| 1913 | 22 mer | | | |
| STAT3- | Unmodified | | UAUGAUUUCAGCAAAUGACAGG | 973 |
| 2154 | 22 mer | | | |
| STAT3- | Unmodified | | UAGAUAAACUUGGUCUUCAGGG | 974 |
| 2327 | 22 mer | | | |
| STAT3- | Unmodified | | UUGUCACACAGAUAAACUUGGG | 975 |
| 2335 | 22 mer | | | |
| STAT3- | Unmodified | | UUCACCAUUAUUUCCAAACUGG | 976 |
| 2418 | 22 mer | | | |
| STAT3- | Unmodified | | UUUCACUCAUUUCUCUAUUUGG | 977 |
| 2692 | 22 mer | | | |
| STAT3- | Unmodified | | UAUUCACUCAUUUCUCUAUUGG | 978 |
| 2693 | 22 mer | | | |
| STAT3- | Unmodified | Hs-Mf- | UAAAAAAUCUGGAACCACAAGG | 979 |
| 2627 | 22 mer | Mm | | |
| STAT3- | Unmodified | Hs-Mf- | UAAAAAUCUGGAACCACAAAGG | 980 |
| 2626 | 22 mer | Mm | | |
| STAT3- | Unmodified | Hs-Mf- | UUCCAAACUGCAUCAAUGAAGG | 981 |
| 2407 | 22 mer | Mm | | |
| STAT3- | Unmodified | Hs-Mf- | UUUAUUUCCAAACUGCAUCAGG | 982 |
| 2412 | 22 mer | Mm | | |
| STAT3- | Unmodified | Hs-Mf- | UAUUUCAGCAAAUGACAUGUGG | 983 |
| 2151 | 22 mer | Mm | | |
| STAT3- | Unmodified | Hs-Mf- | UAAAAUCUGGAACCACAAAGGG | 984 |
| 2625 | 22 mer | Mm | | |
| STAT3- | Unmodified | Hs-Mf- | UACAAUAAUAUAAAUUUUUAGG | 985 |
| 4836 | 22 mer | Mm | | |
| STAT3- | Unmodified | Hs-Mf- | UUUCCAAACUGCAUCAAUGAGG | 986 |
| 2408 | 22 mer | Mm | | |
| STAT3- | Unmodified | Hs-Mf- | UCCAUGAUGAUUUCAGCAAAGG | 987 |
| 2159 | 22 mer | Mm | | |
| STAT3- | Unmodified | Hs-Mf- | UAGCAAAUGACAUGUUGUUCGG | 988 |
| 2146 | 22 mer | Mm | | |
| STAT3- | Unmodified | Hs-Mf- | UUCAGCAAAUGACAUGUUGUGG | 989 |
| 2148 | 22 mer | Mm | | |
| STAT3- | Unmodified | Hs-Mf- | UCAGCAAAUGACAUGUUGUUGG | 990 |
| 2147 | 22 mer | Mm | | |
| STAT3- | Unmodified | Hs-Mf- | UGAAACUGCUUGAUUCUUCGGG | 991 |
| 0461 | 22 mer | Mm | | |
| STAT3- | Unmodified | Hs-Mf- | UAUCACCACAACUGGCAAGGGG | 992 |
| 1584 | 22 mer | Mm | | |
| STAT3- | Unmodified | Hs-Mf- | UUCCAGUUUCUUAAUUUGUUGG | 993 |
| 1047 | 22 mer | Mm | | |
| STAT3- | Unmodified | Hs-Mf- | UACUGGUUGUUUCCAUUCAGGG | 994 |
| 0773 | 22 mer | Mm | | |
| STAT3- | Unmodified | Hs-Mf- | UUCCAUUGGCUUCUCAAGAUGG | 995 |
| 0492 | 22 mer | Mm | | |
| STAT3- | Unmodified | Hs-Mf- | UAGAAACUGCUUGAUUCUUCGG | 996 |
| 0462 | 22 mer | Mm | | |
| STAT3- | Unmodified | Hs-Mf- | UAGAUCACCACAACUGGCAAGG | 997 |
| 1586 | 22 mer | Mm | | |
| STAT3- | Unmodified | Hs-Mf- | UUGGUUGUUUCCAUUCAGAUGG | 998 |
| 0771 | 22 mer | Mm | | |
| STAT3- | Unmodified | Hs-Mf- | UUUCAUUUUCUGUUCUAGAUGG | 999 |
| 0681 | 22 mer | Mm | | |
| STAT3- | Unmodified | Hs-Mf- | UAUUUUCUGUUCUAGAUCCUGG | 1000 |
| 0678 | 22 mer | Mm | | |
| STAT3- | Unmodified | Hs-Mf- | UCACAAUAAUAUAAAUUUUUGG | 1001 |
| 4837 | 22 mer | Mm | | |
| STAT3- | Unmodified | Hs-Mf- | UAUAAUAUAAAUUUUUACACGG | 1002 |
| 4833 | 22 mer | Mm | | |
| STAT3- | Unmodified | Hs | UGAAACUUUUUGCUGCAACUGG | 1003 |
| 1068 | 22 mer | | | |
| STAT3- | Unmodified | Hs | UUAAAAAAGUUUACAUUCUUGG | 1004 |
| 1673 | 22 mer | | | |
| STAT3- | Unmodified | Hs | UAGAACAUUCGACUCUUGCAGG | 1005 |
| 0426 | 22 mer | | | |
| STAT3- | Unmodified | Hs | UAAACUGCAUCAAUGAAUCUGG | 1006 |
| 2404 | 22 mer | | | |
| STAT3- | Unmodified | Hs | UAAACUUUUUGCUGCAACUCGG | 1007 |
| 1067 | 22 mer | | | |
| STAT3- | Unmodified | Hs | UCUGAUAGAGAACAUUCGACGG | 1008 |
| 0433 | 22 mer | | | |
| STAT3- | Unmodified | Hs | UAAAAGUUUACAUUCUUGGGGG | 1009 |
| 1670 | 22 mer | | | |
| STAT3- | Unmodified | Hs | UAUUCUUCCAUGUUCAUCACGG | 1010 |
| 1388 | 22 mer | | | |
| STAT3- | Unmodified | Hs | UUAGAGAACAUUCGACUCUUGG | 1011 |
| 0429 | 22 mer | | | |
| STAT3- | Unmodified | Hs | UCAAACUGCAUCAAUGAAUCGG | 1012 |
| 2405 | 22 mer | | | |
| STAT3- | Unmodified | Hs | UAUAGAGAACAUUCGACUCUGG | 1013 |
| 0430 | 22 mer | | | |
| STAT3- | Unmodified | Hs | UUGAUAGAGAACAUUCGACUGG | 1014 |
| 0432 | 22 mer | | | |
| STAT3- | Unmodified | Hs | UCCUGAAUAAUUCACACCAGGG | 1015 |
| 1815 | 22 mer | | | |
| STAT3- | Unmodified | Hs | UAACAUUCGACUCUUGCAGGGG | 1016 |
| 0424 | 22 mer | | | |
| STAT3- | Unmodified | Hs | UUGAAUCUUAGCAGGAAGGUGG | 1017 |
| 2024 | 22 mer | | | |
| STAT3- | Unmodified | Hs | UUGAAUAAUUCACACCAGGUGG | 1018 |
| 1813 | 22 mer | | | |
| STAT3- | Unmodified | Hs | UGUAAAAAAGUUUACAUUCUGG | 1019 |
| 1674 | 22 mer | | | |
| STAT3- | Unmodified | Hs | UUGACUUUAGUAGUGAACUGGG | 1020 |
| 1241 | 22 mer | | | |
| STAT3- | Unmodified | Hs | UAAAAAAGUUUACAUUCUUGGG | 1021 |
| 1672 | 22 mer | | | |
| STAT3- | Unmodified | Hs | UGAACAUUCGACUCUUGCAGGG | 1022 |
| 0425 | 22 mer | | | |
| STAT3- | Unmodified | Hs | UACCCUGAAUAAUUCACACCGG | 1023 |
| 1817 | 22 mer | | | |
| STAT3- | Unmodified | Hs | UAAAAAGUUUACAUUCUUGGGG | 1024 |
| 1671 | 22 mer | | | |
| STAT3- | Unmodified | Hs-Mm | UAUGUUGUUCAGCUGCUGCUGG | 1025 |
| 2136 | 22 mer | | | |
| STAT3- | Unmodified | Hs-Mm | UAAAUGACAUGUUGUUCAGCGG | 1026 |
| 2143 | 22 mer | | | |
| STAT3- | Unmodified | Hs-Mm | UCAAAUGACAUGUUGUUCAGGG | 1027 |
| 2144 | 22 mer | | | |
| STAT3- | Unmodified | Hs-Mm | UACAUGUUGUUCAGCUGCUGGG | 1028 |
| 2138 | 22 mer | | | |
| STAT3- | Unmodified | Hs-Mm | UUUUAUUUCUGGAAGUUAAAGG | 1029 |
| 4909 | 22 mer | | | |
| STAT3- | Unmodified | Hs-Mm | UGACAUGUUGUUCAGCUGCUGG | 1030 |
| 2139 | 22 mer | | | |
| STAT3- | Unmodified | Hs-Mm | UUAUUUCCAAACUGCAUCAAGG | 1031 |
| 2411 | 22 mer | | | |
| STAT3- | Unmodified | Hs-Mm | UGCAAAUGACAUGUUGUUCAGG | 1032 |
| 2145 | 22 mer | | | |
| STAT3- | Unmodified | Hs-Mm | UAAUAUAAAUUUUUACACUAGG | 1033 |
| 4831 | 22 mer | | | |
| STAT3- | Unmodified | Hs-Mm | UAUCUGGAACCACAAAGUUAGG | 1034 |
| 2622 | 22 mer | | | |
| STAT3- | Unmodified | Hs-Mm | UUGUUGUUCAGCUGCUGCUUGG | 1035 |
| 2135 | 22 mer | | | |
| STAT3- | Unmodified | Hs-Mm | UUCCAUGUUCAUCACUUUUGGG | 1036 |
| 1383 | 22 mer | | | |
| STAT3- | Modified 36 | | [mCs][mC][mA][mG][mG][mA][mU][fG][fA][fC] | 1037 |
| 715 | mer | | [fU][mU][mU][mG][mA][mU][mU][mU][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mA][mG][mG][mA][mU][mG][fA][fC][fU] | 1038 |
| 716 | mer | | [fU][mU][mG][mA][mU][mU][mU][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mG][mG][mA][mU][mG][mA][fC][fU][fU] | 1039 |
| 717 | mer | | [fU][mG][mA][mU][mU][mU][mC][mA][mA] | |
| | | | [mA][mG][mC][ma][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mU][mG][mA][mC][mU][mU][fU][fG][fA] | 1040 |
| 720 | mer | | [fU][mU][mU][mC][mA][mA][mC][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mU][mU][mU][mG][mG][mU][fG][fU][fU] | 1041 |
| 372 | mer | | [fU][mC][mA][mU][mA][mA][mU][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mUs][mG][mA][mC][mU][mU][mU][fG][fA][fU] | 1042 |
| 721 | mer | | [fU][mU][mC][mA][mA][mC][mU][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mGs][mA][mC][mU][mU][mU][mG][fA][fU][fU] | 1043 |
| 722 | mer | | [fU][mC][mA][mA][mC][mU][mA][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mA][mG][mA][mU][mC][mU][fG][fA][fA] | 1044 |
| 768 | mer | | [fU][mG][mG][mA][mA][mA][mC][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mGs][mA][mA][mA][mA][mC][mU][fG][fG][fA] | 1045 |
| 1001 | mer | | [fU][mA][mA][mC][mG][mU][mC][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mU][mG][mG][mA][mU][mA][fA][fC][fG] | 1046 |
| 1006 | mer | | [fU][mC][mA][mU][mU][mA][mG][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mU][mG][mU][mU][mU][mA][fG][fA][fA] | 1047 |
| 1145 | mer | | [fA][mC][mU][mU][mA][mA][mU][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mG][mA][mA][mA][mC][mU][fU][fA][fA] | 1048 |
| 1151 | mer | | [fU][mG][mA][mA][mA][mA][mG][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mGs][mU][mC][mA][mA][mA][mU][fU][fC][fC] | 1049 |
| 1268 | mer | | [fC][mU][mG][mA][mG][mU][mU][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mU][mU][mC][mC][mC][mU][fG][fA][fG] | 1050 |
| 1273 | mer | | [fU][mU][mG][mA][mA][mU][mU][mA][mU] | |
| | | | [mA][mG][mC][mA][mG][mC][mC][mG][ademA- | |
| | | | -GalNAc][ademA-GalNAc][ademA- | |
| | | | GalNAc][mG][mG][mC][mU][mG][mC] | |
| STAT3- | Modified 36 | | [mUs][mG][mA][mG][mU][mU][mG][fA][fA][fU] | 1051 |
| 1279 | mer | | [fU][mA][mU][mC][mA][mG][mC][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mGs][mA][mG][mU][mU][mG][mA][fA][fU][fU] | 1052 |
| 1280 | mer | | [fA][mU][mC][mA][mG][mC][mU][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mG][mU][mU][mG][mA][mA][fU][fU][fA] | 1053 |
| 1281 | mer | | [fU][mC][mA][mG][mC][mU][mU][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mUs][mG][mA][mA][mU][mU][mA][fU][fC][fA] | 1054 |
| 1284 | mer | | [fG][mC][mU][mU][mA][mA][mA][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mA][mU][mU][mA][mU][mC][A][fG][fC] | 1055 |
| 1286 | mer | | [fU][mU][mA][mA][mA][mA][mU][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mU][mU][mA][mU][mC][mA][fG][fC][fU] | 1056 |
| 1287 | mer | | [fU][mA][mA][mA][mA][mU][mU][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mA][mG][mC][mU][mU][mA][fA][fA][fA] | 1057 |
| 1292 | mer | | [fU][mU][mA][mA][mA][mG][mU][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mG][mC][mU][mU][mA][mA][fA][fA][fU] | 1058 |
| 1293 | mer | | [fU][mA][mA][mA][mG][mU][mG][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mUs][mG][mU][mG][mA][mA][mU][fU][fA][fU] | 1059 |
| 1819 | mer | | [fU][mC][mA][mG][mG][mG][mU][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mC][mA][mA][mU][mA][mU][fC][fA][fU] | 1060 |
| 1908 | mer | | [fU][mG][mA][mC][mC][mU][mU][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mA][mU][mA][mU][mC][mA][fU][fU][fG] | 1061 |
| 1910 | mer | | [fA][mC][mC][mU][mU][mG][mU][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mU][mC][mA][mU][mU][mG][fA][fC][fC] | 1062 |
| 1913 | mer | | [fU][mU][mG][mU][mG][mA][mA][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mUs][mG][mU][mC][mA][mU][mU][fU][fG][fC] | 1063 |
| 2154 | mer | | [fU][mG][mA][mA][mA][mU][mC][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mU][mG][mA][mA][mG][mA][fC][fC][fA] | 1064 |
| 2327 | mer | | [fA][mG][mU][mU][mU][mA][mU][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mCs][mA][mA][mG][mU][mU][mU][fA][fU][fC] | 1065 |
| 2335 | mer | | [fU][mG][mU][mG][mU][mG][mA][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mG][mU][mU][mU][mG][mG][fA][fA][fA] | 1066 |
| 2418 | mer | | [fU][mA][mA][mU][mG][mG][mU][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mA][mA][mU][mA][mG][mA][fG][fA][fA] | 1067 |
| 2692 | mer | | [fA][mU][mG][mA][mG][mU][mG][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | | [mAs][mA][mU][mA][mG][mA][mG][fA][fA][fA] | 1068 |
| 2693 | mer | | [fU][mG][mA][mG][mU][mG][mA][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mU][mG][mU][mG][mG][mU][fU][fC][fC] | 1069 |
| 2627 | mer | Mm | [fA][mG][mA][mU][mU][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mU][mU][mG][mU][mG][mG][fU][fU][fC] | 1070 |
| 2626 | mer | Mm | [fC][mA][mG][mA][mU][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mU][mC][mA][mU][mU][mG][fA][fU][fG] | 1071 |
| 2407 | mer | Mm | [fC][mA][mG][mU][mU][mU][mG][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mG][mA][mU][mG][mC][mA][fG][fU][fU] | 1072 |
| 2412 | mer | Mm | [fU][mG][mG][mA][mA][mA][mU][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mC][mA][mU][mG][mU][mC][fA][fU][fU] | 1073 |
| 2151 | mer | Mm | [fU][mG][mC][mU][mG][mA][mA][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mCs][mU][mU][mU][mG][mU][mG][fG][fU][fU] | 1074 |
| 2625 | mer | Mm | [fC][mC][mA][mG][mA][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mA][mA][mA][mA][mA][mU][fU][fU][fA] | 1075 |
| 4836 | mer | Mm | [fU][mA][mU][mU][mA][mU][mU][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mC][mA][mU][mU][mG][mA][fU][fG][fC] | 1076 |
| 2408 | mer | Mm | [fA][mG][mU][mU][mU][mG][mG][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mU][mU][mG][mC][mU][mG][fA][fA][fA] | 1077 |
| 2159 | mer | Mm | [fU][mC][mA][mU][mC][mA][mU][mG][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mGs][mA][mA][mC][mA][mA][mC][fA][fU][fG] | 1078 |
| 2146 | mer | Mm | [fU][mC][mA][mU][mU][mU][mG][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mC][mA][mA][mC][mA][mU][fG][fU][fC] | 1079 |
| 2148 | mer | Mm | [fA][mU][mU][mU][mG][mC][mU][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mA][mC][mA][mA][mC][mA][fU][fG][fU] | 1080 |
| 2147 | mer | Mm | [fC][mA][mU][mU][mU][mG][mC][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mCs][mG][mA][mA][mG][mA][mA][fU][fC][fA] | 1081 |
| 0461 | mer | Mm | [fA][mG][mC][mA][mG][mU][mU][mU][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mCs][mC][mU][mU][mG][mC][mC][fA][fG][fU] | 1082 |
| 1584 | mer | Mm | [fU][mG][mU][mG][mG][mU][mG][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mA][mC][mA][mA][mA][mU][fU][fA][fA] | 1083 |
| 1047 | mer | Mm | [fG][mA][mA][mA][mC][mU][mG][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mCs][mU][mG][mA][mA][mU][mG][fG][fA][fA] | 1084 |
| 0773 | mer | Mm | [fA][mC][mA][mA][mC][mC][mA][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mU][mC][mU][mU][mG][mA][fG][fA][fA] | 1085 |
| 0492 | mer | Mm | [fG][mC][mC][mA][mA][mU][mG][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mGs][mA][mA][mG][mA][mA][mU][fC][fA][fA] | 1086 |
| 0462 | mer | Mm | [fG][mC][mA][mG][mU][mU][mU][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mUs][mU][mG][mC][mC][mA][mG][fU][fU][fG] | 1087 |
| 1586 | mer | Mm | [fU][mG][mG][mU][mG][mA][mU][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mU][mC][mU][mG][mA][mA][fU][fG][fG] | 1088 |
| 0771 | mer | Mm | [fA][mA][mA][mC][mA][mA][mC][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mU][mC][mU][mA][mG][mA][fA][fC][fA] | 1089 |
| 0681 | mer | Mm | [fG][mA][mA][mA][mA][mU][mG][mA][mA] | |
| | | | [mA][mG][mC][mA][mG][mC][mC][mG][ademA- | |
| | | | -GalNAc][ademA-GalNAc][ademA- | |
| | | | GalNAc][mG][mG][mC][mU][mG][mC] | |
| STAT3- | Modified 36 | Hs-Mf- | [mAs][mG][mG][mA][mU][mC][mU][fA][fG][fA] | 1090 |
| 0678 | mer | Mm | [fA][mC][mA][mG][mA][mA][mA][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mAs][mA][mA][mA][mA][mU][mU][fU][fA][fU] | 1091 |
| 4837 | mer | Mm | [fA][mU][mU][mA][mU][mU][mG][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mf- | [mGs][mU][mG][mU][mA][mA][mA][A][fA][fU] | 1092 |
| 4833 | mer | Mm | [fU][mU][mA][mU][mA][mU][mU][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mG][mU][mU][mG][mC][mA][fG][fC][fA] | 1093 |
| 1068 | mer | | [fA][mA][mA][mA][mG][mU][mU][mU][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mA][mG][mA][mA][mU][mG][fU][fA][fA] | 1094 |
| 1673 | mer | | [fA][mC][mU][mU][mU][mU][mU][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mUs][mG][mC][mA][mA][mG][mA][fG][fU][fC] | 1095 |
| 0426 | mer | | [fG][mA][mA][mU][mG][mU][mU][mC][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mG][mA][m][m][m][m][f][fU][fG] | 1096 |
| 2404 | mer | | [fA][mU][mG][mC][mA][mG][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mGs][mA][mG][mU][mU][mG][mC][fA][fG][fC] | 1097 |
| 1067 | mer | | [fA][mA][mA][mA][mA][mG][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mGs][mU][mC][mG][mA][mA][mU][fG][fU][fU] | 1098 |
| 0433 | mer | | [fC][mU][mC][mU][mA][mU][mC][mA][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mC][mC][mA][mA][mG][mA][fA][fU][fG] | 1099 |
| 1670 | mer | | [fU][mA][mA][mA][mC][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mGs][mU][mG][mA][mU][mG][mA][fA][fC][fA] | 1100 |
| 1388 | mer | | [fU][mG][mG][mA][mA][mG][mA][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mA][mG][mA][mG][mU][mC][fG][fA][fA] | 1101 |
| 0429 | mer | | [fU][mG][mU][mU][mC][mU][mC][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mGs][mA][mU][mU][mC][mA][mU][fU][fG][fA] | 1102 |
| 2405 | mer | | [fU][mG][mC][mA][mG][mU][mU][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mG][mA][mG][mU][mC][mG][fA][fA][fU] | 1103 |
| 0430 | mer | | [fG][mU][mU][mC][mU][mC][mU][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mG][mU][mC][mG][mA][mA][fU][fG][fU] | 1104 |
| 0432 | mer | | [fU][mC][mU][mC][mU][mA][mU][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mU][mG][mG][mU][mG][mU][fG][fA][fA] | 1105 |
| 1815 | mer | | [fU][mU][mA][mU][mU][mC][mA][mG][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mC][mU][mG][mC][mA][mA][fG][fA][fG] | 1106 |
| 0424 | mer | | [fU][mC][mG][mA][mA][mU][mG][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mC][mC][mU][mU][mC][mC][fU][fG][fC] | 1107 |
| 2024 | mer | | [fU][mA][mA][mG][mA][mU][mU][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mC][mC][mU][mG][mG][mU][fG][fU][fG] | 1108 |
| 1813 | mer | | [fA][mA][mU][mU][mA][mU][mU][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mAs][mG][mA][mA][mU][mG][mU][fA][fA][fA] | 1109 |
| 1674 | mer | | [fC][mU][mU][mU][mU][mU][mU][mA][mC | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mA][mG][mU][mU][mC][mA][fC][fU][fA] | 1110 |
| 1241 | mer | | [fC][mU][mA][mA][mA][mG][mU][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mA][mA][mG][mA][mA][mU][fG][fU][fA] | 1111 |
| 1672 | mer | | [fA][mA][mC][mU][mU][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mU][mG][mC][mA][mA][mG][A][fG][fU] | 1112 |
| 0425 | mer | | [fC][mG][mA][mA][mU][mG][mU][mU][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mGs][mG][mU][mG][mU][mG][mA][A][fU][fU] | 1113 |
| 1817 | mer | | [fA][mU][mU][mC][mA][mG][G][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs | [mCs][mC][mA][mA][mG][mA][mA][fU][fG][fU] | 1114 |
| 1671 | mer | | [fA][mA][mA][mC][mU][mU][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mAs][mG][mC][mA][mG][mC][mA][fG][fC][fU] | 1115 |
| 2136 | mer | | [fG][mA][mA][mC][mA][mA][mC][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mGs][mC][mU][mG][mA][mA][mC][A][fA][fC] | 1116 |
| 2143 | mer | | [fA][mU][mG][mU][mC][mA][mU][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mCs][mU][mG][mA][mA][mC][mA][fA][fC][fA] | 1117 |
| 2144 | mer | | [fU][mG][mU][mC][mA][mU][mU][mU][mG] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mCs][mA][mG][mC][mA][mG][mC][fU][fG][fA] | 1118 |
| 2138 | mer | | [fA][mC][mA][mA][mC][mA][mU][mG][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mUs][mU][mU][mA][mA][mC][mU][fU][fC][fC] | 1119 |
| 4909 | mer | | [fA][mG][mA][mA][mA][mU][mA][mA][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mAs][mG][mC][mA][mG][mC][mU][fG][fA][fA] | 1120 |
| 2139 | mer | | [fC][mA][mA][mC][mA][mU][mG][mU][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mUs][mU][mG][mA][mU][mG][mC][fA][fG][fU] | 1121 |
| 2411 | mer | | [fU][mU][mG][mG][mA][mA][mA][mU][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mUs][mG][mA][mA][mC][mA][mA][fC][fA][fU] | 1122 |
| 2145 | mer | | [fG][mU][mC][mA][mU][mU][mU][mG][mC] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mUs][mA][mG][mU][mG][mU][mA][fA][fA][fA] | 1123 |
| 4831 | mer | | [fA][mU][mU][mU][mA][mU][mA][mU][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mUs][mA][mA][mC][mU][mU][mU][fG][fU][fG] | 1124 |
| 2622 | mer | | [fG][mU][mU][mC][mC][mA][mG][mA][mU] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mAs][mA][mG][mC][mA][mG][mC][A][fG][fC] | 1125 |
| 2135 | mer | | [fU][mG][mA][mA][mC][mA][mA][mC][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 36 | Hs-Mm | [mCs][mA][mA][mA][mA][mG][mU][fG][fA][fU] | 1126 |
| 1383 | mer | | [fG][mA][mA][mC][mA][mU][mG][mG][mA] | |
| | | | [mA][mG][mC][mA][mG][mC][mC][mG][ademA- | |
| | | | GalNAc][ademA-GalNAc][ademA- | |
| | | | GalNAc][mG][mG][mC][mU][mG][mC] | |
| STAT3- | Modified 22 | | [MePhosphonate-4O- | 1127 |
| 715 | mer | | mUs][fGs][fAs][fA][fA][mU][fC][mA][mA][fA] | |
| | | | [mG][mU][mC][fA][mU][mC][mC][mU][mG] | |
| | | | [mGs][mGs][mG] | |
| STAT3- | Modified 22 | | [MePhosphonate-4O- | 1128 |
| 716 | mer | | mUs][fUs][fGs][fA][fA][mA][fU][mC][mA][fA] | |
| | | | [mA][mG][mU][fC][mA][mU][mC][mC][mU] | |
| | | | [mGs][mGs][mG] | |
| STAT3- | Modified 22 | | [MePhosphonate-4O- | 1129 |
| 717 | mer | | mUs][fUs][fUs][fG][fA][mA][fA][mU][mC][fA] | |
| | | | [mA][mA][mG][fU][mC][mA][mU][mC][mC] | |
| | | | [mUs][mGs][mG] | |
| STAT3- | Modified 22 | | [MePhosphonate-4O- | 1130 |
| 720 | mer | | mUs][fUs][fAs][fG][fU][mU][fG][mA][mA][fA] | |
| | | | [mU][mC][mA][fA][mA][mG][mU][mC][mA] | |
| | | | [mUs][mGs][mG] | |
| STAT3- | Modified 22 | | [MePhosphonate-4O- | 1131 |
| 372 | mer | | mUs][fAs][fGs][fA][fU][mU][fA][mU][mG][fA] | |
| | | | [mA][mA][mC][fA][mC][mC][mA][mA][mA] | |
| | | | [mGs][mGs][mG] | |
| STAT3- | Modified 22 | | [MePhosphonate-4O- | 1132 |
| 721 | mer | | mUs][fAs][fUs][fA][fG][mU][fU][mG][mA][fA] | |
| | | | [mA][mU][mC][fA][mA][mA][mG][mU][mC] | |
| | | | [mAs][mGs][mG] | |
| STAT3- | Modified 22 | | [MePhosphonate-4O- | 1133 |
| 722 | mer | | mUs][fUs][fAs][fU][fA][mG][fU][mU][mG][fA] | |
| | | | [mA][mA][mU][fC][mA][mA][mA][mG][mU] | |
| | | | [mCs][mGs][mG] | |
| STAT3- | Modified 22 | | [MePhosphonate-4O- | 1134 |
| 768 | mer | | mUs][fUs][fUs][fG][fU][mU][fU][mC][mC][fA] | |
| | | | [mU][mU][mC][fA][mG][mA][mU][mC][mU] | |
| | | | [mUs][mGs][mG] | |
| STAT3- | Modified 22 | | [MePhosphonate-4O- | 1135 |
| 1001 | mer | | mUs][fAs][fUs][fG][fA][mC][fG][mU][mU][fA] | |
| | | | [mU][mC][mC][fA][mG][mU][mU][mU][mU] | |
| | | | [mCs][mGs][mG] | |
| STAT3- | Modified 22 | | [MePhosphonate-4O- | 1136 |
| 1006 | mer | | mUs][fUs][fGs][fC][fU][mA][fA][mU][mG][fA] | |
| | | | [mC][mG][mU][fU][mA][mU][mC][mC][mA] | |
| | | | [mGs][mGs][mG] | |
| STAT3- | Modified 22 | | [MePhosphonate-4O- | 1137 |
| 1145 | mer | | mUs][fUs][fCs][fA][fU][mU][fA][mA][mG][fU] | |
| | | | [mU][mU][mC][fU][mA][mA][mA][mC][mA] | |
| | | | [mGs][mGs][mG] | |
| STAT3- | Modified 22 | | [MePhosphonate-4O- | 1138 |
| 1151 | mer | | mUs][fCs][fAs][fC][fU][mU][fU][mU][mC][fA] | |
| | | | [mU][mU][mA][fA][mG][mU][mU][mU][mC] | |
| | | | [mUs][mGs][mG] | |
| STAT3- | Modified 22 | | [MePhosphonate-4O- | 1139 |
| 1268 | mer | | mUs][fUs][fCs][fA][fA][mC][fU][mC][mA][fG] | |
| | | | [mG][mG][mA][fA][mU][mU][mU][mG][mA] | |
| | | | [mCs][mGs][mG] | |
| STAT3- | Modified 22 | | [MePhosphonate-4O- | 1140 |
| 1273 | mer | | mUs][fAs][fUs][fA][fA][mU][fU][mC][mA][fA] | |
| | | | [mC][mU][mC][fA][mG][mG][mG][mA][mA] | |
| | | | [mUs][mGs][mG] | |
| STAT3- | Modified 22 | | [MePhosphonate-4O- | 1141 |
| 1279 | mer | | mUs][fAs][fAs][fG][fC][mU][fG][mA][mU][fA] | |
| | | | [mA][mU][mU][fC][mA][mA][mC][mU][mC] | |
| | | | [mAs][mGs][mG] | |
| STAT3- | Modified 22 | | [MePhosphonate-4O- | 1142 |
| 1280 | mer | | mUs][fUs][fAs][fA][fG][mC][fU][mG][mA][fU] | |
| | | | [mA][mA][mU][fU][mC][mA][mA][mC][mU] | |
| | | | [mCs][mGs][mG] | |
| STAT3- | Modified 22 | | [MePhosphonate-4O- | 1143 |
| 1281 | mer | | mUs][fUs][fUs][fA][fA][mG][fC][mU][mG][fA] | |
| | | | [mU][mA][mA][fU][mU][mC][mA][mA][mC] | |
| | | | [mUs][mGs][mG] | |
| STAT3- | Modified 22 | | [MePhosphonate-4O- | 1144 |
| 1284 | mer | | mUs][fAs][fUs][fU][fU][mU][fA][mA][mG][fC] | |
| | | | [mU][mG][mA][fU][mA][mA][mU][mU][mC] | |
| | | | [mAs][mGs][mG] | |
| STAT3- | Modified 22 | | [MePhosphonate-4O- | 1145 |
| 1286 | mer | | mUs][fUs][fAs][fA][fU][mU][fU][mU][mA][fA] | |
| | | | [mG][mC][mU][fG][mA][mU][mA][mA][mU] | |
| | | | [mUs][mGs][mG] | |
| STAT3- | Modified 22 | | [MePhosphonate-4O- | 1146 |
| 1287 | mer | | mUs][fUs][fUs][fA][fA][mU][fU][mU][mU][fA] | |
| | | | [mA][mG][mC][fU][mG][mA][mU][mA][mA] | |
| | | | [mUs][mGs][mG] | |
| STAT3- | Modified 22 | | [MePhosphonate-4O- | 1147 |
| 1292 | mer | | mUs][fAs][fCs][fA][fC][mU][fU][mU][mA][fA] | |
| | | | [mU][mU][mU][fU][mA][mA][mG][mC][mU] | |
| | | | [mGs][mGs][mG] | |
| STAT3- | Modified 22 | | [MePhosphonate-4O- | 1148 |
| 1293 | mer | | mUs][fCs][fAs][fC][fA][mC][fU][mU][mU][fA] | |
| | | | [mA][mU][mU][fU][mU][mA][mA][mG][mC] | |
| | | | [mUs][mGs][mG] | |
| STAT3- | Modified 22 | | [MePhosphonate-4O- | 1149 |
| 1819 | mer | | mUs][fAs][fCs][fA][fC][mC][fC][mU][mG][fA] | |
| | | | [mA][mU][mA][fA][mU][mU][mC][mA][mC] | |
| | | | [mAs][mGs][mG] | |
| STAT3- | Modified 22 | | [MePhosphonate-4O- | 1150 |
| 1908 | mer | | mUs][fAs][fCs][fA][fA][mG][fG][mU][mC][fA] | |
| | | | [mA][mU][mG][fA][mU][mA][mU][mU][mG] | |
| | | | [mUs][mGs][mG] | |
| STAT3- | Modified 22 | | [MePhosphonate-4O- | 1151 |
| 1910 | mer | | mUs][fUs][fCs][fA][fC][mA][fA][mG][mG][fU] | |
| | | | [mC][mA][mA][fU][mG][mA][mU][mA][mU] | |
| | | | [mUs][mGs][mG] | |
| STAT3- | Modified 22 | | [MePhosphonate-4O- | 1152 |
| 1913 | mer | | mUs][fUs][fUs][fU][fU][mC][fA][mC][mA][fA] | |
| | | | [mG][mG][mU][fC][mA][mA][mU][mG][mA] | |
| | | | [mUs][mGs][mG] | |
| STAT3- | Modified 22 | | [MePhosphonate-4O- | 1153 |
| 2154 | mer | | mUs][fAs][fUs][fG][fA][mU][fU][mU][mC][fA] | |
| | | | [mG][mC][mA][fA][mA][mU][mG][mA][mC] | |
| | | | [mAs][mGs][mG] | |
| STAT3- | Modified 22 | | [MePhosphonate-4O- | 1154 |
| 2327 | mer | | mUs][fAs][fGs][fA][fU][mA][fA][mA][mC][fU] | |
| | | | [mU][mG][mG][fU][mC][mU][mU][mC][mA] | |
| | | | [mGs][mGs][mG] | |
| STAT3- | Modified 22 | | [MePhosphonate-4O- | 1155 |
| 2335 | mer | | mUs][fUs][fGs][fU][fC][mA][fC][mA][mC][fA] | |
| | | | [mG][mA][mU][fA][mA][mA][mC][mU][mU] | |
| | | | [mGs][mGs][mG] | |
| STAT3- | Modified 22 | | [MePhosphonate-4O- | 1156 |
| 2418 | mer | | mUs][fUs][fCs][fA][fC][mC][fA][mU][mU][fA] | |
| | | | [mU][mU][mU][fC][mC][mA][mA][mA][mC] | |
| | | | [mUs][mGs][mG] | |
| STAT3- | Modified 22 | | [MePhosphonate-4O- | 1157 |
| 2692 | mer | | mUs][fUs][fUs][fC][fA][mC][fU][mC][mA][fU] | |
| | | | [mU][mU][mC][fU][mC][mU][mA][mU][mU] | |
| | | | [mUs][mGs][mG] | |
| STAT3- | Modified 22 | | [MePhosphonate-4O- | 1158 |
| 2693 | mer | | mUs][fAs][fUs][fU][fC][mA][fC][mU][mC][fA] | |
| | | | [mU][mU][mU][fC][mU][mC][mU][mA][mU] | |
| | | | [mUs][mGs][mG] | |
| STAT3- | Modified 22 | Hs-Mf- | [MePhosphonate-4O- | 1159 |
| 2627 | mer | Mm | mUs][fAs][fA][fA][fA][mA][fA][mU][mC][fU] | |
| | | | [mG][mG][mA][fA][mC][mC][mA][mC][mA] | |
| | | | [mAs][mGs][mG] | |
| STAT3- | Modified 22 | Hs-Mf- | [MePhosphonate-4O- | 1160 |
| 2626 | mer | Mm | mUs][fAs][fA][fA][fA][mA][fU][mC][mU][fG] | |
| | | | [mG][mA][mA][fC][mC][mA][mC][mA][mA] | |
| | | | [mAs][mGs][mG] | |
| STAT3- | Modified 22 | Hs-Mf- | [MePhosphonate-4O- | 1161 |
| 2407 | mer | Mm | mUs][fUs][fC][fC][fA][mA][fA][mC][mU][fG] | |
| | | | [mC][mA][mU][fC][mA][mA][mU][mG][mA] | |
| | | | [mAs][mGs][mG] | |
| STAT3- | Modified 22 | Hs-Mf- | [MePhosphonate-4O- | 1162 |
| 2412 | mer | Mm | mUs][fUs][fU][fA][fU][mU][fU][mC][mC][fA] | |
| | | | [mA][mA][mC][fU][mG][mC][mA][mU][mC] | |
| | | | [mAs][mGs][mG] | |
| STAT3- | Modified 22 | Hs-Mf- | [MePhosphonate-4O- | 1163 |
| 2151 | mer | Mm | mUs][fAs][fU][fU][fU][mC][fA][mG][mC][fA] | |
| | | | [mA][mA][mU][fG][mA][mC][mA][mU][mG] | |
| | | | [mUs][mGs][mG] | |
| STAT3- | Modified 22 | Hs-Mf- | [MePhosphonate-4O- | 1164 |
| 2625 | mer | Mm | mUs][fAs][fA][fA][fA][mU][fC][mU][mG][fG] | |
| | | | [mA][mA][mC][fC][mA][mC][mA][mA][mA] | |
| | | | [mGs][mGs][mG] | |
| STAT3- | Modified 22 | Hs-Mf- | [MePhosphonate-4O- | 1165 |
| 4836 | mer | Mm | mUs][fAs][fC][fA][fA][mU][fA][mA][mU][fA] | |
| | | | [mU][mA][mA][fA][mU][mU][mU][mU][mU] | |
| | | | [mAs][mGs][mG] | |
| STAT3- | Modified 22 | Hs-Mf- | [MePhosphonate-4O- | 1166 |
| 2408 | mer | Mm | mUs][fUs][fU][fC][fC][mA][fA][mA][mC][fU] | |
| | | | [mG][mC][mA][fU][mC][mA][mA][mU][mG] | |
| | | | [mAs][mGs][mG] | |
| STAT3- | Modified 22 | Hs-Mf- | [MePhosphonate-4O- | 1167 |
| 2159 | mer | Mm | mUs][fCs][fC][fA][fU][mG][fA][mU][mG][fA] | |
| | | | [mU][mU][mU][fC][mA][mG][mC][mA][mA] | |
| | | | [mAs][mGs][mG] | |
| STAT3- | Modified 22 | Hs-Mf- | [MePhosphonate-4O- | 1168 |
| 2146 | mer | Mm | mUs][fAs][fG][fC][fA][mA][fA][mU][mG][fA] | |
| | | | [mC][mA][mU][fG][mU][mU][mG][mU][mU] | |
| | | | [mCs][mGs][mG] | |
| STAT3- | Modified 22 | Hs-Mf- | [MePhosphonate-4O- | 1169 |
| 2148 | mer | Mm | mUs][fUs][fC][fA][fG][mC][fA][mA][mA][fU] | |
| | | | [mG][mA][mC][fA][mU][mG][mU][mU][mG] | |
| | | | [mUs][mGs][mG] | |
| STAT3- | Modified 22 | Hs-Mf- | [MePhosphonate-4O- | 1170 |
| 2147 | mer | Mm | mUs][fCs][fA][fG][fC][mA][fA][mA][mU][fG] | |
| | | | [mA][mC][mA][fU][mG][mU][mU][mG][mU] | |
| | | | [mUs][mGs][mG] | |
| STAT3- | Modified 22 | Hs-Mf- | [MePhosphonate-4O- | 1171 |
| 0461 | mer | Mm | mUs][fGs][fA][fA][fA][mC][fU][mG][mC][fU] | |
| | | | [mU][mG][mA][fU][mU][mC][mU][mU][mC] | |
| | | | [mGs][mGs][mG] | |
| STAT3- | Modified 22 | Hs-Mf- | [MePhosphonate-4O- | 1172 |
| 1584 | mer | Mm | mUs][fAs][fU][fC][fA][mC][fC][mA][mC][fA] | |
| | | | [mA][mC][mU][fG][mG][mC][mA][mA][mG] | |
| | | | [mGs][mGs][mG] | |
| STAT3- | Modified 22 | Hs-Mf- | [MePhosphonate-4O- | 1173 |
| 1047 | mer | Mm | mUs][fUs][fC][fC][fA][mG][fU][mU][mU][fC] | |
| | | | [mU][mU][mA][fA][mU][mU][mU][mG][mU] | |
| | | | [mUs][mGs][mG] | |
| STAT3- | Modified 22 | Hs-Mf- | [MePhosphonate-4O- | 1174 |
| 0773 | mer | Mm | mUs][fAs][fC][fU][fG][mG][fU][mU][mG][fU] | |
| | | | [mU][mU][mC][fC][mA][mU][mU][mC][mA] | |
| | | | [mGs][mGs][mG] | |
| STAT3- | Modified 22 | Hs-Mf- | [MePhosphonate-4O- | 1175 |
| 0492 | mer | Mm | mUs][fUs][fC][fC][fA][mU][fU][mG][mG][fC] | |
| | | | [mU][mU][mC][fU][mC][mA][mA][mG][mA] | |
| | | | [mUs][mGs][mG] | |
| STAT3- | Modified 22 | Hs-Mf- | [MePhosphonate-4O- | 1176 |
| 0462 | mer | Mm | mUs][fAs][fG][fA][fA][mA][fC][mU][mG][fC] | |
| | | | [mU][mU][mG][fA][mU][mU][mC][mU][mU] | |
| | | | [mCs][mGs][mG] | |
| STAT3- | Modified 22 | Hs-Mf- | [MePhosphonate-4O- | 1177 |
| 1586 | mer | Mm | mUs][fAs][fG][fA][fU][mC][fA][mC][mC][fA] | |
| | | | [mC][mA][mA][fC][mU][mG][mG][mC][mA] | |
| | | | [mAs][mGs][mG] | |
| STAT3- | Modified 22 | Hs-Mf- | [MePhosphonate-4O- | 1178 |
| 0771 | mer | Mm | mUs][fUs][fG][fG][fU][mU][fG][mU][mU][fU] | |
| | | | [mC][mC][mA][fU][mU][mC][mA][mG][mA] | |
| | | | [mUs][mGs][mG] | |
| STAT3- | Modified 22 | Hs-Mf- | [MePhosphonate-4O- | 1179 |
| 0681 | mer | Mm | mUs][fUs][fU][fC][fA][mU][fU][mU][mU][fC] | |
| | | | [mU][mG][mU][fU][mC][mU][mA][mG][mA] | |
| | | | [mUs][mGs][mG] | |
| STAT3- | Modified 22 | Hs-Mf- | [MePhosphonate-4O- | 1180 |
| 0678 | mer | Mm | mUs][fAs][fU][fU][fU][mU][fC][mU][mG][fU] | |
| | | | [mU][mC][mU][fA][mG][mA][mU][mC][mC] | |
| | | | [mUs][mGs][mG] | |
| STAT3- | Modified 22 | Hs-Mf- | [MePhosphonate-4O- | 1181 |
| 4837 | mer | Mm | mUs][fCs][fA][fC][fA][mA][fU][mA][mA][fU] | |
| | | | [mA][mU][mA][fA][mA][mU][mU][mU][mU] | |
| | | | [mUs][mGs][mG] | |
| STAT3- | Modified 22 | Hs-Mf- | [MePhosphonate-4O- | 1182 |
| 4833 | mer | Mm | mUs][fAs][fU][fA][fA][mU][fA][mU][mA][fA] | |
| | | | [mA][mU][mU][fU][mU][mU][mA][mC][mA] | |
| | | | [mCs][mGs][mG] | |
| STAT3- | Modified 22 | Hs | [MePhosphonate-4O- | 1183 |
| 1068 | mer | | mUs][fGs][fA][fA][fA][mC][fU][mU][mU][fU] | |
| | | | [mU][mG][mC][fU][mG][mC][mA][mA][mC] | |
| | | | [mUs][mGs][mG] | |
| STAT3- | Modified 22 | Hs | [MePhosphonate-4O- | 1184 |
| 1673 | mer | | mUs][fUs][fA][fA][fA][mA][fA][mA][mG][fU] | |
| | | | [mU][mU][mA][fC][mA][mU][mU][mC][mU] | |
| | | | [mUs][mGs][mG] | |
| STAT3- | Modified 22 | Hs | [MePhosphonate-4O- | 1185 |
| 0426 | mer | | mUs][fAs][fG][fA][fA][mC][fA][mU][mU][fC] | |
| | | | [mG][mA][mC][fU][mC][mU][mU][mG][mC] | |
| | | | [mAs][mGs][mG] | |
| STAT3- | Modified 22 | Hs | [MePhosphonate-4O- | 1186 |
| 2404 | mer | | mUs][fAs][fA][fA][fC][mU][fG][mC][mA][fU] | |
| | | | [mC][mA][mA][fU][mG][mA][mA][mU][mC] | |
| | | | [mUs][mGs][mG] | |
| STAT3- | Modified 22 | Hs | [MePhosphonate-4O- | 1187 |
| 1067 | mer | | mUs][fAs][fA][fA][fC][mU][fU][mU][mU][fU] | |
| | | | [mG][mC][mU][fG][mC][mA][mA][mC][mU] | |
| | | | [mCs][mGs][mG] | |
| STAT3- | Modified 22 | Hs | [MePhosphonate-4O- | 1188 |
| 0433 | mer | | mUs][fCs][fU][fG][fA][mU][fA][mG][mA][fG] | |
| | | | [mA][mA][mC][fA][mU][mU][mC][mG][mA] | |
| | | | [mCs][mGs][mG] | |
| STAT3- | Modified 22 | Hs | [MePhosphonate-4O- | 1189 |
| 1670 | mer | | mUs][fAs][fA][fA][fA][mG][fU][mU][mU][fA] | |
| | | | [mC][mA][mU][fU][mC][mU][mU][mG][mG] | |
| | | | [mGs][mGs][mG] | |
| STAT3- | Modified 22 | Hs | [MePhosphonate-4O- | 1190 |
| 1388 | mer | | mUs][fAs][fU][fU][fC][mU][fU][mC][mC][fA] | |
| | | | [mU][mG][mU][fU][mC][mA][mU][mC][mA] | |
| | | | [mCs][mGs][mG] | |
| STAT3- | Modified 22 | Hs | [MePhosphonate-4O- | 1191 |
| 0429 | mer | | mUs][fUs][fA][fG][fA][mG][fA][mA][mC][fA] | |
| | | | [mU][mU][mC][fG][mA][mC][mU][mC][mU] | |
| | | | [mUs][mGs][mG] | |
| STAT3- | Modified 22 | Hs | [MePhosphonate-4O- | 1192 |
| 2405 | mer | | mUs][fCs][fA][fA][fA][mC][fU][mG][mC][fA] | |
| | | | [mU][mC][mA][fA][mU][mG][mA][mA][mU] | |
| | | | [mCs][mGs][mG] | |
| STAT3- | Modified 22 | Hs | [MePhosphonate-4O- | 1193 |
| 0430 | mer | | mUs][fAs][fU][fA][fG][mA][fG][mA][mA][fC] | |
| | | | [mA][mU][mU][fC][mG][mA][mC][mU][mC] | |
| | | | [mUs][mGs][mG] | |
| STAT3- | Modified 22 | Hs | [MePhosphonate-4O- | 1194 |
| 0432 | mer | | mUs][fUs][fG][fA][fU][mA][fG][mA][mG][fA] | |
| | | | [mA][mC][mA][fU][mU][mC][mG][mA][mC] | |
| | | | [mUs][mGs][mG] | |
| STAT3- | Modified 22 | Hs | [MePhosphonate-4O- | 1195 |
| 1815 | mer | | mUs][fCs][fC][fU][fG][mA][fA][mU][mA][fA] | |
| | | | [mU][mU][mC][fA][mC][mA][mC][mC][mA] | |
| | | | [mGs][mGs][mG] | |
| STAT3- | Modified 22 | Hs | [MePhosphonate-4O- | 1196 |
| 0424 | mer | | mUs][fAs][fA][fC][fA][mU][fU][mC][mG][fA] | |
| | | | [mC][mU][mC][fU][mU][mG][mC][mA][mG] | |
| | | | [mGs][mGs][mG] | |
| STAT3- | Modified 22 | Hs | [MePhosphonate-4O- | 1197 |
| 2024 | mer | | mUs][fUs][fG][fA][fA][mU][fC][mU][mU][fA] | |
| | | | [mG][mC][mA][fG][mG][mA][mA][mG][mG] | |
| | | | [mUs][mGs][mG] | |
| STAT3- | Modified 22 | Hs | [MePhosphonate-4O- | 1198 |
| 1813 | mer | | mUs][fUs][fG][fA][fA][mU][fA][mA][mU][fU] | |
| | | | [mC][mA][mC][fA][mC][mC][mA][mG][mG] | |
| | | | [mUs][mGs][mG] | |
| STAT3- | Modified 22 | Hs | [MePhosphonate-4O- | 1199 |
| 1674 | mer | | mUs][fGs][fU][fA][fA][mA][fA][mA][mA][fG] | |
| | | | [mU][mU][mU][fA][mC][mA][mU][mU][mC] | |
| | | | [mUs][mGs][mG] | |
| STAT3- | Modified 22 | Hs | [MePhosphonate-4O- | 1200 |
| 1241 | mer | | mUs][fUs][fG][fA][fC][mU][fU][mU][mA][fG] | |
| | | | [mU][mA][mG][fU][mG][mA][mA][mC][mU] | |
| | | | [mGs][mGs][mG] | |
| STAT3- | Modified 22 | Hs | [MePhosphonate-4O- | 1201 |
| 1672 | mer | | mUs][fUs][fG][fA][fC][mU][fU][mU][mA][fG] | |
| | | | [mU][mA][mG][fU][mG][mA][mA][mC][mU] | |
| | | | [mGs][mGs][mG] | |
| STAT3- | Modified 22 | Hs | [MePhosphonate-4O- | 1202 |
| 0425 | mer | | mUs][fGs][fA][fA][fC][mA][fU][mU][mC][fG] | |
| | | | [mA][mC][mU][fC][mU][mU][mG][mC][mA] | |
| | | | [mGs][mGs][mG] | |
| STAT3- | Modified 22 | Hs | [MePhosphonate-4O- | 1203 |
| 1817 | mer | | mUs][fAs][fC][fC][fC][mU][fG][mA][mA][fU] | |
| | | | [mA][mA][mU][fU][mC][mA][mC][mA][mC] | |
| | | | [mCs][mGs][mG] | |
| STAT3- | Modified 22 | Hs | [MePhosphonate-4O- | 1204 |
| 1671 | mer | | mUs][fAs][fA][fA][fA][mA][fG][mU][mU][fU] | |
| | | | [mA][mC][mA][fU][mU][mC][mU][mU][mG] | |
| | | | [mGs][mGs][mG] | |
| STAT3- | Modified 22 | Hs-Mm | [MePhosphonate-4O- | 1205 |
| 2136 | mer | | mUs][fAs][fU][fG][fU][mU][fG][mU][mU][fC] | |
| | | | [mA][mG][mC][fU][mG][mC][mU][mG][mC] | |
| | | | [mUs][mGs][mG] | |
| STAT3- | Modified 22 | Hs-Mm | [MePhosphonate-4O- | 1206 |
| 2143 | mer | | mUs][fAs][fA][fA][fU][mG][fA][mC][mA][fU] | |
| | | | [mG][mU][mU][fG][mU][mU][mC][mA][mG] | |
| | | | [mCs][mGs][mG] | |
| STAT3- | Modified 22 | Hs-Mm | [MePhosphonate-4O- | 1207 |
| 2144 | mer | | mUs][fCs][fA][fA][fA][mU][fG][mA][mC][fA] | |
| | | | [mU][mG][mU][fU][mG][mU][mU][mC][mA] | |
| | | | [mGs][mGs][mG] | |
| STAT3- | Modified 22 | Hs-Mm | [MePhosphonate-4O- | 1208 |
| 2138 | mer | | mUs][fAs][fC][fA][fU][mG][fU][mU][mG][fU] | |
| | | | [mU][mC][mA][fG][mC][mU][mG][mC][mU] | |
| | | | [mGs][mGs][mG] | |
| STAT3- | Modified 22 | Hs-Mm | [MePhosphonate-4O- | 1209 |
| 4909 | mer | | mUs][fUs][fU][fU][fA][mU][fU][mU][mC][fU] | |
| | | | [mG][mG][mA][fA][mG][mU][mU][mA][mA] | |
| | | | [mAs][mGs][mG] | |
| STAT3- | Modified 22 | Hs-Mm | [MePhosphonate-4O- | 1210 |
| 2139 | mer | | mUs][fGs][fA][fC][fA][mU][fG][mU][mU][fG] | |
| | | | [mU][mU][mC][fA][mG][mC][mU][mG][mC] | |
| | | | [mUs][mGs][mG] | |
| STAT3- | Modified 22 | Hs-Mm | [MePhosphonate-4O- | 1211 |
| 2411 | mer | | mUs][fUs][fA][fU][fU][mU][fC][mC][mA][fA] | |
| | | | [mA][mC][mU][fG][mC][mA][mU][mC][mA] | |
| | | | [mAs][mGs][mG] | |
| STAT3- | Modified 22 | Hs-Mm | [MePhosphonate-4O- | 1212 |
| 2145 | mer | | mUs][fGs][fC][fA][fA][mA][fU][mG][mA][fC] | |
| | | | [mA][mU][mG][fU][mU][mG][mU][mU][mC] | |
| | | | [mAs][mGs][mG] | |
| STAT3- | Modified 22 | Hs-Mm | [MePhosphonate-4O- | 1213 |
| 4831 | mer | | mUs][fAs][fA][fU][fA][mU][fA][mA][mA][fU] | |
| | | | [mU][mU][mU][fU][mA][mC][mA][mC][mU] | |
| | | | [mAs][mGs][mG] | |
| STAT3- | Modified 22 | Hs-Mm | [MePhosphonate-4O- | 1214 |
| 2622 | mer | | mUs][fAs][fU][fC][fU][mG][fG][mA][mA][fC] | |
| | | | [mC][mA][mC][fA][mA][mA][mG][mU][mU] | |
| | | | [mAs][mGs][mG] | |
| STAT3- | Modified 22 | Hs-Mm | [MePhosphonate-4O- | 1215 |
| 2135 | mer | | mUs][fUs][fG][fU][fU][mG][fU][mU][mC][fA] | |
| | | | [mG][mC][mU][fG][mC][mU][mG][mC][mU] | |
| | | | [mUs][mGs][mG] | |
| STAT3- | Modified 22 | Hs-Mm | [MePhosphonate-4O- | 1216 |
| 1383 | mer | | mUs][fUs][fG][fU][fU][mG][fU][mU][mC][fA] | |
| | | | [mG][mC][mU][fG][mC][mU][mG][mC][mU] | |
| | | | [mUs][mGs][mG] | |
| NM_139276.3 | | | GTCGCAGCCGAGGGAACAAGCCCCAACC | 1217 |
| human | | | GGATCCTGGACAGGCACCCCGGCTTGGC | |
| STAT3 | | | GCTGTCTCTCCCCCTCGGCTCGGAGAGGC | |
| nucleotide | | | CCTTCGGCCTGAGGGAGCCTCGCCGCCC | |
| sequence | | | GTCCCCGGCACACGCGCAGCCCCGGCCT | |
| | | | CTCGGCCTCTGCCGGAGAAACAGTTGGG | |
| | | | ACCCCTGATTTTAGCAGGATGGCCCAATG | |
| | | | GAATCAGCTACAGCAGCTTGACACACGG | |
| | | | TACCTGGAGCAGCTCCATCAGCTCTACAG | |
| | | | TGACAGCTTCCCAATGGAGCTGCGGCAG | |
| | | | TTTCTGGCCCCTTGGATTGAGAGTCAAGA | |
| | | | TTGGGCATATGCGGCCAGCAAAGAATCA | |
| | | | CATGCCACTTTGGTGTTTCATAATCTCCT | |
| | | | GGGAGAGATTGACCAGCAGTATAGCCGC | |
| | | | TTCCTGCAAGAGTCGAATGTTCTCTATCA | |
| | | | GCACAATCTACGAAGAATCAAGCAGTTT | |
| | | | CTTCAGAGCAGGTATCTTGAGAAGCCAA | |
| | | | TGGAGATTGCCCGGATTGTGGCCCGGTG | |
| | | | CCTGTGGGAAGAATCACGCCTTCTACAG | |
| | | | ACTGCAGCCACTGCGGCCCAGCAAGGGG | |
| | | | GCCAGGCCAACCACCCCACAGCAGCCGT | |
| | | | GGTGACGGAGAAGCAGCAGATGCTGGAG | |
| | | | CAGCACCTTCAGGATGTCCGGAAGAGAG | |
| | | | TGCAGGATCTAGAACAGAAAATGAAAGT | |
| | | | GGTAGAGAATCTCCAGGATGACTTTGATT | |
| | | | TCAACTATAAAACCCTCAAGAGTCAAGG | |
| | | | AGACATGCAAGATCTGAATGGAAACAAC | |
| | | | CAGTCAGTGACCAGGCAGAAGATGCAGC | |
| | | | AGCTGGAACAGATGCTCACTGCGCTGGA | |
| | | | CCAGATGCGGAGAAGCATCGTGAGTGAG | |
| | | | CTGGCGGGGCTTTTGTCAGCGATGGAGT | |
| | | | ACGTGCAGAAAACTCTCACGGACGAGGA | |
| | | | GCTGGCTGACTGGAAGAGGCGGCAACAG | |
| | | | ATTGCCTGCATTGGAGGCCCGCCCAACAT | |
| | | | CTGCCTAGATCGGCTAGAAAACTGGATA | |
| | | | ACGTCATTAGCAGAATCTCAACTTCAGAC | |
| | | | CCGTCAACAAATTAAGAAACTGGAGGAG | |
| | | | TTGCAGCAAAAAGTTTCCTACAAAGGGG | |
| | | | ACCCCATTGTACAGCACCGGCCGATGCT | |
| | | | GGAGGAGAGAATCGTGGAGCTGTTTAGA | |
| | | | AACTTAATGAAAAGTGCCTTTGTGGTGG | |
| | | | AGCGGCAGCCCTGCATGCCCATGCATCCT | |
| | | | GACCGGCCCCTCGTCATCAAGACCGGCG | |
| | | | TCCAGTTCACTACTAAAGTCAGGTTGCTG | |
| | | | GTCAAATTCCCTGAGTTGAATTATCAGCT | |
| | | | TAAAATTAAAGTGTGCATTGACAAAGAC | |
| | | | TCTGGGGACGTTGCAGCTCTCAGAGGAT | |
| | | | CCCGGAAATTTAACATTCTGGGCACAAA | |
| | | | CACAAAAGTGATGAACATGGAAGAATCC | |
| | | | AACAACGGCAGCCTCTCTGCAGAATTCA | |
| | | | AACACTTGACCCTGAGGGAGCAGAGATG | |
| | | | TGGGAATGGGGGCCGAGCCAATTGTGAT | |
| | | | GCTTCCCTGATTGTGACTGAGGAGCTGCA | |
| | | | CCTGATCACCTTTGAGACCGAGGTGTATC | |
| | | | ACCAAGGCCTCAAGATTGACCTAGAGAC | |
| | | | CCACTCCTTGCCAGTTGTGGTGATCTCCA | |
| | | | ACATCTGTCAGATGCCAAATGCCTGGGC | |
| | | | GTCCATCCTGTGGTACAACATGCTGACCA | |
| | | | ACAATCCCAAGAATGTAAACTTTTTTACC | |
| | | | AAGCCCCCAATTGGAACCTGGGATCAAG | |
| | | | TGGCCGAGGTCCTGAGCTGGCAGTTCTCC | |
| | | | TCCACCACCAAGCGAGGACTGAGCATCG | |
| | | | AGCAGCTGACTACACTGGCAGAGAAACT | |
| | | | CTTGGGACCTGGTGTGAATTATTCAGGGT | |
| | | | GTCAGATCACATGGGCTAAATTTTGCAA | |
| | | | AGAAAACATGGCTGGCAAGGGCTTCTCC | |
| | | | TTCTGGGTCTGGCTGGACAATATCATTGA | |
| | | | CCTTGTGAAAAAGTACATCCTGGCCCTTT | |
| | | | GGAACGAAGGGTACATCATGGGCTTTAT | |
| | | | CAGTAAGGAGCGGGAGCGGGCCATCTTG | |
| | | | AGCACTAAGCCTCCAGGCACCTTCCTGCT | |
| | | | AAGATTCAGTGAAAGCAGCAAAGAAGGA | |
| | | | GGCGTCACTTTCACTTGGGTGGAGAAGG | |
| | | | ACATCAGCGGTAAGACCCAGATCCAGTC | |
| | | | CGTGGAACCATACACAAAGCAGCAGCTG | |
| | | | AACAACATGTCATTTGCTGAAATCATCAT | |
| | | | GGGCTATAAGATCATGGATGCTACCAAT | |
| | | | ATCCTGGTGTCTCCACTGGTCTATCTCTA | |
| | | | TCCTGACATTCCCAAGGAGGAGGCATTC | |
| | | | GGAAAGTATTGTCGGCCAGAGAGCCAGG | |
| | | | AGCATCCTGAAGCTGACCCAGGTAGCGC | |
| | | | TGCCCCATACCTGAAGACCAAGTTTATCT | |
| | | | GTGTGACACCAACGACCTGCAGCAATAC | |
| | | | CATTGACCTGCCGATGTCCCCCCGCACTT | |
| | | | TAGATTCATTGATGCAGTTTGGAAATAAT | |
| | | | GGTGAAGGTGCTGAACCCTCAGCAGGAG | |
| | | | GGCAGTTTGAGTCCCTCACCTTTGACATG | |
| | | | GAGTTGACCTCGGAGTGCGCTACCTCCCC | |
| | | | CATGTGAGGAGCTGAGAACGGAAGCTGC | |
| | | | AGAAAGATACGACTGAGGCGCCTACCTG | |
| | | | CATTCTGCCACCCCTCACACAGCCAAACC | |
| | | | CCAGATCATCTGAAACTACTAACTTTGTG | |
| | | | GTTCCAGATTTTTTTTAATCTCCTACTTCT | |
| | | | GCTATCTTTGAGCAATCTGGGCACTTTTA | |
| | | | AAAATAGAGAAATGAGTGAATGTGGGTG | |
| | | | ATCTGCTTTTATCTAAATGCAAATAAGGA | |
| | | | TGTGTTCTCTGAGACCCATGATCAGGGGA | |
| | | | TGTGGCGGGGGGTGGCTAGAGGGAGAAA | |
| | | | AAGGAAATGTCTTGTGTTGTTTTGTTCCC | |
| | | | CTGCCCTCCTTTCTCAGCAGCTTTTTGTTA | |
| | | | TTGTTGTTGTTGTTCTTAGACAAGTGCCT | |
| | | | CCTGGTGCCTGCGGCATCCTTCTGCCTGT | |
| | | | TTCTGTAAGCAAATGCCACAGGCCACCT | |
| | | | ATAGCTACATACTCCTGGCATTGCACTTT | |
| | | | TTAACCTTGCTGACATCCAAATAGAAGAT | |
| | | | AGGACTATCTAAGCCCTAGGTTTCTTTTT | |
| | | | AAATTAAGAAATAATAACAATTAAAGGG | |
| | | | CAAAAAACACTGTATCAGCATAGCCTTTC | |
| | | | TGTATTTAAGAAACTTAAGCAGCCGGGC | |
| | | | ATGGTGGCTCACGCCTGTAATCCCAGCAC | |
| | | | TTTGGGAGGCCGAGGCGGATCATAAGGT | |
| | | | CAGGAGATCAAGACCATCCTGGCTAACA | |
| | | | CGGTGAAACCCCGTCTCTACTAAAAGTA | |
| | | | CAAAAAATTAGCTGGGTGTGGTGGTGGG | |
| | | | CGCCTGTAGTCCCAGCTACTCGGGAGGCT | |
| | | | GAGGCAGGAGAATCGCTTGAACCTGAGA | |
| | | | GGCGGAGGTTGCAGTGAGCCAAAATTGC | |
| | | | ACCACTGCACACTGCACTCCATCCTGGGC | |
| | | | GACAGTCTGAGACTCTGTCTCAAAAAAA | |
| | | | AAAAAAAAAAAAAGAAACTTCAGTTAAC | |
| | | | AGCCTCCTTGGTGCTTTAAGCATTCAGCT | |
| | | | TCCTTCAGGCTGGTAATTTATATAATCCC | |
| | | | TGAAACGGGCTTCAGGTCAAACCCTTAA | |
| | | | GACATCTGAAGCTGCAACCTGGCCTTTGG | |
| | | | TGTTGAAATAGGAAGGTTTAAGGAGAAT | |
| | | | CTAAGCATTTTAGACTTTTTTTTATAAAT | |
| | | | AGACTTATTTTCCTTTGTAATGTATTGGC | |
| | | | CTTTTAGTGAGTAAGGCTGGGCAGAGGG | |
| | | | TGCTTACAACCTTGACTCCCTTTCTCCCT | |
| | | | GGACTTGATCTGCTGTTTCAGAGGCTAGG | |
| | | | TTGTTTCTGTGGGTGCCTTATCAGGGCTG | |
| | | | GGATACTTCTGATTCTGGCTTCCTTCCTG | |
| | | | CCCCACCCTCCCGACCCCAGTCCCCCTGA | |
| | | | TCCTGCTAGAGGCATGTCTCCTTGCGTGT | |
| | | | CTAAAGGTCCCTCATCCTGTTTGTTTTAG | |
| | | | GAATCCTGGTCTCAGGACCTCATGGAAG | |
| | | | AAGAGGGGGAGAGAGTTACAGGTTGGAC | |
| | | | ATGATGCACACTATGGGGCCCCAGCGAC | |
| | | | GTGTCTGGTTGAGCTCAGGGAATATGGTT | |
| | | | CTTAGCCAGTTTCTTGGTGATATCCAGTG | |
| | | | GCACTTGTAATGGCGTCTTCATTCAGTTC | |
| | | | ATGCAGGGCAAAGGCTTACTGATAAACT | |
| | | | TGAGTCTGCCCTCGTATGAGGGTGTATAC | |
| | | | CTGGCCTCCCTCTGAGGCTGGTGACTCCT | |
| | | | CCCTGCTGGGGCCCCACAGGTGAGGCAG | |
| | | | AACAGCTAGAGGGCCTCCCCGCCTGCCC | |
| | | | GCCTTGGCTGGCTAGCTCGCCTCTCCTGT | |
| | | | GCGTATGGGAACACCTAGCACGTGCTGG | |
| | | | ATGGGCTGCCTCTGACTCAGAGGCATGG | |
| | | | CCGGATTTGGCAACTCAAAACCACCTTGC | |
| | | | CTCAGCTGATCAGAGTTTCTGTGGAATTC | |
| | | | TGTTTGTTAAATCAAATTAGCTGGTCTCT | |
| | | | GAATTAAGGGGGAGACGACCTTCTCTAA | |
| | | | GATGAACAGGGTTCGCCCCAGTCCTCCTG | |
| | | | CCTGGAGACAGTTGATGTGTCATGCAGA | |
| | | | GCTCTTACTTCTCCAGCAACACTCTTCAG | |
| | | | TACATAATAAGCTTAACTGATAAACAGA | |
| | | | ATATTTAGAAAGGTGAGACTTGGGCTTA | |
| | | | CCATTGGGTTTAAATCATAGGGACCTAG | |
| | | | GGCGAGGGTTCAGGGCTTCTCTGGAGCA | |
| | | | GATATTGTCAAGTTCATGGCCTTAGGTAG | |
| | | | CATGTATCTGGTCTTAACTCTGATTGTAG | |
| | | | CAAAAGTTCTGAGAGGAGCTGAGCCCTG | |
| | | | TTGTGGCCCATTAAAGAACAGGGTCCTC | |
| | | | AGGCCCTGCCCGCTTCCTGTCCACTGCCC | |
| | | | CCTCCCCATCCCCAGCCCAGCCGAGGGA | |
| | | | ATCCCGTGGGTTGCTTACCTACCTATAAG | |
| | | | GTGGTTTATAAGCTGCTGTCCTGGCCACT | |
| | | | GCATTCAAATTCCAATGTGTACTTCATAG | |
| | | | TGTAAAAATTTATATTATTGTGAGGTTTT | |
| | | | TTGTCTTTTTTTTTTTTTTTTTTTTTTGGTA | |
| | | | TATTGCTGTATCTACTTTAACTTCCAGAA | |
| | | | ATAAACGTTATATAGGAACCGTC | |
| XM_005584240.2 | | | TGCATGACGGCGTGCCTCGGCCAGGCTG | 1218 |
| Non- | | | GGGCTGGGCGGGGATTGGCTGAAGGGGC | |
| human | | | TGTAATTCAGCGGTTTCCGGAGCTGCGGC | |
| primate | | | GGCGTAGACCGGGAGGGGGAGCCGGGG | |
| STAT3 | | | GTTCCGACGTAGCAGCCGAGGGAACAAG | |
| nucleotide | | | CCCCAACCGGATCCTGGACAGGCACCCC | |
| sequence | | | GGCTCGGCGCTGTCTCTCCCCCTCGGCTC | |
| | | | GGATAAGCCCTCCGGCCTGAGGGAGCCC | |
| | | | CGTCGCCCGCCCCCGGCGCACGCGCAGC | |
| | | | CCCGGCCTCTCGGCCTCTGCTGGAGAAAC | |
| | | | AGCAGGATGGCCCAATGGAATCAGCTAC | |
| | | | AGCAGCTTGACACACGGTACCTGGAGCA | |
| | | | GCTCCATCAGCTCTACAGTGACAGCTTCC | |
| | | | CAATGGAGTTGCGGCAGTTTCTGGCCCCT | |
| | | | TGGATTGAGAGTCAAGATTGGGCATATG | |
| | | | CGGCCAGCAAAGAATCACATGCCACTTT | |
| | | | GGTGTTTCATAATCTCCTGGGCGAGATTG | |
| | | | ACCAGCAGTATAGCCGCTTCCTGCAAGA | |
| | | | ATCGAATGTTCTCTATCAGCACAATCTAC | |
| | | | GAAGAATCAAGCAGTTTCTTCAGAGCAG | |
| | | | GTATCTTGAGAAGCCAATGGAGATTGCC | |
| | | | CGGATTGTGGCCCGGTGCCTGTGGGAAG | |
| | | | AGTCACGCCTCCTACAGACTGCAGCCACT | |
| | | | GCGGCCCAGCAAGGGGGCCAGGCCAACC | |
| | | | ACCCCACAGCAGCTGTGGTGACGGAGAA | |
| | | | GCAGCAGATGCTGGAGCAGCACCTTCAG | |
| | | | GATGTCCGGAAGAGAGTACAGGATCTAG | |
| | | | AACAGAAAATGAAAGTGGTAGAGAATCT | |
| | | | CCAGGATGACTTTGATTTCAACTATAAAA | |
| | | | CCCTCAAGAGTCAAGGAGACATGCAAGA | |
| | | | TCTGAATGGAAACAACCAGTCAGTGACC | |
| | | | AGGCAGAAGATGCAGCAGCTGGAACAGA | |
| | | | TGCTCACTGCGCTGGACCAGATGCGGAG | |
| | | | AAGCATCGTGAGTGAGCTGGCGGGGCTT | |
| | | | TTGTCAGCGATGGAGTACGTGCAGAAAA | |
| | | | CTCTCACAGACGAGGAGCTGGCTGACTG | |
| | | | GAAGAGGCGGCAACAGATTGCCTGCATT | |
| | | | GGAGGTCCGCCCAACATCTGCCTAGATC | |
| | | | GGCTAGAAAACTGGATAACGTCATTAGC | |
| | | | AGAATCTCAACTTCAGACCCGTCAACAA | |
| | | | ATTAAGAAACTGGAGGAGTTGCAGCAAA | |
| | | | AAGTGTCCTACAAAGGGGACCCCATTGT | |
| | | | ACAGCACCGGCCGATGCTGGAGGAGAGA | |
| | | | ATCGTGGAGCTGTTCAGAAACTTAATGA | |
| | | | AAAGTGCCTTTGTGGTGGAGCGGCAGCC | |
| | | | CTGCATGCCCATGCATCCCGACCGGCCCC | |
| | | | TTGTCATCAAGACCGGCGTCCAGTTCACT | |
| | | | ACCAAAGTCAGGTTGCTGGTCAAATTCCC | |
| | | | TGAGTTAAATTATCAACTTAAAATTAAAG | |
| | | | TGTGCATTGACAAAGACTCTGGGGATGTT | |
| | | | GCAGCTCTCAGAGGATCCCGGAAATTTA | |
| | | | ACATTCTGGGCACAAACACCAAAGTGAT | |
| | | | GAACATGGAAGAGTCCAACAACGGCAGC | |
| | | | CTCTCTGCAGAATTCAAACACTTGACCCT | |
| | | | GAGGGAGCAGAGATGTGGGAATGGGGG | |
| | | | CCGAGCCAATTGTGATGCTTCCCTGATTG | |
| | | | TGACTGAGGAGCTGCACCTGATCACCTTT | |
| | | | GAGACAGAGGTATATCACCAAGGCCTCA | |
| | | | AGATTGACCTAGAGACCCACTCCTTGCCA | |
| | | | GTTGTGGTGATCTCCAACATCTGTCAGAT | |
| | | | GCCAAATGCCTGGGCGTCCATCCTGTGGT | |
| | | | ACAACATGCTGACCAACAACCCCAAGAA | |
| | | | CGTAAACTTTTTTACCAAGCCCCCAATCG | |
| | | | GAACCTGGGATCAAGTGGCCGAGGTCCT | |
| | | | GAGCTGGCAGTTCTCCTCCACCACCAAGC | |
| | | | GAGGACTGAGCATCGAGCAGCTGACTAC | |
| | | | ACTGGCGGAGAAACTCTTGGGACCTGGC | |
| | | | GTGAATTATTCAGGGTGTCAGATCACATG | |
| | | | GGCTAAATTTTGCAAAGAAAACATGGCT | |
| | | | GGCAAGGGCTTCTCCTTCTGGGTCTGGCT | |
| | | | GGACAATATCATTGACCTTGTGAAAAAG | |
| | | | TACATCCTGGCCCTTTGGAATGAAGGGTA | |
| | | | CATCATGGGCTTTATCAGTAAGGAGCGG | |
| | | | GAGCGGGCCATCTTGAGCACCAAGCCTC | |
| | | | CAGGCACCTTTCTGCTAAGATTCAGTGAA | |
| | | | AGCAGCAAAGAAGGCGGCGTCACTTTCA | |
| | | | CTTGGGTGGAGAAGGACATCAGTGGTAA | |
| | | | GACCCAGATCCAGTCCGTGGAACCATAC | |
| | | | ACCAAGCAGCAGTTGAACAACATGTCAT | |
| | | | TTGCTGAAATCATCATGGGCTATAAGATC | |
| | | | ATGGATGCTACCAATATTCTGGTGTCTCC | |
| | | | GCTGGTCTATCTCTACCCTGACATTCCCA | |
| | | | AGGAGGAGGCATTCGGAAAGTATTGTCG | |
| | | | GCCAGAGAGCCAGGAGCATCCTGAAGCT | |
| | | | GACCCAGGCGCCGCCCCATACCTGAAGA | |
| | | | CCAAGTTTATCTGTGTGACACCATTCATT | |
| | | | GATGCAGTTTGGAAATAATGGTGAAGGT | |
| | | | GCTGAACCCTCAGCAGGAGGGCAGTTTG | |
| | | | AGTCCCTCACCTTTGACATGGAGTTGACC | |
| | | | TCGGAGTGTGCTACCTCCCCCATGTGAGG | |
| | | | AGCTGAGAACGGAAGCTGCAAAAGATAC | |
| | | | GACTGAGGCGCCTACCTGTGTTCCGCCAC | |
| | | | CCCTCACACAGCCAAACCCCAGATCATC | |
| | | | TGAAACTACTAACTTTGTGGTTCCAGATT | |
| | | | TTTTTTAATCTCCTACTTCTGCTATCTTTG | |
| | | | AGCAATCTGGGCACTTTTAAAAATAAGA | |
| | | | GAAATGAGTGAATGTGGGTGATCTGCTTT | |
| | | | TATCTAAATGCAAATAAGGATGTGTTCTC | |
| | | | TGAGACCCGTGATGGGGGGATGTGGCGG | |
| | | | GGGGTGGCTAGAGGGAGAAAAAGGAAA | |
| | | | TGTCTTGTGTTGTTTTGTTCCCCTGCCCTC | |
| | | | CTTTCTCAGCAGCTTTTTGTTATTGTTGTT | |
| | | | GTTGTTCTTAGACAAGTGCCTCCTGGTGC | |
| | | | CCGCGGCATCCTTCTGCCTGTTTCTGTAA | |
| | | | GCAAATGCCACAGGCCACCTGTAGCTAC | |
| | | | ATACTCCTGGCATTGCACTTTTTAACCTT | |
| | | | GCTGACATCCAAATAGAAGATAGGACTA | |
| | | | TCTGAGCCCTAGGTTTCTTTTTAAATTAA | |
| | | | GAAATAAGAACAATTAAAGGGCAAAAA | |
| | | | ACACTGTTTCAGCATAGCCTTTCTGTATT | |
| | | | TAAGAAACTTCAGCAGCCGGCCGCAGGG | |
| | | | ACTCACGCCTGTAATCCCAGCACTTTGGG | |
| | | | AGGCCGAGGTGGGTGGATCATGAGGTTA | |
| | | | GGAGATCAAGACTGTCCTGGCTAACATG | |
| | | | GTGAAACCCCGTCTCTACTAACAGTACA | |
| | | | AAAAATTAGCCGGGCGTGGTGGTGGGTG | |
| | | | CCTGTAGTCCCAGCTACTCGGGAGGCTG | |
| | | | AGGCAGGAGAATGGCATGAACCCAAGAG | |
| | | | GCGGAGGTTGCAGTGAGCCAAAATCACA | |
| | | | CCACTGCACTCCAACTCAGGCAACAGTG | |
| | | | TGAGACTCCATCTCAAAAAAAAAAGAAA | |
| | | | AGAAAAAGAAACTTCAGTTAACAGCCTC | |
| | | | CTTGGTGCTTTAAGCATTCAGCTTCCTTC | |
| | | | AGGTTGATAATTTATATAACCCCTGAAAC | |
| | | | AGGCTTCAGGTCAAACCCTTAAAAGACG | |
| | | | TCTGAAGCTGCAGCCTGGCCTTTGATGTT | |
| | | | GAAATAGGAAGGTTTAAGGAGAATCTAA | |
| | | | GCATTTTAGACTTTTTTTTATAAATAGAC | |
| | | | TTCTATTTTCCTTTGTAATGTATTGGTCTT | |
| | | | TTAGTGGGTAAGGCTGGGCAGAGGGTGC | |
| | | | TTACAACCTTGACTCCCTTTCTCCCTGGA | |
| | | | CTTGATCTGCTGTTTCAGAGGCTAGGTTG | |
| | | | TTTCTGTGGGTGCCTTATCAGGGCTGGGA | |
| | | | TACTTCTGATTTGGGCTTCCTTCTTGCCCC | |
| | | | ACCCTCCCGACCCCAGTTCCCCTGACCCT | |
| | | | GCTAGTGGCATGTCTCCTCCCATGTCTGA | |
| | | | AGGTCCCTCGTCCTGTTTGTTTTAGGAAT | |
| | | | CCTGGTCTCAGGACCTCATGGAAGAAGA | |
| | | | GGGGGAGAAAGTTACCAGTTGGATATGA | |
| | | | TGCAGACTATGGGGCCCCAGCGACGTGT | |
| | | | CTGGTTGAGCTCAGGGAATATGGTTCTTA | |
| | | | GCCCAGTTTCTTGGTGATTTCCAGCGGTC | |
| | | | AGTTCAGGCAGGGCAAAGGCTTACTGAT | |
| | | | AAACTTGAGTCTGCCCTCGTATGAGGGTT | |
| | | | ATAGCTGGCCTCCCTCTGAGGCTGGTGAC | |
| | | | TCTTCCCTGCTGGGGCCCCACAGGTGAGA | |
| | | | CAGAACAGGTAGAGGGCCTCCCTGTCTG | |
| | | | CCCGCCTTGGCCAGCTAGCTTGCCTCTCC | |
| | | | TGTGCGTATGGGAACACCTAGCACGTGC | |
| | | | TGGGTGGGCTGCCTCTGACCCAGAGGCA | |
| | | | TGGCCGAATTTGGCGACTCAAAACCACC | |
| | | | TTGCCTCAGCTGATCAGAGTTTCTGTGGA | |
| | | | ATTCTGATTGTTAGATCAAATTAGCTGGC | |
| | | | CTCTGAATTAAGTGGGAGAGGACCTTCTC | |
| | | | TAAGATGAACCGGGTTCGCCCCAGTCCTC | |
| | | | CTGCCTGGAGACAGTTGATGTGTCTTGCA | |
| | | | GAGCTCTCGCTTCCCCAGCAACACTCTTC | |
| | | | AGTACATAATAAGCTTAACTGATAAACA | |
| | | | GAGAGAATATTTAGGAAGGTGAGTCTTG | |
| | | | GGCTTACCATTGGGTTTAAATCATAGGGA | |
| | | | CCTCGGGAAAGGGTTCGGGCTTCTCTGG | |
| | | | AGCAGATATTATGAAGTTCATGGCCTTAG | |
| | | | GTAGCATGTGTATCTGGTCTTAACTCTGA | |
| | | | TTGTAGCAAAAGTTCTGAGAGGAGCTGA | |
| | | | GCCTTGTTCTGGCCCCTTAAAGAACAGGG | |
| | | | TCCTCAGGCCCTGCCCGCTTCCTGTCCAC | |
| | | | TGCCCTCCTGCCCGTCCCCAGCCCAGCTG | |
| | | | AGGGAATCCCGTGGGTTGCTTACCTACCT | |
| | | | ATAAGGTGGTTTATAAGCTGCTGTCCTGG | |
| | | | CCACTGCATTCAAATTCCAATGTGTACTT | |
| | | | CATAGTGTAAAAATTTATATTATTGTGGG | |
| | | | GTTTTTTGTCTTTTTTTTTTTTTTTTTTTTG | |
| | | | GTATATTGCTGTATCTACTTTAACTTCCA | |
| | | | GAAATAAACGTTATATAGGAACCGTC | |
| Forward | | | TTGTGTTTGTGCCCAGAATG | 1219 |
| 1 | | | | |
| Reverse | | | TCCCTGAGTTGAATTATCAGCTT | 1220 |
| 1 | | | | |
| Probe 1 | | | /56- | 1221 |
| | | | FAM/ACGTCCCCA/ZEN/GAGTCTTTGTCA | |
| | | | ATGC/3IABKFQ/ | |
| Forward | | | GATGATTTCAGCAAATGACATGTTG | 1222 |
| 2 | | | | |
| Reverse | | | CAGTGAAAGCAGCAAAGAAGG | 1223 |
| 2 | | | | |
| Probe 2 | | | /56- | 1224 |
| | | | FAM/AGGACATCA/ZEN/GCGGTAAGACCC | |
| | | | AGA/3IABKFQ/ | |
| STAT3- | Modified | | [MePhosphonate-4O- | 1225 |
| 721 | 22 mer | | mUs][fAs][fU][fA][fG][mU][fU][mG][mA][fA] | |
| | | | [mA][mU][mC][fA][mA][mA][mG][mU][mC] | |
| | | | [mAs][mGs][mG] | |
| STAT3- | Modified | | [MePhosphonate-4O- | 1226 |
| 1286 | 22 mer | | mUs][fUs][fA][fA][fU][mU][fU][mU][mA][A] | |
| | | | [mG][mC][mU][fG][mA][mU][mA][mA][mU] | |
| | | | [mUs][mGs][mG] | |
| STAT3- | Modified | | [MePhosphonate-4O- | 1227 |
| 1287 | 22 mer | | mUs][fUs][fU][fA][fA][mU][fU][mU][mU][fA] | |
| | | | [mA][mG][mC][fU][mG][mA][mU][mA][mA] | |
| | | | [mUs][mGs][mG] | |
| STAT3- | Modified | | [MePhosphonate-4O- | 1228 |
| 1388 | 22 mer | | mUs][fAs][fUs][fU][fC][mU][fU][mC][mC][fA] | |
| | | | [mU][mG][mU][fU][mC][mA][mU][mC][mA] | |
| | | | [mCs][mGs][mG] | |
| NM_213659.3 | | | AATTATGCATGGAGGCGTGTCTTGGCCA | 1229 |
| Mus | | | GTGGCGGCTGGGTGGGGATTGGCTGGAG | |
| musculus | | | GGGCTGTAATTCAGCGGTTTCCGGAGCTG | |
| STAT3 | | | CAGTGTAGACAGGGAGGGGGAACCTGGG | |
| nucleotide | | | GTTCCGACGTCGCGGCGGAGGGAACGAG | |
| sequence | | | CCCTAACCGGATCGCTGAGGTACAACCC | |
| | | | CGCTCGGTGTCGCCTGACCGCGTCGGCTA | |
| | | | GGAGAGGCCAGGCGGCCCTCGGGAGCCC | |
| | | | AGCAGCTCGCGCCTGGAGTCAGCGCAGG | |
| | | | CCGGCCAGTCGGGCCTCAGCCCCGGAGA | |
| | | | CAGTCGAGACCCCTGACTGCAGCAGGAT | |
| | | | GGCTCAGTGGAACCAGCTGCAGCAGCTG | |
| | | | GACACACGCTACCTGGAGCAGCTGCACC | |
| | | | AGCTGTACAGCGACAGCTTCCCCATGGA | |
| | | | GCTGCGGCAGTTCCTGGCACCTTGGATTG | |
| | | | AGAGTCAAGACTGGGCATATGCAGCCAG | |
| | | | CAAAGAGTCACATGCCACGTTGGTGTTTC | |
| | | | ATAATCTCTTGGGTGAAATTGACCAGCA | |
| | | | ATATAGCCGATTCCTGCAAGAGTCCAAT | |
| | | | GTCCTCTATCAGCACAACCTTCGAAGAAT | |
| | | | CAAGCAGTTTCTGCAGAGCAGGTATCTTG | |
| | | | AGAAGCCAATGGAAATTGCCCGGATCGT | |
| | | | GGCCCGATGCCTGTGGGAAGAGTCTCGC | |
| | | | CTCCTCCAGACGGCAGCCACGGCAGCCC | |
| | | | AGCAAGGGGGCCAGGCCAACCACCCAAC | |
| | | | AGCCGCCGTAGTGACAGAGAAGCAGCAG | |
| | | | ATGTTGGAGCAGCATCTTCAGGATGTCCG | |
| | | | GAAGCGAGTGCAGGATCTAGAACAGAAA | |
| | | | ATGAAGGTGGTGGAGAACCTCCAGGACG | |
| | | | ACTTTGATTTCAACTACAAAACCCTCAAG | |
| | | | AGCCAAGGAGACATGCAGGATCTGAATG | |
| | | | GAAACAACCAGTCTGTGACCAGACAGAA | |
| | | | GATGCAGCAGCTGGAACAGATGCTCACA | |
| | | | GCCCTGGACCAGATGCGGAGAAGCATTG | |
| | | | TGAGTGAGCTGGCGGGGCTCTTGTCAGC | |
| | | | AATGGAGTACGTGCAGAAGACACTGACT | |
| | | | GATGAAGAGCTGGCTGACTGGAAGAGGC | |
| | | | GGCAGCAGATCGCGTGCATCGGAGGCCC | |
| | | | TCCCAACATCTGCCTGGACCGTCTGGAAA | |
| | | | ACTGGATAACTTCATTAGCAGAATCTCAA | |
| | | | CTTCAGACCCGCCAACAAATTAAGAAAC | |
| | | | TGGAGGAGCTGCAGCAGAAAGTGTCCTA | |
| | | | CAAGGGCGACCCTATCGTGCAGCACCGG | |
| | | | CCCATGCTGGAGGAGAGGATCGTGGAGC | |
| | | | TGTTCAGAAACTTAATGAAGAGTGCCTTC | |
| | | | GTGGTGGAGCGGCAGCCCTGCATGCCCA | |
| | | | TGCACCCGGACCGGCCCTTAGTCATCAA | |
| | | | GACTGGTGTCCAGTTTACCACGAAAGTC | |
| | | | AGGTTGCTGGTCAAATTTCCTGAGTTGAA | |
| | | | TTATCAGCTTAAAATTAAAGTGTGCATTG | |
| | | | ATAAAGACTCTGGGGATGTTGCTGCCCTC | |
| | | | AGAGGGTCTCGGAAATTTAACATTCTGG | |
| | | | GCACGAACACAAAAGTGATGAACATGGA | |
| | | | GGAGTCTAACAACGGCAGCCTGTCTGCA | |
| | | | GAGTTCAAGCACCTGACCCTTAGGGAGC | |
| | | | AGAGATGTGGGAATGGAGGCCGTGCCAA | |
| | | | TTGTGATGCCTCCTTGATCGTGACTGAGG | |
| | | | AGCTGCACCTGATCACCTTCGAGACTGA | |
| | | | GGTGTACCACCAAGGCCTCAAGATTGAC | |
| | | | CTAGAGACCCACTCCTTGCCAGTTGTGGT | |
| | | | GATCTCCAACATCTGTCAGATGCCAAATG | |
| | | | CTTGGGCATCAATCCTGTGGTATAACATG | |
| | | | CTGACCAATAACCCCAAGAACGTGAACT | |
| | | | TCTTCACTAAGCCGCCAATTGGAACCTGG | |
| | | | GACCAAGTGGCCGAGGTGCTCAGCTGGC | |
| | | | AGTTCTCGTCCACCACCAAGCGGGGGCT | |
| | | | GAGCATCGAGCAGCTGACAACGCTGGCT | |
| | | | GAGAAGCTCCTAGGGCCTGGTGTGAACT | |
| | | | ACTCAGGGTGTCAGATCACATGGGCTAA | |
| | | | ATTCTGCAAAGAAAACATGGCTGGCAAG | |
| | | | GGCTTCTCCTTCTGGGTCTGGCTAGACAA | |
| | | | TATCATCGACCTTGTGAAAAAGTATATCT | |
| | | | TGGCCCTTTGGAATGAAGGGTACATCAT | |
| | | | GGGTTTCATCAGCAAGGAGCGGGAGCGG | |
| | | | GCCATCCTAAGCACAAAGCCCCCGGGCA | |
| | | | CCTTCCTACTGCGCTTCAGCGAGAGCAGC | |
| | | | AAAGAAGGAGGGGTCACTTTCACTTGGG | |
| | | | TGGAAAAGGACATCAGTGGCAAGACCCA | |
| | | | GATCCAGTCTGTAGAGCCATACACCAAG | |
| | | | CAGCAGCTGAACAACATGTCATTTGCTG | |
| | | | AAATCATCATGGGCTATAAGATCATGGA | |
| | | | TGCGACCAACATCCTGGTGTCTCCACTTG | |
| | | | TCTACCTCTACCCCGACATTCCCAAGGAG | |
| | | | GAGGCATTTGGAAAGTACTGTAGGCCCG | |
| | | | AGAGCCAGGAGCACCCCGAAGCCGACCC | |
| | | | AGGTAGTGCTGCCCCGTACCTGAAGACC | |
| | | | AAGTTCATCTGTGTGACACCAACGACCTG | |
| | | | CAGCAATACCATTGACCTGCCGATGTCCC | |
| | | | CCCGCACTTTAGATTCATTGATGCAGTTT | |
| | | | GGAAATAACGGTGAAGGTGCTGAGCCCT | |
| | | | CAGCAGGAGGGCAGTTTGAGTCGCTCAC | |
| | | | GTTTGACATGGATCTGACCTCGGAGTGTG | |
| | | | CTACCTCCCCCATGTGAGGAGCTGAAAC | |
| | | | CAGAAGCTGCAGAGACGTGACTTGAGAC | |
| | | | ACCTGCCCCGTGCTCCACCCCTAAGCAGC | |
| | | | CGAACCCCATATCGTCTGAAACTCCTAAC | |
| | | | TTTGTGGTTCCAGATTTTTTTTTTTAATTT | |
| | | | CCTACTTCTGCTATCTTTGGGCAATCTGG | |
| | | | GCACTTTTTAAAATAGAGAAATGAGTGA | |
| | | | GTGTGGGTGATAAACTGTTATGTAAAGA | |
| | | | GGAGAGCACCTCTGAGTCTGGGGATGGG | |
| | | | GCTGAGAGCAGAAGGGAGCAAGGGGAA | |
| | | | CACCTCCTGTCCTGCCCGCCTGCCCTCCT | |
| | | | TTTTCAGCAGCTCGGGGTTGGTTGTTAGA | |
| | | | CAAGTGCCTCCTGGTGCCCATGGCATCCT | |
| | | | GTTGCCCCACTCTGTGAGCTGATACCCCA | |
| | | | GGCTGGGAACTCCTGGCTCTGCACTTTCA | |
| | | | ACCTTGCTAATATCCACATAGAAGCTAG | |
| | | | GACTAAGCCCAGAGGTTCCTCTTTAAATT | |
| | | | AAAAAAAAAAAAAATAAGAATTAAAGG | |
| | | | GCAAAACACACTGACACAGCATAGCCTT | |
| | | | TCCATATCAAGGAATACTCAGTTAACAG | |
| | | | CCTCTCCAGCGCTGTCTTCAGGCTGATCA | |
| | | | TCTATATAAACCCTGGAATGGTTGCAGAT | |
| | | | CAAATCTGTAAAAGAGATCCGAGAGCTG | |
| | | | TGGCTTGGCCTCTGGTTCAAACACAAAG | |
| | | | GCTAGAGAGAACCTAGATATCCCTGGGT | |
| | | | TTTGTTTACCCAGTATGCTTGTCGGTTGG | |
| | | | AGGTGTGAGGTAGGCCAAGGGCACTGGA | |
| | | | AAGCCTTTGTCATCACCCTACTCCCTCCC | |
| | | | CAACCCAGACTCCAGACCCTGTTTCAGG | |
| | | | GTCAGCCTGCCCTGTGGGTGCCTTACTGG | |
| | | | GCCTAGGGTCAACCTGCCTTCCTTTCCCA | |
| | | | CTTGACCTTGCTGGTAGTATGTCCCCTTC | |
| | | | CCATGTCCAAAGGCCCTCTGTCCTGCTTC | |
| | | | TATTGGGAATCCCTGCCTCAGGACCTTGT | |
| | | | GTCGAGAGGGATTGCCTTACAGGTTTGA | |
| | | | ACCTGCCTCAGACTACAGGCCCTCAGCA | |
| | | | AAGCTCAGGGAGTATGGTCCTTATTCTAT | |
| | | | GCGCTTGGTTCCCAGGGATATCTGTAACC | |
| | | | ACAGGGCAAAAGCTGACATATACTCCAG | |
| | | | GTCTGCCCTCATATGAGTGGTGTATTCTT | |
| | | | GGCCTCCCCTGAGACTGGCAACTGTCTGC | |
| | | | TCCCCATTGGGTCTCCCAGGTGAGGTGGA | |
| | | | ACACAGTTCCTGCACCTACTGTGGCCTCC | |
| | | | ATGTCGCTTGCTTGCTTCGCTCACTCAGC | |
| | | | TTACTGGAACACTGAGTGTTCAAGGCAA | |
| | | | GCCTTTCCTGACAGAGGCATGGCTAGATT | |
| | | | CAGTGACTCAAAGCCACCTCATTCAGCTG | |
| | | | ATCAGTGTCTGTGGAATTGTTTCCTTCCA | |
| | | | GTTAACCAGTGTCTGAATTAAGGGCAGT | |
| | | | GAGGACATTGTCTCCAAGACGAACTGCT | |
| | | | TGCCTTGACCACCCCAGCCTTCTGCTTCG | |
| | | | AGACAGTTACTGCTCTCCCACCCCATCAA | |
| | | | TGTTCTTTAGTTATACAATAAGCTGAACT | |
| | | | TATAAACTGAAAGGGTATTTAGGAAGGC | |
| | | | AAGGCTTGGGCATTTTTATGGCTTTCAAT | |
| | | | CCTGGGGACCCAGGAACAAGGTGAGGGC | |
| | | | TTCTCTGGGGCTGGTGTTGTACCTCAGGG | |
| | | | GCTCTGGGAAGTCTGTGTGCCTGGGTTAA | |
| | | | CCACCCATAGTGAGCCCCTGGAACTGCC | |
| | | | CACTTTCCCTCTCCTTGGCCCCACTTGGC | |
| | | | CCCAGCCTCACCCAGCCTGCAGACTGCTT | |
| | | | AGCCTTTCAGTGCAGTGGCTTGTGTTCTG | |
| | | | GCCACTGCACTCAGATTCCAATGTAAACT | |
| | | | TTCTAGTGTAAAAATTTATATTATTGTGG | |
| | | | GTTGTTTTTTGTTGTTGTTTGTTTTTGTAT | |
| | | | ATTGCTGTAACTACTTTAACTTCCAGAAA | |
| | | | TAAAGATTATATAGGAACTGTCTGGC | |

1.-183. (canceled)

184. An oligonucleotide for reducing STAT3 expression, comprising an antisense strand comprising the nucleotide sequence of SEQ ID NO: 965 and a sense strand comprising the nucleotide sequence of SEQ ID NO: 875, wherein the sense strand comprises a saturated C18 hydrocarbon chain conjugated to the 5′ terminal nucleotide of the sense strand, and wherein each of the antisense and sense strands comprise at least one 2′-modified nucleotide and at least one modified internucleotide linkage.

185. The oligonucleotide of claim 184 comprising an antisense strand comprising the nucleotide sequence of SEQ ID NO: 1145 and a sense strand comprising the nucleotide sequence of SEQ ID NO: 1055.

186. The oligonucleotide of claim 184, wherein the sense strand comprises at its 3′ end a stem-loop set forth as: S1-L-S2, wherein 51 is complementary to S2, and wherein L forms a loop between 51 and S2 of 3 to 5 nucleotides in length.

187. The oligonucleotide of claim 186, wherein L is a tetraloop, optionally wherein L is 4 nucleotides in length.

188. The oligonucleotide of claim 186, wherein L comprises a sequence set forth as GAAA.

189. The oligonucleotide of claim 184, wherein the 2′-modification comprises 2′-aminoethyl, 2′-fluoro, 2′-O-methyl, 2′-O-methoxyethyl, or 2′-deoxy-2′-fluoro-β-d-arabinonucleic acid.

190. The oligonucleotide of claim 184, wherein the sense strand comprises 36 nucleotides with positions 1-36 from 5′ to 3′ and wherein:
a) one or more positions 8-11 comprise 2′-fluoro modification, preferably all positions 8-11 comprise 2′-fluoro modification; and/or
b) one or more positions 1-7, 12-36 comprise 2′-O-methyl modification, preferably all positions 1-7, 12-36 comprise 2′-O-methyl modification.

191. The oligonucleotide of claim 184, wherein the antisense strand comprises 22 nucleotides with positions 1-22 from 5′ to 3′, and wherein:
a) one or more positions 2, 3, 4, 5, 7, 10 and 14 comprise 2′-fluoro modification, preferably all positions 2, 3, 4, 5, 7, 10 and 14 comprise 2′-fluoro modification; and/or
b) one or more positions 1, 6, 8, 9, 11-13, 15-22 comprise 2′-O-methyl modification, preferably all positions 1, 6, 8, 9, 11-13, 15-22 comprise 2′-O-methyl modification.

192. The oligonucleotide of claim 184, wherein a 4′-carbon of the sugar of the 5′-nucleotide of the antisense strand comprises a phosphate analog.

193. The oligonucleotide of claim 184, wherein at least one nucleotide of the oligonucleotide is conjugated to one or more targeting ligands, optionally, wherein the one or more targeting ligands is a saturated or unsaturated fatty acid moiety.

194. An oligonucleotide for reducing STAT3 expression, the oligonucleotide comprising an antisense of SEQ ID NO: 1145 and further comprising a sense strand of SEQ ID NO: 1055, wherein:
the antisense strand comprises
[MePhosphonate-4O-mUs][fUs][fAs][fA][fU][mU][fU][mU][mA][fA][mG][mC][mU][fG][mA][mU][mA][m A][mU][mUs][mGs][mG]
and the sense strand comprises
[mAs][mA][mU][mU][mA][mU][mC][fA][fG][fC][fU][mU][mA][mA][mA][mA][mU][mU][mA][mA][mG][mC][mA][mG][mC][mC][mG][ademA-GalNAc][ademA-GalNAc][ademA-GalNAc][mG][mG][mC][mU][mG][mC].

195. A pharmaceutical composition comprising the oligonucleotide or oligonucleotide-ligand conjugate of claim 184 and a pharmaceutically acceptable carrier, delivery agent or excipient.

196. A composition for use in treating a disorder or condition associated with STAT3 expression in a patient in need thereof, wherein the composition comprises the oligonucleotide or oligonucleotide-ligand conjugate of claim 184.

197. The composition of claim 196, wherein the disorder or condition associated with STAT3 expression is cancer.

198. The composition of claim 197, wherein the cancer comprises a carcinoma, sarcoma, melanoma, lymphoma, and leukemia, prostate cancer, breast cancer, hepatocellular carcinoma (HCC), colorectal cancer, pancreatic cancer or a glioblastoma.

199. A method of treating a subject having a disease, disorder or condition associated with STAT3 expression, the method comprising administering to the subject a therapeutically effective amount of the oligonucleotide or oligonucleotide-ligand conjugate of claim 184.

200. The method of claim 199, wherein the disorder or condition is cancer.

201. The method of claim 199, wherein the disorder or condition is carcinoma, sarcoma, melanoma, lymphoma, and leukemia, prostate cancer, breast cancer, hepatocellular carcinoma (HCC), colorectal cancer, pancreatic cancer or glioblastoma.

202. A kit comprising the oligonucleotide or oligonucleotide-ligand conjugate of claim 184, an optional pharmaceutically acceptable carrier, and a package insert comprising instructions for administration to a subject having a disease, disorder or condition associated with STAT3 expression.
